(19) United States
(12) Patent Application Publication
(10) Pub. No.: US 2008/0242663 A1
(43) Pub. Date: Oct. 2, 2008

(54) NOVEL PYRIMIDINE DERIVATIVES 698

(75) Inventors: Susan Elizabeth Ashton, Macclesfield (GB); Darren Anthony Edward Cross, Macclesfield (GB); Simon John East, Macclesfield (GB); Jason Grant Kettle, Macclesfield (GB); Mark Andrew Pearson, Macclesfield (GB); Stephen Robert Wedge, Macclesfield (GB); Bernard Christophe Barlaam, Reims (FR); Richard Ducray, Reims (FR); Stuart Charles Purkiss, Macclesfield (GB)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 12/039,030

(22) Filed: Feb. 28, 2008

(30) Foreign Application Priority Data
Feb. 28, 2007 (EP) 07300832.8
Feb. 28, 2007 (EP) 07300833.6
Apr. 18, 2007 (EP) 07300960.7
Jul. 24, 2007 (EP) 07301269.2
Jul. 24, 2007 (EP) 07301270.0

(51) Int. Cl. A61K 31/5377; C07D 413/14; A61P 35/04

(52) U.S. Cl. 514/232.2; 544/122

(57) ABSTRACT

The invention concerns compounds of Formula I, or a pharmaceutically acceptable salt thereof,

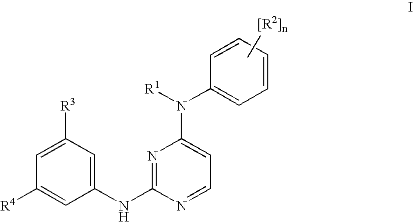

where R¹, n, R², R³, and R⁴ are as defined in the description. The present invention also relates to processes for the preparation of such compounds, pharmaceutical compositions containing them and their use in the manufacture of a medicament for use as an antiproliferative agent in the prevention or treatment of tumours or other proliferative conditions which are sensitive to the inhibition of EphB4 kinases.

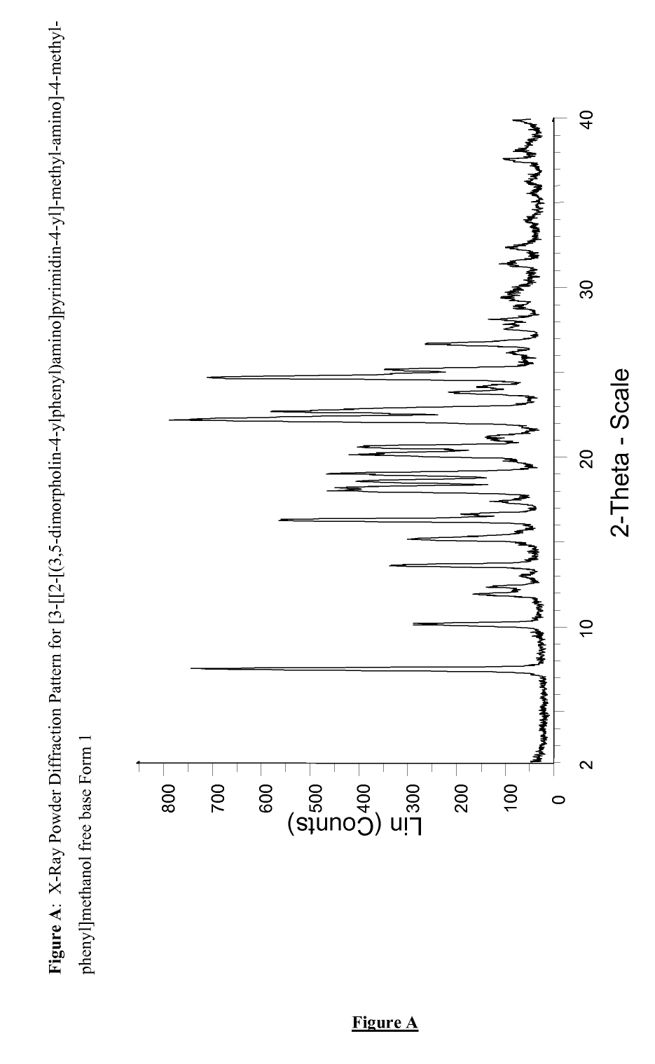

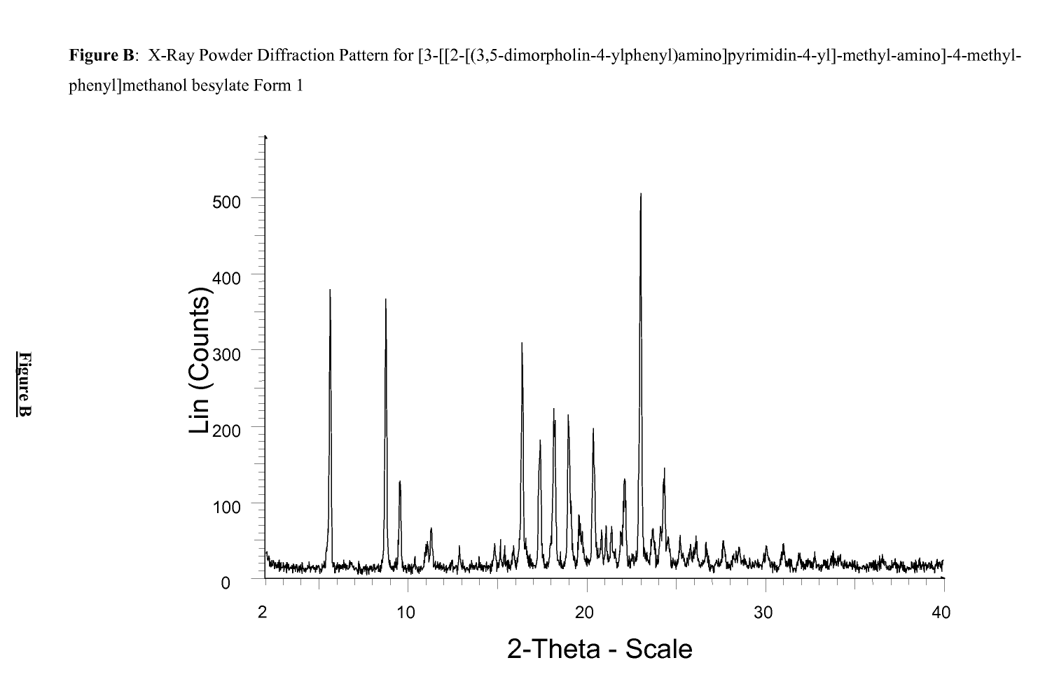

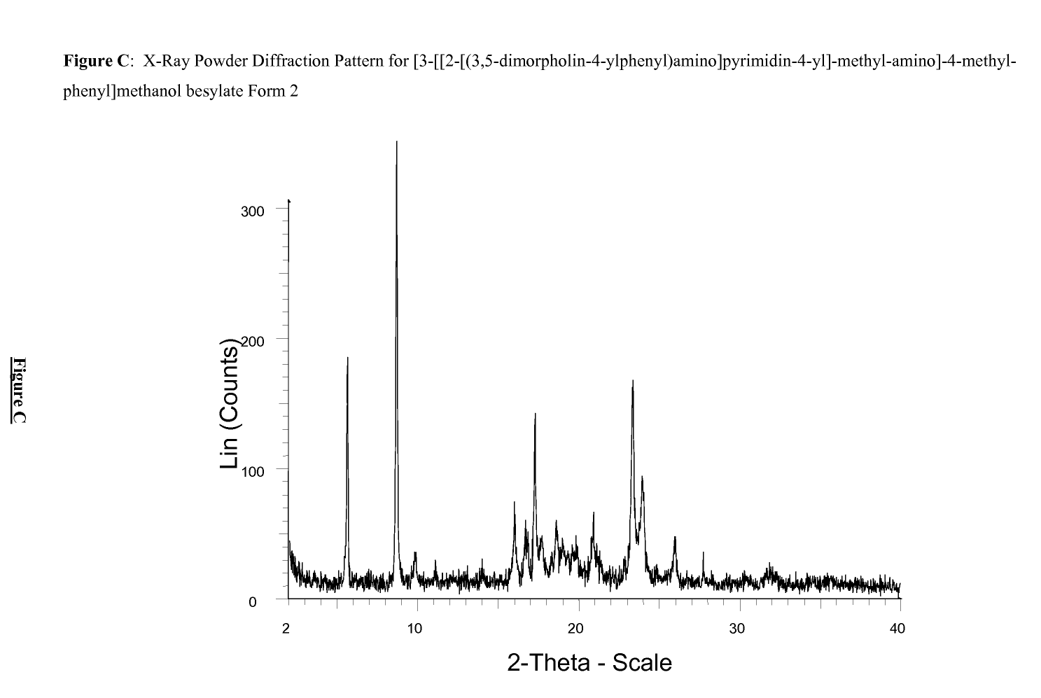

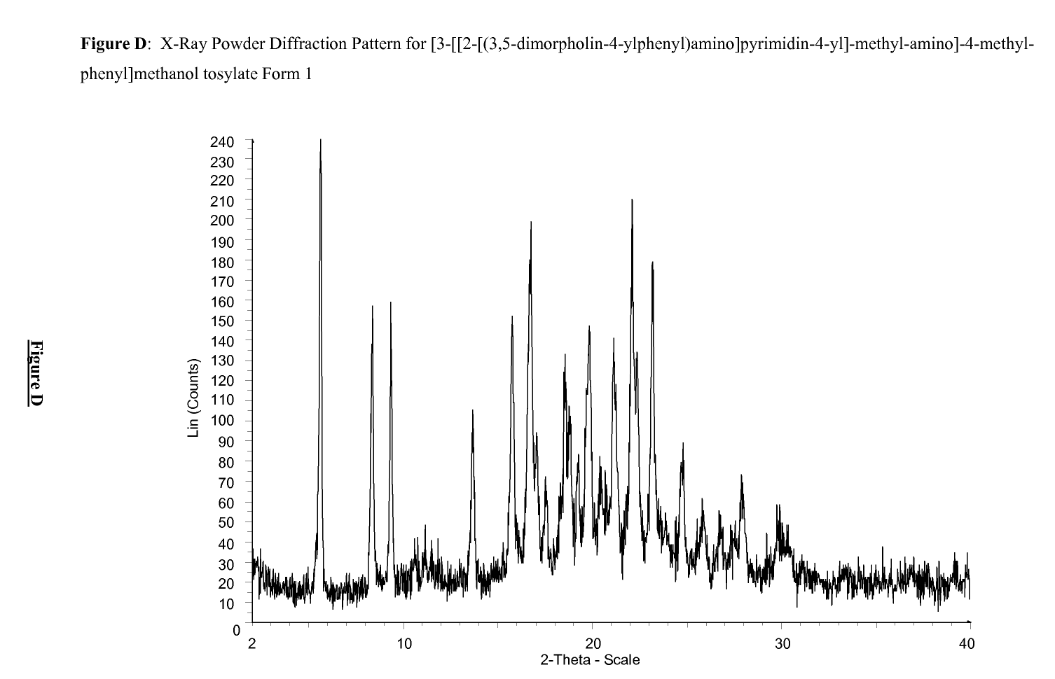

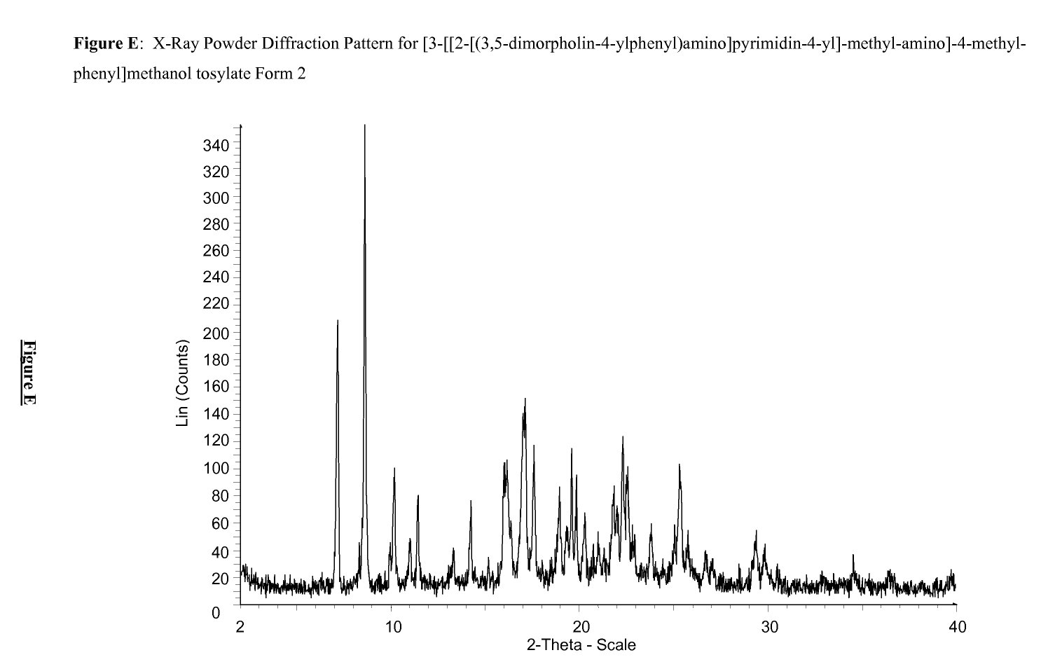

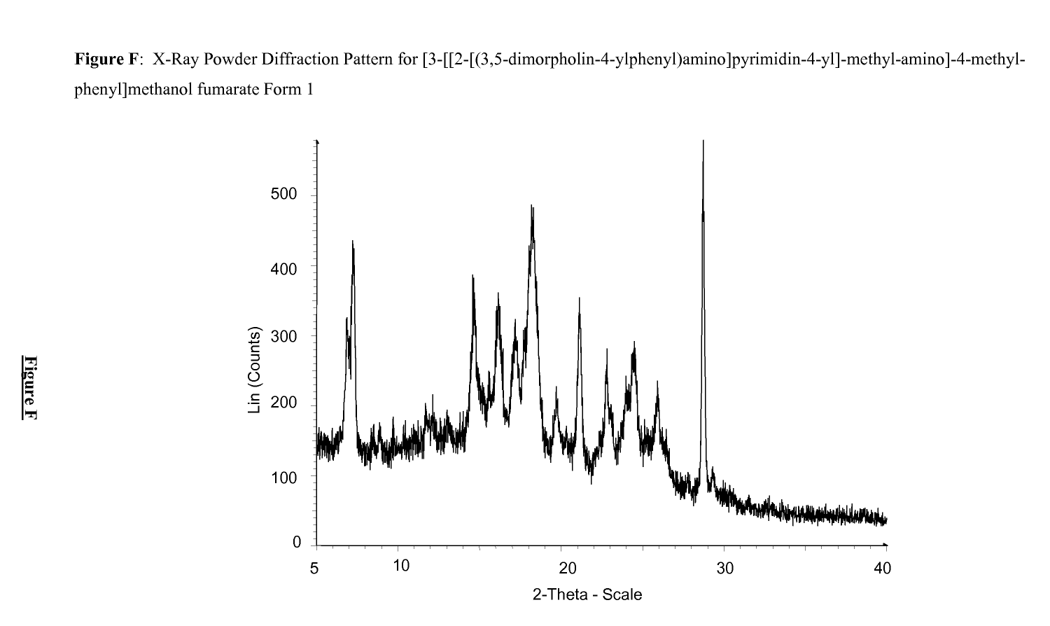

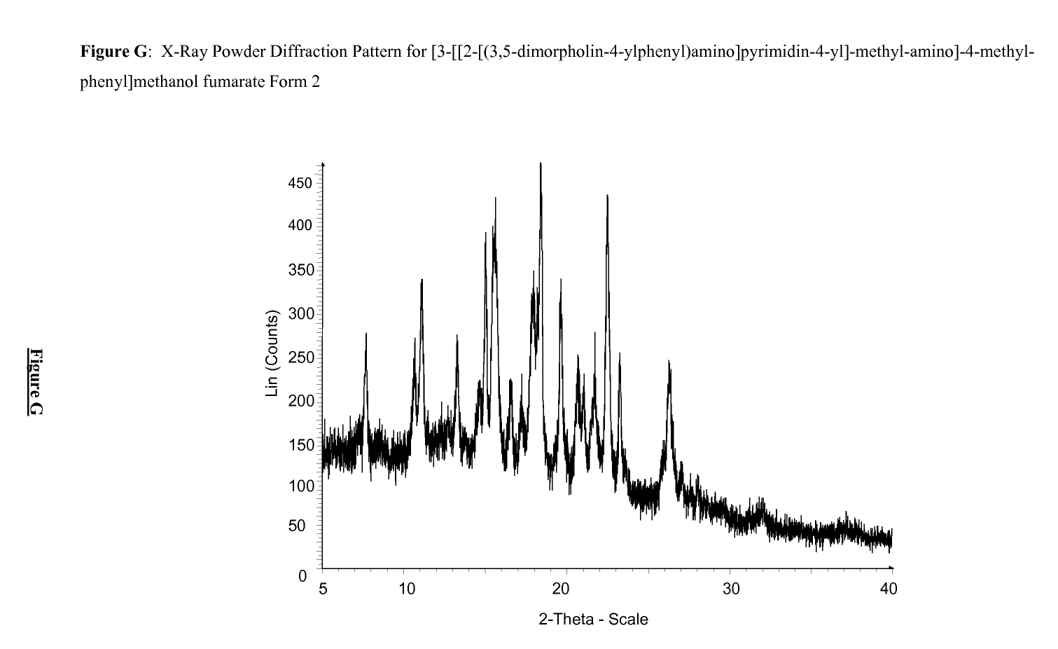

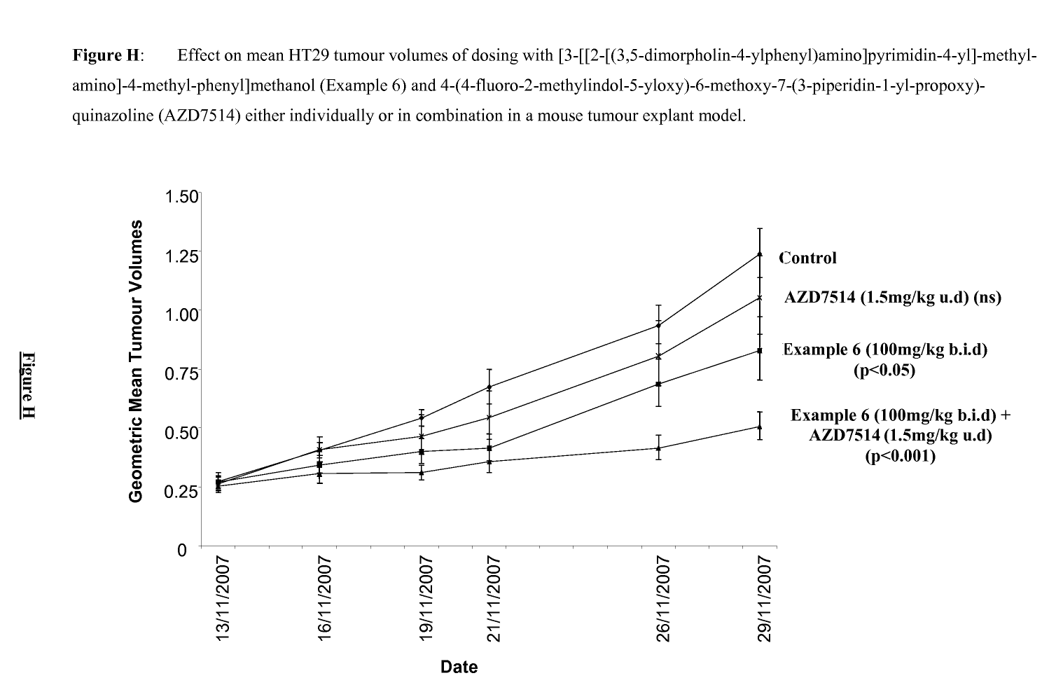

This application claims the benefit under 35 U.S.C. § 119(a)-(d) of Application No. 07300832.8 (EP sort 1) filed on 28 Feb. 2007, Application No. 07301269.2 (EP sort 2) filed on 24 Jul. 2007, Application No. 07300833.6 (EP sort 1) filed on 28 Feb. 2007, Application No. 07300960.7 (EP sort 2) filed 18 Apr. 2007 and Application No. 07301270.0 (EP sort 3) filed on 24 Jul. 2007.

### BACKGROUND OF THE INVENTION

The present invention relates to novel pyrimidine derivatives, to pharmaceutical compositions containing these derivatives and to their use in therapy, in particular in the prevention and treatment of cancer, in a warm blooded animal such as man.

Many of the current treatment regimes for cell proliferation diseases such as psoriasis and cancer utilise compounds which inhibit DNA synthesis. Such compounds are generally toxic to all cells, but their toxic effects on rapidly dividing cells, such as tumour cells, can be beneficial.

In recent years it has been discovered that a cell may become cancerous by virtue of the transformation of a portion of its DNA into an oncogene i.e. a gene which, on activation, leads to the formation of malignant tumour cells (Bradshaw, Mutagenesis, 1986, 1, 91). Several such oncogenes give rise to the production of peptides which are receptors for growth factors. Activation of the growth factor receptor results in an increase in cell proliferation. It is known, for example, that several oncogenes encode tyrosine kinase enzymes and that certain growth factor receptors are also tyrosine kinase enzymes (Yarden et al., Ann. Rev. Biochem. 1988, 57, 443; Larsen et al., Ann. Reports in Med. Chem., 1989, Chpt. 13).

Receptor tyrosine kinases play an important role in the transmission of biochemical signals, which initiate a variety of cell responses—including cell proliferation, survival and migration. They are large enzymes which span the cell membrane and possess an extracellular binding domain for growth factors, such as epidermal growth factor (EGF), and an intracellular portion which functions as a kinase to phosphorylate tyrosine amino acids in proteins and thereby influence cell proliferation. A large number of receptor tyrosine kinases are known (Wilks, Advances in Cancer Research, 1993, 60 43-73) and are classified on the basis of the family of growth factors that bind to the extracellular domain. This classification includes Class I receptor tyrosine kinases comprising the EGF family of receptor tyrosine kinases such as the EGF, TGFα, Neu and erbB receptors, Class II receptor tyrosine kinases comprising the insulin family of receptor tyrosine kinases such as the insulin and IGF1 receptors and insulin-related receptor (IRR), and Class III receptor tyrosine kinases comprising the platelet-derived growth factor (PDGF) family of receptor tyrosine kinases such as the PDGFα, PDGFβ and colony-stimulating factor 1 (CSF1) receptors.

The Eph family is the largest known family of receptor tyrosine kinases, with 14 receptors and 8 cognate ephrin ligands identified in mammals (reviewed in Kullander and Klein, Nature Reviews Molecular Cell Biology, 2002, 3, 475-486). The receptor family is further sub-divided into two sub-families defined largely by homology of extracellular domains and affinity towards a particular ligand type. In general, all Eph receptors contain an intracellular tyrosine kinase domain and an extracellular Ig-like domain with a cysteine-rich region with 19 conserved cysteines and two fibronectin type III domains. The A-class of Eph receptors consists of 8 receptors termed EphA1-8, which generally bind to their cognate ephrinA class of ligands termed ephrinA1-5. The B-class consists of 6 receptors termed EphB1-6, which bind to their cognate ephrinB ligands termed ephrinB1-3. Eph receptor ligands are unusual and differ to most other receptor tyrosine kinase ligands in that they are also tethered to cells, via a glycosylphosphatidylinositol linker in ephrinA ligands or an integral transmembrane region in ephrinB ligands. The binding of ephrin ligand to the Eph receptor induces a conformational change within the Eph intracellular domain that enables phosphorylation of tyrosine residues within an auto-inhibitory juxtamembrane region, which relieves this inhibition of catalytic site and enables additional phosphorylation to stabilise the active conformation and generate more docking sites for downstream signalling effectors.

Furthermore, evidence indicates that Eph/ephrin signalling can regulate other cell responses, such as proliferation and survival.

There is growing evidence that Eph receptor signalling may contribute to tumourigenesis in a wide variety of human cancers, either on tumour cells directly or indirectly via modulation of vascularisation. For instance, many Eph receptors are over-expressed in various tumour types (Reviewed in Surawska et al., Cytokine & Growth Factor Reviews, 2004, 15, 419-433, Nakamoto and Bergemann, Microscopy Res and Technique, 2002, 59, 58-67). The expression of EphB receptors, including EphB4, is up-regulated in tumours such as neuroblastomas, leukemias, breast, liver, lung and colon. Furthermore, various in vitro and in vivo studies particularly relating to EphB4 have indicated that over-expression of Eph receptors on cancer cells is able to confer tumourigenic phenotypes such as proliferation and invasion, consistent with the speculated role in oncogenesis.

For instance, inhibition of EphB4 expression using interfering-RNA or antisense oligodeoxynucleotides inhibited proliferation, survival and invasion of PC3 prostate cancer cells in vitro and in vivo xenograft model (Xia et al., Cancer Res., 2005, 65, 4623-4632.

In addition to a compelling role for Eph receptors on tumour cells, there is good evidence that EphB4 may contribute to tumour vascularisation (Reviewed in Brantley-Sieders et al., Current Pharmaceutical Design, 2004, 10, 3431-3442, Cheng et al., Cytokine and Growth Factor Reviews, 2002, 13, 75-85). Members of the Eph family including EphB4 are expressed on endothelial cells. Transgenic studies have shown that disruption of EphB4 (Gerety et al., Molecular Cell, 1999, 4, 403-414) or its ligand ephrinB2 (Wang et al., Cell, 1998, 93, 741-753) causes embryonic lethality associated with vascular modelling defects consistent with a critical role in vessel development. EphB4 activation stimulates endothelial cell proliferation and migration in vitro (Steinle et al., J. Biol. Chem., 2002, 277, 43830-43835).

Moreover, inhibition of EphB4 signalling using soluble extracellular-domains of EphB4 have been shown to inhibit tumour growth and angiogenesis in in vivo xenograft studies (Martiny-Baron et al., Neoplasia, 2004, 6, 248-257, Kertesz et al., Blood, 2005, Pre-published online).

Accordingly it has been recognised that an inhibitor of Eph receptors, particularly EphB4, should be of value as a selective inhibitor of the proliferation and survival of tumour cells by either targeting the tumour cells directly or via their effects on tumour vascularisation. Thus, such inhibitors should be valuable therapeutic agents for the containment and/or treatment of tumour disease.

The applicants have found that certain pyrimidine compounds are useful in the inhibition of EphB4 and therefore may be useful in therapy for the treatment of disease states in which increased EphB4 activity is implicated.

### GENERAL DESCRIPTION OF THE INVENTION

According to a first aspect of the invention, there is provided a compound of formula I

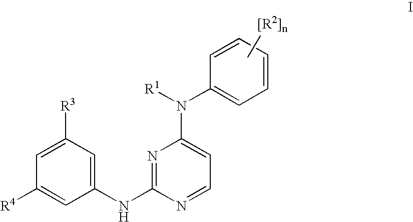

wherein:
R¹ is a (1-4C)alkyl, (3-4C)cycloalkyl or cyclopropylmethyl group which is optionally substituted by one or more substituent groups selected from —OR⁵ (wherein R⁵ is selected from hydrogen or (1-2C)alkyl), cyano, halo, or —NR⁶R⁷ (where R⁶ and R⁷ are independently selected from hydrogen, (1-2C)alkyl or (1-2C)alkanoyl);
n is 0, 1, 2 or 3;
each R² group present is independently selected from (1-2C)alkyl, (1-2C)alkoxy, fluoro, chloro, cyano, hydroxy(1-2C)alkyl, or a group of sub-formula:

-Q-R⁸

where Q is selected from —CO—, —NR^{a}—, —NR^{a}CO—, —NR^{a}—COO—, —NR^{a}CONR^{b}, —CONR^{a}—, —S(O)_{z}— (where z is 0, 1 or 2); —SO₂NR^{a}—, and —NR^{a}SO₂—, R^{a} and R^{b} are each independently selected from hydrogen or methyl, and R⁸ is hydrogen or (1-2C)alkyl;
R³ is selected from:
(i) hydrogen, halo, nitro, cyano, or hydroxy;
(ii) an optionally substituted (1-6C)alkyl, (2-6C)alkenyl, or (2-6C)alkynyl group wherein the optional substituents are selected from:
   cyano;
   halo;
   a group of sub-formula:

—W—R⁹
wherein W is selected from —O—, —S(O)ₚ— (where p is 0, 1 or 2), —CO—, —NR^{b}CO—, —CONR^{b}—, —NR^{b}CONR^{b}—, —SO₂NR^{b}—, —NR^{b}SO₂—, or —NR^{b}COO—;
R^{b} is selected from hydrogen or (1-2C)alkyl;
and R⁹ is selected from hydrogen or (1-4C)alkyl;
or —NR¹⁰R¹¹, where R¹⁰ and R¹¹ are independently selected from hydrogen, or a (1-4C)alkyl, (3-6C)cycloalkyl or (3-6C)cycloalkyl(1-2C)alkyl group which is optionally substituted by halo, hydroxy, cyano, or (1-4C)alkoxy, or R¹⁰ and R¹¹ are linked to form a 4, 5, 6 or 7 membered heterocyclic ring which optionally comprises, in addition to the nitrogen atom to which R¹⁰ and R¹¹ are attached, one or two further heteroatoms selected from O, N or S, and wherein any S atoms that are present may be optionally oxidised to form an SO and SO₂ group, and wherein any carbon atom present in the ring is optionally substituted by oxo, halo, hydroxy, cyano, (1-4C)alkyl, hydroxy(1-4C)alkyl, (1-4C)alkoxy, (1-2C)alkoxy-(1-4C)alkyl, (1-4C)alkanoyl, (1-4C)alkanesulfonyl, (1-4C)alkoxycarbonyl, (1-6C)alkylaminocarbonyl or di-(1-6C)alkylaminocarbonyl and any available nitrogen atom present in the ring is optionally substituted by (1-4C)alkyl, hydroxy(1-4C)alkyl, (1-2C)alkoxy-(1-4C)alkyl, or (1-4C)alkanoyl;
(iii) a group —NR¹²R¹³, wherein R¹² and R¹³ are each independently selected from hydrogen or a (1-6C)alkyl (3-6C)cycloalkyl or (3-6C)cycloalkyl(1-2C)alkyl group which is optionally substituted by halo, hydroxy, cyano, or (1-4C)alkoxy, or R¹² and R¹³ are linked to form a 4, 5, 6 or 7-membered heterocyclic ring which optionally comprises, in addition to the nitrogen atom to which R¹² and R¹³ are attached, one or two further heteroatoms selected from O, N or S, and wherein any S atoms that are present may be optionally oxidised to form an SO and SO₂ group, and wherein any carbon atom present in the ring is optionally substituted by oxo, halo, hydroxy, cyano, (1-4C)alkyl, hydroxy(1-4C)alkyl, (1-4C)alkoxy, (1-2C)alkoxy-(1-4C)alkyl, (1-4C)alkanoyl, (1-4C)alkanesulfonyl, (1-4C)alkoxycarbonyl, (1-6C)alkylaminocarbonyl or di-(1-6C)alkylaminocarbonyl and any available nitrogen atom present in the ring is optionally substituted by (1-4C)alkyl, hydroxy(1-4C)alkyl, (1-2C)alkoxy-(1-4C)alkyl, or (1-4C)alkanoyl; or
(iv) a group of formula (II):

—X—R¹⁴
wherein X is selected from —O—, —S(O)ₚ— (where p is 0, 1 or 2), —CO—, —NR^{c}CO—, —CONR^{c}—, —NR^{c}COO—, and —NR^{c}SO₂—,
where R^{c} is selected hydrogen or (1-2C)alkyl;
R¹⁴ is a (1-4C)alkyl, (3-6C)cycloalkyl, (3-6C)cycloalkyl(1-2C)alkyl, oxanyl or oxolanyl group which is optionally substituted by halo, hydroxy, cyano, or (1-4C)alkoxy, or R¹⁴ is

—NR¹⁵R¹⁶
where R¹⁵ and R¹⁶ are independently selected from hydrogen, (1-2C)alkanoyl or (1-2C)alkyl, or R¹⁵ and R¹⁶ are linked to form a 4, 5, 6 or 7-membered heterocyclic ring which optionally comprises, in addition to the nitrogen atom to which R¹⁵ and R¹⁶ are attached, one or two further heteroatoms selected from O, N or S, and wherein any S atoms that are present may be optionally oxidised to form an SO and SO₂ group, and wherein any carbon atom present in the ring is optionally substituted by oxo, halo, hydroxy, cyano, (1-4C)alkyl, hydroxy(1-4C)alkyl, (1-4C)alkoxy, (1-2C)alkoxy-(1-4C)alkyl, (1-4C)alkanoyl, (1-4C)alkanesulfonyl, (1-4C)alkoxycarbonyl, (1-6C)alkylaminocarbonyl or di-(1-6C)alkylaminocarbonyl and any available nitrogen atom is optionally substituted by (1-4C)alkyl, hydroxy(1-4C)alkyl, (1-2C)alkoxy-(1-4C)alkyl, or (1-4C)alkanoyl;
   R⁴ is a group —NR¹⁷R¹⁸, wherein R¹⁷ and R¹⁸ are linked to form a 4, 5, 6 or 7 membered heterocyclic ring which optionally comprises, in addition to the nitrogen atom to which R¹⁷ and R¹⁸ are attached, one or two further heteroatoms selected from O, N or S, and wherein any S atoms that are present may be optionally oxidised to form an SO or SO₂ group, and wherein any carbon atom present in the ring is optionally substituted by oxo, halo, hydroxy, cyano, (1-4C)alkyl, hydroxy(1-4C)alkyl, (1-4C)alkoxy, (1-2C)alkoxy-(1-4C)alkyl, (1-4C)alkanoyl, (1-4C)alkanesulfonyl, (1-4C)alkoxycarbonyl, (1-6C)alkylaminocarbonyl or di-(1-6C)alkylaminocarbonyl and any available nitrogen atom present in the ring is optionally substituted by (1-4C)alkyl, hydroxy(1-4C)alkyl, (1-2C)alkoxy-(1-4C)alkyl, or (1-4C)alkanoyl; subject to the following provisos:
when n is 1 and R² is (1-2C)alkoxy, the alkoxy group is not located in the para or 4-position relative to the —NR¹— group:
when n is 1 and R² is ethoxy, the ethoxy group is not located in the meta or 3-position relative to the —NR¹— group;
R⁴ is not a 4-methylpiperazin-1-yl group when R² is a group of sub-formula -Q-R⁸, in which Q is —NR^{a}—CO—, R^{a} is hydrogen, and R⁸ is (1-2C)alkyl;
   or a pharmaceutically acceptable salt thereof.

According to a second aspect of the invention, there is provided a compound of formula I above wherein:

R¹ is a (1-4C)alkyl, (3-4C)cycloalkyl or cyclopropylmethyl group which is optionally substituted by one or more substituent groups selected from —OR⁵ (wherein R⁵ is selected from hydrogen or (1-2C)alkyl), cyano, halo, or —NR⁶R⁷ (where R⁶ and R⁷ are independently selected from hydrogen, (1-2C)alkyl or (1-2C)alkanoyl);
n is 0, 1, 2 or 3;
each R² group present is independently selected from (1-2C)alkyl, (1-2C)alkoxy, fluoro, chloro, cyano, hydroxy(1-2C)alkyl, or a group of sub-formula:

-Q-R⁸

where Q is selected from —CO—, —NR^{a}—, —NR^{a}—CO—, —NR^{a}—COO—, NR^{a}CONR^{b}, —CONR^{a}—, —S(O)_{z}— (where z is 0, 1 or 2); —SO₂NR^{a}—, and —NR^{a}SO₂—, R^{a} and R^{b} are each independently selected from hydrogen or methyl, and R⁸ is hydrogen or (1-2C)alkyl;
R³ is selected from:
(v) hydrogen, halo, nitro, cyano, or hydroxy;
(vi) an optionally substituted (1-6C)alkyl, (2-6C)alkenyl, or (2-6C)alkynyl group wherein the optional substituents are selected from:
   cyano;
   halo;
   a group of sub-formula:

—W—R⁹
wherein W is selected from —O—, —S(O)ₚ— (where p is 0, 1 or 2), —CO—, —NR^{b}CO—, —CONR^{b}—, —NR^{b}CONR^{b}—, —SO₂NR^{b}—, —NR^{b}SO₂—, or —NR^{b}COO—;
R^{b} is selected from hydrogen or (1-2C)alkyl;
and R⁹ is selected from hydrogen or (1-4C)alkyl;
or —NR¹⁰R¹¹, where R¹⁰ and R¹¹ are independently selected from hydrogen, or a (1-4C)alkyl, (3-6C)cycloalkyl or (3-6C)cycloalkyl(1-2C)alkyl group which is optionally substituted by halo, hydroxy, cyano, or (1-4C)alkoxy, or R¹⁰ and R¹¹ are linked to form a 4, 5, 6 or 7 membered heterocyclic ring which optionally comprises, in addition to the nitrogen atom to which R¹⁰ and R¹¹ are attached, one or two further heteroatoms selected from O, N or S, and wherein any S atoms that are present may be optionally oxidised to form an SO and SO₂ group, and wherein any carbon atom present in the ring is optionally substituted by oxo, halo, hydroxy, cyano, (1-4C)alkyl, hydroxy(1-4C)alkyl, (1-4C)alkoxy, (1-2C)alkoxy-(1-4C)alkyl, (1-4C)alkanoyl, (1-4C)alkanesulfonyl, (1-4C)alkoxycarbonyl, (1-6C)alkylaminocarbonyl or di-(1-6C)alkylaminocarbonyl and any available nitrogen atom present in the ring is optionally substituted by (1-4C)alkyl, hydroxy(1-4C)alkyl, (1-2C)alkoxy-(1-4C)alkyl, or (1-4C)alkanoyl;
(vii) a group —NR¹²R¹³, wherein R¹² and R¹³ are each independently selected from hydrogen or a (1-6C)alkyl (3-6C)cycloalkyl or (3-6C)cycloalkyl(1-2C)alkyl group which is optionally substituted by halo, hydroxy, cyano, or (1-4C)alkoxy, or R¹² and R¹³ are linked to form a 4, 5, 6 or 7-membered heterocyclic ring which optionally comprises, in addition to the nitrogen atom to which R¹² and R¹³ are attached, one or two further heteroatoms selected from O, N or S, and wherein any S atoms that are present may be optionally oxidised to form an SO and SO₂ group, and wherein any carbon atom present in the ring is optionally substituted by oxo, halo, hydroxy, cyano, (1-4C)alkyl, hydroxy(1-4C)alkyl, (1-4C)alkoxy, (1-2C)alkoxy-(1-4C)alkyl, (1-4C)alkanoyl, (1-4C)alkanesulfonyl, (1-4C)alkoxycarbonyl, (1-6C)alkylaminocarbonyl or di-(1-6C)alkylaminocarbonyl and any available nitrogen atom present in the ring is optionally substituted by (1-4C)alkyl, hydroxy(1-4C)alkyl, (1-2C)alkoxy-(1-4C)alkyl, or (1-4C)alkanoyl; or
(viii) a group of formula (II):

—X—R¹⁴
wherein X is selected from —O—, —S(O)ₚ— (where p is 0, 1 or 2), —CO—, —NR^{c}CO—, —CONR^{c}—, —NR^{c}COO—, and —NR^{c}SO₂—,
where R^{c} is selected hydrogen or (1-2C)alkyl;
R¹⁴ is a (1-4C)alkyl, (3-6C)cycloalkyl or (3-6C)cycloalkyl(1-2C)alkyl group which is optionally substituted by halo, hydroxy, cyano, or (1-4C)alkoxy, or R¹⁴ is

—NR¹⁵R¹⁶
where R¹⁵ and R¹⁶ are independently selected from hydrogen, (1-2C)alkanoyl or (1-2C)alkyl, or R¹⁵ and R¹⁶ are linked to form a 4, 5, 6 or 7-membered heterocyclic ring which optionally comprises, in addition to the nitrogen atom to which R¹⁵ and R¹⁶ are attached, one or two further heteroatoms selected from O, N or S, and wherein any S atoms that are present may be optionally oxidised to form an SO and SO₂ group, and wherein any carbon atom present in the ring is optionally substituted by oxo, halo, hydroxy, cyano, (1-4C)alkyl, hydroxy(1-4C)alkyl, (1-4C)alkoxy, (1-2C)alkoxy-(1-4C)alkyl, (1-4C)alkanoyl, (1-4C)alkanesulfonyl, (1-4C)alkoxycarbonyl, (1-6C)alkylaminocarbonyl or di-(1-6C)alkylaminocarbonyl and any available nitrogen atom is optionally substituted by (1-4C)alkyl, hydroxy(1-4C)alkyl, (1-2C)alkoxy-(1-4C)alkyl, or (1-4C)alkanoyl;
   R⁴ is a group —NR¹⁷R¹⁸, wherein R¹⁷ and R¹⁸ are linked to form a 4, 5, 6 or 7 membered heterocyclic ring which optionally comprises, in addition to the nitrogen atom to which R¹⁷ and R¹⁸ are attached, one or two further heteroatoms selected from O, N or S, and wherein any S atoms that are present may be optionally oxidised to form an SO or SO₂ group, and wherein any carbon atom present in the ring is optionally substituted by oxo, halo, hydroxy, cyano, (1-4C)alkyl, hydroxy(1-4C)alkyl, (1-4C)alkoxy, (1-2C)alkoxy-(1-4C)alkyl, (1-4C)alkanoyl, (1-4C)alkanesulfonyl, (1-4C)alkoxycarbonyl, (1-6C)alkylaminocarbonyl or di-(1-6C)alkylaminocarbonyl and any available nitrogen atom present in the ring is optionally substituted by (1-4C)alkyl, hydroxy(1-4C)alkyl, (1-2C)alkoxy-(1-4C)alkyl, or (1-4C)alkanoyl; subject to the following provisos:
when R² is (1-2C)alkoxy, the alkoxy group is not located in the para or 4-position relative to the —NR¹— group;
R⁴ is not a 4-methylpiperazin-1-yl group when R² is a group of sub-formula -Q-R⁸, in which Q is —NR^{a}—CO—, R^{a} is hydrogen, and R⁸ is (1-2C)alkyl;
   or a pharmaceutically acceptable salt thereof.

According to a third aspect of the invention, there is provided a compound of formula I above wherein:

R¹ is a (1-4C)alkyl group which is optionally substituted by one or more substituent groups selected from —OR⁵ (wherein R⁵ is selected from hydrogen or (1-2C)alkyl), cyano, halo, or —NR⁶R⁷ (where R⁶ and R⁷ are independently selected from hydrogen, (1-2C)alkyl or (1-2C)alkanoyl);
n is 0, 1, 2 or 3;
each R² group present is independently selected from (1-2C)alkyl, (1-2C)alkoxy, fluoro, chloro, cyano, hydroxy(1-2C)alkyl, or a group of sub-formula:

-Q-R⁸

where Q is selected from —CO—, —NR^{a}, —NR^{a}—CO—, —NR^{a}—COO—, NR^{a}CONR^{b}, —CONR^{a}—, —S(O)_{z}— (where z is 0, 1 or 2); —SO₂NR^{a}—, and —NR^{a}SO₂—, R^{a} and R^{b} are each independently selected from hydrogen or methyl, and R⁸ is hydrogen or (1-2C)alkyl;
R³ is selected from:
(i) hydrogen, halo, nitro, cyano, or hydroxy;
(ii) an optionally substituted (1-6C)alkyl, (2-6C)alkenyl, or (2-6C)alkynyl group wherein the optional substituents are selected from:
   cyano;
   halo;
   a group of sub-formula:

—W—R⁹
wherein W is selected from —O—, —S(O)ₚ— (where p is 0, 1 or 2), —CO—, —NR^{b}CO—, —CONR^{b}—, —NR^{b}CONR^{b}—, —SO₂NR^{b}—, —NR^{b}SO₂—, or —NR^{b}COO—;
R¹ is selected from hydrogen or (1-2C)alkyl;
and R⁹ is selected from hydrogen or (1-4C)alkyl;
or —NR¹⁰R¹¹, where R¹⁰ and R¹¹ are independently selected from hydrogen, or (1-2C)alkyl, or R¹⁰ and R¹¹ are linked to form a 4, 5, 6 or 7 membered heterocyclic ring which optionally comprises, in addition to the nitrogen atom to which R¹⁰ and R¹¹ are attached, one or two further heteroatoms selected from O, N or S, and wherein any S atoms that are present may be optionally oxidised to form an SO and SO₂ group, and wherein any carbon atom present in the ring is optionally substituted by oxo, halo, hydroxy, cyano, (1-4C)alkyl, hydroxy(1-4C)alkyl, (1-4C)alkoxy, (1-2C)alkoxy-(1-4C)alkyl, (1-4C)alkanoyl, (1-4C)alkanesulfonyl, (1-4C)alkoxycarbonyl, (1-6C)alkylaminocarbonyl or di-(1-6C)alkylaminocarbonyl and any available nitrogen atom present in the ring is optionally substituted by (1-4C)alkyl, hydroxy(1-4C)alkyl, (1-2C)alkoxy-(1-4C)alkyl, or (1-4C)alkanoyl;
(iii) a group —NR¹²R¹³, wherein R¹² and R¹³ are each independently selected from hydrogen or (1-6C)alkyl, or R¹² and R¹³ are linked to form a 4, 5, 6 or 7-membered heterocyclic ring which optionally comprises, in addition to the nitrogen atom to which R¹² and R¹³ are attached, one or two further heteroatoms selected from O, N or S, and wherein any S atoms that are present may be optionally oxidised to form an SO and SO₂ group, and wherein any carbon atom present in the ring is optionally substituted by oxo, halo, hydroxy, cyano, (1-4C)alkyl, hydroxy(1-4C)alkyl, (1-4C)alkoxy, (1-2C)alkoxy-(1-4C)alkyl, (1-4C)alkanoyl, (1-4C)alkanesulfonyl, (1-4C)alkoxycarbonyl, (1-6C)alkylaminocarbonyl or di-(1-6C)alkylaminocarbonyl and any available nitrogen atom present in the ring is optionally substituted by (1-4C)alkyl, hydroxy(1-4C)alkyl, (1-2C)alkoxy-(1-4C)alkyl, or (1-4C)alkanoyl; or
(iv) a group of formula (II):

—X—R¹⁴
wherein X is selected from —O—, —S(O)ₚ— (where p is 0, 1 or 2), —CO—, —NR^{c}CO—, —CONR^{c}—, —NR^{c}COO—, and —NR^{c}SO₂—,
where R¹ is selected hydrogen or (1-2C)alkyl;
R¹⁴ is a (1-4C)alkyl group which is optionally substituted by halo, hydroxy, cyano, (1-4C)alkoxy, or R¹⁴ is

—NR¹⁵R¹⁶
where R¹⁵ and R¹⁶ are independently selected from hydrogen, (1-2C)alkanoyl or (1-2C)alkyl, or R¹⁵ and R¹⁶ are linked to form a 4, 5, 6 or 7-membered heterocyclic ring which optionally comprises, in addition to the nitrogen atom to which R¹⁵ and R¹⁶ are attached, one or two further heteroatoms selected from O, N or S, and wherein any S atoms that are present may be optionally oxidised to form an SO and SO₂ group, and wherein any carbon atom present in the ring is optionally substituted by oxo, halo, hydroxy, cyano, (1-4C)alkyl, hydroxy(1-4C)alkyl, (1-4C)alkoxy, (1-2C)alkoxy-(1-4C)alkyl, (1-4C)alkanoyl, (1-4C)alkanesulfonyl, (1-4C)alkoxycarbonyl, (1-6C)alkylaminocarbonyl or di-(1-6C)alkylaminocarbonyl and any available nitrogen atom is optionally substituted by (1-4C)alkyl, hydroxy(1-4C)alkyl, (1-2C)alkoxy-(1-4C)alkyl, or (1-4C)alkanoyl;
   R⁴ is a group —NR¹⁷R¹⁸, wherein R¹⁷ and R¹⁸ are linked to form a 4, 5, 6 or 7 membered heterocyclic ring which optionally comprises, in addition to the nitrogen atom to which R¹⁷ and R¹⁸ are attached, one or two further heteroatoms selected from O, N or S, and wherein any S atoms that are present may be optionally oxidised to form an SO or SO₂ group, and wherein any carbon atom present in the ring is optionally substituted by oxo, halo, hydroxy, cyano, (1-4C)alkyl, hydroxy(1-4C)alkyl, (1-4C)alkoxy, (1-2C)alkoxy-(1-4C)alkyl, (1-4C)alkanoyl, (1-4C)alkanesulfonyl, (1-4C)alkoxycarbonyl, (1-6C)alkylaminocarbonyl or di-(1-6C)alkylaminocarbonyl and any available nitrogen atom present in the ring is optionally substituted by (1-4C)alkyl, hydroxy(1-4C)alkyl, (1-2C)alkoxy-(1-4C)alkyl, or (1-4C)alkanoyl; subject to the following provisos:
when R² is (1-2C)alkoxy, the alkoxy group is not located in the para or 4-position relative to the —NR¹— group;
R⁴ is not a 4-methylpiperazin-1-yl group when R² is a group of sub-formula -Q-R⁸, in which Q is —NR^{a}—CO—, R^{a} is hydrogen, and R⁸ is (1-2C)alkyl;
   or a pharmaceutically acceptable salt thereof.

### LIST OF FIGURES

Figure A, X-Ray Powder Diffraction Pattern for [3-[[2-[(3,5-dimorpholin-4-ylphenyl)amino]pyrimidin-4-yl]-methyl-amino]-4-methyl-phenyl]methanol free base Form 1.

Figure B, X-Ray Powder Diffraction Pattern for [3-[[2-[(3,5-dimorpholin-4-ylphenyl)amino]pyrimidin-4-yl]-methyl-amino]-4-methyl-phenyl]methanol besylate Form 1.

Figure C, X-Ray Powder Diffraction Pattern for [3-[[2-[(3,5-dimorpholin-4-ylphenyl)amino]pyrimidin-4-yl]-methyl-amino]-4-methyl-phenyl]methanol besylate Form 2.

Figure D, X-Ray Powder Diffraction Pattern for [3-[[2-[(3,5-dimorpholin-4-ylphenyl)amino]pyrimidin-4-yl]-methyl-amino]-4-methyl-phenyl]methanol tosylate Form 1.

Figure E, X-Ray Powder Diffraction Pattern for [3-[[2-[(3,5-dimorpholin-4-ylphenyl)amino]pyrimidin-4-yl]-methyl-amino]-4-methyl-phenyl]methanol tosylate Form 2.

Figure F, X-Ray Powder Diffraction Pattern for [3-[[2-[(3,5-dimorpholin-4-ylphenyl)amino]pyrimidin-4-yl]-methyl-amino]-4-methyl-phenyl]methanol fumarate Form 1.

Figure G, X-Ray Powder Diffraction Pattern for [3-[[2-[(3,5-dimorpholin-4-ylphenyl)amino]pyrimidin-4-yl]-methyl-amino]-4-methyl-phenyl]methanol fumarate Form 2.

Figure H, Effect on mean HT29 tumour volumes of dosing with [3-[[2-[(3,5-dimorpholin-4-ylphenyl)amino]pyrimidin-4-yl]-methyl-amino]-4-methyl-phenyl]methanol (Example 6) and 4-(4-fluoro-2-methylindol-5-yloxy)-6-methoxy-7-(3-piperidin-1-yl-propoxy)-quinazoline (AZD7514) either individually or in combination in a mouse tumour explant model.

### DETAILED DESCRIPTION

It is to be understood that, insofar as certain of the compounds of Formula I defined above may exist in optically active or racemic forms by virtue of one or more asymmetric carbon atoms, the invention includes in its definition any such optically active or racemic form which possesses the above-mentioned activity. The synthesis of optically active forms may be carried out by standard techniques of organic chemistry well known in the art, for example by synthesis from optically active starting materials or by the resolution of a racemic form. Similarly, the above-mentioned activity may be evaluated using the standard laboratory techniques referred to hereinafter.

It is to be understood that certain compounds of Formula I defined above may exhibit the phenomenon of tautomerism. In particular, tautomerism may affect any heterocyclic groups that bear 1 or 2 oxo substituents. It is also to be understood that the present invention includes in its definition any such tautomeric form, or a mixture thereof, which possesses the above-mentioned activity and is not to be limited merely to any one tautomeric form utilised within the formulae drawings or named in the Examples.

It is to be understood that certain compounds of Formula I above may exist in unsolvated forms as well as solvated forms, such as, for example, hydrated forms. It is also to be understood that the present invention encompasses all such solvated forms that possess anticancer or antitumour activity.

It is also to be understood that certain compounds of the Formula I may exhibit polymorphism, and that the present invention encompasses all such forms which possess anticancer or antitumour activity.

It is further to be understood that any R² group that is present on the phenyl moiety of the aniline group that is located at the 4-position on the pyrimidine ring may be located at any available position on said phenyl moiety, with the exception that if n is 1 and R² is (1-2C)alkoxy, in which case it cannot be located in the para or 4-position (relative to the aniline nitrogen) of the phenyl moiety, or if n is 1 and R² is ethoxy, in which case the ethoxy group cannot be located in the meta or 3-position (relative to the aniline nitrogen) of the phenyl moiety. When multiple R² groups are present, each R² group may be the same or different.

It will also be understood that R⁴ is a group —NR¹⁷R¹⁸ as defined above, but cannot be a 4-methylpiperazin-1-yl group when R² is a group of sub-formula -Q-R⁸, in which Q is —NR^{a}—CO—, R^{a} is hydrogen, and R⁸ is (1-2C)alkyl.

In this specification the generic term “alkyl” includes both straight-chain and branched-chain alkyl groups such as propyl, isopropyl and tert-butyl. However references to individual alkyl groups such as “propyl” are specific for the straight-chain version only, references to individual branched-chain alkyl groups such as “isopropyl” are specific for the branched-chain version only. An analogous convention applies to other generic terms, for example (1-4C)alkoxy includes methoxy, ethoxy and isopropoxy.

The term “halo” refers to fluoro, chloro, bromo, or iodo.

The term “heterocyclic ring”, unless otherwise defined herein, refers to saturated, partially saturated or unsaturated monocyclic rings containing 4, 5, 6 or 7 ring atoms. In particular compounds of the invention, “heterocyclic rings” are saturated monocyclic rings that contain 4, 5, 6 or 7 ring atoms, and especially 5 or 6 ring atoms.

Examples and suitable values of the term “heterocyclic ring” used herein are pyrrolidinyl, imidazolidinyl, pyrazolidinyl, piperidinyl, piperazinyl, morpholin-4-yl, homomorpholinyl, thiomorpholin-4-yl, 1,4-oxazepan-4-yl, diazepanyl and oxazolidinyl.

In compounds of the invention in which R³ is a (1-4C)alkyl group optionally substituted with a group —NR¹⁰R¹¹ wherein R¹⁰ and R¹¹ are linked to form a heterocyclic ring, the heterocyclic ring so formed is suitably a 5 or 6-membered heterocyclic ring system. Suitably, the heterocyclic rings formed when R¹⁰ and R¹¹ are linked are saturated 5 or 6-membered heterocyclic rings.

Suitable examples of —NR¹⁰R¹¹ groups include pyrrolidinyl, imidazolidinyl, pyrazolidinyl, piperidinyl, piperazinyl, morpholin-4-yl, thiomorpholin-4-yl, 1,4-oxazepan-4-yl, diazepanyl and oxazolidinyl. Particular examples include pyrrolidinyl, imidazolidinyl, piperidinyl, piperazinyl, morpholin-4-yl, or thiomorpholin-4-yl.

In compounds of the invention in which R³ is a group —NR¹²R¹³ wherein R¹² and R¹³ are linked to form a heterocyclic ring, the heterocyclic ring so formed is suitably a 5, 6 or 7 membered ring. Suitably, the heterocyclic rings formed when R¹⁰ and R¹¹ are linked are saturated 5, 6 or 7-membered heterocyclic rings.

Suitable examples of —NR¹²R¹³ groups include pyrrolidinyl, imidazolidinyl, pyrazolidinyl, piperidinyl, piperazinyl, morpholin-4-yl, homomorpholinyl, thiomorpholin-4-yl, 1,4-oxazepan-4-yl, diazepanyl and oxazolidinyl. Particular examples include pyrrolidinyl, imidazolidinyl, piperidinyl, piperazinyl, morpholin-4-yl, homomorpholinyl, or thiomorpholin-4-yl.

In compounds of the invention in which R¹⁴ is a group —NR¹⁵R¹⁶ wherein R¹⁵ and R¹⁶ are linked to form a 5 or 6-membered heterocyclic ring, the heterocyclic ring so formed is suitably a 5 or 6 membered ring. Suitably, the heterocyclic rings formed when R¹⁰ and R¹¹ are linked are saturated 5 or 6-membered heterocyclic rings.

Suitable examples of —NR¹⁵R¹⁶ groups include pyrrolidinyl, imidazolidinyl, pyrazolidinyl, piperidinyl, piperazinyl, morpholin-4-yl, thiomorpholin-4-yl, 1,4-oxazepan-4-yl, diazepanyl and oxazolidinyl. Particular examples include pyrrolidinyl, imidazolidinyl, piperidinyl, piperazinyl, morpholin-4-yl, or thiomorpholin-4-yl.

Particular novel compounds of the invention include, for example, compounds of Formula I, or pharmaceutically-acceptable salts thereof, wherein, unless otherwise stated, each of R¹, n, R², R³ or R⁴ has any of the meanings defined hereinbefore or in paragraphs (1) to (41) hereinafter:—

(1) R¹ is a (1-4C)alkyl group which is optionally substituted by one or more substituent groups selected from —OR⁵ (wherein R⁵ is selected from hydrogen or (1-2C)alkyl), or cyclopropylmethyl;
(2) R¹ is a (1-4C)alkyl group which is optionally substituted by one or more substituent groups selected from —OR⁵ (wherein R⁵ is selected from hydrogen or (1-2C)alkyl);
(2.1) R¹ is selected from methyl, ethyl, propyl, isopropyl, 2-methylpropyl, cyclopropylmethyl or 2-methoxyethyl;
(2.2) R¹ is selected from methyl or 2-methoxyethyl;

### (3) R¹ is (1-4C)alkyl;

(4) R¹ is selected from methyl, ethyl, propyl, isopropyl, 2-methylpropyl or cyclopropylmethyl;
(5) R¹ is selected from methyl, ethyl, isopropyl or cyclopropylmethyl;
(6) R¹ is methyl;
(7) R¹ is isopropyl;
(8) R¹ is cyclopropylmethyl;
(9) R¹ is ethyl;
(10) n is 1, 2 or 3;
(11) n is 2 or 3;
(12) n is 1;
(13) n is 2;
(14) n is 3;
(15) each R² group present is independently selected from (1-2C)alkyl, (1-2C)alkoxy, fluoro, chloro, cyano, hydroxy(1-2C)alkyl, or a group of sub-formula:

-Q-R⁸
where Q is selected from —NR^{a}—CO—, —S(O)_{z}— (where z is 0, 1 or 2); R^{a} is selected from hydrogen or methyl, and R⁸ is hydrogen or (1-2C)alkyl;
   (16) each R² group present is independently selected from (1-2C)alkyl, (1-2C)alkoxy, fluoro, chloro, cyano, hydroxy(1-2C)alkyl, or a group of sub-formula:

-Q-R⁸
where Q is selected from —NR^{a}—CO—, —S(O)_{z}— (where z is 0, 1 or 2); R^{a} is selected from hydrogen or methyl, and R⁸ is hydrogen or (1-2C)alkyl;
   (16.1) each R² group present is independently selected from methyl, fluoro, chloro, cyano, hydroxymethyl, methoxy, acetamido, methylthio, ethanol, or ethanone;
   (17) each R² group present is independently selected from methyl, fluoro, chloro, hydroxymethyl, methoxy, acetamido, or methylthio;
   (18) each R² group present is independently selected from methyl, fluoro, chloro, hydroxymethyl, or methoxy;
   (19) each R² group present is independently selected from fluoro or chloro;
   (20) each R² group present is independently selected from methyl or hydroxymethyl;
   (21) each R² group present is methyl;
   (22) each R² group present in hydroxymethyl;
   (23) each R² group present is independently selected from acetamido or methoxy;
   (24) each R² group present is methoxy;
   (25) R³ is selected from:
(i) hydrogen, halo, nitro, cyano, or hydroxy;
(ii) an optionally substituted (1-6C)alkyl group, wherein the optional substituents are selected from cyano, halo,
   or a group of sub-formula:

—W—R⁹
wherein W is selected from —O—, —S(O)ₚ— (where p is 0, 1 or 2), —CO—, —NR^{b}CO—, or —CONR^{b}—; R^{b} is selected from hydrogen or (1-2C)alkyl; and R⁹ is selected from hydrogen or (1-4C)alkyl;
or —NR¹⁰R¹¹ where R¹⁰ and R¹¹ are independently selected from hydrogen, (1-2C)alkanoyl or (1-2C)alkyl, or R¹⁰ and R¹¹ are linked to form a 5, or 6 membered heterocyclic ring which optionally comprises, in addition to the nitrogen atom to which R¹⁰ and R¹¹ are attached, one or two further heteroatoms selected from O, N or S, and wherein the ring is optionally substituted on any available carbon atom by one or two substituent groups selected from oxo, halo, hydroxy, cyano, (1-4C)alkyl, or (1-4C)alkanesulfonyl, and any available nitrogen atom present in the ring is optionally substituted by (1-4C)alkyl or (1-4C)alkanoyl;
(iii) a group —NR¹²R¹³, wherein R¹² and R¹³ are each independently selected from hydrogen or (1-6C)alkyl, or R¹² and R¹³ are linked to form a 5, 6 or 7-membered heterocyclic ring which comprises, in addition to the nitrogen atom to which R¹² and R¹³ are attached, one or two further heteroatoms selected from O, N or S, and wherein the ring is optionally substituted on any available carbon atom by one or two substituent groups selected from oxo, halo, hydroxy, cyano, (1-4C)alkyl, or (1-4C)alkanesulfonyl, and any available nitrogen atom present in the ring is optionally substituted by (1-4C)alkyl or (1-4C)alkanoyl; or
(iv) a group of formula (II):

—X—R¹⁴
wherein X is selected from —O—, —S(O)ₚ— (where p is 0, 1 or 2), —CO—, NR^{c}CO—, —CONR^{c}, or —NR^{c}COO—,
where R^{c} is selected hydrogen or (1-2C)alkyl;
R¹⁴ is a (1-4C)alkyl group which is optionally substituted by halo, hydroxy, cyano, (1-4C)alkoxy, or R¹⁴ is

—NR¹⁵R¹⁶
where R¹⁵ and R¹⁶ are independently selected from hydrogen, (1-2C)alkanoyl or (1-2C)alkyl, or R¹⁵ and R¹⁶ are linked to form a 5, or 6-membered heterocyclic ring which optionally comprises, in addition to the nitrogen atom to which R¹⁵ and R¹⁶ are attached, one or two further heteroatoms selected from O, N or S, and wherein the ring is optionally substituted on any available carbon atom by one or two substituent groups selected from oxo, halo, hydroxy, cyano, (1-4C)alkyl, or (1-4C)alkanesulfonyl, and any available nitrogen atom present in the ring is optionally substituted by (1-4C)alkyl or (1-4C)alkanoyl;
   (26) R³ is selected from:
(i) hydrogen, halo, cyano, or hydroxy;
(ii) an optionally substituted (1-4C)alkyl group wherein the optional substituents are selected from:
   cyano;
   halo;
   a group of sub-formula:

—W—R⁹
wherein W is selected from —O—, —S(O)ₚ— (where p is 0, 1 or 2), —CO—, —NR^{b}CO—, —CONR^{b}—,
R^{b} is selected from hydrogen or (1-2C)alkyl,
and R⁹ is selected from hydrogen or (1-2C)alkyl;
or —NR¹⁰R¹¹, where R¹⁰ and R¹¹ are independently selected from hydrogen or (1-2C)alkyl, or R¹⁰ and R¹¹ are linked to form a 5 or 6 membered heterocyclic ring which optionally comprises, in addition to the nitrogen atom to which R¹⁰ and R¹¹ are attached, one or two further heteroatoms selected from O, N or S, and wherein the ring is optionally substituted on any available carbon atom by one or two substituent groups selected from oxo, halo, hydroxy, cyano, (1-4C)alkyl, or (1-4C)alkyl-S(O)₃— (where a is 0, 1 or 2) and any available nitrogen atom is optionally substituted by (1-4C)alkyl or (1-4C)alkanoyl;
(iii) a group —NR¹²R¹³, wherein R¹² and R¹³ are each independently selected from hydrogen or (1-2C)alkyl, or R¹² and R¹³ are linked to form a 5, 6 or 7-membered heterocyclic ring, and wherein, in addition to the nitrogen atom to which R¹² and R¹³ are attached, the ring optionally comprises one or two further heteroatoms selected from O, N or S, and wherein the ring is optionally substituted on any available carbon atom by one or two substituent groups selected from oxo, halo, hydroxy, cyano, (1-4C)alkyl, or (1-4C)alkyl-S(O)_{b}— (where b is 0, 1 or 2), and any available nitrogen atom is optionally substituted by (1-4C)alkyl or (1-4C)alkanoyl; or
(iv) a group of formula (II):

—X—R¹⁴
wherein X is selected from —O—, —S(O)_{q}— (where q is 0, 1 or 2), or —CO—,
R¹⁴ is a (1-4C)alkyl, (3-4C)cycloalkyl or (3-4C)cycloalkyl(1-2C)alkyl group which is optionally substituted by halo, hydroxy, cyano, (1-4C)alkoxy, or R¹⁴ is

—NR¹⁵R¹⁶
where R¹⁵ and R¹⁶ are independently selected from hydrogen or a (1-4C)alkyl (3-4C)cycloalkyl or (3-4C)cycloalkyl(1-2C)alkyl group which is optionally substituted by halo, hydroxy, cyano, (1-4C)alkoxy, or R¹⁵ and R¹⁶ are linked to form a 5 or 6-membered heterocyclic ring which optionally comprises, in addition to the nitrogen atom to which R¹⁵ and R¹⁶ are attached, one or two further heteroatoms selected from O, N or S, and wherein the ring is optionally substituted on any available carbon atom by one or two substituent groups selected from oxo, halo, hydroxy, cyano, (1-4C)alkyl, or (1-4C)alkyl-S(O)_{c}— (where c is 0, 1 or 2), and any available nitrogen atom is optionally substituted by (1-4C)alkyl or (1-4C)alkanoyl;
   (27) R³ is selected from:
(i) hydrogen, halo, or cyano;
(ii) an optionally substituted (1-2C)alkyl group wherein the optional substituents are selected from cyano, halo, a group of sub-formula:

—W—R⁹
wherein W is selected from —O—, —S(O)ₚ— (where p is 0, 1 or 2), —CO—, —NR^{b}CO—, —CONR^{b}—; R^{b} is selected from hydrogen or (1-2C)alkyl and R⁹ is selected from hydrogen or (1-4C)alkyl;
or —NR¹⁰R¹¹, where R¹⁰ and R¹¹ are independently selected from hydrogen or (1-2C)alkyl), or R¹⁰ and R¹¹ are linked to form a 5 or 6 membered heterocyclic ring which optionally comprises, in addition to the nitrogen atom to which R¹⁰ and R¹¹ are attached, one or two further heteroatoms selected from O, N or S, and wherein the ring is optionally substituted on any available carbon atom by one or two substituent groups selected from oxo, halo, hydroxy, cyano, or (1-4C)alkyl, and any available nitrogen atom present in the ring is optionally substituted by (1-4C)alkyl;
(iii) a group —NR¹²R¹³, wherein R¹² and R¹³ are each independently selected from hydrogen or (1-6C)alkyl, or R¹² and R¹³ are linked to form a 5, 6 or 7-membered heterocyclic ring, and wherein, in addition to the nitrogen atom to which R¹² and R¹³ are attached, the ring optionally comprises one or two further heteroatoms selected from O, N or S, and wherein the ring is optionally substituted on any available carbon atom by one or two substituent groups selected from oxo, halo, hydroxy, cyano, or (1-4C)alkyl, and any available nitrogen atom present in the ring is optionally substituted by (1-4C)alkyl; or
(iv) a group of formula (II):

—X—R¹⁴
wherein X is selected from —O—, —S(O)ₚ— (where p is 0, 1 or 2), or —CONR^{c}—,
where R^{c} is selected hydrogen or (1-2C)alkyl;
R¹⁴ is a (1-4C)alkyl group which is optionally substituted by halo, hydroxy, cyano, (1-4C)alkoxy;
   (28) R¹³ is a group —NR¹²R¹³, wherein R¹² and R¹³ are each independently selected from hydrogen or (1-6C)alkyl, or R¹² and R¹³ are linked to form a 5, 6 or 7-membered heterocyclic ring, and wherein, in addition to the nitrogen atom to which R¹² and R¹³ are attached, the ring optionally comprises one or two further heteroatoms selected from O, N or S, and wherein the ring is optionally substituted on any available carbon atom by one or two substituent groups selected from oxo, halo, hydroxy, cyano, (1-4C)alkyl, or (1-4C)alkanesulfonyl, and any available nitrogen atom present in the ring is optionally substituted by (1-4C)alkyl or (1-4C)alkanoyl;
   (29) R³ is a group —NR¹²R¹³, wherein R¹² and R¹³ are linked to form a 5, 6 or 7-membered heterocyclic ring, and wherein, in addition to the nitrogen atom to which R¹² and R¹³ are attached, the ring optionally comprises one or two further heteroatoms selected from O, N or S, and wherein the ring is optionally substituted on any available carbon atom by one or two substituent groups selected from oxo, halo, hydroxy, cyano, (1-4C)alkyl, or (1-4C)alkanesulfonyl, and any available nitrogen atom present in the ring is optionally substituted by (1-4C)alkyl or (1-4C)alkanoyl;
   (30) R³ is a group —NR¹²R¹³, wherein R¹² and R¹³ are linked to form a 5, 6 or 7-membered heterocyclic ring, and wherein, in addition to the nitrogen atom to which R¹² and R¹³ are attached, the ring optionally comprises one or two further heteroatoms selected from O, N or S;
   (30.1) R³ is morpholin-4-yl, 1,4-oxazepan-4-yl, 4-methylpiperazin-1-yl or 4-hydroxypiperidin-1-yl;
   (31) R³ is thiomorpholin-4-yl or morpholin-4-yl;
   (32) R³ is morpholin-4-yl;
   (33) R⁴ is a group —NR¹⁷R¹⁸, wherein R¹⁷ and R¹⁸ are linked to form a 5 or 6 membered heterocyclic ring which optionally comprises, in addition to the nitrogen atom to which R¹⁷ and R¹⁸ are attached, one or two further heteroatoms selected from O, N or S, and wherein any S atoms that are present may be optionally oxidised to form an SO or SO₂ group, and wherein the ring is optionally substituted on any available carbon atom by one or two substituent groups selected from oxo, halo, hydroxy, cyano, (1-4C)alkyl, or (1-4C)alkanesulfonyl, and any available nitrogen atom is optionally substituted by (1-4C)alkyl, hydroxy(1-4C)alkyl, or (1-4C)alkanoyl;
   (34) R⁴ is a group —NR¹⁷R¹⁸, wherein R¹⁷ and R¹⁸ are linked to form a 6 membered heterocyclic ring which optionally comprises, in addition to the nitrogen atom to which R¹⁷ and R¹⁸ are attached, one or two further heteroatoms selected from O, N or S, and wherein the ring is optionally substituted on any available carbon atom by one or two substituent groups selected from oxo, halo, hydroxy, cyano, or (1-4C)alkyl, and any available nitrogen atom is optionally substituted by (1-4C)alkyl, hydroxy(1-4C)alkyl or (1-4C)alkanoyl;
   (35) R⁴ is a group of formula:

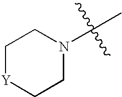

wherein Y is selected from O, S, NR^{y}, or CR^{z}, where R^{y} is selected from hydrogen, (1-2C)alkyl, hydroxy(1-2C)alkyl, (1-2C)alkoxy(1-2C)alkyl, or (1-2C)alkanoyl, and R^{z} is selected from hydrogen, hydroxy, (1-2C)alkyl, hydroxy(1-2C)alkyl, (1-2C)alkoxy(1-2C)alkyl, or (1-2C)alkanoyl;
   (36) R⁴ is a group of formula:

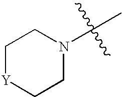

wherein Y is selected from O, NR^{y}, or CR^{z}, where R^{y} is selected from hydrogen or (1-2C)alkyl, and R^{z} is selected from hydrogen or hydroxy;
   (37) R⁴ is a group of formula:

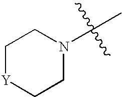

wherein Y is selected from O or NR^{y}, where R^{y} is selected from hydrogen or methyl;
   (38) R⁴ is a group of formula:

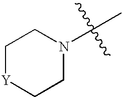

wherein Y is O;
   (39) R⁴ is selected from morpholin-4-yl, 4-methylpiperazin-1-yl, or 4-hydroxypiperidin-1-yl;
   (40) R⁴ is morpholin-4-yl;
   (41) R⁴ is selected from morpholin-4-yl, (R)-3-methyl-morpholin-4-yl, (S)-3-methyl-morpholin-4-yl or 4-oxopiperidin-1-yl.

In a particular sub-group of compounds of the Formula I, R¹ is an alkyl group as defined in any one of paragraphs (1) to (9) above. In a further sub-group of compounds of the invention, R¹ is methyl.

In a further sub-group of compounds of Formula I, n is an integer selected from 0, 1, 2 or 3, and each R² group that may be present is as defined in any one of paragraphs (15) to (24) above. In a particular sub-group of compounds of the invention, each R² group present is as defined in any one of paragraphs (18) to (24) above.

In a further sub-group of compounds of Formula I, n is 3 and each R² group is selected from fluoro or chloro.

In a further sub-group of compounds of Formula I, the aniline in the 4-position of the pyrimidine ring has the following structure:

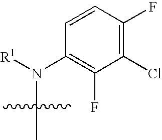

wherein R¹ has any one of the definitions set out herein.

In a further sub-group of compounds of Formula I, n is 2 and each R² group present is selected from methyl or hydroxymethyl.

In a further sub-group of compounds of Formula I, the aniline in the 4-position of the pyrimidine ring has the following structure:

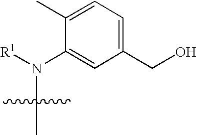

wherein R¹ has any one of the definitions set out herein.

In a further sub-group of compounds of Formula I, n is 1 and the R² group present is methoxy (subject to the proviso that the methoxy group is not located in the para or 4-position of the aniline).

In a further sub-group of compounds of Formula I, the aniline in the 4-position of the pyrimidine ring having the following structure:

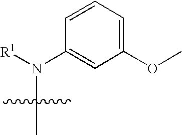

wherein R¹ has any one of the definitions set out herein.

In further sub-groups of compounds of Formula I, R³ is as defined in any one of paragraphs (25) to (32) above, and is especially as defined in any one of paragraphs (28) to (32) above.

In a particular sub-group of compounds of the invention, R⁴ is as defined in any one of paragraphs (33) to (40) above. In a further particular sub-group of compounds of the invention, R⁴ is as defined in either paragraph (39) or (40). Suitably, R⁴ is morpholin-4-yl.

In a sub-group of compounds of formula I, n, R¹ and R³ have any one of the definitions set out hereinbefore, R⁴ is a group of formula:

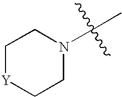

wherein Y is —NR^{y}—, and R^{y} is selected from hydrogen or (1-2C)alkyl, and each R² group present is independently selected from (1-2C)alkyl, (1-2C)alkoxy, fluoro, chloro, cyano, hydroxy(1-2C)alkyl, or a group of sub-formula:

-Q-R⁸

where Q is selected from —CO—, —NR^{a}—, or —S(O)_{z}— (where z is 0, 1 or 2); R^{a} is selected from hydrogen or methyl, and R⁸ is hydrogen or (1-2C)alkyl.

In a further sub-group of compounds of formula I, n, R¹ and R³ have any one of the definitions set out hereinbefore, R⁴ is a group of formula:

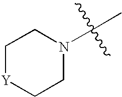

wherein Y is —NR^{y}—, and R^{y} is selected from hydrogen or (1-2C)alkyl, and each R² group present is independently selected from (1-2C)alkyl, (1-2C)alkoxy, fluoro, chloro, cyano, or hydroxy(1-2C)alkyl.

In a sub-group of compounds of Formula I, R⁴ is a group of formula:

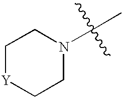

wherein Y is O or —CR^{z}—, and R^{z} is selected from hydrogen or hydroxy, and R¹, R², n and R³ each have any one of the definitions set out hereinbefore.

A particular sub-group of compounds of the invention have the structural formula IA:

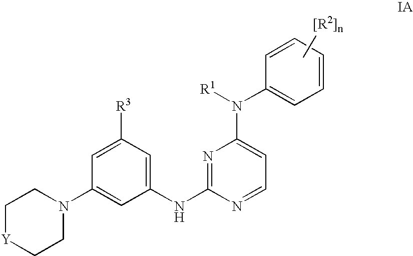

wherein:
Y is selected from O, S, NR^{y}, or CR^{z}, where R^{y} is selected from hydrogen, (1-2C)alkyl, hydroxy(1-2C)alkyl, (1-2C)alkoxy(1-2C)alkyl, or (1-2C)alkanoyl, and R^{Z} is selected from hydrogen, hydroxy, (1-2C)alkyl, hydroxy(1-2C)alkyl, (1-2C)alkoxy(1-2C)alkyl, or (1-2C)alkanoyl;
R¹ is a (1-4C)alkyl group;
n is 0, 1, 2 or 3;
each R² group present is independently selected from (1-2C)alkyl, (1-2C)alkoxy, fluoro, chloro, cyano, hydroxy(1-2C)alkyl, or a group of sub-formula:

-Q-R⁸

where Q is selected from —CO—, —NR^{a}—, —NR^{a}—CO—, —CONR^{a}—, S(O)_{z}— (where z is 0, 1, or 2); R^{a} is selected from hydrogen or methyl, and R⁸ is hydrogen or (1-2C)alkyl;
R³ is selected from:
(i) hydrogen, halo, nitro, cyano, or hydroxy;
(ii) an optionally substituted (1-4C)alkyl group wherein the optional substituents are selected from cyano, halo, or a group of sub-formula:

—W—R⁹
wherein W is selected from —O—, —S(O)ₚ— (where p is 0, 1 or 2), —CO—, —NR^{b}CO—, —CONR^{b}—;
R^{b} is selected from hydrogen or (1-2C)alkyl
and R⁹ is selected from hydrogen or (1-2C)alkyl;
or —NR¹⁰R¹¹, where R¹⁰ and R¹¹ are independently selected from hydrogen or (1-2C)alkyl, or R¹⁰ and R¹¹ are linked to form a 5, or 6 membered heterocyclic ring which optionally comprises, in addition to the nitrogen atom to which R¹⁰ and R¹¹ are attached, one or two further heteroatoms selected from O, N or S, and wherein the ring is optionally substituted on any available carbon atom by one or two substituent groups selected from oxo, halo, hydroxy, cyano, (1-4C)alkyl, or (1-4C)alkanesulfonyl, and any available nitrogen atom is optionally substituted by (1-4C)alkyl or (1-4C)alkanoyl;
(iii) a group —NR¹²R¹³, wherein R¹² and R¹³ are each independently selected from hydrogen or (1-2C)alkyl, or R¹² and R¹³ are linked to form a 5, 6 or 7-membered heterocyclic ring, and wherein, in addition to the nitrogen atom to which R¹² and R¹³ are attached, the ring optionally comprises one or two further heteroatoms selected from O, N or S, and wherein the ring is optionally substituted on any available carbon atom by one or two substituent groups selected from oxo, halo, hydroxy, cyano, (1-4C)alkyl, or (1-4C)alkanesulfonyl, and any available nitrogen atom is optionally substituted by (1-4C)alkyl or (1-4C)alkanoyl; or
(iv) a group of formula (II):

—X—R¹⁴
wherein X is selected from —O—, —S(O)ₚ— (where p is 0, 1 or 2), —CO—, —NR^{c}CO—, or —CONR^{c}—,
R^{c} is selected hydrogen or (1-2C)alkyl, and
R¹⁴ is a (1-4C)alkyl group which is optionally substituted by halo, hydroxy, cyano, (1-4C)alkoxy, or R¹⁴ is

—NR¹⁵R¹⁶
where R¹⁵ and R¹⁶ are independently selected from hydrogen or (1-2C)alkyl, or R¹⁵ and R¹⁶ are linked to form a 5, or 6-membered heterocyclic ring which optionally comprises, in addition to the nitrogen atom to which R¹⁵ and R¹⁶ are attached, one or two further heteroatoms selected from O, N or S, and wherein the ring is optionally substituted on any available carbon atom by one or two substituent groups selected from oxo, halo, hydroxy, cyano, (1-4C)alkyl, or (1-4C)alkanesulfonyl, and any available nitrogen atom is optionally substituted by (1-4C)alkyl or (1-4C)alkanoyl;
   subject to the following provisos:
when R² is (1-2C)alkoxy, it is not located in the para or 4-position relative to the —NR¹— group;
Y is not NR^{y} in which R^{y} is methyl when R² is a group of sub-formula -Q-R⁸, in which Q is —NR^{a}—CO—, R^{a} is hydrogen, and R⁸ is (1-2C)alkyl;
or a pharmaceutically acceptable salt thereof.

A further particular sub-group of compounds of the invention are of formula IA, wherein:

R¹ is a (1-4C)alkyl, (3-4C)cycloalkyl or cyclopropylmethyl group which is optionally substituted by one or more substituent groups selected from —OR⁵ (wherein R⁵ is selected from hydrogen or (1-2C)alkyl), cyano, halo, or —NR⁶R⁷ (where R⁶ and R⁷ are independently selected from hydrogen, (1-2C)alkyl or (1-2C)alkanoyl);
n is 0, 1, 2 or 3;
each R² group present is independently selected from (1-2C)alkyl, (1-2C)alkoxy, fluoro, chloro, cyano, hydroxy(1-2C)alkyl, or a group of sub-formula:

-Q-R⁸

where Q is selected from —CO—, —NR^{a}—, —NR^{a}—CO—, —CONR^{a}—, —S(O)_{z}— (where z is 0, 1 or 2); R^{a} is selected from hydrogen or methyl, and R⁸ is hydrogen or (1-2C)alkyl;
R³ is selected from:
(i) hydrogen, halo, nitro, cyano, or hydroxy;
(ii) an optionally substituted (1-4C)alkyl group wherein the optional substituents are selected from:
   cyano;
   halo;
   a group of sub-formula:

—W—R⁹
wherein W is selected from —O—, —S(O)ₚ— (where p is 0, 1 or 2), —CO—, —NR^{b}CO—, —CONR^{b}—
R^{b} is selected from hydrogen or (1-2C)alkyl,
and R⁹ is selected from hydrogen or (1-2C)alkyl;
or —NR¹⁰R¹¹, where R¹⁰ and R¹¹ are independently selected from hydrogen or (1-2C)alkyl, or R¹⁰ and R¹¹ are linked to form a 5 or 6 membered heterocyclic ring which optionally comprises, in addition to the nitrogen atom to which R¹⁰ and R¹¹ are attached, one or two further heteroatoms selected from O, N or S, and wherein the ring is optionally substituted on any available carbon atom by one or two substituent groups selected from oxo, halo, hydroxy, cyano, (1-4C)alkyl, or (1-4C)alkyl-S(O)₃— (where a is 0, 1 or 2) and any available nitrogen atom is optionally substituted by (1-4C)alkyl or (1-4C)alkanoyl;
(iii) a group —NR¹²R¹³, wherein R¹² and R¹³ are each independently selected from hydrogen or a (1-4C)alkyl, (3-4C)cycloalkyl or (3-4C)cycloalkyl(1-2C)alkyl group which is optionally substituted by halo, hydroxy, cyano, or (1-4C)alkoxy, or R¹² and R¹³ are linked to form a 5, 6 or 7-membered heterocyclic ring, and wherein, in addition to the nitrogen atom to which R¹² and R¹³ are attached, the ring optionally comprises one or two further heteroatoms selected from O, N or S, and wherein the ring is optionally substituted on any available carbon atom by one or two substituent groups selected from oxo, halo, hydroxy, cyano, (1-4C)alkyl, or (1-4C)alkyl-S(O)_{b}-(where b is 0, 1 or 2), and any available nitrogen atom is optionally substituted by (1-4C)alkyl or (1-4C)alkanoyl; or
(iv) a group of formula (II):

—X—R¹⁴
wherein X is selected from —O—, —S(O)_{q}— (where q is 0, 1 or 2), —CO—, —NR^{c}CO—, —NR^{c}COO—, and —NR^{c}SO₂—;
where R^{c} is selected hydrogen or (1-2C)alkyl;
R¹⁴ is a (1-6C)alkyl, (3-6C)cycloalkyl or (3-6C)cycloalkyl(1-2C)alkyl group which is optionally substituted by halo, hydroxy, cyano, or (1-4C)alkoxy, or R¹⁴ is

—NR¹⁵R¹⁶
where R¹⁵ and R¹⁶ are independently selected from hydrogen or a (1-6C)alkyl, (3-6C)cycloalkyl or (3-6C)cycloalkyl(1-2C)alkyl group which is optionally substituted by halo, hydroxy, cyano, (1-4C)alkoxy, or R¹⁵ and R¹⁶ are linked to form a 5 or 6-membered heterocyclic ring which optionally comprises, in addition to the nitrogen atom to which R¹⁵ and R¹⁶ are attached, one or two further heteroatoms selected from O, N or S, and wherein the ring is optionally substituted on any available carbon atom by one or two substituent groups selected from oxo, halo, hydroxy, cyano, (1-4C)alkyl, or (1-4C)alkyl-S(O)_{c}— (where c is 0, 1 or 2), and any available nitrogen atom is optionally substituted by (1-4C)alkyl or (1-4C)alkanoyl; and
   Y is selected from O, S, NR^{y}, or CR^{z}, where R^{y} is selected from hydrogen, (1-2C)alkyl, hydroxy(1-2C)alkyl, (1-2C)alkoxy(1-2C)alkyl, or (1-2C)alkanoyl, and R^{z} is selected from hydrogen, hydroxy, (1-2C)alkyl, hydroxy(1-2C)alkyl, (1-2C)alkoxy, (1-2C)alkoxy(1-2C)alkyl, or (1-2C)alkanoyl; subject to the following provisos:
when R² is (1-2C)alkoxy, it is not located in the para or 4-position relative to the —NR¹— group;
Y is not NR^{y} in which R^{y} is methyl when R² is a group of sub-formula -Q-R⁸, in which Q is —NR^{a}—CO—, R^{a} is hydrogen, and R⁸ is (1-2C)alkyl;
   or a pharmaceutically acceptable salt thereof.

In a particular sub-group of compounds of Formula IA, Y is selected from O, NR^{y} or CR^{z}, where R^{y} is selected from hydrogen or (1-2C)alkyl, and R^{z} is selected from hydrogen or hydroxy. In a further sub-group of compounds of Formula IA, Y is selected from O or NR^{y}, where R^{y} is selected from hydrogen or (1-2C)alkyl. In a further sub-group of compounds of Formula IA, Y is O.

In compounds of Formula IA, R¹ suitably has any one of the definitions set out in paragraphs (4) to (9) above. In a particular sub-group of compounds of Formula IA, R¹ is methyl.

In a particular sub-group of compounds of Formula IA, n is as defined in any one of paragraphs (10) to (14) above and R² has any one of the definitions set out herein before or has any one of the definitions set out in paragraphs (15) to (24) above (subject to the proviso that if R² is (1-2C)alkoxy, then it is not located in the para position of the aniline).

In a particular sub-group of compounds of Formula IA, R³ is as defined in any one of paragraphs (25) or (26) above.

In a further sub-group of compounds of Formula IA, Y is not NR^{y} when R² is a group of sub-formula -Q-R⁸, in which Q is —NR^{a}—CO—, R^{a} is hydrogen, and R⁸ is (1-2C)alkyl. In a further sub-group of compounds of Formula IA, Y is not NR^{y} when R² is a group of sub-formula -Q-R⁸.

A further sub-group of compounds of the invention have the structural formula IB shown below

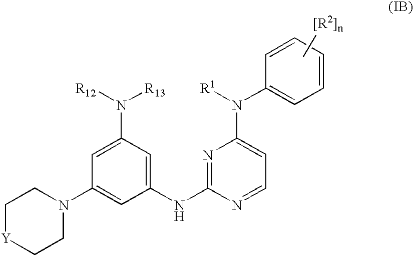

wherein:

Y, R¹, n and R² each have any one of the definitions set out above in relation to Formula I; and

R¹² and R¹³ are each independently selected from hydrogen or (1-2C)alkyl, or R¹² and R¹³ are linked to form a 5, 6 or 7-membered heterocyclic ring, and wherein, in addition to the nitrogen atom to which R¹² and R¹³ are attached, the ring optionally comprises one or two further heteroatoms selected from O, N or S, and wherein the ring is optionally substituted on any available carbon atom by one or two substituent groups selected from oxo, halo, hydroxy, cyano, (1-4C)alkyl, or (1-4C)alkanesulfonyl, and any available nitrogen atom is optionally substituted by (1-4C)alkyl or (1-4C)alkanoyl;

subject to the following provisos:
when R² is (1-2C)alkoxy, it is not located in the para position;
Y is not NR^{y} in which R^{y} is methyl when R² is a group of sub-formula -Q-R⁸, in which Q is —NR^{a}—CO—, R^{a} is hydrogen, and R⁸ is (1-2C)alkyl;
or a pharmaceutically acceptable salt thereof.

In compounds of Formula IB, R¹² and R¹³ are suitably linked to form a 5, 6 or 7-membered heterocyclic ring, and wherein, in addition to the nitrogen atom to which R¹² and R¹³ are attached, the ring optionally comprises one or two further heteroatoms selected from O, N or S, and wherein the ring is optionally substituted on any available carbon atom by one or two substituent groups selected from oxo, halo, hydroxy, cyano, (1-4C)alkyl, or (1-4C)alkanesulfonyl, and any available nitrogen atom is optionally substituted by (1-4C)alkyl or (1-4C)alkanoyl.

In a further sub-group of compounds of Formula IB, R¹² and R¹³ are linked to form a 5, 6 or 7-membered heterocyclic ring, and wherein, in addition to the nitrogen atom to which R¹² and R¹³ are attached, the ring optionally comprises one further heteroatom selected from O, N or S, and wherein the ring is optionally substituted on any available carbon atom by one or two substituent groups selected from oxo, halo, hydroxy, cyano, (1-2C)alkyl, or (1-2C)alkanesulfonyl, and any available nitrogen atom is optionally substituted by (1-2C)alkyl or (1-2C)alkanoyl.

A further particular sub-group of compounds of Formula I have the structural formula IC shown below

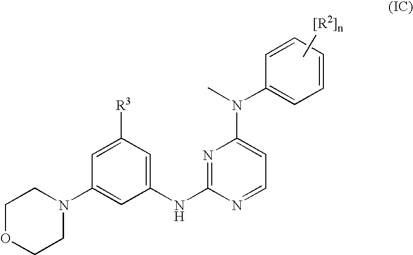

wherein n, R², and R³ have any one of the definitions set out above in relation to Formula I,
(subject to the proviso that when R² is (1-2C)alkoxy, it is not located in the para or 4-position)
or a pharmaceutically acceptable salt thereof.

A further particular sub-group of compounds of the invention have the structural formula (ID) shown below

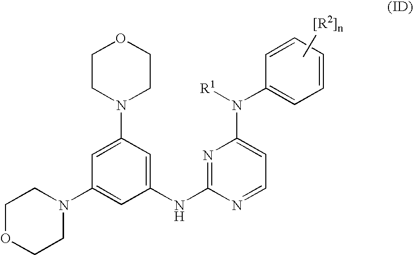

wherein R¹, R² and n have any one of the definitions set out hereinbefore (subject to the proviso that when R² is (1-2C)alkoxy, it is not located in the para or 4-position).

In a particular sub-group of compounds of the formula (ID), R¹ is (1-4C)alkyl, particularly methyl.

A further particular sub-group of compounds of the invention, or pharmaceutically acceptable salts thereof, have the structural formula (IE) shown below

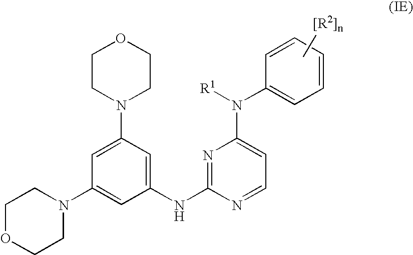

wherein:
R¹ is a (1-4C)alkyl, (3-4C)cycloalkyl or cyclopropylmethyl group which is optionally substituted by one or more substituent groups selected from —OR⁵ (wherein R⁵ is selected from hydrogen or (1-2C)alkyl), cyano, halo, or —NR⁶R⁷ (where R⁶ and R⁷ are independently selected from hydrogen, (1-2C)alkyl or (1-2C)alkanoyl);
n is 0, 1, 2 or 3; and
each R² group present is independently selected from (1-2C)alkyl, fluoro, chloro, cyano, hydroxy(1-2C)alkyl, or a group of sub-formula:

-Q-R⁸

where Q is selected from —CO—, —NR^{a}—, —NR^{a}CO—, —NR^{a}—COO—, NR^{a}CONR^{b}, —CONR^{a}—, —S(O)_{z}— (where z is 0, 1 or 2); —SO₂NR^{a}—, and —NR^{a}SO₂—, R^{a} and R^{b} are each independently selected from hydrogen or methyl, and R⁸ is hydrogen or (1-2C)alkyl.

In a particular sub-group of compounds of the formula (IE), R¹ is (1-4C)alkyl optionally substituted by —OR⁵ (wherein R⁵ is selected from (1-2C)alkyl).

In a particular sub-group of compounds of the formula (IE), R¹ is methyl or 2-methoxyethyl.

In another particular sub-group of compounds of the formula (IE), n is 1, 2 or 3.

In another particular sub-group of compounds of the formula (IE), n is 1 or 2.

In a further particular sub-group of compounds of the formula (IE), each R² group present is independently selected from (1-2C)alkyl, fluoro, chloro, cyano or hydroxy(1-2C)alkyl.

Particular novel compounds of the invention include any one of the following:
N′-(3-chloro-2,4-difluoro-phenyl)-N′-methyl-N-(3-morpholin-4-yl-5-thiomorpholin-4-yl-phenyl)pyrimidine-2,4-diamine;
N′-(3-chloro-2,4-difluoro-phenyl)-N-(3,5-dimorpholin-4-ylphenyl)-N′-methyl-pyrimidine-2,4-diamine;
N′-(3-chloro-2,4-difluoro-phenyl)-N-(3-fluoro-5-morpholin-4-yl-phenyl)-N′-methyl-pyrimidine-2,4-diamine;
N′-(3-chloro-2,4-difluoro-phenyl)-N′-methyl-N-(3-morpholin-4-ylphenyl)pyrimidine-2,4-diamine;
N′-(3-chloro-2,4-difluoro-phenyl)-N-(3-methoxy-5-morpholin-4-yl-phenyl)-N′-methyl-pyrimidine-2,4-diamine;
3-[[4-[(3-chloro-2,4-difluoro-phenyl)-methyl-amino]pyrimidin-2-yl]amino]-5-morpholin-4-yl-benzonitrile;
N′-(3-chloro-2,4-difluoro-phenyl)-N′-methyl-N-(3-methylsulfonyl-5-morpholin-4-yl-phenyl)pyrimidine-2,4-diamine;
[3-[[4-[(3-chloro-2,4-difluoro-phenyl)-methyl-amino]pyrimidin-2-yl]amino]-5-morpholin-4-yl-phenyl]methanol;
3-[[4-[(3-chloro-2,4-difluoro-phenyl)-methyl-amino]pyrimidin-2-yl]amino]-N,N-dimethyl-5-morpholin-4-yl-benzamide;
N′-(3-chloro-2,4-difluoro-phenyl)-N-[3-(2-methoxyethoxy)-5-morpholin-4-yl-phenyl]-N′-methyl-pyrimidine-2,4-diamine;
N′-(3-chloro-2,4-difluoro-phenyl)-N′-methyl-N-[3-morpholin-4-yl-5-(1,4-oxazepan-4-yl)phenyl]pyrimidine-2,4-diamine;
1-[3-[[4-[(3-chloro-2,4-difluoro-phenyl)-methyl-amino]pyrimidin-2-yl]amino]-5-methylsulfonyl-phenyl]piperidin-4-ol;
1-[4-[3-[[4-[(3-chloro-2,4-difluoro-phenyl)-methyl-amino]pyrimidin-2-yl]amino]-5-methylsulfonyl-phenyl]piperazin-1-yl]ethanone;
2-[4-[3-[[4-[(3-chloro-2,4-difluoro-phenyl)-methyl-amino]pyrimidin-2-yl]amino]-5-methylsulfonyl-phenyl]piperazin-1-yl]ethanol;
N′-(3-chloro-2,4-difluoro-phenyl)-N-[3-(methoxymethyl)-5-morpholin-4-yl-phenyl]-N′-methyl-pyrimidine-2,4-diamine;
N′-(3-chloro-2,4-difluoro-phenyl)-N′-methyl-N-[3-morpholin-4-yl-5-(propan-2-yloxymethyl)phenyl]pyrimidine-2,4-diamine;
N′-(3-chloro-2,4-difluoro-phenyl)-N′-methyl-N-[3-morpholin-4-yl-5-(morpholin-4-ylmethyl)phenyl]pyrimidine-2,4-diamine;
N′-(3-chloro-2,4-difluoro-phenyl)-N′-methyl-N-[3-morpholin-4-yl-5-(pyrrolidin-1-ylmethyl)phenyl]pyrimidine-2,4-diamine;
N′-(3-chloro-2,4-difluoro-phenyl)-N′-methyl-N-[3-[(4-methylpiperazin-1-yl)methyl]-5-morpholin-4-yl-phenyl]pyrimidine-2,4-diamine;
1-[[3-[[4-[(3-chloro-2,4-difluoro-phenyl)-methyl-amino]pyrimidin-2-yl]amino]-5-morpholin-4-yl-phenyl]methyl]piperidin-4-ol;
N′-(3-chloro-2,4-difluoro-phenyl)-N′-methyl-N-[3-(4-methylpiperazin-1-yl)-5-methylsulfonyl-phenyl]pyrimidine-2,4-diamine;
N′-(3-chloro-2,4-difluoro-phenyl)-N′-methyl-N-[3-(4-methylpiperazin-1-yl)phenyl]pyrimidine-2,4-diamine;
N′-(3-chloro-2,4-difluoro-phenyl)-N′-methyl-N-[3-(4-methylpiperazin-1-yl)-5-morpholin-4-yl-phenyl]pyrimidine-2,4-diamine;
[3-[[2-[(3,5-dimorpholin-4-ylphenyl)amino]pyrimidin-4-yl]-methyl-amino]-4-methyl-phenyl]methanol;
[4-methyl-3-[methyl-[2-[[3-morpholin-4-yl-5-(1,4-oxazepan-4-yl)phenyl]amino]pyrimidin-4-yl]amino]phenyl]methanol;
[4-methyl-3-[methyl-[2-[[3-(4-methylpiperazin-1-yl)-5-morpholin-4-yl-phenyl]amino]pyrimidin-4-yl]amino]phenyl]methanol;
[4-methyl-3-[methyl-[2-[(3-morpholin-4-yl-5-pyrrolidin-1-yl-phenyl)amino]pyrimidin-4-yl]amino]phenyl]methanol;
[4-methyl-3-[methyl-[2-[[3-morpholin-4-yl-5-(1-piperidyl)phenyl]amino]pyrimidin-4-yl]amino]phenyl]methanol;
[4-methyl-3-[methyl-[2-[[3-morpholin-4-yl-5-(morpholin-4-ylmethyl)phenyl]amino]pyrimidin-4-yl]amino]phenyl]methanol;
[4-methyl-3-[methyl-[2-[(3-methylsulfonyl-5-morpholin-4-yl-phenyl)amino]pyrimidin-4-yl]amino]phenyl]methanol;
1-[3-[[4-[[5-(hydroxymethyl)-2-methyl-phenyl]-methyl-amino]pyrimidin-2-yl]amino]-5-morpholin-4-yl-phenyl]piperidin-4-ol;
(3S)-1-[3-[[4-[[5-(hydroxymethyl)-2-methyl-phenyl]-methyl-amino]pyrimidin-2-yl]amino]-5-morpholin-4-yl-phenyl]pyrrolidin-3-ol;
(3R)-1-[3-[[4-[[5-(hydroxymethyl)-2-methyl-phenyl]-methyl-amino]pyrimidin-2-yl]amino]-5-morpholin-4-yl-phenyl]pyrrolidin-3-ol;
3-[[4-[[5-(hydroxymethyl)-2-methyl-phenyl]-methyl-amino]pyrimidin-2-yl]amino]-N,N-dimethyl-5-morpholin-4-yl-benzamide;
[3-[ethyl-[2-[(3-methylsulfonyl-5-morpholin-4-yl-phenyl)amino]pyrimidin-4-yl]amino]-4-methyl-phenyl]methanol;
[4-methyl-3-[[2-[(3-methylsulfonyl-5-morpholin-4-yl-phenyl)amino]pyrimidin-4-yl]-propan-2-yl-amino]phenyl]methanol;
[4-methyl-3-[2-methylpropyl-[2-[(3-methylsulfonyl-5-morpholin-4-yl-phenyl)amino]pyrimidin-4-yl]amino]phenyl]methanol;
[3-[cyclopropylmethyl-[2-[(3-methylsulfonyl-5-morpholin-4-yl-phenyl)amino]pyrimidin-4-yl]amino]-4-methyl-phenyl]methanol;
N-(3,5-dimorpholin-4-ylphenyl)-N′-(3-methoxyphenyl)-N′-methyl-pyrimidine-2,4-diamine;
N-(3,5-dimorpholin-4-ylphenyl)-N′-methyl-N′-(4-methylphenyl)pyrimidine-2,4-diamine;
N′-(3-chlorophenyl)-N-(3,5-dimorpholin-4-ylphenyl)-N′-methyl-pyrimidine-2,4-diamine;
N-(3,5-dimorpholin-4-ylphenyl)-N′-methyl-N′-phenyl-pyrimidine-2,4-diamine;
[3-[[2-[(3,5-dimorpholin-4-ylphenyl)amino]pyrimidin-4-yl]-methyl-amino]phenyl]methanol;
N-(3,5-dimorpholin-4-ylphenyl)-N′-(3-fluorophenyl)-N′-methyl-pyrimidine-2,4-diamine;
N-(3,5-dimorpholin-4-ylphenyl)-N′-methyl-N′-(3-methylphenyl)pyrimidine-2,4-diamine;
N-(3,5-dimorpholin-4-ylphenyl)-N′-methyl-N′-(3-methylsulfanylphenyl)pyrimidine-2,4-diamine;
N′-(3,5-dimethylphenyl)-N-(3,5-dimorpholin-4-ylphenyl)-N′-methyl-pyrimidine-2,4-diamine;
N′-(2,5-dimethylphenyl)-N-(3,5-dimorpholin-4-ylphenyl)-N′-methyl-pyrimidine-2,4-diamine;
3-[[2-[(3,5-dimorpholin-4-ylphenyl)amino]pyrimidin-4-yl]-methyl-amino]benzonitrile;
N′-(3,4-dichlorophenyl)-N-(3,5-dimorpholin-4-ylphenyl)-N′-methyl-pyrimidine-2,4-diamine;
N′-(4-chlorophenyl)-N-(3,5-dimorpholin-4-ylphenyl)-N′-methyl-pyrimidine-2,4-diamine;
N-(3,5-dimorpholin-4-ylphenyl)-N′-(5-methoxy-2-methyl-phenyl)-N′-methyl-pyrimidine-2,4-diamine;
N′-(5-methoxy-2-methyl-phenyl)-N′-methyl-N-(3-methylsulfonyl-5-morpholin-4-yl-phenyl)pyrimidine-2,4-diamine;
N-[2-[[2-[(3,5-dimorpholin-4-ylphenyl)amino]pyrimidin-4-yl]-methyl-amino]-4-methoxy-phenyl]acetamide;
N-[4-methoxy-2-[methyl-[2-[(3-methylsulfonyl-5-morpholin-4-yl-phenyl)amino]pyrimidin-4-yl]amino]phenyl]acetamide; or
[3-[[2-[(3-methoxy-5-morpholin-4-yl-phenyl)amino]pyrimidin-4-yl]-methyl-amino]-4-methyl-phenyl]methanol;
[3-[[4-[[5-(hydroxymethyl)-2-methyl-phenyl]-methyl-amino]pyrimidin-2-yl]amino]-5-morpholin-4-yl-phenyl]methanol;
[3-[[2-[[3-(2-methoxyethoxy)-5-morpholin-4-yl-phenyl]amino]pyrimidin-4-yl]-methyl-amino]-4-methyl-phenyl]methanol;
3-[[4-[[5-(hydroxymethyl)-2-methyl-phenyl]-methyl-amino]pyrimidin-2-yl]amino]-5-morpholin-4-yl-benzonitrile;
N-(3,5-dimorpholin-4-ylphenyl)-N′-(2-methoxyphenyl)-N′-methyl-pyrimidine-2,4-diamine;
N-(3,5-dimorpholin-4-ylphenyl)-N′-methyl-N′-(2,4,6-trimethylphenyl)pyrimidine-2,4-diamine;
N′-(2,4-difluorophenyl)-N-(3,5-dimorpholin-4-ylphenyl)-N′-methyl-pyrimidine-2,4-diamine;
N-(3,5-dimorpholin-4-ylphenyl)-N′-methyl-N′-(2-methylphenyl)pyrimidine-2,4-diamine;
N-(3,5-dimorpholin-4-ylphenyl)-N′-(2-fluorophenyl)-N′-methyl-pyrimidine-2,4-diamine;
4-[[2-[(3,5-dimorpholin-4-ylphenyl)amino]pyrimidin-4-yl]-methyl-amino]benzonitrile;
[2-chloro-5-[[2-[(3,5-dimorpholin-4-ylphenyl)amino]pyrimidin-4-yl]-methyl-amino]phenyl]methanol;
[4-chloro-3-[[2-[(3,5-dimorpholin-4-ylphenyl)amino]pyrimidin-4-yl]-methyl-amino]phenyl]methanol;
[3-chloro-5-[[2-[(3,5-dimorpholin-4-ylphenyl)amino]pyrimidin-4-yl]-methyl-amino]phenyl]methanol;
[3-[[2-[(3,5-dimorpholin-4-ylphenyl)amino]pyrimidin-4-yl]-methyl-amino]-5-methoxy-phenyl]methanol;
1-(3-((2-(3,5-dimorpholinophenylamino)pyrimidin-4-yl)(methyl)amino)-4-methylphenyl)ethanol;
3-[[4-[(5-methoxy-2-methyl-phenyl)-methyl-amino]pyrimidin-2-yl]amino]-N,N-dimethyl-5-morpholin-4-yl-benzamide;
N′-(5-methoxy-2-methyl-phenyl)-N′-methyl-N-[3-(4-methylpiperazin-1-yl)-5-morpholin-4-yl-phenyl]pyrimidine-2,4-diamine;
N′-(2,3-difluorophenyl)-N-(3,5-dimorpholin-4-ylphenyl)-N′-methyl-pyrimidine-2,4-diamine;
N′-(2,4-dichlorophenyl)-N-(3,5-dimorpholin-4-ylphenyl)-N′-methyl-pyrimidine-2,4-diamine;
4-[[2-[(3,5-dimorpholin-4-ylphenyl)amino]pyrimidin-4-yl]-methyl-amino]-2-methoxy-benzonitrile;
N′-(3,4-dimethoxyphenyl)-N-(3,5-dimorpholin-4-ylphenyl)-N′-methyl-pyrimidine-2,4-diamine;
N′-(2,5-dimethoxyphenyl)-N-(3,5-dimorpholin-4-ylphenyl)-N′-methyl-pyrimidine-2,4-diamine;
N′-(3,5-dimethoxyphenyl)-N-(3,5-dimorpholin-4-ylphenyl)-N′-methyl-pyrimidine-2,4-diamine;
2-chloro-6-[[2-[(3,5-dimorpholin-4-ylphenyl)amino]pyrimidin-4-yl]-methyl-amino]benzonitrile;
N′-(3,4-difluorophenyl)-N-(3,5-dimorpholin-4-ylphenyl)-N′-methyl-pyrimidine-2,4-diamine;
N′-(2,5-difluorophenyl)-N-(3,5-dimorpholin-4-ylphenyl)-N′-methyl-pyrimidine-2,4-diamine;
N-(3,5-dimorpholin-4-ylphenyl)-N′-methyl-N′-(2,3,4-trifluorophenyl)pyrimidine-2,4-diamine;
N-(3,5-dimorpholin-4-ylphenyl)-N′-(3-methoxy-4-methyl-phenyl)-N′-methyl-pyrimidine-2,4-diamine;
N-(3,5-dimorpholin-4-ylphenyl)-N′-(4-methoxy-2-methyl-phenyl)-N′-methyl-pyrimidine-2,4-diamine;
N-(3,5-dimorpholin-4-ylphenyl)-N′-(2-methoxy-4-methyl-phenyl)-N′-methyl-pyrimidine-2,4-diamine;
N-(3,5-dimorpholin-4-ylphenyl)-N′-(2-methoxy-5-methyl-phenyl)-N′-methyl-pyrimidine-2,4-diamine;
N′-(3-chloro-4-methoxy-phenyl)-N-(3,5-dimorpholin-4-ylphenyl)-N′-methyl-pyrimidine-2,4-diamine;
N′-(3-chloro-2-fluoro-phenyl)-N-(3,5-dimorpholin-4-ylphenyl)-N′-methyl-pyrimidine-2,4-diamine;
N′-(4-chloro-3-fluoro-phenyl)-N-(3,5-dimorpholin-4-ylphenyl)-N′-methyl-pyrimidine-2,4-diamine;
N′-(4-chloro-2-fluoro-phenyl)-N-(3,5-dimorpholin-4-ylphenyl)-N′-methyl-pyrimidine-2,4-diamine;
N′-(2-chloro-5-methyl-phenyl)-N-(3,5-dimorpholin-4-ylphenyl)-N′-methyl-pyrimidine-2,4-diamine;
N′-(3-chloro-4-methyl-phenyl)-N-(3,5-dimorpholin-4-ylphenyl)-N′-methyl-pyrimidine-2,4-diamine;
N′-(2-chloro-6-methyl-phenyl)-N-(3,5-dimorpholin-4-ylphenyl)-N′-methyl-pyrimidine-2,4-diamine;
N′-(2,3-dichlorophenyl)-N-(3,5-dimorpholin-4-ylphenyl)-N′-methyl-pyrimidine-2,4-diamine;
[3-[[2-[(3-ethoxy-5-morpholin-4-yl-phenyl)amino]pyrimidin-4-yl]-methyl-amino]-4-methyl-phenyl]methanol;
[4-methyl-3-[methyl-[2-[(3-morpholin-4-yl-5-propoxy-phenyl)amino]pyrimidin-4-yl]amino]phenyl]methanol;
[4-methyl-3-[methyl-[2-[(3-morpholin-4-yl-5-propan-2-yloxy-phenyl)amino]pyrimidin-4-yl]amino]phenyl]methanol;
[4-methyl-3-[methyl-[2-[[3-(2-methylpropoxy)-5-morpholin-4-yl-phenyl]amino]pyrimidin-4-yl]amino]phenyl]methanol;
[3-[[2-[[3-(cyclopropylmethoxy)-5-morpholin-4-yl-phenyl]amino]pyrimidin-4-yl]-methyl-amino]-4-methyl-phenyl]methanol;
[3-[[2-[(3-cyclobutyloxy-5-morpholin-4-yl-phenyl)amino]pyrimidin-4-yl]-methyl-amino]-4-methyl-phenyl]methanol;
[3-[[2-[[3-(3-methoxybutoxy)-5-morpholin-4-yl-phenyl]amino]pyrimidin-4-yl]-methyl-amino]-4-methyl-phenyl]methanol;
[3-[[2-[[3-(2-ethoxyethoxy)-5-morpholin-4-yl-phenyl]amino]pyrimidin-4-yl]-methyl-amino]-4-methyl-phenyl]methanol;
[3-[[4-[[5-(hydroxymethyl)-2-methyl-phenyl]-methyl-amino]pyrimidin-2-yl]amino]-5-morpholin-4-yl-phenyl]-morpholin-4-yl-methanone;
[3-[[4-[[5-(hydroxymethyl)-2-methyl-phenyl]-methyl-amino]pyrimidin-2-yl]amino]-5-morpholin-4-yl-phenyl]-pyrrolidin-1-yl-methanone;
3-[[4-[[5-(hydroxymethyl)-2-methyl-phenyl]-methyl-amino]pyrimidin-2-yl]amino]-N-(2-methylpropyl)-5-morpholin-4-yl-benzamide;
N-ethyl-3-[[4-[[5-(hydroxymethyl)-2-methyl-phenyl]-methyl-amino]pyrimidin-2-yl]amino]-N-methyl-5-morpholin-4-yl-benzamide;
N-(cyclopropylmethyl)-3-[[4-[[5-(hydroxymethyl)-2-methyl-phenyl]-methyl-amino]pyrimidin-2-yl]amino]-5-morpholin-4-yl-benzamide;
3-[[4-[[5-(hydroxymethyl)-2-methyl-phenyl]-methyl-amino]pyrimidin-2-yl]amino]-N-methyl-5-morpholin-4-yl-N-propan-2-yl-benzamide;
3-[[4-[[5-(hydroxymethyl)-2-methyl-phenyl]-methyl-amino]pyrimidin-2-yl]amino]-N-(2-methoxyethyl)-N-methyl-5-morpholin-4-yl-benzamide;
3-[[4-[(3-chlorophenyl)-methyl-amino]pyrimidin-2-yl]amino]-N,N-dimethyl-5-morpholin-4-yl-benzamide;
3-[[4-[(3-chlorophenyl)-methyl-amino]pyrimidin-2-yl]amino]-N-(2-hydroxyethyl)-N-methyl-5-morpholin-4-yl-benzamide;
3-[[4-[(3-chlorophenyl)-methyl-amino]pyrimidin-2-yl]amino]-N-(2-methoxyethyl)-N-methyl-5-morpholin-4-yl-benzamide;
3-[[4-[(3-chlorophenyl)-methyl-amino]pyrimidin-2-yl]amino]-N-(3-hydroxypropyl)-N-methyl-5-morpholin-4-yl-benzamide;
3-[[4-[(3-chlorophenyl)-methyl-amino]pyrimidin-2-yl]amino]-N-[(2S)-2-hydroxypropyl]-N-methyl-5-morpholin-4-yl-benzamide;
3-[[4-[(3-chlorophenyl)-methyl-amino]pyrimidin-2-yl]amino]-N-[(2S)-1-hydroxypropan-2-yl]-N-methyl-5-morpholin-4-yl-benzamide;
3-[[4-[(3-chlorophenyl)-methyl-amino]pyrimidin-2-yl]amino]-N-ethyl-N-methyl-5-morpholin-4-yl-benzamide;
3-[[4-[(3-chlorophenyl)-methyl-amino]pyrimidin-2-yl]amino]-N-methyl-5-morpholin-4-yl-N-propan-2-yl-benzamide;
[3-[[4-[(3-chlorophenyl)-methyl-amino]pyrimidin-2-yl]amino]-5-morpholin-4-yl-phenyl]-pyrrolidin-1-yl-methanone;
[3-[[4-[(3-chlorophenyl)-methyl-amino]pyrimidin-2-yl]amino]-5-morpholin-4-yl-phenyl]-morpholin-4-yl-methanone;
[3-[[4-[(3-chlorophenyl)-methyl-amino]pyrimidin-2-yl]amino]-5-morpholin-4-yl-phenyl]-(4-methylpiperazin-1-yl)methanone;
[3-[[4-[(3-chlorophenyl)-methyl-amino]pyrimidin-2-yl]amino]-5-morpholin-4-yl-phenyl]-(4-hydroxy-1-piperidyl)methanone;
3-[[4-[(3-chlorophenyl)-methyl-amino]pyrimidin-2-yl]amino]-N-cyclobutyl-5-morpholin-4-yl-benzamide;
3-[[4-[(3-chlorophenyl)-methyl-amino]pyrimidin-2-yl]amino]-5-morpholin-4-yl-N-propan-2-yl-benzamide;
3-[[4-[(3-chlorophenyl)-methyl-amino]pyrimidin-2-yl]amino]-N-(1-methoxypropan-2-yl)-5-morpholin-4-yl-benzamide;
3-[[4-[(3-chlorophenyl)-methyl-amino]pyrimidin-2-yl]amino]-N-(cyclopropylmethyl)-5-morpholin-4-yl-benzamide;
3-[[4-[(3-chlorophenyl)-methyl-amino]pyrimidin-2-yl]amino]-N-(2-methylpropyl)-5-morpholin-4-yl-benzamide;
3-[[4-[(3-chlorophenyl)-methyl-amino]pyrimidin-2-yl]amino]-N-(2-methoxyethyl)-5-morpholin-4-yl-benzamide;
3-[[4-[(3-chlorophenyl)-methyl-amino]pyrimidin-2-yl]amino]-N-(1-hydroxy-2-methyl-propan-2-yl)-5-morpholin-4-yl-benzamide;
3-[[4-[(3-chlorophenyl)-methyl-amino]pyrimidin-2-yl]amino]-5-morpholin-4-yl-N-tert-butyl-benzamide;
3-[[4-[(3-chlorophenyl)-methyl-amino]pyrimidin-2-yl]amino]-N-[(2S)-1-hydroxypropan-2-yl]-5-morpholin-4-yl-benzamide;
[3-[[4-[(3-chlorophenyl)-methyl-amino]pyrimidin-2-yl]amino]-5-morpholin-4-yl-phenyl]-[(3R)-3-hydroxypyrrolidin-1-yl]methanone;
3-[[4-[(3-chlorophenyl)-methyl-amino]pyrimidin-2-yl]amino]-N-[(2R)-1-hydroxypropan-2-yl]-5-morpholin-4-yl-benzamide;
N2-(3,5-dimorpholinophenyl)-N4-isopropyl-N4-(3-methoxyphenyl)pyrimidine-2,4-diamine;
(3-((2-(3,5-dimorpholinophenylamino)pyrimidin-4-yl)(2-methoxyethyl)amino)-4-methylphenyl)methanol;
[3-[[2-[(3,5-dimorpholin-4-ylphenyl)amino]pyrimidin-4-yl]-propan-2-yl-amino]-4-methyl-phenyl]methanol;
[3-[[2-[(3,5-dimorpholin-4-ylphenyl)amino]pyrimidin-4-yl]-ethyl-amino]-4-methyl-phenyl]methanol;
2-[[2-[(3,5-dimorpholin-4-ylphenyl)amino]pyrimidin-4-yl]-[5-(hydroxymethyl)-2-methyl-phenyl]amino]ethanol;
N-(3,5-dimorpholin-4-ylphenyl)-N′-(2-methoxyethyl)-N′-(5-methoxy-2-methyl-phenyl)pyrimidine-2,4-diamine;
N-(3,5-dimorpholin-4-ylphenyl)-N′-(5-methoxy-2-methyl-phenyl)-N′-propan-2-yl-pyrimidine-2,4-diamine;
N-(3,5-dimorpholin-4-ylphenyl)-N′-ethyl-N′-(5-methoxy-2-methyl-phenyl)pyrimidine-2,4-diamine;
2-[[2-[(3,5-dimorpholin-4-ylphenyl)amino]pyrimidin-4-yl]-(5-methoxy-2-methyl-phenyl)amino]ethanol; or
4-[3-[[4-[[5-(hydroxymethyl)-2-methyl-phenyl]-methyl-amino]pyrimidin-2-yl]amino]-5-morpholin-4-yl-phenyl]morpholin-3-one;
(S)-(4-methyl-3-(methyl(2-(3-(3-methylmorpholino)-5-morpholinophenylamino)pyrimidin-4-yl)amino)phenyl)methanol;
(R)-(4-methyl-3-(methyl(2-(3-(3-methylmorpholino)-5-morpholinophenylamino)pyrimidin-4-yl)amino)phenyl)methanol;
1-(3-(4-((5-(hydroxymethyl)-2-methylphenyl)(methyl)amino)pyrimidin-2-ylamino)-5-morpholinophenyl)piperidin-4-one;
1-(3-(4-((5-methoxy-2-methylphenyl)(methyl)amino)pyrimidin-2-ylamino)-5-morpholinophenyl)piperidin-4-ol;
N4-(5-methoxy-2-methylphenyl)-N4-methyl-N2-(3-morpholino-5-(piperazin-1-yl)phenyl)pyrimidine-2,4-diamine;
4-[3-[[4-[(5-methoxy-2-methyl-phenyl)-methyl-amino]pyrimidin-2-yl]amino]-5-morpholin-4-yl-phenyl]morpholin-3-one;
(S)—N4-(5-methoxy-2-methylphenyl)-N4-methyl-N2-(3-(3-methylmorpholino)-5-morpholino phenyl)pyrimidine-2,4-diamine;
N′-(4-fluoro-3-chloro-phenyl)-N-(3,5-dimorpholin-4-ylphenyl)-N′-methyl-pyrimidine-2,4-diamine;
[4-methyl-3-[methyl-[2-[[3-morpholin-4-yl-5-(oxolan-3-yloxy)phenyl]amino]pyrimidin-4-yl]amino]phenyl]methanol;
[4-methyl-3-[methyl-[2-[[3-morpholin-4-yl-5-[(3S)-oxolan-3-yl]oxyphenyl]amino]pyrimidin-4-yl]amino]phenyl]methanol;
[4-methyl-3-[methyl-[2-[[3-morpholin-4-yl-5-(oxan-4-yloxy)phenyl]amino]pyrimidin-4-yl]amino]phenyl]methanol;
3-[[4-[(3-chlorophenyl)-methylamino]pyrimidin-2-yl]amino]-5-morpholin-4-yl-N-(oxan-4-yl)benzamide;
3-[[4-[(3-chlorophenyl)-methylamino]pyrimidin-2-yl]amino]-N-ethyl-5-morpholin-4-ylbenzamide;
1-[3-({4-[(3-chlorophenyl)(methyl)amino]pyrimidin-2-yl}amino)-5-morpholin-4-ylphenyl]piperidin-4-ol;

N4-(3-chlorophenyl)-N4-methyl-N2-[3-(4-methylpiperazin-1-yl)-5-morpholin-4-ylphenyl]pyrimidine-2,4-diamine;
(2-((2-(3,5-dimorpholinophenyl amino)pyrimidin-4-yl)(methyl)amino)-3-methylphenyl)methanol;

N2-(3,5-dimopholinophenyl)-N4-(2-methoxy-6-methylphenyl)-N4-methylpyrimidine-2,4-diamine;
(3-((2-(3,5-dimorpholinophenyl amino)pyrimidin-4-yl)(methyl)amino)-4-fluorophenyl)methanol;
(3-((2-(3,5-dimorpholinophenylamino)pyrimidin-4-yl)(methyl)amino)-2-fluorophenyl)methanol;
1-(3-((2-(3,5-dimorpholinophenyl amino)pyrimidin-4-yl)(methyl)amino)-4-methylphenyl)ethanone;
N2-(3,5-dimorpholinophenyl)-N4-(2-fluoro-5-methoxy phenyl)-N4-methylpyrimidine-2,4-diamine;
(2-((2-(3,5-dimorpholinophenylamino)pyrimidin-4-yl)(methyl)amino)-4-methoxyphenyl)methanol;
(4-chloro-2-((2-(3,5-dimorpholinophenylamino) pyrimidin-4-yl)(methyl)amino)phenyl)methanol;
   or a pharmaceutically acceptable salt thereof

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

The compounds of the invention may be administered in the form of a pro-drug that is a compound that is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the Formula I, IA, IB, IC, ID, or IE and in vivo cleavable amide derivatives that may be formed at a carboxy group or an amino group in a compound of the Formula I, IA, IB, IC, ID or IE.

Accordingly, the present invention includes those compounds of the Formula I, IA, IB, IC, ID or IE as defined hereinbefore when made available by organic synthesis and when made available within the human or animal body by way of cleavage of a pro-drug thereof. Accordingly, the present invention includes those compounds of the Formula I, IA, IB, IC, ID or IE that are produced by organic synthetic means and also such compounds that are produced in the human or animal body by way of metabolism of a precursor compound, that is a compound of the Formula I, IA, IB, IC, ID or IE may be a synthetically-produced compound or a metabolically-produced compound.

A suitable pharmaceutically-acceptable pro-drug of a compound of the Formula I, IA, IB, IC, ID or IE is one that is based on reasonable medical judgement as being suitable for administration to the human or animal body without undesirable pharmacological activities and without undue toxicity.

Various forms of pro-drug have been described, for example in the following documents:—
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically-acceptable pro-drug of a compound of the Formula I, IA, IB, IC, ID or IE that possesses a carboxy group is, for example, an in vivo cleavable ester thereof. An in vivo cleavable ester of a compound of the Formula I containing a carboxy group is, for example, a pharmaceutically-acceptable ester, which is cleaved in the human or animal body to produce the parent acid. Suitable pharmaceutically-acceptable esters for carboxy include (1-6C)alkyl esters such as methyl, ethyl and tert-butyl, (1-6C)alkoxymethyl esters such as methoxymethyl esters, (1-6C)alkanoyloxymethyl esters such as pivaloyloxymethyl esters, 3-phthalidyl esters, (3-8C)cycloalkylcarbonyloxy-(1-6C)alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and (1-6C)alkoxycarbonyloxy-(1-6C)alkyl esters such as methoxycarbonyloxymethyl and 1-methoxycarbonyloxyethyl esters.

A suitable pharmaceutically-acceptable pro-drug of a compound of the Formula I, IA, IB, IC, ID or IE that possesses a hydroxy group is, for example, an in vivo cleavable ester or ether thereof. An in vivo cleavable ester or ether of a compound of the Formula I, IA, IB, IC, ID or IE containing a hydroxy group is, for example, a pharmaceutically-acceptable ester or ether, which is cleaved in the human or animal body to produce the parent hydroxy compound. Suitable pharmaceutically-acceptable ester forming groups for a hydroxy group include inorganic esters such as phosphate esters (including phosphoramidic cyclic esters). Further suitable pharmaceutically-acceptable ester forming groups for a hydroxy group include (1-10C)alkanoyl groups such as acetyl, benzoyl, phenylacetyl and substituted benzoyl and phenylacetyl groups, (1-10C)alkoxycarbonyl groups such as ethoxycarbonyl, N,N-[di-(1-4C)alkyl]carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(1-4C)alkylpiperazin-1-ylmethyl. Suitable pharmaceutically-acceptable ether forming groups for a hydroxy group include α-acyloxyalkyl groups such as acetoxymethyl and pivaloyloxymethyl groups.

A suitable pharmaceutically-acceptable pro-drug of a compound of the Formula I, IA, IB, IC, ID or IE that possesses an amino group is, for example, an in vivo cleavable amide derivative thereof. Suitable pharmaceutically-acceptable amides from an amino group include, for example an amide formed with (1-10C)alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(1-4C)alkylpiperazin-1-ylmethyl.

The in vivo effects of a compound of the Formula I, IA, IB, IC, ID or IE may be exerted in part by one or more metabolites that are formed within the human or animal body after administration of a compound of the Formula I, IA, IB, IC, ID or IE. As stated hereinbefore, the in vivo effects of a compound of the Formula I, IA, IB, IC, ID or IE may also be exerted by way of metabolism of a precursor compound (a pro-drug).

According to a further aspect of the invention there is provided a pharmaceutical composition, which comprises a compound of the formula I, or a pharmaceutically acceptable salt thereof, as defined hereinbefore in association with a pharmaceutically-acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

The compound of formula I will normally be administered to a warm-blooded animal at a unit dose within the range 5-5000 mg/m² body area of the animal, i.e. approximately 0.1-100 mg/kg, and this normally provides a therapeutically-effective dose. A unit dose form such as a tablet or capsule will usually contain, for example 1-250 mg of active ingredient. Preferably a daily dose in the range of 1-50 mg/kg is employed. However the daily dose will necessarily be varied depending upon the host treated, the particular route of administration, and the severity of the illness being treated. Accordingly the practitioner who is treating any particular patient may determine the optimum dosage.

### Preparation of Compounds of Formula I

It will be appreciated by a person skilled in the art that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

Furthermore, the synthesis of optically active forms may be carried out by standard techniques of organic chemistry well known in the art, for example by synthesis from optically active starting materials or by resolution of a racemic form.

Compounds of formula I can be prepared by various conventional methods as would be apparent to a chemist. In particular, compounds of formula I may be prepared by reacting a compound of formula (II):

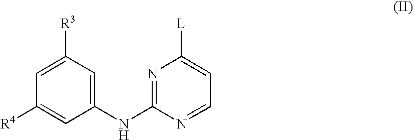

where R³ and R⁴ is as defined in relation to formula I above (with the proviso that any functional groups are optionally protected) and L is a suitable leaving group,

with a compound of formula (III)

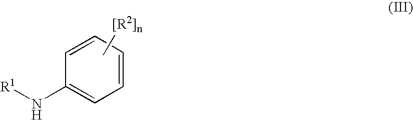

where R¹, n and R² are as defined in relation to formula I (provided that any functional groups are optionally protected).

Thereafter, any protecting groups can be removed using conventional methods, and if required, the compound of formula I can be converted to a different compound of formula I or a pharmaceutically-acceptable salt thereof, again using conventional chemical methods well known in the art.

A suitable leaving group L is halogeno, such as chloro. The reaction is suitably carried out in an organic solvent such as a C₁₋₆alkanol, for instance, n-butanol, isopropanol or 2-pentanol, dimethylacetamide (DMA), or N-methylpyrrolidine (NMP) or mixtures thereof. An acid, and in particular an inorganic acid such as hydrochloric acid, is suitably added to the reaction mixture. The reaction is suitably conducted at elevated temperatures for example at from 80-150° C., conveniently at the reflux temperature of the solvent.

Alternatively, the reaction between (II) and (III) may be catalysed by transition metals complexes, such as palladium catalysts. Examples of suitable palladium catalysts include Pd2(dba)3 (tris(dibenzylideneacetone)dipalladium), Pd(PPh₃)₄ and Pd(OAc)₂. This palladium catalysed reaction conveniently carried out in the presence of a suitable base, such as potassium carbonate, cesium carbonate, potassium phosphate, sodium tert-butoxide, or 1,8-diazabicyclo[5.4.0]undec-7-ene (DBU). Suitable solvents for such a reaction include toluene, dioxane or ethylene glycol dimethylether (DME). Suitable ligands for use in such a reaction include Xantphos (4,5-bis(diphenylphosphino)-9,9-dimethylxanthene), BINAP (2,2′-bis(diphenylphosphino)-1,1′-binaphtyl) or DPPF (1,1′-bis(diphenylphosphino)ferrocene). The reaction is conveniently carried out at an elevated temperature, generally at the reflux temperature of the particular solvent used. A temperature of 90-140° C. would be typical.

Compounds of formula (II) may be prepared by various methods including for example, where L is a halogen, by reacting a compound of formula (IV)

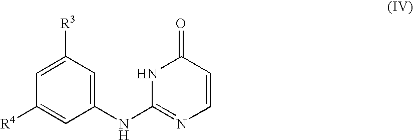

where R⁴ is as defined in relation to formula I, with a suitable halogenating agent such as phosphorus oxychloride.

The reaction is conducted under reactions conditions appropriate to the halogenating agent employed. For instance, it may be conducted at elevated temperatures, for example of from 50-100° C., in an organic solvent such as acetonitrile or DCM (DCM).

Compounds of formula (IV) are suitably prepared by reacting a compound of formula (V)

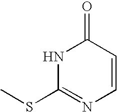

with a compound of formula (VI)

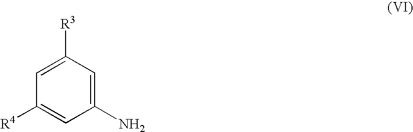

where R³ and R⁴ are as defined in relation to formula I.

The reaction is suitably effected in an organic solvent such as diglyme, again at elevated temperatures, for example from 120-180° C., and conveniently at the reflux temperature of the solvent.

Compounds of formula (II), in which L is chloro, may also be prepared by reacting a compound of formula (XIII)

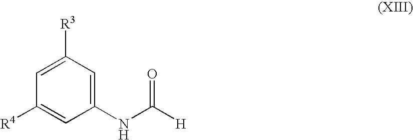

wherein R³ and R⁴ are as defined in relation to Formula I
with 4-Chloro-2-methylsulfonylpyrimidine in the presence of a suitable base, such as sodium hydride.

Alternatively, compounds of formula I may be prepared by reaction a compound of formula (VII)

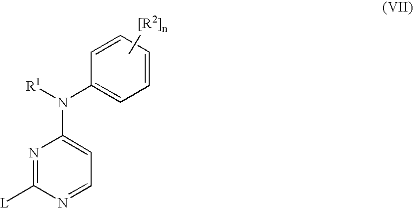

where R¹, n, and R² are as defined in relation to formula I provided that any functional groups can be optionally protected, and L is a suitable leaving group such as halogeno or —SO₂Me,

with a compound of formula (VI) as defined above.

Again, any protecting groups can be removed using conventional methods, and if required, the compound of formula I can be converted to a different compound of formula I or a salt, again using conventional chemical methods.

Suitable conditions for the reaction between (VII) and (VI) are the same as those set out above for the reaction between compounds (II) and (III) described above.

Compounds of formula (VII) are suitably prepared by reacting a compound of formula (III) as defined above with a compound of formula (VIII)

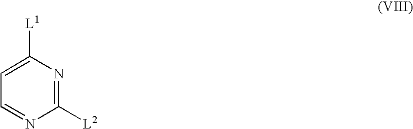

where L¹ and L² are leaving groups such as halogen, and in particular chloro.

The reaction is suitably effected in the presence of an organic base such as triethylamine. The reaction is also suitably carried out at an elevated temperature, for example between 80 and 120° C. in a suitable organic solvent such as a C₁₋₆alkanol, for instance, ethanol. The reaction can also be performed in presence of a strong base such as sodium hydride, in an organic solvent such as DMA. When the basic reaction conditions are used, depressed temperatures, for example from −20° C. to 20° C., conveniently at about 0° C. are suitably employed.

Compounds of formula (VII) can also be prepared by reacting a compound of formula (IX)

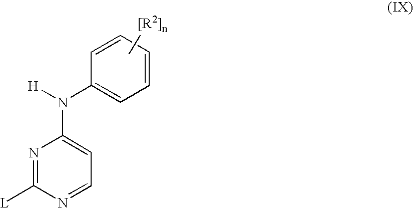

wherein L is a leaving group as defined hereinbefore and R² and n are as defined in relation to Formula I

with a compound

R¹—X

where X is a suitable leaving group such as halogen and R¹ is as defined above in relation to Formula I.

This reaction is conveniently performed using a base such as caesium carbonate in a suitable solvent, such as, for example, dimethylformamide.

Another method to prepare compounds of formula I involves the reaction of a compound formula (X)

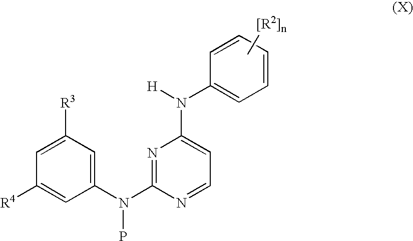

wherein R², n, R³ and R⁴ are as defined above in relation to Formula I;

with a compound

R¹—X

where X is a suitable leaving group such as halogen and R¹ is as defined above in relation to Formula I, and P is a suitable protecting group for this reaction, for example a 4-methoxybenzyl group.

This reaction is conveniently performed using a strong base such as sodium hydride in a suitable solvent, for example dimethylformamide.

Another method to prepare compounds of formula I involves the reaction of a compound of formula (XI)

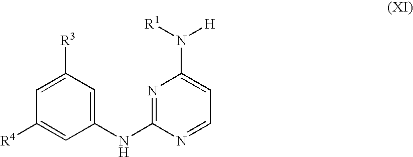

wherein R¹, R³ and R⁴ are as defined above in relation to Formula I;

with a compound of formula (XII)

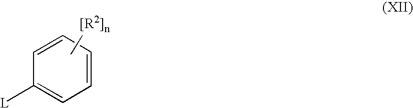

wherein R² and n are as defined above in relation to Formula I and L is halogen, for example bromo.

This reaction is suitably carried out in the presence of a suitable catalyst such as a palladium catalyst. Examples of suitable palladium catalysts include Pd2(dba)3 (tris(dibenzylideneacetone)dipalladium), Pd(PPh₃)₄ and Pd(OAc)₂. This palladium catalysed reaction conveniently carried out in the presence of a suitable base, such as potassium carbonate, cesium carbonate, potassium phosphate, sodium tert-butoxide, or 1,8-diazabicyclo[5.4.0]undec-7-ene (DBU). Suitable solvents for such a reaction include toluene, dioxane or ethylene glycol dimethylether (DME). Suitable ligands for use in such a reaction include Xantphos (4,5-bis(diphenylphosphino)-9,9-dimethylxanthene), BINAP (2,2′-bis(diphenylphosphino)-1,1′-binaphtyl) or DPPF (1,1′-bis(diphenylphosphino)ferrocene). The reaction is conveniently carried out at an elevated temperature, generally at the reflux temperature of the particular solvent used. A temperature of 90-140° C. would be typical.

Compounds of formula (III) are either known compounds or they can be prepared from known compounds using analogous methods, which would be apparent to the skilled chemist.

Compounds of the formula I can be converted into further compounds of the formula I using standard procedures conventional in the art. Examples of the types of conversion reactions that may be used to convert a compound of formula I to a different compound of formula I include introduction of a substituent by means of an aromatic substitution reaction or of a nucleophilic substitution reaction, reduction of substituents, alkylation of substituents and oxidation of substituents. The reagents and reaction conditions for such procedures are well known in the chemical art.

Particular examples of aromatic substitution reactions include the introduction of an alkyl group using an alkyl halide and Lewis acid (such as aluminium trichloride) under Friedel Crafts conditions; and the introduction of a halogeno group. Particular examples of nucleophilic substitution reactions include the introduction of an alkoxy group or of a monoalkylamino group, a dialkylamino group or a N-containing heterocycle using standard conditions. Particular examples of reduction reactions include the reduction of a carbonyl group to a hydroxy group with sodium borohydride or of a nitro group to an amino group by catalytic hydrogenation with a nickel catalyst or by treatment with iron in the presence of hydrochloric acid with heating.

The preparation of particular compounds of formula I, such as compounds of formula I, IA, IB, IC, ID or IE using the above-described methods form a further aspect of the invention.

### Biological Assays

### A) In Vitro EphB4 Enzyme Assay

This assay detects inhibitors of EphB4-mediated phosphorylation of a polypeptide substrate using Alphascreen™ luminescence detection technology. Briefly, recombinant EphB4 was incubated with a biotinylated-polypeptide substrate (biotin-poly-GAT) in presence of magnesium-ATP. The reaction was stopped by addition of EDTA, together with streptavidin-coated donor beads which bind the biotin-substrate containing any phosphorylated tyrosine residues. Anti-phosphotyrosine antibodies present on acceptor beads bind to phosphorylated substrate, thus bringing the donor & acceptor beads into close proximity. Subsequent excitation of the donor beads at 680 nm generated singlet oxygen species that interact with a chemiluminescer on the acceptor beads, leading to light emission at 520-620 nm. The signal intensity is directly proportional to the level of substrate phosphorylation and thus inhibition is measured by a decrease in signal.

Test compounds were prepared as 10 mM stock solutions in DMSO (Sigma-Aldrich Company Ltd, Gillingham, Dorset SP8 4XT Catalogue No. 154938) and serially diluted with 5% DMSO to give a range of test concentrations at 6× the required final concentration. A 2 μl aliquot of each compound dilution was transferred to appropriate wells of low volume white 384-well assay plates (Greiner, Stroudwater Business Park, Stonehouse, Gloucestershire, GL10 3SX, Cat No. 784075) in duplicate. Each plate also contained control wells: maximum signal was created using wells containing 2 μl of 5% DMSO, and minimum signal corresponding to 100% inhibition were created using wells containing 2 μl of 0.5M EDTA (Sigma-Aldrich Company Ltd, Catalogue No. E7889).

For the assay, in addition to 2 μl of compound or control, each well of the assay plate contained; 10 μl of assay mix containing final buffer (10 mM Tris, 100 μM EGTA, 10 mM magnesium acetate, 4 μM ATP, 500 μM DTT, 1 mg/ml BSA), 0.25 ng of recombinant active EphB4 (amino acids 563-987; Swiss-Prot Acc. No. P54760) (ProQinase GmbH, Breisacher Str. 117, D-79106 Freiburg, Germany, Catalogue No 0178-0000-3) and 5 nM of the poly-GAT substrate (CisBio International, BP 84175, 30204 Bagnols/Cèze Cedex, France, Catalogue No. 61GATBLB). Assay plates were then incubated at room temperature for 1 hr. The reaction was then stopped by addition of 5 μl/well stop buffer (10 mM Tris, 495 mM EDTA, 1 mg/ml BSA) containing 0.25 ng each of AlphaScreen anti-phospho Tyrosine-100 acceptor beads and streptavidin-coated donor beads (Perkin Elmer, Catalogue No 6760620M). The plates were sealed under natural lighting conditions, wrapped in aluminium foil and incubated in the dark for a further 20 hrs.

The resulting assay signal was determined on the Perkin Elmer EnVision plate reader. The minimum value was subtracted from all values, and the signal plotted against compound concentration to generate IC₅₀ data. Compounds of the invention were tested in the in vitro EphB4 enzyme assay and the IC₅₀ values so obtained are presented in Table A below.

TABLE A

| Example | EphB4 enzyme |
|---|---|
| Number | assay IC₅₀ value |
| 1 | 0.763 |
| 2.1 | 0.829 |
| 2.2 | 2.18 |
| 2.3 | 0.304 |
| 2.4 | 0.348 |
| 2.5 | 0.937 |
| 2.6 | 0.447 |
| 2.7 | 0.117 |
| 2.8 | 0.335 |
| 2.9 | 0.781 |
| 2.10 | 1.27 |
| 2.11 | 0.106 |
| 2.12 | 0.189 |
| 2.13 | 0.0697 |
| 2.14 | 0.653 |
| 2.15 | 1.11 |
| 2.16 | 0.307 |
| 2.17 | 0.0289 |
| 2.18 | 0.0461 |
| 2.19 | 0.0596 |
| 3 | 0.0651 |
| 4 | 0.0638 |
| 5 | 0.424 |
| 6 | 0.052 |
| 7.1 | 0.361 |
| 7.2 | 0.0431 |
| 7.3 | 0.657 |
| 7.4 | 0.51 |
| 7.5 | 0.113 |
| 7.7 | 0.144 |
| 7.8 | 0.0759 |
| 7.9 | 0.151 |
| 7.10 | 0.0135 |
| 7.11 | 0.0601 |
| 7.12 | 0.116 |
| 7.13 | 0.0326 |
| 7.14 | 0.0659 |
| 7.15 | 0.195 |
| 7.16 | 0.0222 |
| 7.17 | 0.00313 |
| 7.18 | 0.00645 |
| 7.19 | 0.00973 |
| 8.1 | 0.414 |
| 8.2 | 0.32 |
| 8.3 | 0.917 |
| 8.4 | 1.07 |
| 9 | 0.0565 |
| 10.1 | 0.402 |
| 10.2 | 0.0453 |
| 10.3 | 0.0758 |
| 10.4 | 0.0481 |
| 10.5 | 0.34 |
| 10.6 | 0.511 |
| 10.7 | 0.638 |
| 10.8 | 1.1 |
| 10.9 | 0.543 |
| 10.10 | 0.527 |
| 10.11 | 0.66 |
| 10.12 | 0.107 |
| 10.13 | 0.811 |
| 10.14 | 0.381 |
| 10.15 | 0.116 |
| 10.16 | 0.237 |
| 10.17 | 9.78 |
| 10.18 | 0.0968 |
| 10.19 | 0.15 |
| 10.20 | 0.338 |
| 10.21 | 0.147 |
| 10.22 | 0.055 |
| 11 | |
| 12.1 | 0.0175 |
| 12.2 | 0.0294 |
| 12.3 | 0.455 |
| 12.4 | 0.171 |
| 12.5 | 0.00295 |
| 12.6 | 0.0034 |
| 12.7 | 0.305 |
| 12.8 | 0.00582 |
| 13.1 | 0.47 |
| 13.2 | 0.426 |
| 14 | 0.37 |
| 15.1 | 34.1 |
| 15.2 | 0.206 |
| 15.3 | 1 |
| 15.4 | 0.68 |
| 15.5 | 1.12 |
| 15.6 | 0.417 |
| 15.7 | 0.707 |
| 15.8 | 0.338 |
| 15.9 | 0.602 |
| 15.10 | 7.73 |
| 15.11 | 1.32 |
| 15.12 | 1.4 |
| 15.13 | 0.722 |
| 15.14 | 0.5 |
| 15.15 | 0.696 |
| 15.16 | 0.634 |
| 15.17 | 1.15 |
| 15.18 | 1.39 |
| 15.19 | 1.73 |
| 15.20 | 1.75 |
| 15.21 | 1.31 |
| 15.22 | 0.316 |
| 16 | 0.132 |
| 17.1 | 0.265 |
| 17.2 | 0.171 |
| 17.3 | 1.91 |
| 17.4 | 1.09 |
| 17.5 | 0.951 |
| 17.6 | 1.21 |
| 17.7 | 0.639 |
| 17.8 | 0.481 |
| 17.9 | 0.443 |
| 17.10 | 0.0646 |
| 18.1 | 0.0338 |
| 18.2 | 0.0623 |
| 18.3 | 0.246 |
| 18.4 | 0.0753 |
| 18.5 | 0.477 |
| 18.6 | 0.342 |
| 18.7 | 0.118 |
| 19.1 | 0.419 |
| 19.2 | 0.395 |
| 19.3 | 0.571 |
| 19.4 | 0.295 |
| 19.5 | 0.181 |
| 19.6 | 0.339 |
| 19.7 | 0.648 |
| 19.8 | 1.45 |
| 19.9 | 0.754 |
| 19.10 | 0.656 |
| 19.11 | 0.207 |
| 19.12 | 0.234 |
| 19.13 | 2.08 |
| 19.14 | 1.18 |
| 19.15 | 1.1 |
| 19.16 | 1.97 |
| 19.17 | 2.83 |
| 19.18 | 0.614 |
| 19.19 | 1.05 |
| 19.20 | 3.09 |
| 19.21 | 0.527 |
| 19.22 | 0.119 |
| 19.23 | 1.31 |
| 19.24 | 1 |
| 19.25 | 0.875 |
| 20 | 0.912 |
| 21 | 0.0532 |
| 21.1 | 0.0433 |
| 21.2 | 0.0274 |
| 21.3 | 0.025 |
| 22.1 | 1.48 |
| 22.2 | 1.86 |
| 22.3 | 1.19 |
| 22.4 | 0.802 |
| 23 | 0.00171 |
| 24 | 0.000581 |
| 25.1 | 0.0528 |
| 25.2 | 0.0769 |
| 25.3 | 0.0115 |
| 25.4 | 0.0116 |
| 25.5 | 0.127 |
| 25.6 | 0.00788 |
| 25.7 | 0.0103 |
| 25.8 | 0.00639 |

### B) In Vitro EphB4 Cell Assay

The assay identifies inhibitors of cellular EphB4 by measuring a decrease in phosphorylation of EphB4 following treatment of cells with compound. The endpoint assay used a sandwich ELISA to detect EphB4 phosphorylation status. Briefly, Myc-tagged EphB4 from treated cell lysate was captured on the ELISA plate via an anti-c-Myc antibody. The phosphorylation status of captured EphB4 was then measured using a generic phosphotyrosine antibody conjugated to HRP via a colourimetric output catalysed by HRP, with level of EphB4 phosphorylation directly proportional to the colour intensity. Absorbance was measured spectrophotometrically at 450 nm.

Full length human EphB4 (Swiss-Prot Acc. No. P54760) was cloned using standard techniques from cDNA prepared from HUVEC using RT-PCR. The cDNA fragment was then sub-cloned into a pcDNA3.1 expression vector containing a Myc-His epitope tag to generate full-length EphB4 containing a Myc-His tag at the C-terminus (Invitrogen Ltd. Paisley, UK). CHO-K1 cells (LGC Promochem, Teddington, Middlesex, UK, Catalogue No. CCL-61) were maintained in HAM's F12 medium (Sigma-Aldrich Company Ltd, Gillingham, Dorset SP8 4XT, Catalogue No. N4888) containing 10% heat-inactivated foetal calf serum (PAA lab GmbH, Pasching, Austria Catalogue No. PAA-A15-043) and 1% glutamax-1 (Invitrogen Ltd., Catalogue No. 35050-038) at 37° C. with 5% CO₂. CHO-K1 cells were engineered to stably express the EphB4-Myc-His construct using standard stable transfection techniques, to generate cells hereafter termed EphB4-CHO.

For each assay, 10,000 EphB4-CHO cells were seeded into each well of Costar 96-well tissue-culture plate (Fisher Scientific UK, Loughborough, Leicestershire, UK., Catalogue No. 3598) and cultured overnight in full media. On day 2, the cells were incubated overnight in 90 μl/well of media containing 0.1% Hyclone stripped-serum (Fisher Scientific UK, Catalogue No. SH30068.02). Test compounds were prepared as 10 mM stock solutions in DMSO (Sigma-Aldrich Company Ltd, Gillingham, Dorset SP8 4XT Catalogue No. 154938) and serially diluted with serum-free media to give a range of test concentrations at 10× the required final concentration. A 10 μl aliquot of each compound dilution was transferred to the cell plates in duplicate wells, and the cells incubated for 1 hr at 37° C. Each plate also contained control wells: a maximum signal was created using untreated cells, and minimum signal corresponding to 100% inhibition was created using wells containing a reference compound known to abolish EphB4 activity.

Recombinant ephrin-B2-Fc (R&D Systems, Abingdon Science Park, Abingdon, Oxon OX14 3NB UK, Catalogue No. 496-EB), a Fc-tagged form of the cognate ligand for EphB4, was pre-clustered at a concentration of 3 μg/ml with 0.3 μg/ml anti-human IgG, Fc fragment specific (Jackson ImmunoResearch Labs, Northfield Business Park, Soham, Cambridgeshire, UK CB7 5UE, Catalogue No. 109-005-008) in serum-free media for 30 minutes at 4° C. with occasional mixing. Following compound treatment, cells were stimulated with clustered ephrin-B2 at a final concentration of 1 μg/ml for 20 minutes at 37° C. to induce EphB4 phosphorylation. Following stimulation, the medium was removed and the cells lysed in 100 μl/well of lysis buffer (25 mM Tris HCl, 3 mM EDTA, 3 mM EGTA, 50 mM NaF, 2 mM orthovanadate, 0.27M Sucrose, 10 mM β-glycerophosphate, 5 mM sodium pyrophosphate, 2% Triton X-100, pH 7.4).

Each well of an ELISA Maxisorp 96-well plate (Nunc; Fisher Scientific UK, Loughborough, Leicestershire, UK., Catalogue No. 456537) was coated overnight at 4° C. with 100 μl of anti-c-Myc antibody in Phosphate Buffered Saline (10 μg/ml; produced at AstraZeneca). Plates were washed twice with PBS containing 0.05% Tween-20 and blocked with 250 μl/well 3% TopBlock (Fluka) (Sigma-Aldrich Company Ltd, Gillingham, Dorset SP8 4XT, Catalogue No. 37766) for a minimum of 2 hrs at room temperature. Plates were washed twice with PBS/0.05% Tween-20 and incubated with 100 μl/well cell lysate overnight at 4° C. ELISA plates were washed four times with PBS/0.05% Tween-20 and incubated for 1 hr at room temperature with 100 μl/well HRP-conjugated 4G10 anti-phosphotyrosine antibody (Upstate, Dundee Technology Park, Dundee, UK, DD2 1 SW, Catalogue No. 16-105) diluted 1:6000 in 3% Top Block. ELISA plates were washed four times with PBS/0.05% Tween-20 and developed with 100 μl/well TMB substrate (Sigma-Aldrich Company Ltd, Catalogue No. T0440). The reaction was stopped after 15 minutes with the addition of 25 μl/well 2M sulphuric acid. The absorbances were determined at 450 nm using the Tecan SpectraFluor Plus. The minimum value was subtracted from all values, and the signal plotted against compound concentration to generate IC₅₀ data.

Compounds of the invention were active in the above assays, for instance, generally showing IC₅₀ values of less than 3 μM in Assay A and 0.3M in Assay B. Preferred compounds of the invention generally showing IC₅₀ values of less than 0.1 μM in Assay B. Further illustrative IC₅₀ values obtained using Assay B for a selection of the compounds exemplified in the present application are shown in Table B below.

TABLE B
Mean IC₅₀ values obtained using Assay B

| Example No. | Mean IC₅₀ (μM) |
|---|---|
| 2.2 | 0.074 |
| 2.10 | 0.011 |
| 2.15 | 0.017 |
| 2.17 | 0.021 |
| 4 | 1.71 |
| 7.2 | <0.032 |

### C) In Vivo Tumour Explant Model

Human Colorectal cancer cells (HT29 5×10⁶ (ATCC HTB-38)) were implanted subcutaneously on to the left flank of nude male mice (nu/nu:Alpk). Actively growing tumours approx 0.2-0.3 cm³ were selected and randomised. [3-[[2-[(3,5-dimorpholin-4-ylphenyl)amino]pyrimidin-4-yl]-methyl-amino]-4-methyl-phenyl]methanol was dosed orally at 100 mg/kg b.i.d using a gavage in a formulation of 0.5% w/v Methocel+0.1% w/v Poly (HPMC/Tween) for 16 consecutive days. [3-[[2-[(3,5-dimorpholin-4-ylphenyl)amino]pyrimidin-4-yl]-methyl-amino]-4-methyl-phenyl]methanol was dosed as a stand alone agent or in combination with the VEGF receptor tyrosine kinase inhibitor 4-(4-fluoro-2-methylindol-5-yloxy)-6-methoxy-7-(3-piperidin-1-yl-propoxy)-quinazoline (AZD7514) (dose u.d). Tumours were measured twice weekly and volumes (cm³) calculated (π/6×(length×width×width)/1000). On the final day of the dosing the tumours were measured, percent growth inhibition was then calculated compared to the control (((GMDVT−GMDVC)/(GMDVC−1))*100), where GMDV is the geometric delta volume of either treated (T) or control (C), and a T-Test was performed to establish significance.

Figure H shows the effect on mean tumour volumes of dosing with [3-[[2-[(3,5-dimorpholin-4-ylphenyl)amino]pyrimidin-4-yl]-methyl-amino]-4-methyl-phenyl]methanol and 4-(4-fluoro-2-methylindol-5-yloxy)-6-methoxy-7-(3-piperidin-1-yl-propoxy)-quinazoline (AZD7514) either individually or in combination.

As a result of their activity in the screens described and the effectiveness of [3-[[2-[(3,5-dimorpholin-4-ylphenyl)amino]pyrimidin-4-yl]-methyl-amino]-4-methyl-phenyl]methanol in reducing tumour volume in the in vivo tumour explant model above, the compounds of the present invention are expected to be useful in the treatment of diseases or medical conditions mediated alone or in part by EphB4 enzyme activity, i.e. the compounds may be used to produce an EphB4 inhibitory effect in a warm-blooded animal in need of such treatment. Thus, the compounds of the present invention provide a method for treating the proliferation of malignant cells characterised by inhibition of the EphB4 enzyme, i.e. the compounds may be used to produce an anti-proliferative effect mediated alone or in part by the inhibition of EphB4.

According to another aspect of the present invention there is provided a compound of the formula I, IA, IB, IC, ID or IE, or a pharmaceutically acceptable salt thereof, as defined hereinbefore for use in a method of treatment of the human or animal body by therapy.

Thus according to a further aspect of the invention there is provided a compound of the formula I, IA, IB, IC, ID or IE, or a pharmaceutically acceptable salt thereof, as defined hereinbefore for use as a medicament.

According to a further aspect of the invention there is provided the use of a compound of the formula I, IA, IB, IC, ID or IE, or a pharmaceutically acceptable salt thereof, as defined hereinbefore in the manufacture of a medicament for use in the production of an EphB4 inhibitory effect in a warm-blooded animal such as man.

According to a further feature of this aspect of the invention there is provided a method for producing an EphB4 inhibitory effect in a warm-blooded animal, such as man, in need of such treatment which comprises administering to said animal an effective amount of a compound of the formula I, IA, IB, IC, ID or IE, or a pharmaceutically acceptable salt thereof, as defined hereinbefore.

According to a further aspect of the invention there is provided the use of a compound of the formula I, IA, IB, IC, ID or IE, or a pharmaceutically acceptable salt thereof, as defined hereinbefore in the manufacture of a medicament for use in the production of an anti-angiogenic effect in a warm-blooded animal such as man.

According to a further feature of this aspect of the invention there is provided a method for producing an anti-angiogenic effect in a warm-blooded animal, such as man, in need of such treatment which comprises administering to said animal an effective amount of a compound of the formula I, IA, IB, IC, ID or IE, or a pharmaceutically acceptable salt thereof, as defined hereinbefore.

According to an additional feature of this aspect of the invention there is provided a method of treating cancer in a warm-blooded animal, such as man, in need of such treatment which comprises administering to said animal an effective amount of a compound of the formula I, IA, IB, IC, ID or IE, or a pharmaceutically acceptable salt thereof, as defined hereinbefore.

According to a further feature of the invention there is provided the use of a compound of the formula I, IA, IB, IC, ID or IE, or a pharmaceutically acceptable salt thereof, as defined hereinbefore in the manufacture of a medicament for use in the treatment of cancer.

According to an additional feature of this aspect of the invention there is provided a compound of the formula I, IA, IB, IC, ID or IE, or a pharmaceutically acceptable salt thereof, as defined hereinbefore, for use in the treatment of cancer.

According to an additional feature of this aspect of the invention there is provided the use of a compound of the formula I, IA, IB, IC, ID or IE, or a pharmaceutically acceptable salt thereof, as defined hereinbefore, for use in the manufacture of a medicament for the treatment of cancer.

In a further aspect of the present invention there is provided the use of a compound of the formula I, IA, IB, IC, ID or IE, or a pharmaceutically acceptable salt thereof, as defined hereinbefore, in the manufacture of a medicament for use in the treatment of solid tumour disease, in particular neuroblastomas, breast, liver, lung and colon cancer or leukemias.

In a further aspect of the present invention there is provided a method of treating neuroblastomas, breast, liver, lung and colon cancer or leukemias in a warm-blooded animal, such as man, in need of such treatment which comprises administering to said animal an effective amount of a compound of the formula I, IA, IB, IC, ID or IE, or a pharmaceutically acceptable salt thereof, as defined hereinbefore.

The anti-cancer treatment defined hereinbefore may be applied as a sole therapy or may involve, in addition to the compound of the invention, conventional surgery or radiotherapy or chemotherapy. Such conjoint treatment may be achieved by way of the simultaneous, sequential or separate administration of the individual components of the treatment. In the field of medical oncology it is normal practice to use a combination of different forms of treatment to treat each patient with cancer. In medical oncology the other component(s) of such conjoint treatment in addition to the anti-angiogenic treatment defined hereinbefore may be: surgery, radiotherapy or chemotherapy. Such chemotherapy may include one or more of the following categories of anti-tumour agents:—

(i) other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example antifolates such as fluoropyrimidines like 5-fluorouracil and tegafur, raltitrexed, methotrexate, cytosine arabinoside, hydroxyurea and gemcitabine); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine, taxoids like taxol and taxotere, and polo kinase inhibitors); and topoisomerase inhibitors (for example epipodophyllotoxins like etoposide and teniposide, amsacrine, topotecan and camptothecin);
(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) anti-invasion agents [for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZDO530; International Patent Application WO 01/94341) and bosutinib (SKI-606), and metalloproteinase inhibitors like marimastat and inhibitors of urokinase plasminogen activator receptor function];
(iv) inhibitors of growth factor function: for example such inhibitors include growth factor antibodies and growth factor receptor antibodies [for example the anti-erbB2 antibody trastuzumab and the anti-erbB1 antibodies cetuximab (C225) and panitumumab]; such inhibitors also include, for example, tyrosine kinase inhibitors [for example inhibitors of the epidermal growth factor family (for example EGFR family tyrosine kinase inhibitors such as gefitinib (ZD1839), erlotinib (OSI-774) and CI 1033, and erbB2 tyrosine kinase inhibitors such as lapatinib), inhibitors of the hepatocyte growth factor family, inhibitors of the insulin growth factor receptor, inhibitors of the platelet-derived growth factor family and/or bcr/abl kinase such as imatinib, dasatinib (BMS-354825) and nilotinib (AMN107), inhibitors of cell signalling through MEK, AKT, PI3, c-kit, Flt3, CSF-1R and/or aurora kinases]; such inhibitors also include cyclin dependent kinase inhibitors including CDK2 and CDK4 inhibitors; and such inhibitors also include, for example, inhibitors of serine/threonine kinases (for example Ras/Raf signalling inhibitors such as farnesyl transferase inhibitors, for example sorafenib (BAY 43-9006), tipifarnib (R115777) and lonafarnib (SCH66336);
(v) antiangiogenic agents such as those which inhibit the effects of vascular endothelial growth factor, [for example an anti-vascular endothelial cell growth factor antibody such as bevacizumab (Avastin™) or, for example, a vascular endothelial growth factor (VEGF) receptor tyrosine kinase inhibitor such as vandetanib (ZD6474), vatalanib (PTK787), sunitinib (SU11248), axitinib (AG-013736), pazopanib (GW 786034), 4-(4-fluoro-2-methylindol-5-yloxy)-6-methoxy-7-(3-pyrrolidin-1-ylpropoxy)quinazoline (AZD2171; Example 240 within WO 00/47212) and 4-(4-fluoro-2-methylindol-5-yloxy)-6-methoxy-7-(3-piperidinopropoxy)quinazoline (AZD7514; Example 238 within WO 00/47212), or, for example, a compound that works by another mechanism (for example linomide, inhibitors of integrin αvβ3 function and angiostatin)];
(vi) vascular damaging agents such as Combretastatin A4;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell energy, approaches using transfected immune cells such as cytokine-transfected dendritic cells, approaches using cytokine-transfected tumour cell lines and approaches using anti-idiotypic antibodies.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

According to this aspect of the invention there is provided a combination suitable for use in the treatment of cell proliferative disorders (such as solid tumour disease) comprising a compound of formula I, IA, IB, IC, ID or IE as defined hereinbefore and an additional anti-tumour agent as defined hereinbefore.

According to this aspect of the invention there is also provided a combination suitable for use in the treatment of cell proliferative disorders (such as solid tumour disease) comprising a compound of formula I, IA, IB, IC, ID or IE as defined hereinbefore and an additional antiangiogenic agent.

In one embodiment of this aspect of the invention, there is provided a combination suitable for use in the treatment of cell proliferative disorders (such as solid tumour disease) comprising a compound of formula I, IA, IB, IC, ID or IE as defined hereinbefore and a VEGF receptor tyrosine kinase inhibitor, for example 4-(4-fluoro-2-methylindol-5-yloxy)-6-methoxy-7-(3-pyrrolidin-1-ylpropoxy)quinazoline, or a pharmaceutically acceptable salt thereof, or 4-(4-fluoro-2-methylindol-5-yloxy)-6-methoxy-7-(3-piperidin-1-yl-propoxy)-quinazoline, or a pharmaceutically acceptable salt thereof.

In a further embodiment of this aspect of the invention, there is provided a combination suitable for use in the treatment of cell proliferative disorders (such as solid tumour disease) comprising [3-[[2-[(3,5-dimorpholin-4-ylphenyl)amino]pyrimidin-4-yl]-methyl-amino]-4-methyl-phenyl]methanol, or a pharmaceutically acceptable salt thereof, and a VEGF receptor tyrosine kinase inhibitor, for example 4-(4-fluoro-2-methylindol-5-yloxy)-6-methoxy-7-(3-pyrrolidin-1-ylpropoxy)quinazoline, or a pharmaceutically acceptable salt thereof, or 4-(4-fluoro-2-methylindol-5-yloxy)-6-methoxy-7-(3-piperidin-1-yl-propoxy)-quinazoline, or a pharmaceutically acceptable salt thereof.

In one particular embodiment of this aspect of the invention there is provided a combination suitable for use in the treatment of cell proliferative disorders (such as solid tumour disease) comprising [3-[[2-[(3,5-dimorpholin-4-ylphenyl)amino]pyrimidin-4-yl]-methyl-amino]-4-methyl-phenyl]methanol, or a pharmaceutically acceptable salt thereof, and 4-(4-fluoro-2-methylindol-5-yloxy)-6-methoxy-7-(3-pyrrolidin-1-ylpropoxy)quinazoline, or a pharmaceutically acceptable salt thereof.

A preferred salt of 4-(4-fluoro-2-methylindol-5-yloxy)-6-methoxy-7-(3-pyrrolidin-1-ylpropoxy)quinazoline (AZD2171) is the maleate salt (AZD2171 maleate) which is described in International Patent Application Publication No. WO 05/061488. AZD2171 maleate salt may be synthesised according to the processes described in WO 05/061488.

In one embodiment of this aspect of the invention there is therefore provided a combination suitable for use in the treatment of cell proliferative disorders (such as solid tumour disease) comprising [3-[[2-[(3,5-dimorpholin-4-ylphenyl)amino]pyrimidin-4-yl]-methyl-amino]-4-methyl-phenyl]methanol, or a pharmaceutically acceptable salt thereof, and the maleate salt of 4-(4-fluoro-2-methylindol-5-yloxy)-6-methoxy-7-(3-pyrrolidin-1-ylpropoxy)quinazoline.

According to this aspect of the invention there is also provided a pharmaceutical product comprising a compound of formula I, IA, IB, IC, ID or IE as defined hereinbefore and an additional antiangiogenic agent.

In one embodiment of this aspect of the invention, there is provided a pharmaceutical product comprising a compound of formula I, IA, IB, IC, ID or IE as defined hereinbefore and a VEGF receptor tyrosine kinase inhibitor, for example 4-(4-fluoro-2-methylindol-5-yloxy)-6-methoxy-7-(3-pyrrolidin-1-ylpropoxy)quinazoline, or a pharmaceutically acceptable salt thereof, or 4-(4-fluoro-2-methylindol-5-yloxy)-6-methoxy-7-(3-piperidin-1-yl-propoxy)-quinazoline, or a pharmaceutically acceptable salt thereof.

In a further embodiment of this aspect of the invention, there is provided a pharmaceutical product comprising [3-[[2-[(3,5-dimorpholin-4-ylphenyl)amino]pyrimidin-4-yl]-methyl-amino]-4-methyl-phenyl]methanol, or a pharmaceutically acceptable salt thereof, and a VEGF receptor tyrosine kinase inhibitor, for example 4-(4-fluoro-2-methylindol-5-yloxy)-6-methoxy-7-(3-pyrrolidin-1-ylpropoxy)quinazoline, or a pharmaceutically acceptable salt thereof, or 4-(4-fluoro-2-methylindol-5-yloxy)-6-methoxy-7-(3-piperidin-1-yl-propoxy)-quinazoline, or a pharmaceutically acceptable salt thereof.

In one particular embodiment of this aspect of the invention there is provided a pharmaceutical product comprising [3-[[2-[(3,5-dimorpholin-4-ylphenyl)amino]pyrimidin-4-yl]-methyl-amino]-4-methyl-phenyl]methanol, or a pharmaceutically acceptable salt thereof, and 4-(4-fluoro-2-methylindol-5-yloxy)-6-methoxy-7-(3-pyrrolidin-1-ylpropoxy)quinazoline, or a pharmaceutically acceptable salt thereof.

In one embodiment of this aspect of the invention there is provided a pharmaceutical product comprising [3-[[2-[(3,5-dimorpholin-4-ylphenyl)amino]pyrimidin-4-yl]-methyl-amino]-4-methyl-phenyl]methanol, or a pharmaceutically acceptable salt thereof, and the maleate salt of 4-(4-fluoro-2-methylindol-5-yloxy)-6-methoxy-7-(3-pyrrolidin-1-ylpropoxy)quinazoline.

According to this aspect of the invention there is also provided a pharmaceutical product comprising a compound of formula I, IA, IB, IC, ID or IE as defined hereinbefore and an additional anti-tumour agent as defined hereinbefore for the conjoint treatment of cancer.

As stated above the size of the dose required for the therapeutic or prophylactic treatment of a particular cell-proliferation disease will necessarily be varied depending on the host treated, the route of administration and the severity of the illness being treated. A unit dose in the range, for example, 1-100 mg/kg, preferably 1-50 mg/kg is envisaged.

The combination treatments of the present invention as defined herein are of interest for their antiangiogenic and/or vascular permeability effects. Angiogenesis and/or an increase in vascular permeability is present in a wide range of disease states including cancer (including leukaemia, multiple myeloma and lymphoma), diabetes, psoriasis, rheumatoid arthritis, Kaposi's sarcoma, haemangioma, acute and chronic nephropathies, atheroma, arterial restenosis, autoimmune diseases, acute inflammation, asthma, lymphodema, endometriosis, dysfunctional uterine bleeding and ocular diseases with retinal vessel proliferation including age-related macular degeneration.

Combination treatments of the present invention are expected to be particularly useful in the prophylaxis and treatment of diseases such as cancer and Kaposi's sarcoma. In particular such combination treatments of the invention are expected to be useful in the treatment of cancer, for example cancer of the lung, head and neck, brain, colon, rectum, esophagus, stomach, liver, biliary tract, thyroid, kidney, cervix, ovary, uterus, skin, breast, bladder, prostate, pancreas and including haematological malignancies such as leukaemia, multiple myeloma and lymphoma.

In particular such combination treatments of the invention are expected to slow advantageously the growth of primary and recurrent solid tumours.

More particularly such combination treatments of the invention are expected to inhibit any form of cancer associated with VEGF including leukaemia, multiple myeloma and lymphoma and also, for example, to inhibit the growth of those primary and recurrent solid tumours which are associated with VEGF, especially those tumours which are significantly dependent on VEGF for their growth and spread, including for example, certain tumours of the colon, rectum, pancreas, brain, bladder, ovary, breast, prostate, lung, vulva, liver and skin.

In addition to their use in therapeutic medicine, the compounds of formula I, IA, or IB and their pharmaceutically acceptable salts thereof, are also useful as pharmacological tools in the development and standardisation of in vitro and in vivo test systems for the evaluation of the effects of inhibitors of anti-angiogenic activity in laboratory animals such as cats, dogs, rabbits, monkeys, rats and mice, as part of the search for new therapeutic agents.

The invention will now be illustrated in the following Examples in which, generally:

(i) temperatures are given in degrees Celsius (° C.); operations were carried out at room or ambient temperature, that is, at a temperature in the range of 18 to 25° C.;
(ii) organic solutions were dried over anhydrous magnesium sulfate or anhydrous sodium sulfate; evaporation of solvent was carried out using a rotary evaporator under reduced pressure (600 to 4000 Pascals; 4.5 to 30 mmHg) with a bath temperature of up to 60° C.;
(iii) chromatography means flash chromatography on silica gel; thin layer chromatography (TLC) was carried out on silica gel plates;
(iv) in general, the course of reactions was followed by TLC and/or analytical LC-MS, and reaction times are given for illustration only. The retention times (t_{R}) were measured on a LC/MS Waters 2790/ZMD Micromass system equipped with a Waters Symmetry column (C18, 3.5 μM, 4.6×50 mm) or a Waters Sunfire column (C18, 3.5 μM, 4.6×50 mm); detection UV 254 nM and MS; elution: flow rate 2.5 ml/min, linear gradient from 95% water—5% methanol containing 5% formic acid to 40% water—55% acetonitrile—5% methanol containing 5% formic acid over 3 minutes; then linear gradient to 95% acetonitrile—5% methanol containing 5% formic acid over 1 minute;
(v) final products had satisfactory proton nuclear magnetic resonance (NMR) spectra and/or mass spectral data;
(vi) yields are given for illustration only and are not necessarily those which can be obtained by diligent process development; preparations were repeated if more material was required;
(vii) when given, NMR data is in the form of delta values for major diagnostic protons, given in parts per million (ppm) relative to tetramethylsilane (TMS) as an internal standard, determined at 500 MHz using perdeuterio dimethyl sulfoxide (DMSOd₆) as solvent unless otherwise indicated; the following abbreviations have been used: s, singlet; d, doublet; t, triplet; q, quartet; m, multiplet; br, broad;
(viii) chemical symbols have their usual meanings; SI units and symbols are used;
(ix) solvent ratios are given in volume:volume (v/v) terms; and
(x) mass spectra were run with an electron energy of 70 electron volts in the chemical ionization (CI) mode using a direct exposure probe; where indicated ionization was effected by electron impact (EI), fast atom bombardment (FAB) or electrospray (ESP); values for m/z are given; generally, only ions which indicate the parent mass are reported; and unless otherwise stated, the mass ion quoted is (MH)⁺ which refers to the protonated mass ion; reference to M⁺ is to the mass ion generated by loss of an electron; and reference to M−H⁺ is to the mass ion generated by loss of a proton;
(xi) unless stated otherwise compounds containing an asymmetrically substituted carbon and/or sulfur atom have not been resolved;
(xii) where a synthesis is described as being analogous to that described in a previous example the amounts used are the millimolar ratio equivalents to those used in the previous example;
(xiii) all microwave reactions were carried out in a Personal Chemistry EMRYS™ Optimizer EXP microwave synthesizer;
(xiv) preparative high performance liquid chromatography (HPLC) was performed on a Waters instrument using the following conditions:

| | |
|---|---|
| Column: | 30 mm × 15 cm Xterra Waters, C18, 5 mm |
| Solvent A: | Water with 1% acetic acid or 2 g/l ammonium |
| | carbonate |
| Solvent B: | Acetonitrile |
| Flow rate: | 40 ml/min |
| Run time: | 15 minutes with a 10 minute gradient from 5-95% B |
| Wavelength: | 254 nm |
| Injection volume | 2.0-4.0 ml; |

In addition, the following abbreviations have been used, where necessary:—
DMSO dimethylsulphoxide
NMP 1-methyl-2-pyrrolidinone
DMA N,N-dimethylacetamide
DCM dichloromethane
THF tetrahydrofuran
DEAD diethyl azodicarboxylate
DMF N,N-dimethylformamide
DTAD di-tert-butyl azodicarboxylate
DIPEA di-isopropylethylamine
IPA isopropyl alcohol
Ether diethyl ether
TBTU O-benzotriazol-1-yl-N,N,N′,N′-tetramethyluronium tetrafluoroborate
TFA trifluoroacetic acid.
NH₄OH ammonium hydroxide aqueous
AcOEt ethyl acetate

### EXAMPLE 1

### N′-(3-chloro-2,4-difluoro-phenyl)-N′-methyl-N-(3-morpholin-4-yl-5-thiomorpholin-4-yl-phenyl)pyrimidine-2,4-diamine

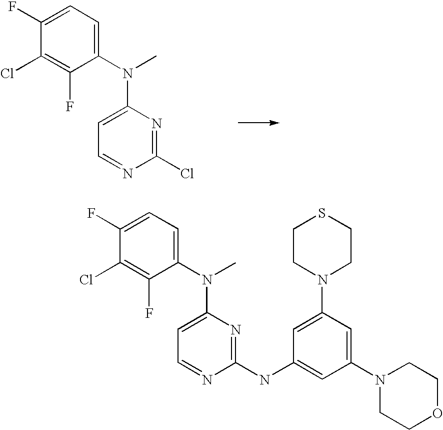

A mixture of 2-chloro-N-(3-chloro-2,4-difluoro-phenyl)-N-methyl-pyrimidin-4-amine (see Method 1, 100 mg, 0.34 mmol), 3-morpholin-4-yl-5-thiomorpholin-4-yl-aniline (see Method 9, 95 mg, 0.34 mmol), 4N HCl in dioxane (100 μL) and 2-propanol (5 mL) is heated at 90° C. for 3 hrs. The reaction mixture was cooled to room temperature and evaporated under reduced pressure. The residue was dissolved in methylene chloride and washed with a saturated solution of sodium bicarbonate. After evaporation, the crude material was purified by chromatography on silica gel (0 to 20% EtOAc/DCM) to give the title compound (107 mg, 58% yield). NMR Spectrum (500 MHz, DMSOd6) 2.60-2.68 (m, 4H), 2.97-3.06 (m, 4H), 3.38 (s, 3H), 3.40-3.47 (m, 4H), 3.67-3.75 (m, 4H), 5.81 (bs, 1H), 6.07 (dd, 1H), 6.89 (s, 1H), 6.91 (s, 1H), 7.46 (dd, 1H), 7.59 (ddd, 1H), 7.95 (d, 1H), 8.87 (s, 1H); Mass Spectrum: MH⁺ 533.

### EXAMPLE 2

The procedure described in example 1 was repeated using the appropriate aniline. Thus were obtained the compounds described below.

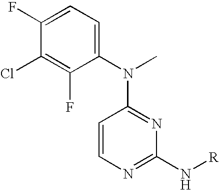

| | | | | NMR Spectrum (500 |
|---|---|---|---|---|
| | | R | Molecular | MHz, DMSOd6 at |
| Example | Name | (starting aniline) | ion (MH⁺) | 297° K) |
| 2.1^{a} | N′-(3-chloro-2,4-difluoro-phenyl)-N-(3,5-dimorpholin-4-ylphenyl)-N′-methyl-pyrimidine-2,4-diamine | 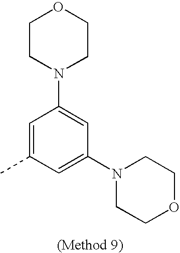 | 517 | 2.98-3.07 (m, 8H),3.38(s, 3H), 3.67-3.74 (m,8H), 5.81 (bs, 1H),6.10 (s, 1H), 6.91 (s,2H), 7.46 (ddd, 1H),7.59 (ddd, 1H), 7.96 (d,1H), 8.88 (s, 1H) |
| 2.2 | N′-(3-chloro-2,4-difluoro-phenyl)-N-(3-fluoro-5-morpholin-4-yl-phenyl)-N′-methyl-pyrimidine-2,4-diamine | 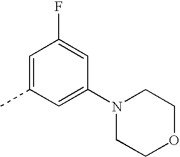 | 450 | 2.98-3.08 (m, 4H), 3.36(s, 3H), 3.67-3.75 (m,4H), 6.12 (bs, 1H),6.27 (d, 1H), 6.80 (bs,1H), 6.94 (bs, 1H),7.45 (dd, 1H), 7.57(ddd, 1H), 8.06 (d, 1H),9.24 (s, 1H) |
| 2.3 | N′-(3-chloro-2,4-difluoro-phenyl)-N′-methyl-N-(3-morpholin-4-ylphenyl)pyrimidine-2,4-diamine | 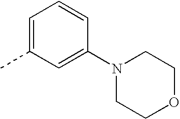 | 432 | 2.95-3.07 (m, 4H), 3.35(s, 3H), 3.68-3.77 (m,8H), 5.98 (bs, 1H),6.49 (dd, 1H), 6.93 (bs,1H), 7.10 (bs, 1H),7.16 (bs, 1H), 7.46(ddd, 1H), 7.54 (ddd,1H), 8.01 (d, 1H), 9.02(s, 1H) |
| 2.4 | N′-(3-chloro-2,4-difluoro-phenyl)-N-(3-methoxy-5-morpholin-4-yl-phenyl)-N′-methyl-pyrimidine-2,4-diamine | 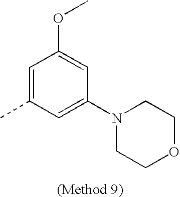 | 462 | 2.96-3.07 (m, 4H), 3.37(s, 3H), 3.65 (s, 3H),3.68-3.75 (m, 4H), 5.96(bs, 1H), 6.05 (t, 1H),6.75 (bs, 1H), 6.87 (bs,1H), 7.43 (ddd, 1H),7.56 (ddd, 1H), 8.00 (d,1H), 8.99 (s, 1H) |
| 2.5 | 3-[[4-[(3-chloro-2,4-difluoro-phenyl)-methyl-amino]pyrimidin-2-yl]amino]-5-morpholin-4-yl-benzonitrile | 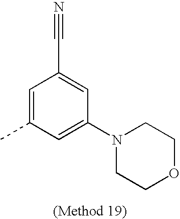 | 457 | 3.06-3.14 (m, 4H), 3.36(s, 3H), 3.69-3.76 (m,4H), 6.10 (bs, 1H),6.89 (s, 1H), 7.38 (bs,1H), 7.44 (ddd, 1H),7.55 (bs, 1H), 7.59(ddd, 1H), 8.08 (d, 1H),9.37 (s, 1H) |
| 2.6 | N′-(3-chloro-2,4-difluoro-phenyl)-N′-methyl-N-(3-methylsulfonyl-5-morpholin-4-yl-phenyl) pyrimidine-2,4-diamine | 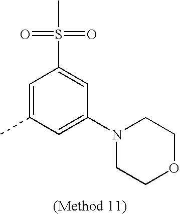 | 510 | 3.11-3.18 (m, 7H), 3.41(s, 3H), 3.73-3.78 (m,4H), 5.90 (bs, 1H),6.99 (s, 1H), 7.44-7.51(m, 2H), 7.61 (ddd,1H), 8.00 (d, 1H), 8.04(bs, 1H), 9.53 (bs, 1H) |
| 2.7 | [3-[[4-[(3-chloro-2,4-difluoro-phenyl)-methyl-amino]pyrimidin-2-yl]amino]-5-morpholin-4-yl-phenyl] methanol | 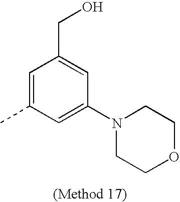 | 462 | 2.98-3.08 (m, 4H), 3.37(s, 3H), 3.67-3.78 (m,4H), 4.30 (d, 2H), 5.10(s, 1H), 5.90 (bs, 1H),6.50 (s, 1H), 7.11 (bs,1H), 7.16 (bs, 1H),7.46 (ddd, 1H), 7.58(ddd, 1H), 7.99 (d, 1H),9.01 (s, 1H) |
| 2.8 | 3-[[4-[(3-chloro-2,4-difluoro-phenyl)-methyl-amino]pyrimidin-2-yl]amino]-N,N-dimethyl-5-morpholin-4-yl-benzamide | 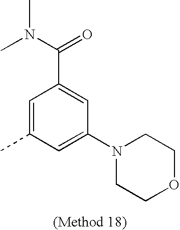 | 503 | 2.87 (bs, 3H), 2.95 (bs,3H), 2.99-3.11 (m,4H), 3.36 (s, 3H),3.66-3.78 (m, 4H), 5.95(bs, 1H), 6.44 (s, 1H),7.20 (s, 1H), 7.28 (bs,1H), 7.42 (dd, 1H),7.57 (ddd, 1H), 8.02(d, 1H), 9.15 (s, 1H) |
| 2.9 | N′-(3-chloro-2,4-difluoro-phenyl)-N-[3-(2-methoxyethoxy)-5-morpholin-4-yl-phenyl]-N′-methyl-pyrimidine-2,4-diamine | 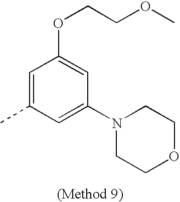 | 506 | 2.99-3.05 (m, 4H), 3.30(s, 3H), 3.37 (s, 3H),3.60-3.64 (m, 2H),3.69-3.73 (m, 4H),3.95-4.00 (m, 2H), 5.90(bs, 1H), 6.07 (dd, 1H),6.87 (bs, 1H), 6.90 (bs,1H), 7.44 (ddd, 1H),7.58 (ddd, 1H), 7.99 (d,1H), 8.99 (s, 1H) |
| 2.10 | N′-(3-chloro-2,4-difluoro-phenyl)-N′-methyl-N-[3-morpholin-4-yl-5-(1,4-oxazepan-4-yl)phenyl]pyrimidine-2,4-diamine | 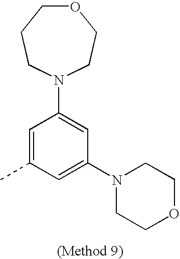 | 531 | 1.86-1.93 (m, 2H),2.98-3.04 (m, 4H), 3.38(s, 3H), 3.47-3.52 (m,2H), 3.53-3.58 (m,2H), 3.67-3.73 (m,8H), 5.78 (bs, 1H),5.98 (dd, 1H), 6.71 (s,1H), 6.78 (s, 1H), 6.45(ddd, 1H), 6.59 (ddd,1H), 6.94 (d, 1H), 8.78(s, 1H) |
| 2.11^{b} | 1-[3-[[4-[(3-chloro-2,4-difluoro-phenyl)-methyl-amino]pyrimidin-2-yl]amino]-5-methylsulfonyl-phenyl] piperidin-4-ol | 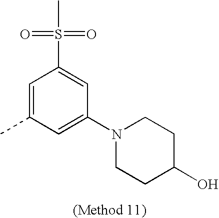 | 524 | 1.41-1.51 (m, 2H),1.77-1.86 (m, 2H),2.88-2.97 (m, 2H), 3.13(s, 3H), 3.40 (s, 3H),3.51-3.60 (m, 2H),3.62-3.71 (m, 1H), 4.71(d, 1H), 5.87 (bs, 1H),6.94 (s, 1H), 7.46 (ddd,1H), 7.48 (s, 1H), 7.61(ddd, 1H), 7.99 (s, 1H),8.00 (s, 1H), 9.40 (s,1H) |
| 2.12^{b} | 1-[4-[3-[[4-[(3-chloro-2,4-difluoro-phenyl)-methyl-amino] pyrimidin-2-yl]amino]-5-methylsulfonyl-phenyl] piperazin-1-yl] ethanone | 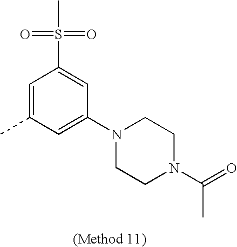 | 551 | 2.05 (s, 3H), 3.14 (s,3H), 3.16 (bs, 2H),3.19-3.25 (m, 2H), 3.41(s, 3H), 3.56-3.64 (m,4H), 5.88 (bs, 1H),6.99 (s, 1H), 7.46 (ddd,1H), 7.50 (s, 1H), 7.61(ddd, 1H), 8.01 (d, 1H),8.06 (bs, 1H), 9.46 (s,1H) |
| 2.13^{b} | 2-[4-[3-[[4-[(3-chloro-2,4-difluoro-phenyl)-methyl-amino] pyrimidin-2-yl]amino]-5-methylsulfonyl-phenyl] piperazin-1-yl] ethanol | 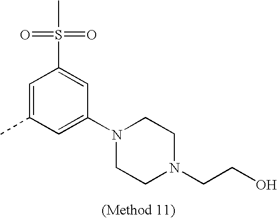 | 553 | 2.44 (t, 2H), 2.53-2.60(m, 4H), 3.13 (s, 3H),3.15-3.20 (m, 4H), 3.40(s, 3H), 3.51-3.58 (m,2H), 4.44 (t, 1H), 5.87(bs, 1H), 6.95 (s, 1H),7.46 (ddd, 1H), 7.48 (s,1H), 7.61 (ddd, 1H),8.00 (d, 1H), 8.03 (bs,1H), 9.42 (s, 1H) |
| 2.14^{c} | N′-(3-chloro-2,4-difluoro-phenyl)-N-[3-(methoxymethyl)-5-morpholin-4-yl-phenyl]-N′-methyl-pyrimidine-2,4-diamine | 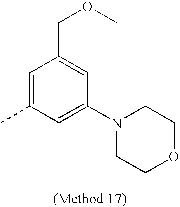 | 476 | 2.98-3.07 (m, 4H), 3.25(s, 3H), 3.37 (s, 3H),3.68-3.75 (m, 4H), 4.17(bs, 2H), 5.94 (bs, 1H),6.45 (s, 1H), 7.10 (bs,1H), 7.17 (bs, 1H),7.46 (ddd, 1H), 7.59(ddd, 1H), 8.00 (d, 1H),9.05 (s, 1) |
| 2.15^{c} | N′-(3-chloro-2,4-difluoro-phenyl)-N′-methyl-N-[3-morpholin-4-yl-5-(propan-2-yloxymethyl)phenyl] pyrimidine-2,4-diamine | 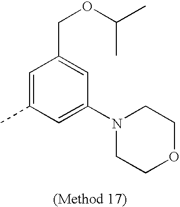 | 504 | 1.12 (d, 6H), 2.97-3.07(m, 4H), 3.37 (s, 3H),3.54-3.63 (m, 1H),3.68-3.77 (m, 4H), 4.24(bs, 2H), 5.92 (bs, 1H),6.46 (s, 1H), 7.10 (bs,1H), 7.19 (bs, 1H),7.46 (ddd, 1H), 7.59(ddd, 1H), 7.99 (d, 1H),9.04 (s, 1H) |
| 2.16^{c} | N′-(3-chloro-2,4-difluoro-phenyl)-N′-methyl-N-[3-morpholin-4-yl-5-(morpholin-4-ylmethyl)phenyl]py-rimidine-2,4-diamine | 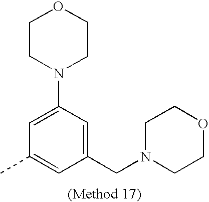 | 531 | 2.31 (bs, 4H), 2.96-3.06(m, 4H), 3.22 (s, 2H),3.38 (s, 3H), 3.52-3.60(m, 4H), 3.68-3.77(m, 4H), 5.93 (bs, 1H),6.47 (s, 1H), 7.08 (bs,1H), 7.18 (bs, 1H),7.46 (ddd, 1H), 7.59(ddd, 1H), 7.99 (d,1H), 9.02 (s, 1H) |
| 2.17^{c} | N′-(3-chloro-2,4-difluoro-phenyl)-N′-methyl-N-[3-morpholin-4-yl-5-(pyrrolidin-1-ylmethyl)phenyl]py-rimidine-2,4-diamine | 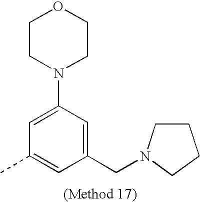 | 515 | 1.63-1.73 (m, 4H),2.37-2.45 (m, 4H),2.98-3.06 (m, 4H),3.33-3.39 (m, 5H),3.69-3.74 (m, 4H), 5.89(d, 1H), 6.46 (s, 1H),7.07 (s, 1H), 7.16 (s,1H), 7.41 (ddd, 1H),7.55 (ddd, 1H), 7.98 (d,1H), 8.82 (s, 1H) |
| 2.18^{c} | N′-(3-chloro-2,4-difluoro-phenyl)-N′-methyl-N-[3-[(4-methylpiperazin-1-yl)methyl]-5-morpholin-4-yl-phenyl] pyrimidine-2,4-diamine | 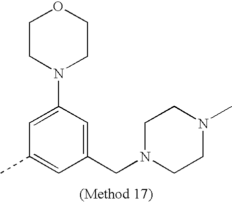 | 544 | 2.14 (s, 3H), 2.30 (bs,8H), 2.97-3.05 (m,2H), 3.20 (bs, 2H),3.38 (s, 3H), 3.68-3.75(m, 4H), 5.92 (ds, 1H),6.45 (s, 1H), 7.07 (s,1H), 7.16 (s, 1H), 7.45(ddd, 1H), 7.59 (ddd,1H), 7.99 (d, 1H), 9.01(s, 1H) |
| 2.19^{c} | 1-[[3-[[4-[(3-chloro-2,4-difluoro-phenyl)-methyl-amino]pyrimidin-2-yl]amino]-5-morpholin-4-yl-phenyl]methyl]piperidin-4-ol | 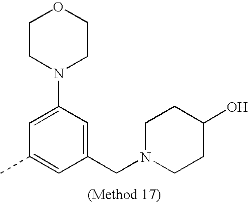 | 545 | 1.31-1.41 (m, 2H),1.64-1.73 (m, 2H),1.88-2.02 (m, 2H),2.58-2.69 (m, 2H),2.97-3.07 (m, 4H), 3.19(s, 2H), 3.37 (s, 3H),3.39-3.47 (m, 1H),3.69-3.75 (m, 4H), 4.51(d, 1H), 5.91 (bs, 1H),6.45 (s, 1H), 7.07 (s,1H), 7.16 (s, 1H), 7.45(ddd, 1H), 7.59 (ddd,1H), 7.99 (d, 1H), 9.01(s, 1H) |

^{a}The reaction mixture was heated at 80° C. for 16 hrs.
^{b}2-pentanol was used as the solvent and the reaction mixture was heated at 110° C. for 3 hrs.
^{c}The reaction mixture was heated at 120° C. for 15 min in a micro-wave oven.

### EXAMPLE 3

### N′-(3-chloro-2,4-difluoro-phenyl)-N′-methyl-N-[3-(4-methylpiperazin-1-yl)-5-methylsulfonyl-phenyl]pyrimidine-2,4-diamine

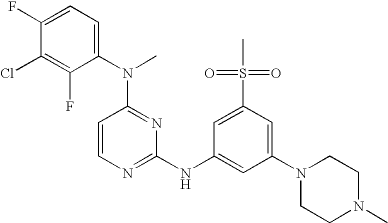

A mixture of 2-chloro-N-(3-chloro-2,4-difluoro-phenyl)-N-methyl-pyrimidin-4-amine (Method 1, 120 mg, 0.41 mmol), 3-methylsulfonyl-5-morpholin-4-yl-aniline (Method 11, 110 mg, 0.41 mmol), potassium carbonate (571 mg, 4.14 mmol), Pd2dba3 (tris(dibenzylideneacetone)dipalladium) (24 mg, 0.041 mmol) and Xantphos (48 mg, 0.083 mmol) in toluene degassed with argon (3 ml) was refluxed for 16 hrs. The solvent was removed under vacuum and the residue taken up in DMF and purified on a preparative HPLC-MS system (Column: C18, 5 microns, 19 mm diameter, 100 mm length, elution with a gradient of water and acetonitrile containing 2 g/l of ammonium carbonate). Evaporation of the collected fractions gave the title compound (101 mg, 46% yield); NMR Spectrum (500 MHz, DMSOd6) 2.24 (s, 3H), 2.43-2.51 (m, 4H), 3.14 (s, 3H), 3.16-3.23 (m, 4H), 3.41 (s, 3H), 5.88 (bs, 1H), 6.96 (s, 1H), 7.47 (ddd, 1H), 7.50 (s, 1H), 7.61 (ddd, 1H), 8.01 (d, 1H), 8.05 (bs, 1H), 8.43 (s, 1H). Mass Spectrum: MH⁺ 523.

### EXAMPLE 4

### N′-(3-chloro-2,4-difluoro-phenyl)-N′-methyl-N-[3-(4-methylpiperazin-1-yl)phenyl]pyrimidine-2,4-diamine

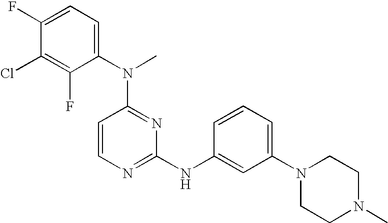

Prepared as in example 3 using 3-(4-methylpiperazin-1-yl)aniline (J. Med. Chem. 2005, Vol. 48, p. 8261-8269) as the starting aniline (33% yield); NMR Spectrum (500 MHz, DMSOd6) 2.21 (s, 3H), 2.40-2.47 (m, 4H), 3.02-3.10 (m, 4H), 3.37 (s, 3H), 5.99 (bs, 1H), 6.48 (dd, 1H), 6.91 (dd, 1H), 7.08 (d, 1H), 7.17 (bs, 1H), 7.46 (ddd, 1H), 7.58 (ddd, 1H), 8.02 (d, 1H), 9.00 (s, 1H). Mass Spectrum: MH⁺ 445.

### EXAMPLE 5

### N′-(3-chloro-2,4-difluoro-phenyl)-N′-methyl-N-[3-(4-methylpiperazin-1-yl)-5-morpholin-4-yl-phenyl]pyrimidine-2,4-diamine

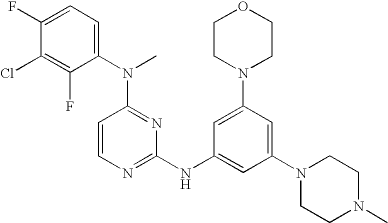

Prepared as in example 3 using 3-(4-methylpiperazin-1-yl)-5-morpholin-4-yl-aniline (Method 9) as the aniline, Caesium carbonate as the base, and Pd(OAc)₂/BINAP as the catalyst system (9% yield); NMR Spectrum (500 MHz, DMSOd6) 2.21 (s, 3H), 2.39-2.46 (m, 4H), 2.97-3.04 (m, 4H), 3.04-3.10 (m, 4H), 3.39 (s, 3H), 3.67-3.76 (m, 4H), 5.81 (bs, 1H), 6.10 (dd, 1H), 6.90 (s, 1H), 6.92 (s, 1H), 7.46 (ddd, 1H), 7.60 (ddd, 1H), 7.96 (d, 1H), 8.86 (s, 1H). Mass Spectrum: MH⁺ 530.

### EXAMPLE 6

### [3-[[2-[(3,5-dimorpholin-4-ylphenyl)amino]pyrimidin-4-yl]-methyl-amino]-4-methyl-phenyl]methanol

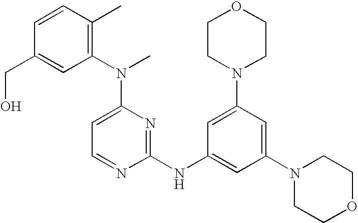

A mixture of [3-[(2-chloropyrimidin-4-yl)-methyl-amino]-4-methyl-phenyl]methanol (Method 2, 700 mg, 2.6 mmol), 3,5-dimorpholin-4-ylaniline (Method 9, 700 mg, 2.6 mmol), 4N HCl in dioxane (200 uL) and 2-propanol (15 mL) is heated at 140° C. for 15 min in a Personal Chemistry EMRS™ Optimizer EXP microwave synthesizer. The reaction mixture was cooled to room temperature and evaporated under reduced pressure. The residue was dissolved in a mixture of water, ethyl acetate and ammonium hydroxide (to bring the solution to pH 8) then the organic layer was washed with water and brine. After evaporation, the crude material was purified on silica gel (1 to 7% MeOH in DCM). Evaporation of the collected fractions and trituration in diethyl ether and petroleum ether gave the title compound as a white solid (810 mg, 62% yield); NMR Spectrum (500 MHz, DMSOd6) 2.08 (s, 3H), 3.07 (bs, 8H), 3.37 (s, 3H), 3.68-3.77 (m, 8H), 4.49 (d, 2H), 5.22 (t, 1H), 5.25 (bs, 1H), 6.11 (s, 1H), 7.03 (bs, 2H), 7.18 (s, 1H), 7.26 (d, 1H), 7.34 (d, 1H), 7.78 (bs, 1H), 8.85 (bs, 1H). Mass Spectrum: MH⁺ 491.

### EXAMPLE 6.1

### [3-[[2-[(3,5-dimorpholin-4-ylphenyl)amino]pyrimidin-4-yl]-methyl-amino]-4-methyl-phenyl]methanol freebase Form 1

Amorphous [3-[[2-[(3,5-dimorpholin-4-ylphenyl)amino]pyrimidin-4-yl]-methyl-amino]-4-methyl-phenyl]methanol (about 20 mg) was slurried in diethyl ether at 25° C. for 5 days with stirring. The crystalline [3-[[2-[(3,5-dimorpholin-4-ylphenyl)amino]pyrimidin-4-yl]-methyl-amino]-4-methyl-phenyl]methanol so obtained was used as a crystalline seed and the same method scaled up and repeated to give [3-[[2-[(3,5-dimorpholin-4-ylphenyl)amino]pyrimidin-4-yl]-methyl-amino]-4-methyl-phenyl]methanol freebase Form 1 crystalline material.

[3-[[2-[(3,5-dimorpholin-4-ylphenyl)amino]pyrimidin-4-yl]-methyl-amino]-4-methyl-phenyl]methanol freebase Form 1 is characterised by providing a X-ray powder diffraction pattern, substantially as shown in Figure A. The peaks are shown in Table B below.

[3-[[2-[(3,5-dimorpholin-4-ylphenyl)amino]pyrimidin-4-yl]-methyl-amino]-4-methyl-phenyl]methanol freebase Form 1 may thus also be characterised by providing at least one of the following 2θ values measured using CuKa radiation: 7.47, 22.21, 22.67 and 24.69.

TABLE B

| Angle | |
|---|---|
| 2-Theta ° (2θ) | Intensity % |
| 7.472 | 94.3 |
| 10.12 | 32.7 |
| 11.884 | 20.8 |
| 12.337 | 16.6 |
| 12.962 | 8.7 |
| 13.587 | 42.6 |
| 15.15 | 37.9 |
| 16.274 | 71 |
| 16.614 | 24 |
| 17.353 | 16.5 |
| 18.02 | 58.9 |
| 18.562 | 51.3 |
| 19.008 | 59.1 |
| 19.76 | 12.9 |
| 20.166 | 53.2 |
| 20.59 | 51.1 |
| 21.051 | 17.1 |
| 22.214 | 100 |
| 22.675 | 73.6 |
| 23.808 | 27.1 |
| 24.15 | 19.8 |
| 24.69 | 90.2 |
| 25.152 | 44 |
| 26.162 | 12.2 |
| 26.672 | 33.3 |
| 27.57 | 12.4 |
| 28.13 | 16.9 |
| 28.836 | 9 |
| 29.4 | 13.7 |
| 31.499 | 11.9 |
| 32.338 | 10.6 |
| 33.956 | 6.6 |
| 35.55 | 6.7 |
| 36.249 | 6.7 |
| 37.542 | 12.8 |
| 38.102 | 9.9 |

According to the present invention there is therefore provided a crystalline form of [3-[[2-[(3,5-dimorpholin-4-ylphenyl)amino]pyrimidin-4-yl]-methyl-amino]-4-methyl-phenyl]methanol freebase, Form 1, which has an X-ray powder diffraction pattern with at least one specific peak at about 2θ=7.5° when measured using CuKa radiation, more particularly 7.5° plus or minus 0.5° 2θ.

According to the present invention there is therefore provided a crystalline form of [3-[[2-[(3,5-dimorpholin-4-ylphenyl)amino]pyrimidin-4-yl]-methyl-amino]-4-methyl-phenyl]methanol freebase, Form 1, which has an X-ray powder diffraction pattern with at least one specific peak at about 2θ=22.2° when measured using CuKa radiation.

According to the present invention there is therefore provided a crystalline form of [3-[[2-[(3,5-dimorpholin-4-ylphenyl)amino]pyrimidin-4-yl]-methyl-amino]-4-methyl-phenyl]methanol freebase, Form 1, which has an X-ray powder diffraction pattern with at least one specific peak at about 2θ=22.7° when measured using CuKa radiation.

According to the present invention there is therefore provided a crystalline form of [3-[[2-[(3,5-dimorpholin-4-ylphenyl)amino]pyrimidin-4-yl]-methyl-amino]-4-methyl-phenyl]methanol freebase, Form 1, which has an X-ray powder diffraction pattern with at least one specific peak at about 2θ=24.7° when measured using CuKa radiation, more particularly 24.7° plus or minus 0.5° 2θ.

According to the present invention there is therefore provided a crystalline form of [3-[[2-[(3,5-dimorpholin-4-ylphenyl)amino]pyrimidin-4-yl]-methyl-amino]-4-methyl-phenyl]methanol freebase, Form 1, which has an X-ray powder diffraction pattern with specific peaks at about 2θ=7.5, 22.2, 22.7 and 24.7° when measured using CuKa radiation, more particularly wherein said values may be plus or minus 0.5° 2θ.

According to the present invention there is therefore provided a crystalline form of [3-[[2-[(3,5-dimorpholin-4-ylphenyl)amino]pyrimidin-4-yl]-methyl-amino]-4-methyl-phenyl]methanol freebase, Form 1, which has an X-ray powder diffraction pattern with specific peaks at 2θ=7.5, 10.1, 11.9, 12.3, 13.0, 13.6, 15.2, 16.3, 16.6, 17.4, 18.0, 18.6, 19.0, 19.8, 20.2, 20.6, 21.1, 22.2, 22.7, 23.8, 24.2, 24.7, 25.2, 26.2, 26.7, 27.6, 28.1, 28.8, 29.4, 31.5, 32.3, 34.0, 35.6, 36.2, 37.5 and 38.1° when measured using CuKa radiation, more particularly wherein said values may be plus or minus 0.5° 2θ.

According to the present invention there is therefore provided a crystalline form of [3-[[2-[(3,5-dimorpholin-4-ylphenyl)amino]pyrimidin-4-yl]-methyl-amino]-4-methyl-phenyl]methanol freebase, Form 1, which has an X-ray powder diffraction pattern substantially the same as the X-ray powder diffraction pattern shown in Figure A.

DSC analysis showed that [3-[[2-[(3,5-dimorpholin-4-ylphenyl)amino]pyrimidin-4-yl]-methyl-amino]-4-methyl-phenyl]methanol freebase Form 1 has an onset of melting at 179.01° C. and a peak at 182.29° C. The heat of fusion of [3-[[2-[(3,5-dimorpholin-4-ylphenyl)amino]pyrimidin-4-yl]-methyl-amino]-4-methyl-phenyl]methanol freebase Form 1 was found to be 71.28 J/g.

### Salt Synthesis

### EXAMPLE 6.2

### [3-[[2-[(3,5-dimorpholin-4-ylphenyl)amino]pyrimidin-4-yl]-methyl-amino]-4-methyl-phenyl]methanol besylate

A solution of benzenesulfonic acid (395 mg, 2.45 mmol) in acetonitrile (2 ml) was added dropwise to a stirred solution of (3-((2-(3,5-dimorpholinophenylamino)pyrimidin-4-yl)(methyl)amino)-4-methylphenyl)methanol (1200 mg, 2.45 mmol) in warm acetonitrile. The solution was then stirred at 20° C. and the salt precipitated after 30 min. and the mixture stirred overnight. The white solid was collected by filtration and dried at 45° C. under vacuum overnight to afford the desired benzenesulfonate salt (1.315 g). m.p.: 224.6-224-8° C.

NMR Spectrum (500 MHz, DMSOd6) 2.12 (s, 3H), 3.08-3.17 (m, 8H), 3.45 (s, 3H), 3.70-3.77 (m, 8H), 4.52 (s, 2H), 5.28 (bs, 1H), 6.39 (s, 1H), 6.63 (bs, 2H), 7.26 (s, 1H), 7.28-7.38 (m, 4H), 7.41 (d, 1H), 7.57-7.63 (m, 2H), 7.70 (d, 1H), 10.17 (bs, 1H).

Elemental Analysis Found C, 60.29; H, 6.24; N, 12.81.

C₂₇H₃₄N₆O₃H₂O 0.36 mol. C₆H₅SO₃H 1.0 mol Requires C, 60.49; H, 6.26; N, 12.83%.

### EXAMPLE 6.3

### [3-[[2-[(3,5-dimorpholin-4-ylphenyl)amino]pyrimidin-4-yl]-methyl-amino]-4-methyl-phenyl]methanol besylate Form 1

1 ml of acetonitrile was added to about 50 mg [3-[[2-[(3,5-dimorpholin-4-ylphenyl)amino]pyrimidin-4-yl]-methyl-amino]-4-methyl-phenyl]methanol to form a solution. About 18.5 mg of benzenesulfonic acid was dissolved in 1 ml of acetonitrile. The 3-[[2-[(3,5-dimorpholin-4-ylphenyl)amino]pyrimidin-4-yl]-methyl-amino]-4-methyl-phenyl]methanol/acetonitrile solution was added to the counter-ion solution and the resultant mixture shaken with heat. After about 1 hour the solution precipitated and the precipitate filtered and analysed to give [3-[[2-[(3,5-dimorpholin-4-ylphenyl)amino]pyrimidin-4-yl]-methyl-amino]-4-methyl-phenyl]methanol besylate Form 1.

[3-[[2-[(3,5-dimorpholin-4-ylphenyl)amino]pyrimidin-4-yl]-methyl-amino]-4-methyl-phenyl]methanol besylate Form 1 is characterised by providing a X-ray powder diffraction pattern, substantially as shown in Figure B. The peaks are shown in Table C below.

[3-[[2-[(3,5-dimorpholin-4-ylphenyl)amino]pyrimidin-4-yl]-methyl-amino]-4-methyl-phenyl]methanol besylate Form 1 may thus also be characterised by providing at least one of the following 2θ values measured using CuKa radiation: 5.56, 8.70, 16.39, 18.15, 18.97, 20.35 and 22.98.

TABLE C

| Angle | |
|---|---|
| 2-Theta ° (2θ) | Intensity % |
| 5.569 | 74.9 |
| 8.701 | 72.5 |
| 9.505 | 25.1 |
| 10.325 | 5.3 |
| 11.002 | 8.9 |
| 11.265 | 12.8 |
| 12.858 | 8.3 |
| 14.798 | 8.9 |
| 15.14 | 9.9 |
| 15.38 | 8.3 |
| 15.858 | 8.3 |
| 16.349 | 61.1 |
| 17.333 | 35.8 |
| 18.152 | 40.9 |
| 18.972 | 39.3 |
| 19.566 | 16.2 |
| 20.358 | 38.7 |
| 20.794 | 12.5 |
| 21.061 | 13.4 |
| 21.376 | 13.2 |
| 22.106 | 25.7 |
| 22.983 | 100 |
| 23.7 | 12.6 |
| 24.319 | 28.5 |
| 24.58 | 9.9 |
| 25.232 | 10.9 |
| 26.118 | 10.7 |
| 26.671 | 9.1 |
| 27.662 | 9.5 |
| 28.54 | 7.9 |
| 30.054 | 8.3 |
| 31.008 | 8.7 |
| 31.909 | 5.9 |
| 33.752 | 6.3 |

According to the present invention there is therefore provided a crystalline form of [3-[[2-[(3,5-dimorpholin-4-ylphenyl)amino]pyrimidin-4-yl]-methyl-amino]-4-methyl-phenyl]methanol besylate, Form 1, which has an X-ray powder diffraction pattern with specific peaks at about 2θ=5.6, 8.7, 16.3, 18.2, 19.0, 20.4 and 23.0° when measured using CuKa radiation, more particularly wherein said values may be plus or minus 0.5° 2θ.

According to the present invention there is therefore provided a crystalline form of [3-[[2-[(3,5-dimorpholin-4-ylphenyl)amino]pyrimidin-4-yl]-methyl-amino]-4-methyl-phenyl]methanol besylate, Form 1, which has an X-ray powder diffraction pattern with specific peaks at 2θ=5.6, 8.7, 9.5, 10.3, 11.0, 11.3, 12.9, 14.8, 15.1, 15.4, 15.9, 16.3, 17.3, 18.2, 19.0, 19.6, 20.4, 20.8, 21.1, 21.4, 22.1, 23.0, 23.7, 24.3, 24.6, 25.2, 26.1, 26.7, 27.7, 28.5, 30.1, 31.0, 31.9 and 33.8° when measured using CuKa radiation, more particularly wherein said values may be plus or minus 0.5° 2θ.

According to the present invention there is therefore provided a crystalline form of [3-[[2-[(3,5-dimorpholin-4-ylphenyl)amino]pyrimidin-4-yl]-methyl-amino]-4-methyl-phenyl]methanol besylate, Form 1, which has an X-ray powder diffraction pattern substantially the same as the X-ray powder diffraction pattern shown in Figure B.

DSC analysis showed that [3-[[2-[(3,5-dimorpholin-4-ylphenyl)amino]pyrimidin-4-yl]-methyl-amino]-4-methyl-phenyl]methanol besylate Form 1 has an onset of melting at 225.38° C. and a peak at 228.26° C. The heat of fusion of [3-[[2-[(3,5-dimorpholin-4-ylphenyl)amino]pyrimidin-4-yl]-methyl-amino]-4-methyl-phenyl]methanol besylate Form 1 was found to be 64.40 J/g.

### EXAMPLE 6.4

### [3-[[2-[(3,5-dimorpholin-4-ylphenyl)amino]pyrimidin-4-yl]-methyl-amino]-4-methyl-phenyl]methanol besylate Form 2

To a 100 ml 3-necked round bottom flask under nitrogen was charged (3-((2-(3,5-dimorpholinophenylamino)pyrimidin-4-yl)(methyl)amino)-4-methylphenyl)methanol (2 g, 4.08 mmol) (1 mol eq) and acetonitrile (20 mL) (10 rel vol). The slurry was then heated to reflux but did not dissolve so further acetonitrile (10 mL) (5 rel vol) was added and eventually dissolved in this volume. To this was then added benzenesulfonic acid (0.645 g, 4.08 mmol) (1 mol eq) in acetonitrile (10 mL) (5 rel vol) dropwise via a pipette. No precipitation was seen at this point or on further stirring at reflux so the solution was allowed to slowly cool back to room temperature overnight. Solid had precipitated out to give a thick slurry which was no longer agitating efficiently. This was filtered off, washing with MeCN (2×10 ml, 2×5 rel vol), pulled dry on the filter then dried in a vacuum oven at about 40° C.

Yield: 2.2 g (83.2% of theory), white solid

1H-NMR analysis in d6-DMSO showed the desired product to have been synthesised.

LCMS analysis showed the desired product at 99% purity.

[3-[[2-[(3,5-dimorpholin-4-ylphenyl)amino]pyrimidin-4-yl]-methyl-amino]-4-methyl-phenyl]methanol besylate Form 2 is characterised by providing a X-ray powder diffraction pattern, substantially as shown in Figure C. The peaks are shown in Table D below.

[3-[[2-[(3,5-dimorpholin-4-ylphenyl)amino]pyrimidin-4-yl]-methyl-amino]-4-methyl-phenyl]methanol besylate Form 2 may thus also be characterised by providing at least one of the following 2θ values measured using CuKa radiation: 5.58, 8.64, 15.98, 17.26, 20.87, 23.33 and 23.94°.

TABLE D

| Angle | |
|---|---|
| 2-Theta ° (2θ) | Intensity % |
| 5.581 | 52.6 |
| 8.645 | 100 |
| 9.815 | 9.9 |
| 11.074 | 8.2 |
| 15.988 | 21 |
| 16.661 | 17 |
| 17.261 | 40.3 |
| 17.675 | 13.6 |
| 18.614 | 15.6 |
| 20.871 | 18.8 |
| 23.331 | 47.7 |
| 23.946 | 26.7 |
| 25.961 | 13.4 |
| 27.748 | 9.9 |

According to the present invention there is therefore provided a crystalline form of [3-[[2-[(3,5-dimorpholin-4-ylphenyl)amino]pyrimidin-4-yl]-methyl-amino]-4-methyl-phenyl]methanol besylate, Form 2, which has an X-ray powder diffraction pattern with specific peaks at about 2θ=5.6, 8.6, 16.0, 17.3, 20.9, 23.3 and 23.9° when measured using CuKa radiation, more particularly wherein said values may be plus or minus 0.5° 2θ.

According to the present invention there is therefore provided a crystalline form of [3-[[2-[(3,5-dimorpholin-4-ylphenyl)amino]pyrimidin-4-yl]-methyl-amino]-4-methyl-phenyl]methanol besylate, Form 2, which has an X-ray powder diffraction pattern with specific peaks at 2θ=5.6, 8.6, 9.8, 11.1, 16.0, 16.7, 17.3, 17.7, 18.6, 20.9, 23.3, 23.9, 26.0 and 27.7° when measured using CuKa radiation, more particularly wherein said values may be plus or minus 0.5° 2θ.

According to the present invention there is therefore provided a crystalline form of [3-[[2-[(3,5-dimorpholin-4-ylphenyl)amino]pyrimidin-4-yl]-methyl-amino]-4-methyl-phenyl]methanol besylate, Form 2, which has an X-ray powder diffraction pattern substantially the same as the X-ray powder diffraction pattern shown in Figure C.

DSC analysis showed that [3-[[2-[(3,5-dimorpholin-4-ylphenyl)amino]pyrimidin-4-yl]-methyl-amino]-4-methyl-phenyl]methanol besylate Form 2 has an onset of melting at 226.12° C. and a peak at 229.01° C. The heat of fusion of [3-[[2-[(3,5-dimorpholin-4-ylphenyl)amino]pyrimidin-4-yl]-methyl-amino]-4-methyl-phenyl]methanol besylate Form 2 was found to be 76.55 J/g.

### EXAMPLE 6.5

### (3-((2-(3,5-dimorpholinophenylamino)pyrimidin-4-yl)(methyl)amino)-4-methylphenyl)methanol tosylate

A hot solution of p-toluenesulfonic acid (441 mg, 2.32 mmol) in ethyl acetate (5 ml) was slowly added to a stirred solution of (3-((2-(3,5-dimorpholinophenylamino)pyrimidin-4-yl)(methyl)amino)-4-methylphenyl)methanol (1100 mg, 2.24 mmol) in ethyl acetate (11 ml)) with a slight heating (45° C.). The solution was stirred at 20° C., the salt precipitated after 30 min. and the mixture stirred overnight. The white solid was collected by filtration and dried at 50° C. under vacuum for 24 h to afford the desired p-toluenesulfonate salt (1.3 g). mp: 202.2-202.3° C.

NMR Spectrum (500 MHz, DMSOd6) 2.12 (s, 3H), 2.29 (s, 3H), 3.9-3.17 (m, 8H), 3.45 (s, 3H), 3.70-3.77 (m, 8H), 4.51 (s, 2H), 5.30 (bs, 1H), 5.51 (d, 1H), 6.40 (s, 1H), 6.63 (bs, 2H), 7.11 (d, 2H), 7.27 (s, 1H), 7.35 (d, 1H), 7.41 (d, 1H), 7.47 (d, 2H), 7.70 (d, 1H), 10.17 (bs, 1H).

Elemental Analysis: Found C, 60.96; H, 6.56; N, 12.43; C₂₇H₃₄N₆O₃H₂O 0.17 mol, C₇H₈O₃S 1.02 mol Requires C, 61.26; H, 6.41; N, 12.54%.

### EXAMPLE 6.6

### (3-((2-(3,5-dimorpholinophenylamino)pyrimidin-4-yl)(methyl)amino)-4-methylphenyl)methanol tosylate Form 1

A hot solution of p-toluenesulfonic acid (80 mg, 0.42 mmol), in ethyl acetate (1 ml), was added to a stirred solution of (3-((2-(3,5-dimorpholinophenylamino)pyrimidin-4-yl)(methyl)amino)-4-methylphenyl)methanol (200 mg, 0.41 mmol), in ethyl acetate (2 ml)) with a slight heating (45° C.). The solution was stirred at 20° C. and the salt precipitated after 30 min. The crystalline mixture was stirred during 4 h, to slurry material. White solid was filtered, put in ethyl alcohol (2 ml), the mixture was stirred overnight, filtered, dried at 50° C. under vacuum for 24 hours to afford (3-((2-(3,5-dimorpholinophenylamino)pyrimidin-4-yl)(methyl)amino)-4-methylphenyl)methanol tosylate Form l, m=170 mg, melting point: 147.8-149.7° C.

[3-[[2-[(3,5-dimorpholin-4-ylphenyl)amino]pyrimidin-4-yl]-methyl-amino]-4-methyl-phenyl]methanol tosylate Form 1 is characterised by providing a X-ray powder diffraction pattern, substantially as shown in Figure D. The peaks are shown in Table E below.

TABLE E

| Angle | |
|---|---|
| 2-Theta ° (2θ) | Intensity % |
| 5.601 | 100 |
| 8.338 | 65.4 |
| 9.335 | 66.3 |
| 10.576 | 17.1 |
| 11.135 | 20 |
| 13.667 | 43.8 |
| 15.753 | 63.3 |
| 16.71 | 75 |
| 17.024 | 39.2 |
| 17.532 | 30 |
| 18.559 | 55.4 |
| 18.788 | 44.6 |
| 19.228 | 32.9 |
| 19.828 | 61.3 |
| 20.415 | 34.2 |
| 21.139 | 58.8 |
| 22.109 | 87.5 |
| 22.367 | 55.8 |
| 23.193 | 74.6 |
| 24.768 | 31.7 |
| 25.871 | 22.9 |
| 26.746 | 22.9 |
| 27.923 | 30.4 |
| 29.877 | 20.8 |

According to the present invention there is therefore provided a crystalline form of [3-[[2-[(3,5-dimorpholin-4-ylphenyl)amino]pyrimidin-4-yl]-methyl-amino]-4-methyl-phenyl]methanol tosylate, Form 1, which has an X-ray powder diffraction pattern with specific peaks at 2θ=5.6, 8.3, 9.3, 10.6, 11.1, 13.7, 15.8, 16.7, 17.0, 17.5, 18.6, 18.8, 19.2, 19.8, 20.4, 21.1, 22.1, 22.4, 23.2, 24.8, 25.9, 26.7, 27.9 and 29.9° when measured using CuKa radiation, more particularly wherein said values may be plus or minus 0.5° 2θ.

According to the present invention there is therefore provided a crystalline form of [3-[[2-[(3,5-dimorpholin-4-ylphenyl)amino]pyrimidin-4-yl]-methyl-amino]-4-methyl-phenyl]methanol tosylate, Form 1, which has an X-ray powder diffraction pattern substantially the same as the X-ray powder diffraction pattern shown in Figure D.

### EXAMPLE 6.7

### (3-((2-(3,5-dimorpholinophenylamino)pyrimidin-4-yl)(methyl)amino)-4-methylphenyl)methanol tosylate Form 2

A hot solution of p-toluenesulfonic acid (441 mg, 2.32 mmol), in ethyl acetate (5 ml), was slowly added to a stirred solution of (3-((2-(3,5-dimorpholinophenylamino)pyrimidin-4-yl)(methyl)amino)-4-methylphenyl)methanol (1100 mg, 2.24 mmol), in ethyl acetate (11 ml)) with a slight heating (45° C.). The solution was stirred at 20° C. and the salt precipitated after 30 min. The crystalline mixture was stirred overnight to slurry material. White solid was filtered, dried at 50° C. under vacuum for 24 H to afford (3-((2-(3,5-dimorpholinophenylamino)pyrimidin-4-yl)(methyl)amino)-4-methylphenyl)methanol tosylate Form 2, m=1.3 g, melting point: 202.2-202.3° C.

[3-[[2-[(3,5-dimorpholin-4-ylphenyl)amino]pyrimidin-4-yl]-methyl-amino]-4-methyl-phenyl]methanol tosylate Form 2 is characterised by providing a X-ray powder diffraction pattern, substantially as shown in Figure E. The peaks are shown in Table F below.

TABLE F

| Angle | |
|---|---|
| 2-Theta ° (2θ) | Intensity % |
| 7.141 | 59.2 |
| 8.586 | 100 |
| 10.163 | 28.3 |
| 10.983 | 13.6 |
| 11.413 | 22.7 |
| 13.292 | 11.6 |
| 14.206 | 18.7 |
| 16.125 | 26.9 |
| 17.071 | 41.1 |
| 17.58 | 33.1 |
| 18.931 | 24.4 |
| 19.327 | 16.1 |
| 19.59 | 32.3 |
| 19.821 | 26.9 |
| 20.275 | 19 |
| 21.013 | 12.5 |
| 21.808 | 22.9 |
| 22.306 | 34.8 |
| 22.54 | 26.9 |
| 22.88 | 13 |
| 23.807 | 16.7 |
| 25.345 | 29.2 |
| 25.757 | 15.3 |
| 26.716 | 11 |
| 27.072 | 9.3 |
| 29.365 | 12.5 |
| 29.837 | 12.5 |
| 34.58 | 10.2 |

According to the present invention there is therefore provided a crystalline form of [3-[[2-[(3,5-dimorpholin-4-ylphenyl)amino]pyrimidin-4-yl]-methyl-amino]-4-methyl-phenyl]methanol tosylate, Form 2, which has an X-ray powder diffraction pattern with specific peaks at 2θ=7.1, 8.6, 10.2, 11.0, 11.4, 13.3, 14.2, 16.1, 17.1, 17.6, 18.9, 19.3, 19.6, 19.8, 20.3, 21.0, 21.8, 22.3, 22.5, 22.9, 23.8, 25.3, 25.8, 26.7, 27.1, 29.4, 29.8 and 34.6° when measured using CuKa radiation, more particularly wherein said values may be plus or minus 0.5° 2θ.

According to the present invention there is therefore provided a crystalline form of [3-[[2-[(3,5-dimorpholin-4-ylphenyl)amino]pyrimidin-4-yl]-methyl-amino]-4-methyl-phenyl]methanol tosylate, Form 2, which has an X-ray powder diffraction pattern substantially the same as the X-ray powder diffraction pattern shown in Figure E.

### EXAMPLE 6.8

### (3-((2-(3,5-dimorpholinophenylamino)pyrimidin-4-yl)(methyl)amino)-4-methylphenyl)methanol fumarate Form 1

1 ml of acetonitrile was added to about 50 mg of (3-((2-(3,5-dimorpholinophenylamino)pyrimidin-4-yl)(methyl)amino)-4-methylphenyl)methanol to form a solution. About 12.22 mg of fumaric acid was dissolved up into 1 ml of acetonitrile. The (3-((2-(3,5-dimorpholinophenylamino)pyrimidin-4-yl)(methyl)amino)-4-methylphenyl)methanol/acetonitrile solution was added to the counter-ion solution and the resultant mixture shaken with heat. The solution precipitated immediately and the precipitate was filtered and analysed.

[3-[[2-[(3,5-dimorpholin-4-ylphenyl)amino]pyrimidin-4-yl]-methyl-amino]-4-methyl-phenyl]methanol fumarate Form 1 is characterised by providing a X-ray powder diffraction pattern, substantially as shown in Figure F. The peaks are shown in Table G below.

TABLE G

| Angle | |
|---|---|
| 2-Theta ° (2θ) | Intensity % |
| 6.845 | 56.2 |
| 7.211 | 75.2 |
| 8.837 | 30.2 |
| 11.689 | 33.4 |
| 14.621 | 65.9 |
| 15.569 | 42.9 |
| 16.132 | 62.2 |
| 17.176 | 55.7 |
| 17.723 | 49.3 |
| 18.241 | 78.6 |
| 19.706 | 36 |
| 21.126 | 57.1 |
| 22.8 | 48.4 |
| 24.012 | 41.7 |
| 24.48 | 48.3 |
| 25.928 | 40.5 |
| 28.735 | 100 |
| 29.313 | 19.3 |

According to the present invention there is therefore provided a crystalline form of [3-[[2-[(3,5-dimorpholin-4-ylphenyl)amino]pyrimidin-4-yl]-methyl-amino]-4-methyl-phenyl]methanol fumarate, Form 1, which has an X-ray powder diffraction pattern with specific peaks at 2θ=6.8, 7.2, 8.8, 11.7, 14.6, 15.6, 16.1, 17.2, 17.7, 18.2, 19.7, 21.1, 22.8, 24.0, 24.5, 25.9, 28.7 and 29.3° when measured using CuKa radiation, more particularly wherein said values may be plus or minus 0.5° 2θ.

According to the present invention there is therefore provided a crystalline form of [3-[[2-[(3,5-dimorpholin-4-ylphenyl)amino]pyrimidin-4-yl]-methyl-amino]-4-methyl-phenyl]methanol fumarate, Form 1, which has an X-ray powder diffraction pattern substantially the same as the X-ray powder diffraction pattern shown in Figure F.

### EXAMPLE 6.9

### (3-((2-(3,5-dimorpholinophenylamino)pyrimidin-4-yl)(methyl)amino)-4-methylphenyl)methanol fumarate Form 2

About 20 mg of (3-((2-(3,5-dimorpholinophenylamino)pyrimidin-4-yl)(methyl)amino)-4-methylphenyl)methanol fumarate Form 1 was slurried in EtOAc and also in MeOH for 2 days at 25° C. with stirring. The resultant materials were air dried and analysed, each showing to be Form 2.

[3-[[2-[(3,5-dimorpholin-4-ylphenyl)amino]pyrimidin-4-yl]-methyl-amino]-4-methyl-phenyl]methanol fumarate Form 2 is characterised by providing a X-ray powder diffraction pattern, substantially as shown in Figure G. The peaks are shown in Table H below.

TABLE H

| Angle | |
|---|---|
| 2-Theta ° (2θ) | Intensity % |
| 7.581 | 47.8 |
| 7.66 | 57.5 |
| 10.644 | 56.7 |
| 11.083 | 71.1 |
| 13.23 | 57.5 |
| 13.292 | 56 |
| 15.007 | 79.3 |
| 15.479 | 75.9 |
| 15.693 | 75.6 |
| 16.538 | 46.6 |
| 17.163 | 41.2 |
| 17.822 | 65.9 |
| 17.953 | 73.3 |
| 18.399 | 100 |
| 19.627 | 68.8 |
| 20.535 | 40.1 |
| 20.763 | 48.9 |
| 21.046 | 47.4 |
| 21.74 | 58.2 |
| 22.515 | 92 |
| 23.265 | 53 |
| 26.215 | 44.8 |

According to the present invention there is therefore provided a crystalline form of [3-[[2-[(3,5-dimorpholin-4-ylphenyl)amino]pyrimidin-4-yl]-methyl-amino]-4-methyl-phenyl]methanol fumarate, Form 2, which has an X-ray powder diffraction pattern with specific peaks at 2θ=7.6, 7.7, 10.6, 11.1, 13.2, 13.3, 15.0, 15.5, 15.7, 16.5, 17.2, 17.8, 18.0, 18.4, 19.6, 20.5, 20.8, 21.0, 21.7, 22.5, 23.3, and 26.2° when measured using CuKa radiation, more particularly wherein said values may be plus or minus 0.5° 2θ.

According to the present invention there is therefore provided a crystalline form of [3-[[2-[(3,5-dimorpholin-4-ylphenyl)amino]pyrimidin-4-yl]-methyl-amino]-4-methyl-phenyl]methanol fumarate, Form 2, which has an X-ray powder diffraction pattern substantially the same as the X-ray powder diffraction pattern shown in Figure G.

### X-Ray Powder Diffraction (XRPD)

The X-ray powder diffraction patterns of the polymorphic forms of the freebase and salts of Example 6 were determined by mounting a sample of the crystalline material on Siemens single silicon crystal (SSC) wafer mounts and spreading out the sample into a thin layer with the aid of a microscope slide. The sample was spun at 30 revolutions per minute (to improve counting statistics) and irradiated with X-rays generated by a copper long-fine focus tube operated at 40 kV and 40 mA with a wavelength of 1.5418 Angstroms using a Bruker D5000 powder X-ray diffractometer (Bruker AXS, Banner Lane Coventry CV4 9 GH). The collimated X-ray source was passed through an automatic variable divergence slit set at V20 and the reflected radiation directed through a 2 mm antiscatter slit and a 0.2 mm detector slit. The sample was exposed for 1 second per 0.02 degree 2-theta increment (continuous scan mode) over the range 2 degrees to 40 degrees 2-theta in theta-theta mode. The instrument was equipped with a scintillation counter as detector. Control and data capture was by means of a Dell Optiplex 686 NT 4.0 Workstation operating with Diffrac+software.

(Note that for Forms 1 and 2 of the fumarate salt of Example 6, the samples were analysed using a Bruker D8 X-ray diffractometer. The samples were exposed for 0.2 second per 0.0071° θ over the range 5° to 40° 2θ in continuous scan, theta-theta mode.)

The skilled person is aware that an X-ray powder diffraction pattern may be obtained which has one or more measurement errors depending on measurement conditions (such as equipment, sample preparation or machine used). In particular, it is generally known that intensities in an X-ray powder diffraction pattern may fluctuate depending on measurement conditions and sample preparation. For example, the skilled person will realize that the relative intensity of peaks can be affected by, for example, grains above 30 microns in size and non-unitary aspect ratios, which may affect analysis of samples. The skilled person will also realize that the position of reflections can be affected by the precise height at which the sample sits in the diffractometer and the zero calibration of the diffractometer. The surface planarity of the sample may also have a small effect. Hence a person skilled in the art will appreciate that the diffraction pattern data presented herein is not to be construed as absolute (for further information see Jenkins, R & Snyder, R. L. ‘Introduction to X-Ray Powder Diffractometry’ John Wiley & Sons, 1996). Therefore, it shall be understood that the crystalline form is not limited to the crystals that provide X-ray powder diffraction patterns identical to the X-ray powder diffraction patterns shown in Figures A to G and any crystals providing X-ray powder diffraction patterns substantially the same as that shown in Figures A to G fall within the scope of the present invention. A person skilled in the art of X-ray powder diffraction is able to judge the substantial identity of X-ray powder diffraction patterns.

### Differential Scanning Calorimetry (DSC)

DSC was recorded using a Thermal Analysis Q1000 system. Typically less than 5 mg of material, contained in an aluminium pan fitted with a sealed lid, was heated over the temperature range 25° C. to 325° C. at a constant heating rate of 10° C. per minute. A nitrogen purge gas was used with flow rate 50 ml per minute.

### EXAMPLE 7

The procedure described in example 6 was repeated using the appropriate aniline. Thus were obtained the compounds described below.

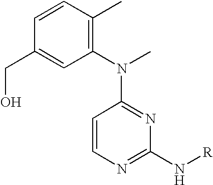

| | | R | | |
|---|---|---|---|---|
| | | (Starting | Molecular | |
| Example | Name | aniline) | ion (MH⁺) | NMR Spectrum |
| 7.1 | [4-methyl-3-[methyl-[2-[[3-morpholin-4-yl-5-(1,4-oxazepan-4-yl)phenyl]amino]pyrimidin-4-yl]amino]phenyl]methanol | 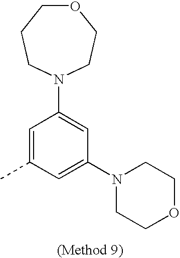 | 505 | 1.85-1.97 (m, 2H), 2.08 (s,3H), 2.95-3.15 (m, 4H),3.36 (s, 3H), 3.47-3.61 (m,6H), 3.66-3.78 (m, 6H),4.49 (d, 2H), 5.22 (t, 1H),5.24 (bs, 1H), 5.89 (s, 1H),6.82 (bs, 1H), 6.89 (bs,1H), 7.18 (s, 1H), 7.26 (d,1H), 7.34 (d, 1H), 7.77 (bs,1H), 8.74 (bs, 1H) |
| 7.2 | [4-methyl-3-[methyl-[2-[[3-(4-methylpiperazin-1-yl)-5-morpholin-4-yl-phenyl]amino]pyrimidin-4-yl]amino]phenyl]methanol | 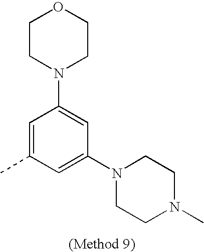 | 504 | 2.08 (s, 3H), 2.20 (s, 3H),2.38-2.47 (m, 4H), 3.06(bs, 4H), 3.09 (bs, 4H),3.36 (s, 3H), 3.68-3.76 (m,4H), 4.49 (s, 2H), 5.22 (s,1H), 5.25 (bs, 1H), 6.09 (s,1H), 7.00 (bs, 1H), 7.03(bs, 1H), 7.18 (s, 1H), 7.25(d, 1H), 7.34 (d, 1H), 7.77(bs, 1H), 8.81 (bs, 1H) |
| 7.3 | [4-methyl-3-[methyl-[2-[(3-morpholin-4-yl-5-pyrrolidin-1-yl-phenyl)amino]pyrimidin-4-yl]amino]phenyl]methanol | 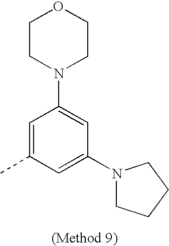 | 475 | 1.87-1.98 (m, 4H), 2.08 (s,3H), 2.98-3.10 (m, 4H),3.14-3.26 (m, 4H), 3.37 (s,3H), 3.68-3.77 (m, 4H),4.49 (d, 2H), 5.21 (t, 1H),5.23 (bs, 1H), 5.70 (s, 1H),6.77 (bs, 2H), 7.18 (s, 1H),7.25 (d, 1H), 7.33 (d, 1H),7.77 (bs, 1H), 8.74 (bs,1H) |
| 7.4 | [4-methyl-3-[methyl-[2-[[3-morpholin-4-yl-5-(1-piperidyl)phenyl]amino]pyrimidin-4-yl]amino]phenyl]methanol | 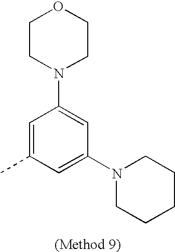 | 489 | 1.46-1.55 (m, 2H), 1.56-1.64(m, 4H), 2.08 (s, 3H),3.00-3.07 (m, 4H), 3.07-3.15(m, 4H), 3.37 (s, 3H),3.68-3.76 (m, 4H), 4.49 (d,2H), 5.22 (t, 1H), 5.25 (bs,1H), 6.08 (s, 1H), 6.97 (bs,1H), 7.04 (bs, 1H), 7.18 (s,1H), 7.26 (d, 1H), 7.34 (d,1H), 7.77 (bs, 1H), 8.81(bs, 1H) |
| 7.5 | [4-methyl-3-[methyl-[2-[[3-morpholin-4-yl-5-(morpholin-4-ylmethyl)phenyl]amino]pyrimidin-4-yl]amino]phenyl]methanol | 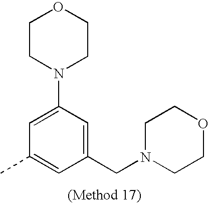 | 505 | 2.08 (s, 3H), 2.30-2.42 (m,4H), 3.00-3.13 (m, 4H),3.36 (bs, 2H), 3.39 (bs,3H), 3.53-3.61 (m, 4H),3.69-3.78 (m, 4H), 4.50 (d,2H), 5.22 (t, 1H), 5.26 (bs,1H), 6.46 (s, 1H), 7.18 (s,1H), 7.26 (d, 1H), 7.33 (s,1H), 7.35 (s, 1H), 7.39 (bs,1H), 7.78 (bs, 1H), 8.98(bs, 1H) |
| 7.7 | [4-methyl-3-[methyl-[2-[(3-methylsulfonyl-5-morpholin-4-yl-phenyl)amino]pyrimidin-4-yl]amino]phenyl]methanol | 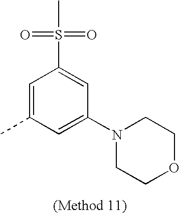 | 484 | 2.09 (s, 3H), 3.17 (s, 3H),3.18 (bs, 4H), 3.40 (s, 3H),3.73-3.80 (m, 4H), 4.49 (s,2H), 5.23 (bs, 1H), 5.35 (d,1H), 7.00 (s, 1H), 7.19 (s,1H), 7.28 (d, 1H), 7.36 (d,1H), 7.55 (s, 1H), 7.83 (d,1H), 8.17 (s, 1H), 9.53 (bs,1H) |
| 7.8 | 1-[3-[[4-[[5-(hydroxymethyl)-2-methyl-phenyl]-methyl-amino]pyrimidin-2-yl]amino]-5-morpholin-4-yl-phenyl]piperidin-4-ol | 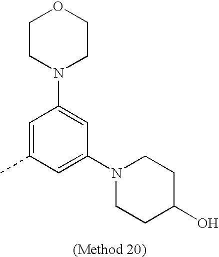 | 505 | 1.38-1.53 (m, 2H), 1.72-1.85(m, 2H), 2.08 (s, 3H),2.73-2.85 (m, 2H), 2.95-3.14(m, 4H), 3.37 (s, 3H),3.43-3.54 (m, 2H), 3.54-3.64(m, 1H), 3.65-3.78(m, 4H), 4.49 (d, 2H), 4.64(d, 1H), 5.22 (t, 1H), 5.25(bs, 1H), 6.09 (s, 1H), 6.96(s, 1H), 7.03 (s, 1H), 7.26(d, 1H), 7.34 (d, 1H), 7.78(s, 1H), 8.81 (s, 1H) |
| 7.9 | (3S)-1-[3-[[4-[[5-(hydroxymethyl)-2-methyl-phenyl]-methyl-amino]pyrimidin-2-yl]amino]-5-morpholin-4-yl-phenyl]pyrrolidin-3-ol | 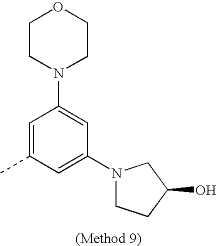 | 491 | 1.81-1.90 (m, 1H), 1.96-2.07(m, 1H), 2.08 (s, 3H),2.99-3.11 (m, 5H), 3.19-3.29(m, 1H), 3.31-3.43(m, 5H), 3.67-3.78 (m,4H), 4.32-4.40 (m, 1H),4.49 (d, 2H), 4.90 (d, 1H),5.21 (t, 1H), 5.24 (bs, 1H),5.67 (s, 1H), 6.71-6.81 (m,2H), 7.18 (s, 1H), 7.26 (d,1H), 7.34 (d, 1H), 7.77 (bs,1H), 8.75 (bs, 1H) |
| 7.10 | (3R)-1-[3-[[4-[[5-(hydroxymethyl)-2-methyl-phenyl]-methyl-amino]pyrimidin-2-yl]amino]-5-morpholin-4-yl-phenyl]pyrrolidin-3-ol | 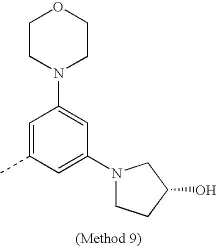 | 491 | 1.81-1.90 (m, 1H), 1.96-2.06(m, 1H), 2.08 (s, 3H),2.99-3.11 (m, 5H), 3.20-3.28(m, 1H), 3.31-3.43(m, 5H), 3.69-3.76 (m,4H), 4.36 (bs, 1H), 4.49 (d,2H), 4.90 (d, 1H), 5.21 (t,1H), 5.24 (bs, 1H), 5.68 (s,1H), 6.71-6.81 (m, 2H),7.18 (s, 1H), 7.26 (d, 1H),7.34 (d, 1H), 7.77 (bs, 1H),8.75 (bs, 1H) |
| 7.11 | 3-[[4-[[5-(hydroxymethyl)-2-methyl-phenyl]-methyl-amino]pyrimidin-2-yl]amino]-N,N-dimethyl-5-morpholin-4-yl-benzamide | 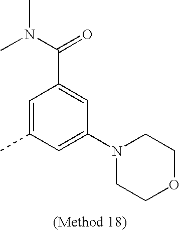 | 477 | 2.08 (s, 3H), 2.93 (bs, 3H),2.95 (bs, 3H), 3.11 (m,4H), 3.36 (bs, 3H), 3.68-3.79(m, 4H), 4.49 (d, 2H),5.22 (t, 1H), 5.29 (bs, 1H),6.49 (s, 1H), 7.18 (s, 1H),7.26 (d, 1H), 7.29-7.38 (m,2H), 7.57 (bs, 1H), 7.80(bs, 1H), 9.15 (bs, 1H) |
| 7.12^{a} | [3-[[2-[(3-methoxy-5-morpholin-4-yl-phenyl)amino]pyrim-idin-4-yl]-methyl-amino]-4-methyl-phenyl]methanol | 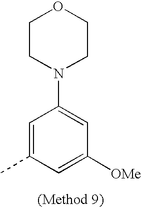 | 436 | 2.08 (s, 3H), 3.07 (bs, 4H),3.37 (s, 3H), 3.71 (s, 3H),3.72 (bs, 4H), 4.49 (d, 2H),5.22 (t, 1H), 5.27 (bs, 1H),6.08 (s, 1H), 7.03 (bs, 1H),7.14 (bs, 1H), 7.18 (s, 1H),7.26 (d, 1H), 7.34 (d, 1H),7.79 (bs, 1H), 8.98 (bs,1H) |
| 7.13^{a} | [3-[[4-[[5-(hydroxymethyl)-2-methyl-phenyl]-methyl-amino]pyrimidin-2-yl]amino]-5-morpholin-4-yl-phenyl]methanol | 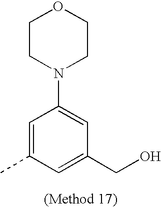 | 436 | 2.09 (s, 3H), 3.09 (bs, 4H),3.37 (s, 3H), 3.68-3.80 (m,4H), 4.41 (bs, 2H), 4.49 (d,2H), 5.04 (bs, 1H), 5.22 (t,1H), 5.26 (bs, 1H), 6.49 (s,1H), 7.18 (s, 1H), 7.26 (d,1H), 7.29-7.50 (m, 3H),7.78 (s, 1H), 8.98 (s, 1H) |
| 7.14^{a} | [3-[[2-[[3-(2-methoxyethoxy)-5-morpholin-4-yl-phenyl]amino]pyrim-idin-4-yl]-methyl-amino]-4-methyl-phenyl]methanol | 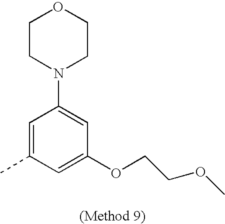 | 480 | 2.08 (s, 3H), 3.07 (bs, 4H),3.31 (s, 3H), 3.37 (bs, 3H),3.61-.66 (m, 2H), 3.68-3.75(m, 4H), 4.03 (bs,2H), 4.49 (d, 2H), 5.22 (t,1H), 5.27 (bs, 1H), 6.09(bs, 1H), 7.06 (bs, 1H),7.10 (bs, 1H), 7.18 (s, 1H),7.26 (d, 1H), 7.34 (d, 1H),7.78 (bs, 1H), 8.98 (bs,1H) |
| 7.15^{b} | 3-[[4-[[5-(hydroxymethyl)-2-methyl-phenyl]-methyl-amino]pyrimidin-2-yl]amino]-5-morpholin-4-yl-benzonitrile | 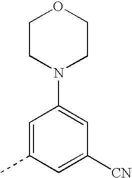 | 431 | 2.09 (s, 3H), 3.15 (bs, 4H),3.37 (s, 3H), 3.73 (bs, 4H),4.49 (d, 2H), 5.23 (s, 1H),5.33 (s, 1H), 6.93 (s, 1H),7.19 (s, 1H), 7.28 (d, 1H),7.35 (d, 1H), 7.71 (s, 1H),7.79 (s, 1H), 7.83 (s, 1H),9.37 (s, 1H) |
| 7.16^{c} | 4-[3-[[4-[[5-(hydroxymethyl)-2-methyl-phenyl]-methyl-amino]pyrimidin-2-yl]amino]-5-morpholin-4-yl-phenyl]morpholin-3-one | 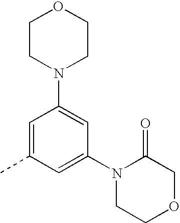 | 505 | RMO-00069-70-02DMSOd6 at 323° K: 2.08(s, 3H), 3.06-3.12 (m, 4H),3.34 (s, 3H), 3.65 (bs, 2H),3.70-3.76 (m, 4H), 3.93-3.97(m, 2H), 4.16 (s, 2H),4.49 (d, 2H), 5.06 (t, 1H),5.37 (bs, 1H), 6.50 (s, 1H),7.16 (s, 1H), 7.20-7.44 (m,4H), 7.82 (d, 1H), 8.90 (s,1H) |
| 7.17^{d} | (S)-(4-methyl-3-(methyl(2-(3-(3-methylmorpholino)-5-morpholinophenyl-amino)pyrimidin-4-yl)amino)phenyl)methanol | 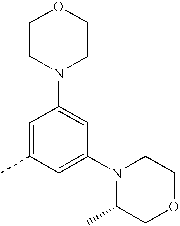 | 505 | RMN-00069-76-02DMSOd6: 0.98 (d, 3H),2.08 (s, 3H), 2.96-3.12 (m,6H), 3.36 (s partiallyhidden by H2O, 3H), 3.55(ddd, 1H), 3.62 (dd, 1H),3.67-3.77 (m, 6H), 3.86(ddd, 1H), 4.49 (d, 2H),5.22 (t, 1H), 5.25 (bs, 1H),6.07 (s, 1H), 6.95-7.05 (m,2H), 7.18 (s, 1H), 7.26 (d,1H), 7.34 (d, 1H), 7.78 (d,1H), 8.83 (s, 1H) |
| 7.18^{e} | (R)-(4-methyl-3-(methyl(2-(3-(3-methylmorpholino)-5-morpholinophenyl-amino)pyrimidin-4-yl)amino)phenyl)methanol | 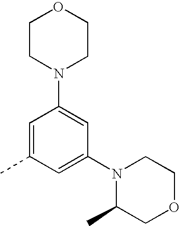 | 505 | RMO-00069-83-1DMSOd6: 0.98 (d, 3H),2.08 (s, 3H), 2.96-3.12 (m,6H), 3.36 (s partiallyhidden by H2O, 3H), 3.55(ddd, 1H), 3.62 (dd, 1H),3.67-3.75 (m, 6H), 3.86(ddd, 1H), 4.49 (d, 2H),5.22 (t, 1H), 5.25 (bs, 1H),6.07 (s, 1H), 6.95-7.05 (m,2H), 7.18 (s, 1H), 7.26 (d,1H), 7.34 (d, 1H), 7.78 (d,1H), 8.83 (s, 1H) |
| 7.19^{f} | 1-(3-(4-((5-(hydroxymethyl)-2-methylphenyl)(methyl)amino)pyrimidin-2-ylamino)-5-morpholinophenyl)piperidin-4-one | 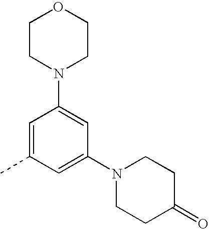 | 503 | RMO-00069-87-2DMSOd6: 2.08 (s, 3H),2.38-2.44 (m, 4H), 3.08(bs, 4H), 3.37 (s partiallyhidden by H2O, 3H), 3.56(bs, 4H), 3.69-3.76 (m,4H), 4.49 (d, 2H), 5.23 (t,1H), 5.26 (bs, 1H), 6.19 (s,1H), 7.02 (s, 1H), 7.12 (s,1H), 7.18 (s, 1H), 7.26 (d,1H), 7.34 (d, 1H), 7.78 (bs,1H), 8.85 (s, 1H) |

^{a}The mixture was heated in an oil bath at 85° C. for 18 hrs. After cooling, 7 N methanolic ammonia was added. The solvents were evaporated under vacuum. After evaporation of the solvents, the residue was injected on an HPLC column (C18, 5 microns, 19 mm diameter, 100 mm length) of a preparative HPLC-MS system eluting with a mixture of water and acetonitrile containing 2 g/l of ammonium carbonate (gradient).
^{b}as for note a, except that the compound was isolated by aqueous treatment with NaHCO3 and extraction with DCM, followed by chromatography on silica gel (50 to 100% EtOAc in DCM).
^{c}Stirred at 80° C. for 2 hrs. The reaction mixture was concentrated to dryness and diluted with DCM:methanolic ammonia 7 N (95:5, 10 mL). The resulting precipitate was removed by filtration and washed with DCM. The filtrate was concentrated down and purified by flash chromatography on silica gel eluting with a 0 to 5% methanol gradient in a 1:1 mixture of ethyl acetate:DCM.

The 4-(3-amino-5-morpholinophenyl)morpholin-3-one used as starting material was made as follows:—

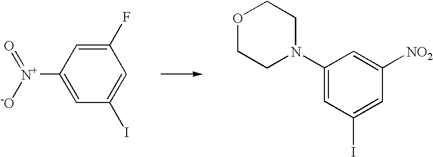

A solution of 1-fluoro-3-iodo-5-nitrobenzene (10.486 g, 39.27 mmol) and morpholine (7.21 mL, 82.48 mmol) in DMSO (28 mL) was stirred at 90° C. overnight. The reaction mixture was poured into water (70 mL), the precipitate was collected by filtration, washed with water and dried to afford 4-(3-iodo-5-nitrophenyl)morpholine (12.90 g, 98%) as a yellow solid, which was used without further purification. Mass Spectrum: M+H⁺ 335. NMR Spectrum (CDCl3): 3.22-3.27 (m, 4H), 3.84-3.89 (m, 4H), 7.46 (dd, 1H), 7.66 (dd, 1H), 7.98 (dd, 1H)

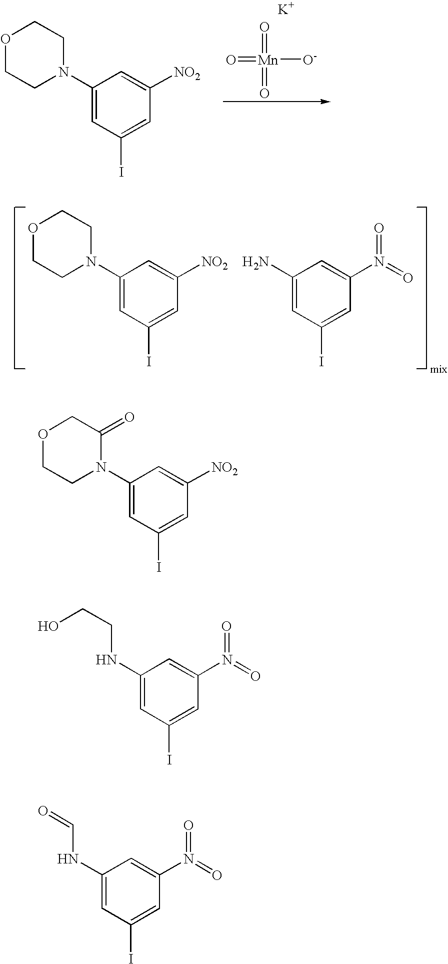

Potassium permanganate (4.26 g, 26.94 mmol) was added portionwise to 4-(3-iodo-5-nitrophenyl)morpholine (3 g, 8.98 mmol) and benzyltriethylammonium chloride (5.93 g, 26.04 mmol) dissolved in DCM (40 ml). The resulting slurry was stirred at 25° C. for 1 hr then heated to reflux for 4 hrs. Further potassium permanganate (2.84 g, 17.96 mmol) was added at 25° C. and the reaction mixture was stirred at reflux for an additional 8 hrs. The reaction mixture was allowed to cool to room temperature under stirring, quenched with water (90 ml) and sodium sulfite (18.11 g, 143.67 mmol) was added portionwise at 5° C. The mixture was filtered through a pad of celite, the aqueous phase was extracted with DCM and the organic phases combined and washed with water, brine, dried over magnesium sulphate and concentrated down. The crude product was purified by flash chromatography on silica gel eluting with 0 to 15% ethyl acetate in DCM to afford 4-(3-iodo-5-nitrophenyl)morpholin-3-one (0.418 g, 13.37%) as a beige solid. Mass Spectrum: M+H⁺ 349. NMR Spectrum (DMSOd6): 3.81-3.87 (m, 2H), 3.96-4.01 (m, 2H), 4.25 (s, 2H), 8.28 (dd, 1H), 8.36 (dd, 1H), 8.39 (dd, 1H)

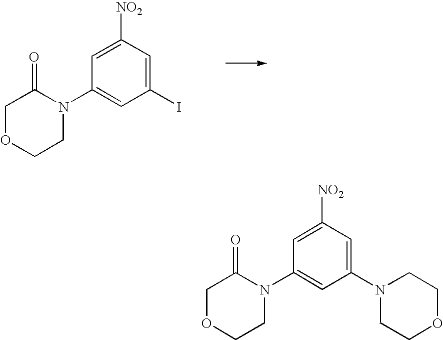

A mixture of 4-(3-iodo-5-nitrophenyl)morpholin-3-one (395 mg, 1.13 mmol), cesium carbonate (1109 mg, 3.40 mmol), palladium (II) acetate (12.74 mg, 0.06 mmol) and 2,2′-bis(diphenylphosphino)-1,1′-binaphthyl (35.3 mg, 0.06 mmol) in toluene (4 ml) was degassed with argon then morpholine (0.199 ml, 2.27 mmol) stirred at reflux for 6 hrs. The reaction was diluted with DCM and insolubles were removed by filtration. The filtrate was concentrated down. The crude product was purified by flash chromatography on silica gel eluting with 0 to 100% ethyl acetate in DCM. The solvent was evaporated to dryness to afford 4-(3-morpholino-5-nitrophenyl)morpholin-3-one (243 mg, 69.7%) as an yellow solid. Mass Spectrum: M+H⁺ 308. NMR Spectrum (CDCl3): 3.25-3.29 (m, 4H), 3.79-3.83 (m, 2H), 3.85-3.89 (m, 4H), 4.04-4.09 (m, 2H), 4.36 (s, 2H), 7.28 (dd, 1H), 7.60 (dd, 1H), 7.64 (dd, 1H)

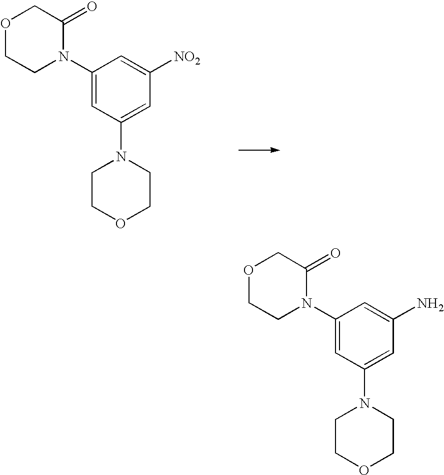

A solution of 4-(3-morpholino-5-nitrophenyl)morpholin-3-one (245 mg, 0.80 mmol) and ADAMS' platinum oxide (18.10 mg, 0.08 mmol) in ethyl acetate (2 ml) and ethanol (2 ml) was hydrogenated under 55 psi at room temperature for 1 hr. The resulting solution was filtered through a pad of celite and the filtrate was concentrated to dryness to afford the crude 4-(3-amino-5-morpholinophenyl)morpholin-3-one (235 mg, 106%) as a off-white foam. Mass Spectrum: M+H⁺ 278. NMR Spectrum (CDCl3): 3.10-3.16 (m, 4H), 3.67-3.72 (m, 2H), 3.73 (bs, 2H), 3.80-3.85 (m, 4H), 3.97-4.02 (m, 2H), 4.31 (s, 2H), 6.15 (dd, 1H), 6.16 (dd, 1H), 6.26 (dd, 1H)

^{d}: The procedure used was similar to the one described in note (c). The (S)-3-(3-methylmorpholino)-5-morpholinoaniline as starting material was made as follows:—

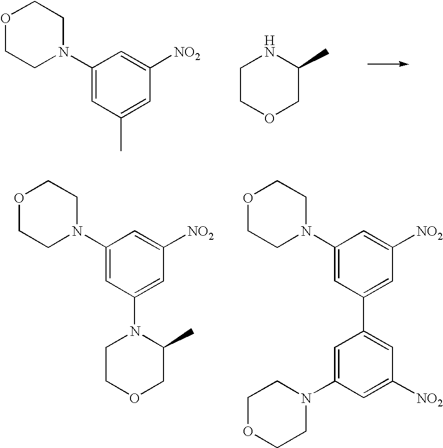

A mixture of 4-(3-iodo-5-nitrophenyl)morpholine (670 mg, 2.01 mmol), (S)-3-methylmorpholine (406 mg, 4.01 mmol), cesium carbonate (1960 mg, 6.02 mmol), palladium (II) acetate (22.51 mg, 0.10 mmol) and 2,2′-bis(diphenylphosphino)-1,1′-binaphthyl (62.4 mg, 0.10 mmol) in toluene (6 ml) was stirred at reflux for 5 hrs. The reaction was diluted with DCM and insolubles were removed by filtration. The filtrate was concentrated down. The crude product was purified by flash chromatography on silica gel eluting with 0 to 5% ethyl acetate in DCM. The solvent was evaporated to dryness to afford (S)-3-methyl-4-(3-morpholino-5-nitrophenyl)morpholine (350 mg, 53.9%) as a dark yellow solid. It contained 5 mol % of 4,4′-(5,5′-dinitrobiphenyl-3,3′-diyl)dimorpholine. Mass Spectrum: M+H⁺ 308. NMR Spectrum (CDCl3): 1.14 (d, 3H), 3.15 (ddd, 1H), 3.18-3.25 (m, 5H), 3.69 (ddd, 1H), 3.76 (d, 1H), 3.79-3.90 (m, 6H), 4.02 (ddd, 1H), 6.60 (dd, 1H), 7.22-7.25 (m, 2H)

The reduction of the nitro function was done as described in note (c). The crude product was purified by flash chromatography on silica gel eluting with 0 to 5% methanol in DCM/ethyl acetate (1/1). The solvent was evaporated to dryness to afford (S)-3-(3-methylmorpholino)-5-morpholinoaniline (250 mg, 70.6%) as a white foam. Mass Spectrum: M+H⁺ 278. NMR Spectrum (CDCl3): 1.07 (s, 3H), 3.03 (ddd, 1H), 3.07-3.19 (m, 5H), 3.58 (bs, 2H), 3.61-3.74 (m, 3H), 3.79-3.86 (m, 5H), 3.93 (ddd, 1H), 5.84 (bs, 2H), 5.92 (bs, 1H)

^{e}: made using a procedure similar to the one described in example 7.17 using the R enantiomer of 3-methylmorpholine instead of the S. The R enantiomer was made according to Bettoni, G.; Franchini, C.; Perrone, R. and Tortorella Tetrahedron 1980, 36, 409. The S enantiomer is commercially available.

^{f}: A solution of (4-methyl-3-(methyl(2-(3-morpholino-5-(1,4-dioxa-8-azaspiro[4.5]decan-8-yl)phenylamino)pyrimidin-4-yl)amino)phenyl)methanol (166 mg, 0.30 mmol) and p-toluenesulfonic acid (63.5 mg, 0.33 mmol) in water (0.750 ml) and acetone (1 ml) was stirred at 70° C. for 12 hrs. The reaction mixture was allowed to cool to room temperature, quenched with water (5 ml), basified with a saturated aqueous solution of sodium hydrogencarbonate (5 ml). The resulting precipitate was collected by filtration, washed with water and dried to afford the crude product, which was purified by flash chromatography on silica gel eluting with 0 to 5% methanol in DCM/ethyl acetate (1/1). The solvent was evaporated to dryness to afford the product as a gum, which was further purified by preparative HPLC using a Waters X-Terra reverse-phase column (5 microns silica, 30 mm diameter, 150 mm length) and decreasingly polar mixtures of water (containing 0.2% ammonium carbonate) and acetonitrile as eluent. The fractions were evaporated to dryness to afford 1-(3-(4-((5-(hydroxymethyl)-2-methylphenyl)(methyl)amino)pyrimidin-2-ylamino)-5-morpholinophenyl)piperidin-4-one (50 mg, 0.10 mmol, 32.8%) as a off-white solid.

The (4-methyl-3-(methyl(2-(3-morpholino-5-(1,4-dioxa-8-azaspiro[4.5]decan-8-yl)phenylamino)pyrimidin-4-yl)amino)phenyl)methanol used as starting material was made as follows:—

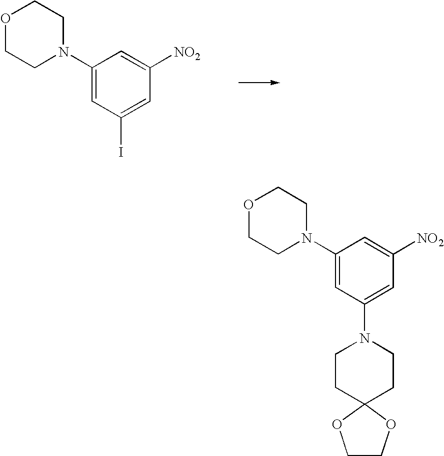

A mixture of 4-(3-iodo-5-nitrophenyl)morpholine (600 mg, 1.80 mmol), 1,4-dioxa-8-azaspiro[4.5]decane (0.460 ml, 3.59 mmol) was reacted as described in note (c) excepted that reflux was maintained for only 5 hrs and that toluene was used in place of DMSO. The reaction mixture was allowed to cool to room temperature under stirring and DCM was added. The insoluble was removed by filtration and the filtrate was concentrated down. The crude product was purified by flash chromatography on silica gel eluting with 0 to 10% ethyl acetate in DCM. The solvent was evaporated to afford 8-(3-morpholino-5-nitrophenyl)-1,4-dioxa-8-azaspiro[4.5]decane (404 mg, 64.4%) as a dark yellow gum. Mass Spectrum: M+H⁺ 349. NMR Spectrum (CDCl3): 1.81-1.86 (m, 4H), 3.18-3.24 (m, 4H), 3.37-3.42 (m, 4H), 3.84-3.89 (m, 4H), 4.00 (s, 4H), 6.67 (dd, 1H), 7.21 (dd, 1H), 7.29 (dd, 1H)

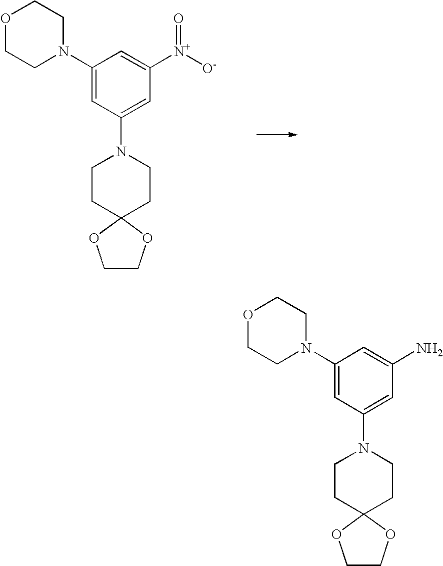

: 8-(3-morpholino-5-nitrophenyl)-1,4-dioxa-8-azaspiro[4.5]decane (260 mg, 0.74 mmol) was hydrogenated as described in note (c). The crude product was purified by flash chromatography on silica gel eluting with 0 to 5% methanol in DCM. The solvent was evaporated to dryness. The foam was triturated in EtOAc until it crystallized and the solid collected by filtration, washed with diethyl ether to afford 3-morpholino-5-(1,4-dioxa-8-azaspiro[4.5]decan-8-yl)aniline (230 mg, 97%). Mass Spectrum: M+H⁺ 320. NMR Spectrum (CDCl3): 1.78-1.87 (m, 4H), 3.07-3.15 (m, 4H), 3.23-3.31 (m, 4H), 3.57 (bs, 2H), 3.79-3.87 (m, 4H), 3.98 (s, 4H), 5.81 (s, 1H), 5.88 (s, 1H), 5.97 (s, 1H)

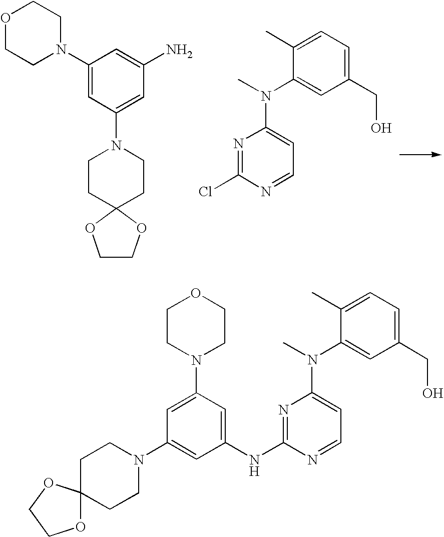

The coupling reaction was done using conditions similar to the ones described in note (c) (example 7.16) excepted that the heating was maintained during 6 hrs. Mass Spectrum: M+H⁺ 547. NMR Spectrum (CDCl3): 1.64-1.74 (m, 4H), 2.08 (s, 3H), 3.06 (m, 4H), 3.23 (m, 4H), 3.37 (s, 3H), 3.67-3.75 (m, 4H), 3.90 (s, 4H), 4.49 (d, 2H), 5.22 (t, 1H), 5.25 (bs, 1H), 6.11 (s, 1H), 7.00 (s, 1H), 7.04 (s, 1H), 7.18 (s, 1H), 7.26 (d, 1H), 7.34 (d, 1H), 7.77 (bs, 1H), 8.81 (s, 1H)

### EXAMPLE 8

The procedure of Example 6 was repeated using the corresponding chloropyrimidine (Method 3, 100 mg, 0.33 mmol) and aniline (Method 11, 0.33 mmol) to give the following compounds.

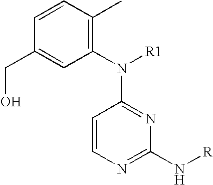

| | | | | Molecular | |
|---|---|---|---|---|---|
| Example | Name | R | R1 | ion (MH⁺) | NMR Spectrum |
| 8.1 | [3-[ethyl-[2-[(3-methylsulfonyl-5-morpholin-4-yl-phenyl)amino]pyrimidin-4-yl]amino]-4-methyl-phenyl]methanol | 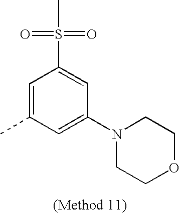 | Et | 498 | 1.19 (t, 3H), 2.09(s, 3H), 3.17 (s,3H), 3.19 (bs, 4H),3.68-3.78 (m, 1H),3.75-3.80 (m, 4H),4.12-4.24 (m, 1H),4.51 (d, 2H), 5.25(t, 1H), 5.26 (bs,1H), 6.99 (s, 1H),7.17 (s, 1H), 7.29(d, 1H), 7.37 (d,1H), 7.53 (d, 1H),7.83 (d, 1H), 8.19(s, 1H), 9.41 (s, 1H) |
| 8.2 | [4-methyl-3-[[2-[(3-methylsulfonyl-5-morpholin-4-yl-phenyl)amino]pyrimidin-4-yl]-propan-2-yl-amino]phenyl]methanol | 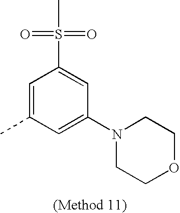 | i-Pr | 512 | 0.96 (d, 3H), 1.31(d, 3H), 2.07 (s,3H), 3.16 (s, 3H),3.16-3.20 (m, 4H),3.74-3.80 (m, 4H),4.51 (d, 2H), 5.12(bs, 1H), 5.20-5.29(m, 2H), 6.98 (s,1H), 7.11 (s, 1H),7.30 (d, 1H), 7.37(d, 1H), 7.53 (s,1H), 7.80 (d, 1H),8.16 (s, 1H), 9.39(bs, 1H) |
| 8.3 | [4-methyl-3-[2-methylpropyl-[2-[(3-methylsulfonyl-5-morpholin-4-yl-phenyl)amino]pyrimidin-4-yl]amino]phenyl]methanol | 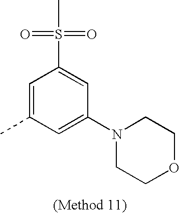 | i-Bu | 526 | 0.93 (d, 3H), 0.95(d, 3H), 1.78-1.88(m, 1H), 2.07 (s,3H), 3.14-3.22 (m,7H), 3.39 (dd, 1H),3.73-3.81 (m, 4H),4.18 (bs, 1H), 4.50(d, 2H), 5.23 (t,1H), 5.25 (bs, 1H),6.99 (s, 1H), 7.16(s, 1H), 7.27 (d,1H), 7.36 (d, 1H),7.49 (s, 1H), 7.82(d, 1H), 8.11 (s,1H), 8.31 (bs, 1H) |
| 8.4 | [3-[cyclopropyl-methyl-[2-[(3-methylsulfonyl-5-morpholin-4-yl-phenyl)amino]pyrimidin-4-yl]amino]-4-methyl-phenyl]methanol | 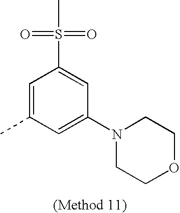 | c-Pr-CH₂— | 524 | 0.07-0.19 (m, 2H),0.33-0.45 (m, 2H),1.03-1.13 (m, 1H),2.12 (s, 3H), 3.17(s, 3H), 3.20 (bs,4H), 4.73-4.82 (m,5H), 4.85 (dd, 1H),4.51 (d, 2H), 5.23(t, 1H), 5.25 (bs,1H), 6.98 (s, 1H),7.22 (s, 1H), 7.27(d, 1H), 7.35 (d,1H), 7.60 (s, 1H),7.84 (d, 1H), 8.03(s, 1H), 9.38 (s, 1H) |

### EXAMPLE 9

### N-(3,5-dimorpholin-4-ylphenyl)-N′-(3-methoxyphenyl)-N′-methyl-pyrimidine-2,4-diamine

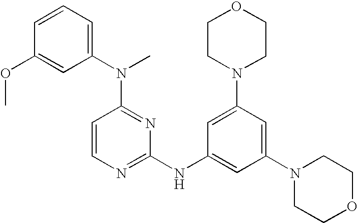

4-chloro-N-(3,5-dimorpholin-4-ylphenyl)pyrimidin-2-amine (Method 21, 70 mg, 0.19 mmol) and N-methyl m-anisidine (25 μl, 0.22 mmol) were mixed in 2-pentanol (1 ml). 4M HCl in dioxane was added (100 μl) and the mixture was heated at 100° C. for 15 hrs. The solvent was removed under vacuum and the residue was dissolved in DMF (1 ml) and purified on a preparative HPLC-MS system (Column: C18, 5 microns, 19 mm diameter, 100 mm length, elution with a gradient of water and acetonitrile containing 2 g/l of ammonium carbonate). Evaporation of the collected fractions gave the title compound (62 mg, 68% yield); NMR Spectrum (500 MHz, DMSOd6) 3.04-3.06 (m, 8H), 3.44 (s, 3H), 3.71-3.73 (m, 8H), 3.77 (s, 3H), 5.78 (d, 1H), 6.11 (s, 1H), 6.91 (s, 1H), 6.93 (s, 2H), 6.99 (s, 2H), 7.38 (t, 1H), 7.85 (d, 1H), 8.85 (s, 1H). Mass Spectrum: MH⁺ 477.

### EXAMPLE 10

The procedure described in Example 9 was repeated using the appropriate aniline. Thus were obtained the compounds described below.

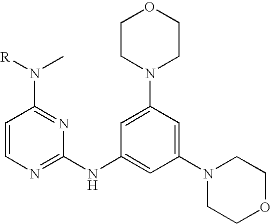

| | | | Molecular | |
|---|---|---|---|---|
| Example | Name | R | ion (MH⁺) | NMR Spectrum |
| 10.1 | N-(3,5-dimorpholin-4-ylphenyl)-N′-methyl-N′-(4-methylphenyl)pyrimidine-2,4-diamine | 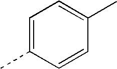 | 461 | 2.35 (s, 3H), 3.01-3.08 (m,8H), 3.42 (s, 3H), 3.69-3.76(m, 8H), 5.69 (d, 1H), 6.11(t, 1H), 6.99 (d, 2H), 7.23 (d,2H), 7.29 (d, 2H), 7.82 (d,1H), 8.83 (s, 1H) |
| 10.2 | N′-(3-chlorophenyl)-N-(3,5-dimorpholin-4-ylphenyl)-N′-methyl-pyrimidine-2,4-diamine | 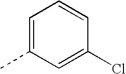 | 481 | 2.99-3.07 (m, 8H), 3.45 (s,3H), 3.68-3.76 (m, 8H), 5.85(d, 1H), 6.11 (t, 1H), 6.96 (d,2H), 6.36 (ddd, 1H), 6.39(ddd, 1H), 7.47-7.52 (m,2H), 7.92 (d, 1H), 8.89 (s,1H) |
| 10.3 | N-(3,5-dimorpholin-4-ylphenyl)-N′-methyl-N′-phenyl-pyrimidine-2,4-diamine | 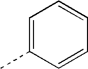 | 447 | 3.00-3.08 (m, 8H), 3.45 (s,3H), 3.67-3.78 (m, 8H), 5.73(d, 1H), 6.11 (t, 1H), 6.99 (d,2H), 7.34 (t, 1H), 7.36 (d,2H), 7.49 (t, 2H), 7.85 (d,1H), 8.85 (s, 1H) |
| 10.4^{c} | [3-[[2-[(3,5-dimorpholin-4-ylphenyl)amino]pyrim-idin-4-yl]-methyl-amino]phenyl]methanol | 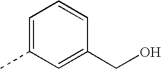 | 477 | 3.01-3.10 (m, 8H), 3.44 (s,3H), 3.67-3.77 (m, 8H), 4.53(d, 2H), 5.27 (t, 1H), 5.72 (d,1H), 6.11 (t, 1H), 6.99 (d,2H), 7.20 (d, 1H), 7.25-7.30(m, 2H), 7.43 (dd, 1H), 7.83(d, 1H), 8.84 (s, 1H) |
| 10.5 | N-(3,5-dimorpholin-4-ylphenyl)-N′-(3-fluorophenyl)-N′-methyl-pyrimidine-2,4-diamine | 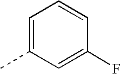 | 465 | 2.99-3.07 (m, 8H), 3.44 (s,3H), 3.67-3.75 (m, 8H), 5.86(s, 1H), 6.10 (t, 1H), 6.95 (d,2H), 7.16 (ddd, 1H), 7.21(dd, 1H), 7.28 (ddd, 1H),7.50 (dd, 1H), 7.90 (d, 1H),8.88 (s, 1H) |
| 10.6 | N-(3,5-dimorpholin-4-ylphenyl)-N′-methyl-N′-(3-methylphenyl)pyrimidine-2,4-diamine | 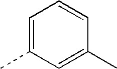 | 461 | 2.33 (s, 3H), 3.01-3.08 (m,8H), 3.43 (s, 3H), 3.68-3.75(m, 8H), 5.71 (d, 1H), 6.10(t, 1H), 6.98 (d, 2H),7.11-7.19 (m, 3H), 7.36 (dd,1H), 7.83 (d, 1H), 8.83 (s, 1H) |
| 10.7 | N-(3,5-dimorpholin-4-ylphenyl)-N′-methyl-N′-(3-methylsulfanylphenyl)pyrimidine-2,4-diamine | 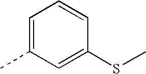 | 493 | 2.48 (s, 3H), 3.00-3.07 (m,8H), 3.44 (s, 3H), 3.68-3.74(m, 8H), 5.76 (d, 1H), 6.10(t, 1H), 6.97 (d, 2H), 7.11(dd, 1H), 7.21 (ddd, 1H),7.23 (d, 1H), 7.40 (dd, 1H),7.86 (d, 1H), 8.85 (s, 1H) |
| 10.8^{d} | N′-(3,5-dimethylphenyl)-N-(3,5-dimorpholin-4-ylphenyl)-N′-methyl-pyrimidine-2,4-diamine | 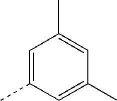 | 475 | 2.29 (s, 6H), 3.01-3.09 (m,8H), 3.41 (s, 3H), 3.67-3.75(m, 8H), 5.70 (d, 1H), 6.10(t, 1H), 6.95 (s, 2H), 6.97 (s,1H), 6.99 (d, 2H), 7.81 (d,1H), 8.82 (s, 1H) |
| 10.9^{e} | N′-(2,5-dimethylphenyl)-N-(3,5-dimorpholin-4-ylphenyl)-N′-methyl-pyrimidine-2,4-diamine | 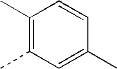 | 475 | 2.05 (s, 3H), 2.30 (s, 3H),3.06 (bs, 8H), 3.35 (s, 3H),3.67-3.77 (m, 8H), 5.25 (bs,1H), 6.11 (s, 1H), 7.02 (bs,2H), 7.07 (s, 1H), 7.13 (d,1H), 7.26 (d, 1H), 7.77 (bs,1H), 8.84 (bs, 1H) |
| 10.10 | 3-[[2-[(3,5-dimorpholin-4-ylphenyl)amino]pyrimidin-4-yl]-methyl-amino]benzonitrile | 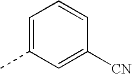 | 472 | 2.95-3.05 (m, 8H), 3.45 (s,3H), 3.66-3.75 (m, 8H), 5.91(d, 1H), 6.10 (t, 1H), 6.92 (d,2H), 7.64 (dd, 1H), 7.74 (dd,1H), 7.75 (d, 1H), 7.91 (dd,1H), 7.95 (d, 1H), 8.90 (s,1H) |
| 10.11 | N′-(3,4-dichlorophenyl)-N-(3,5-dimorpholin-4-ylphenyl)-N′-methyl-pyrimidine-2,4-diamine | 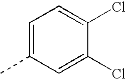 | 515 | 2.96-3.04 (m, 8H), 3.42 (s,3H), 3.66-3.74 (m, 8H), 5.94(d, 1H), 6.09 (t, 1H), 6.92 (d,2H), 7.39 (dd, 1H), 7.69 (d,1H), 7.71 (d, 1H), 7.95 (d,1H), 8.90 (s, 1H) |
| 10.12^{f} | N-(3,5-dimorpholin-4-ylphenyl)-N′-(2-methoxyphenyl)-N′-methyl-pyrimidine-2,4-diamine | 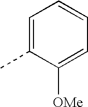 | 477 | 3.01-3.09 (m, 8H), 3.45 (s,3H), 3.69-3.75 (m, 8H), 3.78(s, 3H), 5.78 (d, 1H), 6.11 (s,1H), 6.89-6.93 (m, 3H), 6.99(d, 2H), 7.39 (dd, 1H), 7.85(d, 1H), 8.86 (s, 1H) |
| 10.13^{g} | N-(3,5-dimorpholin-4-ylphenyl)-N′-methyl-N′-(2,4,6-trimethylphenyl)pyrimidine-2,4-diamine | 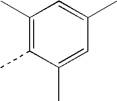 | 489 | 2.04 (s, 6H), 2.28 (s, 3H),3.03-3.11 (m, 8H), 3.30 (s,3H), 3.69-3.76 (m, 8H), 5.17(d, 1H), 6.11 (s, 1H), 7.02 (s,2H), 7.05 (d, 2H), 7.75 (s,1H), 8.84 (s, 1H) |
| 10.14^{f} | N′-(2,4-difluorophenyl)-N-(3,5-dimorpholin-4-ylphenyl)-N′-methyl-pyrimidine-2,4-diamine | 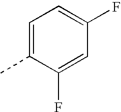 | 483 | 2.99-3.09 (m, 8H), 3.38 (s,3H), 3.68-3.76 (m, 8H), 5.69(bs, 1H), 6.11 (s, 1H), 6.94(s, 2H), 7.23 (ddd, 1H), 7.47(ddd, 1H), 7.57 (ddd, 1H),7.92 (d, 1H), 8.88 (s, 1H) |
| 10.15^{f} | N-(3,5-dimorpholin-4-ylphenyl)-N′-methyl-N′-(2-methylphenyl)pyrimidine-2,4-diamine | 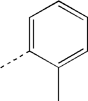 | 461 | 2.11 (s, 3H), 3.06 (bs, 8H),3.37 (s, 3H), 3.68-3.77 (m,8H), 5.24 (bs, 1H), 6.11 (s,1H), 7.02 (bs, 2H), 7.23-7.28(m, 1H), 7.29-7.36 (m, 2H),7.39 (bs, 1H), 7.79 (bs, 1H),8.84 (bs, 1H) |
| 10.16^{f} | N-(3,5-dimorpholin-4-ylphenyl)-N′-(2-fluorophenyl)-N′-methyl-pyrimidine-2,4-diamine | 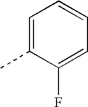 | 465 | 2.98-3.07 (m, 8H), 3.40 (s,3H), 3.67-3.76 (m, 8H), 5.64(d, 1H), 6.10 (s, 1H), 6.95 (s,2H), 7.32 (ddd, 1H), 7.38(ddd, 1H), 7.43 (ddd, 1H),7.49 (ddd, 1H), 7.90 (d, 1H),8.87 (s, 1H) |
| 10.17^{h} | 4-[[2-[(3,5-dimorpholin-4-ylphenyl)amino]pyrim-idin-4-yl]-methyl-amino]benzonitrile | 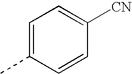 | 472 | 2.94-3.02 (m, 8H), 3.48 (s,3H), 3.66-3.72 (m, 8H), 6.10(t, 1H), 6.12 (d, 1H), 6.88 (d,2H), 7.57 (d, 2H), 7.87 (d,2H), 8.02 (d, 1H), 8.95 (s,1H) |
| 10.18ⁱ | [2-chloro-5-[[2-[(3,5-dimorpholin-4-ylphenyl)amino]pyrimidin-4-yl]-methyl-amino]phenyl]methanol | 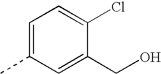 | 511 | 2.99-3.08 (m, 8H), 3.44 (s,3H), 3.67-3.75 (m, 8H), 4.57(d, 2H), 5.49 (t, 1H), 5.79 (d,1H), 6.10 (s, 1H), 6.96 (d,2H), 7.27 (dd, 1H), 7.46-7.51(m, 2H), 7.87 (d, 1H),8.87 (s, 1H) |
| 10.19^{j} | [4-chloro-3-[[2-[(3,5-dimorpholin-4-ylphenyl)amino]pyrimidin-4-yl]-methyl-amino]phenyl]methanol | 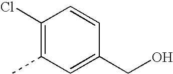 | 511 | 2.99-3.12 (m, 8H), 3.37 (s,3H), 3.68-3.76 (m, 8H), 4.53(d, 2H), 5.33 (bs, 1H), 5.38(t, 1H), 6.11 (s, 1H), 7.00(bs, 2H), 7.39 (d, 1H), 7.42(s, 1H), 7.61 (d, 1H), 7.84(bs, 1H), 8.89 (bs, 1H) |
| 10.20ⁱ | [3-chloro-5-[[2-[(3,5-dimorpholin-4-ylphenyl)amino]pyrimidin-4-yl]-methyl-amino]phenyl]methanol | 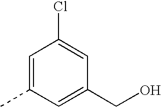 | 511 | 3.00-3.08 (m, 8H), 3.44 (s,3H), 3.68-3.76 (m, 8H), 4.52(d, 2H), 5.40 (t, 1H), 5.83 (d,1H), 6.14 (t, 1H), 6.96 (d,2H), 7.26 (s, 1H), 7.31 (s,1H), 7.34 (s, 1H), 7.90 (d,1H), 8.88 (s, 1H) |
| 10.21ⁱ | [3-[[2-[(3,5-dimorpholin-4-ylphenyl)amino]pyrimidin-4-yl]-methyl-amino]-5-methoxy-phenyl]methanol | 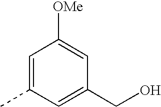 | 507 | 3.01-3.09 (m, 8H), 3.43 (s,3H), 3.68-3.75 (m, 8H), 3.76(s, 3H), 4.49 (d, 2H), 5.27 (t,1H), 5.77 (d, 1H), 6.10 (t,1H), 6.78 (s, 1H), 6.84-6.88(m, 2H), 6.99 (d, 2H), 7.83(d, 1H), 8.84 (s, 1H) |
| 10.22^{k} | 1-(3-((2-(3,5-dimorpholinophenyl-amino)pyrimidin-4-yl)(methyl)amino)-4-methylphenyl) ethanol | 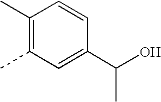 | 505 | 1.32 (d, 3H), 2.07 (s, 3H),3.00-3.14 (m, 8H), 3.36 (s,3H), 3.67-3.78 (m, 8H),4.67-4.75 (m, 1H), 5.18 (d,1H), 5.24 (bs, 1H), 6.11 (s,1H), 7.03 (s, 2H), 7.20 (d,1H), 7.26-7.36 (m, 2H), 7.78(s, 1H), 8.84 (s, 1H) |

^{a}2-propanol was used as the solvent.
^{b}The reaction mixture was heated at 120° C. for 15 min in a Personal Chemistry EMRYS ™ Optimizer EXP microwave synthesisor except stated otherwise.
^{c}see method 22 for the starting aniline
^{d}The mixture was heated for 30 min at 120° C.
^{e}The mixture was heated for 45 min at 140° C.
^{f}The mixture was heated for 20 min at 150° C.
^{g}The mixture was heated for 90 min at 150° C.
^{h}The mixture was heated for 45 min at 150° C.
ⁱThe reaction was run on 0.45 mmol scale (See method 22 for the preparation of the starting aniline). The mixture was heated in an oil bath at 120° C. for 2 hrs. After cooling and evaporation of the solvents, the residue was dissolved in DCM (10 ml) and 7 N methanolic ammonia (1 ml) was added. After filtration, the filtrate was concentrated, purified by chromatography on silica gel (2% methanol in DCM), and triturated in ether/pentane.
^{j}As for i except that the reaction was heated for 18 hrs.
^{k}The reaction was heated in isopropanol (20 ml) - DMA (5 ml) for 11 hrs at 120° C., then for 30 minutes at 140° C. in a Personal Chemistry EMRYS ™ Optimizer EXP microwave synthesisor.

The 1-(4-methyl-3-(methylamino)phenyl)ethanol used as starting material was made as follows:

Imidazole (3.16 g, 46.36 mmol) and tert-butyldimethylsilyl chloride (3.97 ml, 23.18 mmol) were successively added in one portion to a stirred solution of 1-(4-methyl-3-nitrophenyl)ethanol (2.1 g, 11.59 mmol) in DMF (10 ml) at room temperature. The resulting solution was stirred at room temperature for 2 hrs. The reaction mixture was concentrated to dryness, quenched with methanol (5 ml), diluted with diethyl ether, washed with water (2×15 ml), brine (15 ml), dried over magnesium sulfate and concentrated to afford the crude product as an oil. The crude product was purified by flash chromatography on silica gel eluting with 0% to 10% DCM in petroleum ether. The solvent was evaporated to dryness to afford tert-butyldimethyl(1-(4-methyl-3-nitrophenyl)ethoxy)silane (3.15 g, 92%) as a pale beige oil.

A solution of tert-butyldimethyl(1-(4-methyl-3-nitrophenyl)ethoxy)silane (3.15 g, 10.66 mmol) and platinum(IV) oxide (310 mg, 1.37 mmol) in ethanol (30 ml) was hydrogenated under 1 atm of hydrogen at room temperature for 90 minutes. The resulting solution was filtered and the filtrate was concentrated to dryness to afford the crude 5-(1-(tert-butyldimethylsilyloxy)ethyl)-2-methylaniline (2.67 g, 94%) as a pale beige oil. 5-(1-(Tert-butyldimethylsilyloxy)ethyl)-2-methylaniline (2.5 g, 9.42 mmol), and di-tert-butyl dicarbonate (2.60 ml, 11.30 mmol) in acetonitrile (5 ml) were stirred at room temperature for 18 hrs. The reaction mixture was concentrated to dryness and the crude product was purified by flash chromatography on silica gel eluting with DCM. The solvent was evaporated to dryness to afford tert-butyl 5-(1-(tert-butyldimethylsilyloxy)ethyl)-2-methylphenylcarbamate (3.40 g, 99%) as a white solid.

Sodium hydride 60% in oil (0.263 g, 6.56 mmol) was added to a stirred solution of tert-butyl 5-(1-(tert-butyldimethylsilyloxy)ethyl)-2-methylphenylcarbamate (2 g, 5.47 mmol), dissolved in DMF (30 ml) at 0° C. under argon. The resulting solution was stirred at room temperature for 90 minutes. Methyl iodide (0.409 ml, 6.56 mmol) was added in one portion to the mixture. The resulting mixture was stirred at room temperature for 5 hrs. The reaction mixture was quenched with a saturated aqueous solution of ammonium chloride, diluted with water (150 ml) and extracted with diethyl ether (2×40 ml). The combined organic phases were washed with water, a saturated aqueous solution of brine, dried over magnesium sulfate and concentrated. The crude product was purified by flash chromatography on silica gel eluting with 40 to 100% DCM in petroleum ether. The solvent was evaporated to dryness to afford tert-butyl 5-(1-(tert-butyldimethylsilyloxy)ethyl)-2-methylphenyl(methyl)carbamate (1.6 g, 77%) as a colorless oil.

Aqueous 2N hydrochloric acid (6.45 ml, 12.9 mmol) was added to a stirred solution of tert-butyl 5-(1-(tert-butyldimethylsilyloxy)ethyl)-2-methylphenyl(methyl)carbamate (1.4 g, 3.69 mmol) dissolved in methanol (15 ml). The resulting solution was stirred at 50° C. for 60 minutes. The organic solvent was removed in vacuo, the aqueous residue was neutralised with a solution of aqueous 4N sodium hydroxide. The mixture was extracted with ethyl acetate (3×15 ml). The combined organic phases were washed with brine, dried over magnesium sulfate and concentrated. The residue was purified by flash chromatography on silica gel eluting with 0 to 4% methanol in DCM. The solvent was evaporated to dryness to afford 1-(4-methyl-3-(methylamino)phenyl)ethanol (0.595 g, 98%) as a colorless oil. Mass spectrum: MH⁺ 166.

### EXAMPLE 11

### N′-(4-chlorophenyl)-N-(3,5-dimorpholin-4-ylphenyl)-N′-methyl-pyrimidine-2,4-diamine

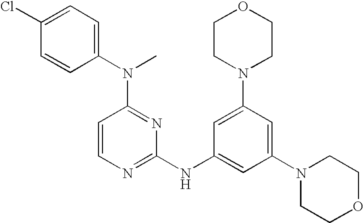

A pressure vessel was charged with 4-chloro-N-(3,5-dimorpholin-4-ylphenyl)pyrimidin-2-amine (Method 21, 500 mg, 1.3 mmol) and 10 ml of a 6N solution of methylamine in methanol. The reaction mixture was heated at 140° C. for 20 hrs and the resulting solution was evaporated. Purification of the residue on silica gel (5% MeOH in DCM) provided N-(3,5-dimorpholin-4-ylphenyl)-N′-methyl-pyrimidine-2,4-diamine as white solid (370 mg, 75%). NMR Spectrum (500 MHz, CDCl3) 2.97 (d, 3H), 3.15-3.17 (m, 8H), 3.84-3.86 (m, 8H), 4.80 (bs, 1H), 5.85 (d, 1H), 6.15 (s, 1H), 6.83 (s, 2H), 7.10 (bs, 1H), 7.90 (bs, 1H). Mass Spectrum: MH⁺ 371.

N-(3,5-dimorpholin-4-ylphenyl)-N′-methyl-pyrimidine-2,4-diamine (37 mg, 0.10 mmol), 4-chloro-bromobenzene (57 mg, 0.30 mmol), potassium carbonate (276 mg, 2.0 mmol), Pd2 dba3 (3 mg, 0.005 mmol), and Xantphos (6 mg, 0.01 mmol) were mixed under nitrogen in 3 ml of dry toluene degassed with nitrogen. The reaction mixture was heated at 120° C. for 20 hrs then filtered and evaporated. The residue was purified on a preparative HPLC-MS system (Column: C18, 5 microns, 19 mm diameter, 100 mm length, elution with a gradient of water and acetonitrile containing 2 g/l of ammonium carbonate) to give 10 mg (21% yield) of the title compound. NMR Spectrum (500 MHz, DMSO) 3.01-3.03 (m, 8H), 3.42 (s, 3H), 3.70-3.72 (m, 8H), 5.81 (d, 1H), 6.10 (s, 1H), 6.94 (s, 2H), 7.39 (d, 2H), 7.51 (d, 2H), 7.89 (d, 2H), 8.86 (s, 1H). Mass Spectrum: MH⁺ 481.

### EXAMPLE 12

The following compounds were prepared using the procedure described in example 6.

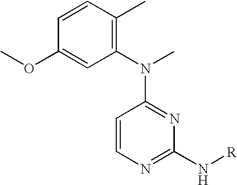

| | | R | | |
|---|---|---|---|---|
| | | (Starting | Molecular | |
| Example | Name | aniline) | ion (MH⁺) | NMR Spectrum |
| 12.1 | N-(3,5-dimorpholin-4-ylphenyl)-N′-(5-methoxy-2-methyl-phenyl)-N′-methyl-pyrimidine-2,4-diamine | 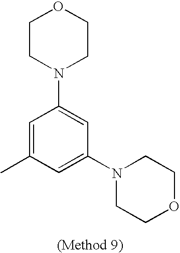 | 491 | (DMSO-d6) 2.03 (s,3 H), 3.04-3.12 (m,8 H), 3.39 (s, 3 H),3.71-3.76 (m, 8 H),3.76 (s, 3 H), 5.39 (bs,1 H), 6.11 (s, 1 H), 6.85(d, 1 H), 6.92 (dd, 1 H),7.00 (bs, 2 H), 7.29 (d,1 H), 7.81 (d, 1 H), 8.66(s, 1 H) |
| 12.2 | N′-(5-methoxy-2-methyl-phenyl)-N′-methyl-N-(3-methylsulfonyl-5-morpholin-4-yl-phenyl)pyrimidine-2,4-diamine | 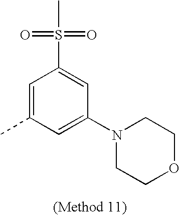 | 484 | (DMSO-d6) 2.02 (s,3 H), 3.13 (s, 3 H), 3.17(bs, 4 H), 3.38 (s, 3 H),3.73-3.78 (m, 7 H),6.43 (bs, 1 H), 6.84 (d,1 H), 6.91 (dd, 1 H),6.96 (s, 1 H), 7.28 (d,1 H), 7.56 (s, 1 H), 7.85(d, 1 H), 8.14 (s, 1 H),9.25 (s, 1 H) |
| 12.3 | 3-[[4-[(5-methoxy-2-methyl-phenyl)-methyl-amino]pyrimidin-2-yl]amino]-N,N-dimethyl-5-morpholin-4-yl-benzamide | 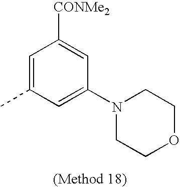 | 477 | (DMSOd6 at 323° K.)2.01 (s, 3 H), 2.94 (s,6 H), 3.04-3.13 (m,4 H), 3.34 (s, 3 H),3.70-3.77 (m, 7 H),5.42 (bs, 1 H), 6.45 (s,1 H), 6.82 (d, 1 H), 6.89(dd, 1 H), 7.26 (d, 1 H),7.29 (s, 1 H), 7.47 (bs,1 H), 7.83 (d, 1 H), 8.83(s, 1 H) |
| 12.4 | N′-(5-methoxy-2-methyl-phenyl)-N′-methyl-N-[3-(4-methylpiperazin-1-yl)-5-morpholin-4-yl-phenyl]pyrimidine-2,4-diamine | 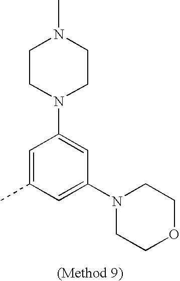 | 504 | (DMSOd6 at 297° K.)2.01 (s, 3 H), 2.21 (s,3 H), 2.40-2.46 (m,4 H), 3.05 (bs, 4 H),3.09 (bs, 4 H), 3.36 (s,3 H), 3.68-3.73 (m,4 H), 3.74 (s, 3 H), 5.29(bs, 1 H), 6.09 (s, 1 H),6.86 (d, 1 H), 6.91 (d,1 H), 6.99 (bs, 1 H),7.03 (bs, 1 H), 7.28 (d,1 H), 7.79 (bs, 1 H),8.81 (bs, 1 H) |
| 12.5 | 1-(3-(4-((5-methoxy-2-methylphenyl)(methyl)amino)pyrim-idin-2-ylamino)-5-morpholinophenyl)piperidin-4-ol | 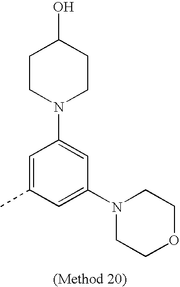 | 505 | (DMSOd6): 1.40-1.41(m, 2 H), 1.74-1.85 (m,2 H), 2.01 (s, 3 H),2.74-2.84 (m, 2 H),2.99-3.11 (m, 4 H),3.36 (s partially hiddenby H2O, 3 H), 3.44-3.54 (m, 2 H), 3.55-3.64 (m, 1 H), 3.68-3.73 (m, 4 H), 3.74 (s,3 H), 4.64 (d, 1 H), 5.29(bs, 1 H), 6.08 (s, 1 H),6.86 (d, 1 H), 6.91 (dd,1 H), 6.96 (bs, 1 H),7.04 (bs, 1 H), 7.28 (d,1 H), 7.79 (bs, 1 H),8.80 (bs, 1 H) |
| 12.6^{a} | N4-(5-methoxy-2-methylphenyl)-N4-methyl-N2-(3-morpholino-5-(piperazin-1-yl)phenyl)pyrimidine-2,4-diamine | 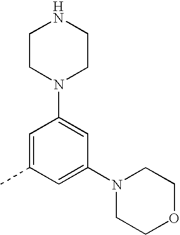 | 490 | |
| 12.7^{b} | 4-[3-[[4-[(5-methoxy-2-methyl-phenyl)-methyl-amino]pyrimidin-2-yl]amino]-5-morpholin-4-yl-phenyl]morpholin-3-one | 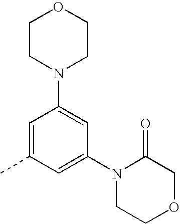 | 505 | |
| 12.8^{c} | (S)-N4-(5-methoxy-2-methylphenyl)-N4-methyl-N2-(3-(3-methylmorpholino)-5-morpholinophenyl)pyrimidine2,4-diamine | 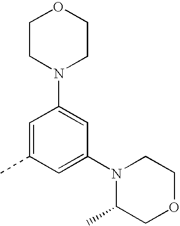 | 505 | DMSOd6: 0.98 (d,3 H), 2.01 (s, 3 H),2.94-3.12 (m, 6 H),3.36 (s, 3 H), 3.55(ddd, 1 H), 3.62 (dd,1 H), 3.66-3.78 (m,6 H), 3.74 (s, 3 H), 3.86(d, 1 H), 5.59 (bs, 1 H),6.07 (s, 1 H), 6.82 (d,1 H), 6.91 (dd, 1 H),7.00 (bs, 2 H), 7.28 (d,1 H), 7.79 (bs, 1 H),8.82 (bs, 1 H) |

Notes:
^{a}A solution of tert-butyl 4-(3-(4-((5-methoxy-2-methylphenyl)(methyl)amino) pyrimidin-2-ylamino)-5 -morpholinophenyl)piperazine-1-carboxylate (280 mg, 0.47 mmol) in DCM (3 ml) and HCl/ isopropanol 7N (1 ml) was stirred at 25° C. over a period of 2 hrs. The mixture was evaporated, dissolved in DMF and NH4OH aqueous (0.5 ml) then purified by preparative HPLC using a reverse-phase column with acetonitrile and aqueous ammonium carbonate as eluent. The fractions containing the desired compound were evaporated to dryness. The resulting foam was triturated with diethyl ether and pentane. The resulting solid was dried at 50° C. to afford N4-(5-methoxy-2-methylphenyl)-N4-methyl-N2-(3-morpholino-5-(piperazin-1-yl)phenyl)pyrimidine-2,4-diamine (105 mg, 45.2%) as a beige solid. NMR Spectrum (DMSOd6): 2.01 (s, 3 H), 2.57 (bs, 1 H), 2.77- 2.83 (m, 4 H), 3.00 (bs, 4 H), 3.05 (bs, 4 H), 3.37 (s, 3 H), 3.68-3.74 (m, 4 H), 3.74 (s, 3 H), 5.29 (bs, 1 H), 6.07 (s, 1 H), 6.85 (d, 1 H), 6.91 (dd, 1 H), 6.98 (bs, 1 H), 7.01 (bs, 1 H), 7.28 (d, 1 H), 7.79 (bs, 1 H), 8.80 (bs, 1 H) The above tert-butyl 4-(3-(4-((5-methoxy-2-methylphenyl)(methyl)amino) pyrimidin-2- ylamino)-5-morpholinophenyl)piperazine-1-carboxylate was prepared as follows:- A mixture of 4-(3-iodo-5-nitrophenyl)morpholine (1 g, 2.99 mmol), tert-butyl piperazine-1-carboxylate (1.115 g, 5.99 mmol), cesium carbonate (4.88 g, 14.97 mmol), palladium (II) acetate (0.067 g, 0.30 mmol) and 2,2′-bis(diphenylphosphino)-1,1′-binaphthyl (0.093 g, 0.15 mmol) dissolved in toluene (25 ml) was degased with nitrogen and heated to reflux for 2 hrs. The reaction was complete. After cooling at room temperature, the suspension was diluted with DCM (20 ml). The insoluble was filtered. The filtrate was concentrated to dryness to give an orange solid which was dissolved in DCM and purified by flash chromatography on silica gel eluting with DCM and 3% AcOEt/DCM. The solvent was evaporated to dryness to afford tert-butyl 4-(3-morpholino-5-nitrophenyl)piperazine-1-carboxylate (0.920 g, 78%) as an orange solid. Mass Spectrum: [M − H]⁻ 392. NMR Spectrum (CDCl3): 1.49 (s, 9 H), 3.17-3.26 (m, 8 H), 3.56-3.63 (m, 4 H), 3.84-3.90 (m, 4 H), 6.65 (dd, 1 H), 7.25-7.27 (m partially hidden by CHCl3, 2 H)
A suspension of tert-butyl 4-(3-morpholino-5-nitrophenyl)piperazine-1-carboxylate (870 mg, 2.22 mmol), ADAMS' platinum oxide (50.3 mg, 0.22 mmol) in EtOH (30 ml) was hydrogenated at 1100 mbar for 2H30. A white precipitate was formed. The resulting suspension was dissolved with AcOEt. The catalyseur was filtered and the filtrate concentrated to dryness to afford the crude tert-butyl 4-(3-amino-5-morpholinophenyl) piperazine-1-carboxylate (783 mg, 97%) as a beige solid. Mass Spectrum: M + H⁺ 363. NMR Spectrum (CDCl3): 1.48 (s, 9 H), 3.02-3.16 (m, 8 H), 3.50-3.58 (m, 4 H), 3.60 (bs, 2 H), 3.78-3.87 (m, 4 H), 5.83-5.87 (m, 2 H), 5.94 (dd, 1 H)
A suspension of 2-chloro-N-(5-methoxy-2-methylphenyl)-N-methylpyrimidin-4-amine (180 mg, 0.68 mmol), tert-butyl 4-(3-amino-5-morpholinophenyl)piperazine-1-carboxylate (260 mg, 0.72 mmol) and HCl/dioxane 4N (5.97 μl, 0.02 mmol) in 2-pentanol (2 ml) was stirred at 120° C. over a period of 1H30. The mixture was concentrated to dryness, diluted with DCM (10 ml) and a 5N solution of NH3 in methanol (2 ml). The insoluble was filtered and the filtrate was concentrated in dryness then dissolved in DCM and purified by flash chromatography on silica gel eluting with 40% AcOEt/DCM to afford tert-butyl 4-(3-(4-((5-methoxy-2-methylphenyl)(methyl)amino)pyrimidin-2-ylamino)-5-morpholinophenyl) piperazine-1-carboxylate (280 mg, 69.6%) as a foam. Mass Spectrum: M + H⁺ 590.
^{b}Stirred at 80° C. for 2 hrs. The reaction mixture was concentrated to dryness and diluted with DCM: methanolic ammonia 7N (95:5, 10 mL). The resulting precipitate was removed by filtration and washed with DCM. The filtrate was concentrated down, dissolved in DCM and purified by flash chromatography on silica gel eluting with 0 to 5% methanol in DCM. NMR Spectrum (DMSOd6 at 323° K.): 2.01 (s, 3 H), 3.05-3.13 (m, 4 H), 3.34 (s, 3 H), 3.65 (bs, 2 H), 3.70-3.77 (m, 4 H), 3.76 (s, 3 H), 3.92-3.97 (m, 2 H), 4.16 (s, 2 H), 5.42 (bs, 1 H), 6.50 (s, 1 H), 6.82 (d, 1 H), 6.90 (dd, 1 H), 7.27 (d, 1 H), 7.30 (bs, 1 H), 7.32 (bs, 1 H), 7.82 (d, 1 H), 8.89 (s, 1 H)
^{c}Prepared as described for the synthesis of 7.17.

### EXAMPLE 13

The following compounds were prepared using the procedure described in example 6 but the reaction was carried out in 2-pentanol at 120° C. for 1 hr. The crude materials were purified on a preparative HPLC-MS system (Column: C18, 5 microns, 19 mm diameter, 100 mm length, elution with a gradient of water and acetonitrile containing 2 g/l of ammonium carbonate)

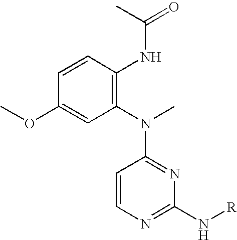

| | | R | Molecular | |
|---|---|---|---|---|
| Example | Name | (Starting aniline) | ion (MH⁺) | NMR Spectrum |
| 13.1 | N-[2-[[2-[(3,5-dimorpholin-4-ylphenyl)amino]pyrimidin-4-yl]-methyl-amino]-4-methoxy-phenyl]acetamide | 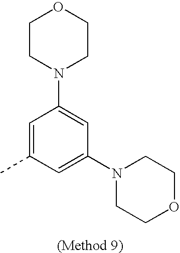 | 534 | (DMSO-d6) 1.92 (s, 3 H),3.06 (bs, 8 H), 3.34 (shidden by H2O, 3 H),3.70-3.73 (m, 8 H), 3.74(s, 3H), 5.45 (bs, 1 H),6.10 (s, 1 H), 6.89 (s,1 H), 6.93 (dd, 1 H), 7.03(s, 2 H), 7.67 (d, 1 H),7.80 (bs, 1 H), 8.83 (s,1 H), 9.16 (s, 1 H) |
| 13.2 | N-[4-methoxy-2-[methyl-[2-[(3-methylsulfonyl-5-morpholin-4-yl-phenyl)amino]pyrimidin-4-yl]amino]phenyl]acetamide | 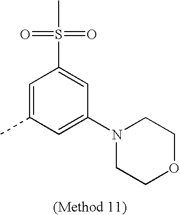 | 527 | (DMSO-d6 + TFAd) 1.96(s, 3 H), 3.24 (s, 3 H),3.25-3.29 (m, 4 H), 3.44(s, 3 H), 3.76-3.82 (m,7 H), 5.80 (d, 1 H), 7.05(d, 1 H), 7.07 (dd, 1 H),7.28 (s, 1 H), 7.35 (s,1 H), 7.65 (dd, 1 H), 7.88(dd, 1 H), 7.91 (bs, 1 H) |

### EXAMPLE 14

### N′-(2,3-difluorophenyl)-N-(3,5-dimorpholin-4-ylphenyl)-N′-methyl-pyrimidine-2,4-diamine

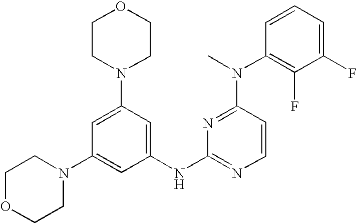

A mixture of 4-chloro-N-(3,5-dimorpholin-4-ylphenyl)-N-[(4-methoxyphenyl)methyl]pyrimidin-2-amine (120 mg, 0.24 mmol, Method 25), 2,3-difluoroaniline (0.27 mmol) and 4M hydrogen chloride in dioxane (0.11 ml) in 2-pentanol (1.5 ml) in a sealed tube was heated at 120° C. for 3 hrs. After cooling, the solvent was evaporated to dryness. The residue was dissolved in DCM (3 ml). 7N Methanolic ammonia (0.3 ml) was added. The insoluble was removed by filtration and the resulting solution was evaporated to dryness.

The residue was dissolved in DMF (0.5 ml). A slurry of sodium hydride (20 mg) in DMF (1 ml) was added and the mixture was stirred for 1 hr followed by addition of methyl iodide (10 μl) in DMF (5001). The resulting mixture was stirred at room temperature for 24 hrs and was evaporated to dryness. The residue was dissolved in trifluoroacetic acid (1 ml) and anisole (3 drops). The mixture was stirred at room temperature for 24 hrs. The mixture was evaporated to dryness, 7N methanolic ammonia (1 ml) was slowly added The insoluble was removed by filtration and the resulting solution was evaporated to dryness. The mixture was directly injected on an HPLC column (C18, 5 microns, 19 mm diameter, 100 mm length) of a preparative HPLC-MS system eluting with a mixture of water and acetonitrile containing 2 g/l of ammonium carbonate (gradient) to give the title compound (46 mg).

NMR Spectrum: (DMSOd₆) 2.97-3.08 (m, 8H), 3.41 (s, 3H), 3.67-3.75 (m, 8H), 5.82 (d, 1H), 6.11 (t, 1H), 6.93 (d, 2H), 7.31-7.37 (m, 2H), 7.47 (ddd, 1H), 7.97 (d, 1H), 8.90 (s, 1H); Mass spectrum: MH⁺ 483.

### EXAMPLE 15

The procedure described in example 14 was repeated using the appropriate aniline. Thus were obtained the compounds described below.

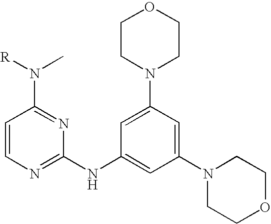

| | | | Molecular | |
|---|---|---|---|---|
| Example | Name | R | ion (MH⁺) | NMR Spectrum |
| 15.1^{a} | N′-(2,4-dichlorophenyl)-N-(3,5-dimorpholin-4-ylphenyl)-N′-methyl-pyrimidine-2,4-diamine | 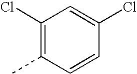 | 515 | 2.99-3.07 (m, 8 H),3.34 (s, 3 H), 3.66-3.77 (m, 8 H), 5.55(bs, 1 H), 6.09 (s,1 H), 6.93 (s, 2 H),7.50-7.58 (m, 2 H),7.78 (d, 1 H), 7.89(d, 1 H), 8.68 (s, 1 H) |
| 15.2 | 4-[[2-[(3,5-dimorpholin-4-ylphenyl)amino]pyrimidin-4-yl]-methyl-amino]-2-methoxy-benzonitrile | 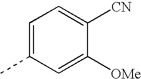 | 502 | 2.96-3.02 (m, 8 H),3.50 (s, 3 H), 3.67-3.72 (m, 8 H), 3.85(s, 3 H), 6.11 (t, 1 H),6.17 (d, 1 H), 6.89(d, 2 H), 7.08 (dd,1 H), 7.26 (d, 1 H),7.75 (d, 1 H), 8.03(d, 1 H), 8.97 (s, 1 H) |
| 15.3 | N′-(3,4-dimethoxyphenyl)-N-(3,5-dimorpholin-4-ylphenyl)-N′-methyl-pyrimidine-2,4-diamine | 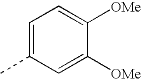 | 507 | 3.02-3.09 (m, 8 H),3.42 (s, 3 H), 3.70-3.74 (m, 8 H), 3.75(s, 3 H), 3.80 (s, 3 H),5.68 (d, 1 H), 6.11 (t,1 H), 6.86 (dd, 1 H),6.95 (d, 1 H), 7.00(d, 2 H), 7.03 (d,1 H), 7.81 (d, 1 H),8.82 (s, 1 H) |
| 15.4 | N′-(2,5-dimethoxyphenyl)-N-(3,5-dimorpholin-4-ylphenyl)-N′-methyl-pyrimidine-2,4-diamine | 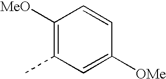 | 507 | 3.01-3.012 (m, 8 H),3.34 (s partiallyhidden by H2O,3 H), 3.69-3.75 (m,14 H), 5.48 (bs, 1 H),6.11 (t, 1 H), 6.91 (d,1 H), 6.96 (dd, 1 H),7.02 (bs, 2 H), 7.12(d, 1 H), 7.79 (d,1 H), 8.80 (bs, 1 H) |
| 15.5 | N′-(3,5-dimethoxyphenyl)-N-(3,5-dimorpholin-4-ylphenyl)-N′-methyl-pyrimidine-2,4-diamine | 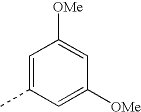 | 507 | 3.02-3.08 (m, 8 H),3.43 (s, 3 H), 3.70-3.75 (m, 8 H), 3.76(s, 6 H), 5.82 (d, 1 H),6.11 (t, 1 H), 6.48 (t,1 H), 6.52 (d, 2 H),6.99 (d, 2 H), 7.85(d, 1 H), 8.85 (s, 1 H) |
| 15.6 | 2-chloro-6-[[2-[(3,5-dimorpholin-4-ylphenyl)amino]pyrimidin-4-yl]-methyl-amino]benzonitrile | 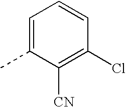 | 506 | 2.99-3.05 (m, 8 H),3.45 (s, 3 H), 3.68-3.75 (m, 8 H), 5.87(d, 1 H), 6.11 (t, 1 H),6.90 (d, 2 H), 7.62(d, 1 H), 7.75 (d,1 H), 7.85 (dd, 1 H),8.02 (d, 1 H), 8.93(bs, 1 H) |
| 15.7 | N′-(3,4-difluorophenyl)-N-(3,5-dimorpholin-4-ylphenyl)-N′-methyl-pyrimidine-2,4-diamine | 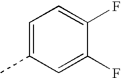 | 483 | 3.00-3.06 (m, 8 H),3.42 (s, 3 H), 3.69-3.73 (m, 8 H), 5.84(d, 1 H), 6.11 (t, 1 H),6.95 (d, 2 H), 7.22-7.27 (m, 1 H), 7.53(dd, 1 H), 7.57 (ddd,1 H), 7.91 (d, 1 H),8.8 (s, 1 H) |
| 15.8 | N′-(2,5-difluorophenyl)-N-(3,5-dimorpholin-4-ylphenyl)-N′-methyl-pyrimidine-2,4-diamine | 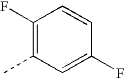 | 483 | 2.99-3.07 (m, 8 H),3.40 (s, 3 H), 3.69-3.75 (m, 8 H), 5.79(d, 1 H) 6.11 (t, 1 H)6.93 (d, 2 H), 7.26-7.33 (m, 1 H), 7.44(ddd, 1 H), 7.49(ddd, 1 H), 7.96 (d,1 H), 8.89 (s, 1 H) |
| 15.9 | N-(3,5-dimorpholin-4-ylphenyl)-N′-methyl-N′-(2,3,4-trifluorophenyl)pyrimidine-2,4-diamine | 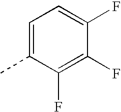 | 501 | 2.97-3.08 (m, 8 H),3.39 (s, 3 H), 3.68-3.75 (m, 8 H), 5.87(bs, 1 H), 6.11 (t,1 H), 6.91 (s, 2 H),7.38-7.50 (m, 2 H),7.97 (d, 1 H), 8.89(bs, 1 H) |
| 15.10 | N-(3,5-dimorpholin-4-ylphenyl)-N′-(3-methoxy-4-methyl-phenyl)-N′-methyl-pyrimidine-2,4-diamine | 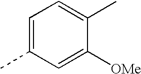 | 491 | 2.18 (s, 3 H), 3.01-3.09 (m, 8 H), 3.44(s, 3 H), 3.68-3.76(m, 8 H), 3.78 (s,3 H), 5.75 (s, 1 H),6.11 (t, 1 H), 6.81(dd, 1 H), 6.93 (d,1 H), 7.00 (d, 2 H),7.22 (d, 1 H), 7.83(d, 1 H), 8.83 (s, 1 H) |
| 15.11 | N-(3,5-dimorpholin-4-ylphenyl)-N′-(4-methoxy-2-methyl-phenyl)-N′-methyl-pyrimidine-2,4-diamine | 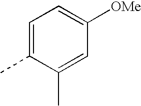 | 491 | 2.08 (s, 3 H), 2.99-3.17 (m, 8 H), 3.35 (spartially hidden byH2O, 3 H), 3.67-3.77(m, 8 H), 3.78 (s,3 H), 5.27 (bs, 1 H),6.11 (s, 1 H), 6.88 (d,1 H), 6.96 (s, 1 H),7.04 (s, 2 H), 7.16 (d,1 H), 7.77 (bs, 1 H),8.83 (bs, 1 H) |
| 15.12 | N-(3,5-dimorpholin-4-ylphenyl)-N′-(2-methoxy-4-methyl-phenyl)-N′-methyl-pyrimidine-2,4-diamine | 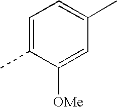 | 491 | 2.17 (s, 3 H), 2.83-2.91 (m, 8 H), 3.31(s, partially hiddenby H2O, 3 H), 3.49-3.58 (m, 8 H), 3.56(s, 3 H), 5.20 (bs,1 H), 5.87 (bs, 1 H),5.91 (s, 1 H), 6.66 (d,1 H), 6.82 (s, 2 H),6.95 (d, 1 H), 7.56(d, 1 H), 8.59 (bs,1 H) |
| 15.13 | N-(3,5-dimorpholin-4-ylphenyl)-N′-(2-methoxy-5-methyl-phenyl)-N′-methyl-pyrimidine-2,4-diamine | 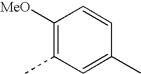 | 491 | 2.27 (s, 3 H), 3.02-3.14 (m, 8 H), 3.69-3.77 (m, 11 H), 5.42(bs, 1 H), 6.11 (s,1 H), 7.02 (s, 2 H),7.08(d, 1 H), 7.11(d, 1 H), 7.19 (dd,1 H), 7.77 (d, 1 H),8.79 (bs, 1 H) |
| 15.14 | N′-(3-chloro-4-methoxy-phenyl)-N-(3,5-dimorpholin-4-ylphenyl)-N′-methyl-pyrimidine-2,4-diamine | 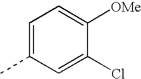 | 511 | 2.99-3.09 (m, 8 H),3.40 (s, 3 H), 3.68-3.75 (m, 8 H), 3.90(s, 3 H), 5.70 (d, 1 H),6.11 (t, 1 H), 6.98 (d,2 H), 7.24 (d, 1 H),7.32 (dd, 1 H), 7.49(d, 1 H), 7.86 (d,1 H), 8.85 (s, 1H) |
| 15.15 | N′-(3-chloro-2-fluoro-phenyl)-N-(3,5-dimorpholin-4-ylphenyl)-N′-methyl-pyrimidine-2,4-diamine | 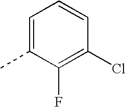 | 499 | 2.99-3.07 (m, 8 H),3.40 (s, 3 H), 3.68-3.76 (m, 8 H), 5.77(d, 1 H), 6.11 (t, 1 H),6.93 (s, 2 H), 7.35(ddd, 1 H), 7.51(ddd, 1 H), 7.62(ddd, 1 H), 7.96 (d,1 H), 8.90 (s, 1 H) |
| 15.16 | N′-(4-chloro-3-fluoro-phenyl)-N-(3,5-dimorpholin-4-ylphenyl)-N′-methyl-pyrimidine-2,4-diamine | 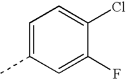 | 499 | 2.98-3.05 (m, 8 H),3.44 (s, 3 H), 3.67-3.75 (m, 8 H), 5.98(d, 1 H), 6.11 (t, 1 H),6.92 (d, 2 H), 7.27(dd, 1 H), 7.54 (dd,1 H), 7.65 (dd, 1 H),7.96 (d, 1 H), 8.91 (s,1 H) |
| 15.17 | N′-(4-chloro-2-fluoro-phenyl)-N-(3,5-dimorpholin-4-ylphenyl)-N′-methyl-pyrimidine-2,4-diamine | 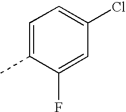 | 499 | 2.99-3.06 (m, 8H),3.38 (s, 3H), 3.68-3.75 (m, 8H), 5.78(bs, 1H), 6.11 (t,1H), 6.92 (s, 2H),7.41 (dd, 1H), 7.55(dd, 1H), 7.63 (dd,1H), 7.95 (d, 1H),8.88 (s, 1H) |
| 15.18 | N′-(2-chloro-5-methyl-phenyl)-N-(3,5-dimorpholin-4-ylphenyl)-N′-methyl-pyrimidine-2,4-diamine | 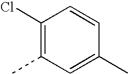 | 495 | 2.35 (s, 3 H), 3.04-3.11 (m, 8 H), 3.37(s, 3 H), 3.70-3.77(m, 8 H), 5.44 (bs,1 H), 6.11 (t, 1 H),6.99 (s, 2 H), 7.27(dd, 1 H), 7.33 (d,1 H), 7.52 (d, 1 H),7.86 (d, 1 H), 8.69(bs, 1 H) |
| 15.19 | N′-(3-chloro-4-methyl-phenyl)-N-(3,5-dimorpholin-4-ylphenyl)-N′-methyl-pyrimidine-2,4-diamine | 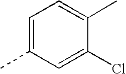 | 495 | 2.37 (s, 3 H), 3.00-3.06 (m, 8 H), 3.42(s, 3 H), 3.69-3.74(m, 8 H), 5.79 (d,1 H), 6.11 (t, 1 H),6.96 (d, 2 H), 7.25(dd, 1 H), 7.44 (d,1 H), 7.46 (d, 1 H),7.89 (d, 1 H), 8.87 (s,1 H) |
| 15.20 | N′-(2-chloro-6-methyl-phenyl)-N-(3,5-dimorpholin-4-ylphenyl)-N′-methyl-pyrimidine-2,4-diamine | 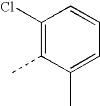 | 495 | 2.18 (s, 3 H), 3.04-3.12 (m, 8 H), 3.35 (spartially hidden byH2H, 3 H), 3.71-3.76(m, 8 H), 5.21 (d,1 H), 6.13 (t, 1 H),7.04 (d, 2H), 7.35-7.43 (m, 2H), 7.51(dd, 1 H), 7.83 (d,1 H), 8.93 (s, 1 H) |
| 15.21 | N′-(2,3-dichlorophenyl)-N-(3,5-dimorpholin-4-ylphenyl)-N′-methyl-pyrimidine-2,4-diamine | 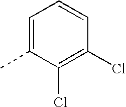 | 515 | 2.98-3.11 (m, 8 H),3.38 (s, 3 H), 3.68-3.76 (m, 8 H), 5.41(bs, 1 H), 6.12 (s,1 H), 6.93-7.03 (m,2 H), 7.48-7.57 (m,2 H), 7.73 (d, 1 H),7.89 (bs, 1 H), 8.91(bs, 1 H) |
| 15.22 | N′-(4-fluoro-3-chloro-phenyl)-N-(3,5-dimorpholin-4-ylphenyl)-N′-methyl-pyrimidine-2,4-diamine | 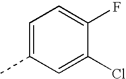 | 499 | DMSOd6: 2.99-3.08(m, 8 H), 3.42 (s,3 H), 3.68-3.75 (m,8 H), 5.83 (d, 1 H),6.12 (s, 1 H), 6.93 (s,2H), 7.41 (ddd, 1 H),7.52 (dd, 1 H), 6.69(dd, 1 H), 7.91 (d,1 H), 8.93 (bs, 1 H) |

^{a}The reaction was run on 200 mg scale. The deprotection step with TFA was run at 130° C. for 15 min.

### EXAMPLE 16

### [3-[[2-[(3-ethoxy-5-morpholin-4-yl-phenyl)amino]pyrimidin-4-yl]-methyl-amino]-4-methyl-phenyl]methanol

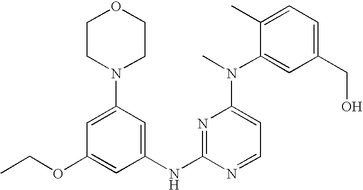

A mixture of triphenyl phosphine (414 mg, 1.58 mmol) and DEAD (0.246 ml, 1.58 mmol) in THF (3 ml) was stirred at room temperature for 1 hr. The mixture was then cooled at 0° C. and ethanol (92 μl, 1.58 mmol) was added. After stirring at room temperature for 30 minutes, [3-[[2-[acetyl-(3-hydroxy-5-morpholin-4-yl-phenyl)amino]pyrimidin-4-yl]-methyl-amino]-4-methyl-phenyl]methyl acetate (200 mg) was added and the mixture was stirred at room temperature for 2 hrs. Water was added to the mixture and THF was evaporated under vacuum. The resulting mixture was extracted with DCM. The organic layer was evaporated under vacuum and purified by chromatography on silica gel (eluant: 0 to 100% EtOAc in DCM) to give a white foam (144 mg). This solid was dissolved in methanol (4 ml)-water (4 ml) and sodium hydroxide (108 mg, 2.7 mmol) was added. The mixture was stirred at room temperature for 24 hrs. After evaporation of the solvents under vacuum, the mixture was diluted with aqueous sodium bicarbonate and extracted with DCM. The organic layer was evaporated under vacuum and purified by chromatography on silica gel (eluant: 0 to 100% EtOAc in DCM) to give the title compound as a white foam (100 mg, 56%).

NMR Spectrum: (DMSOd₆) 1.30 (t, 3H), 2.08 (s, 3H), 3.06 (bs, 4H), 3.37 (s, 3H), 3.67-3.77 (m, 4H), 3.77 (bs, 2H), 4.49 (d, 2H), 5.22 (t, 1H), 5.27 (bs, 1H), 6.06 (s, 1H), 7.02 (s, 2H), 7.18 (s, 1H), 7.26 (d, 1H), 7.34 (d, 1H), 7.79 (d, 1H), 8.96 (s, 1H); Mass spectrum: 450 MH⁺

The [3-[[2-[acetyl-(3-hydroxy-5-morpholin-4-yl-phenyl)amino]pyrimidin-4-yl]-methyl-amino]-4-methyl-phenyl]methyl acetate was made as follows:

A mixture of 4-(3-methoxy-5-nitro-phenyl)morpholine 5.52 g, 21.8 mmol, Method 8) in 48% aqueous hydrobromic acid (91 ml) was heated at 130° C. for 12 hrs. After cooling, aqueous ammonia was added slowly while cooling the mixture at 0° C. The mixture was concentrated to dryness. The residue was triturated in EtOAc, dried over magnesium sulfate and filtered. Evaporation of the filtrate and chromatography on silica gel (eluant: 0% to 30% EtOAc in DCM) gave 3-morpholin-4-yl-5-nitro-phenol (3.76 g, 77%) as a yellow solid. NMR Spectrum: (DMSOd₆) 3.17 (m, 4H), 3.72 (m, 4H), 6.71 (s, 1H), 7.01 (s, 1H), 7.19 (s, 1H); Mass spectrum: MH⁺ 225.

A mixture of 3-morpholin-4-yl-5-nitro-phenol (3.76 g, 16.8 mmol), benzyl bromide (2.19 ml, 18.4 mmol) and cesium carbonate (6.55 g, 20.1 mmol) in DMF (40 ml) was heated at 90° C. for 2 hrs. After cooling, the mixture was diluted with water. The formed precipitate was filtered, washed with water and dried to give 4-(3-nitro-5-phenylmethoxy-phenyl)morpholine (5.32 g, quantitative) as a yellow solid. NMR Spectrum: (DMSOd₆) 3.23 (m, 4H), 3.73 (m, 4H), 5.21 (s, 2H), 6.99 (s, 1H), 7.24 (s, 1H), 7.48-7.33 (m, 6H); Mass spectrum: MH⁺ 315

A mixture of 4-(3-nitro-5-phenylmethoxy-phenyl)morpholine (5 g, 15.9 mmol), platinum(IV) oxide (500 mg), potassium carbonate (830 mg) in EtOAc (70 ml)-ethanol (70 ml) was hydrogenated at room temperature under 50 PSI for 3 hrs. After filtration of the solids, evaporation of the solvents gave 3-morpholin-4-yl-5-phenylmethoxy-aniline (4.49 g, 99%) as a light brown gum. NMR Spectrum: (DMSOd₆) 2.98 (m, 4H), 3.68 (m, 4H), 4.96-4.90 (m, 4H), 5.76 (s, 2H), 5.80 (s, 1H), 7.42-7.28 (m, 5H); Mass spectrum: MH⁺ 285.

3-Morpholin-4-yl-5-phenylmethoxy-aniline (4.12 g, 14.5 mmol) and [3-[(2-chloropyrimidin-4-yl)-methyl-amino]-4-methyl-phenyl]methanol (3.43 g, 13 mmol, Method 2) were reacted according to procedure in Example 1 to give [4-methyl-3-[methyl-[2-[(3-morpholin-4-yl-5-phenylmethoxy-phenyl)amino]pyrimidin-4-yl]amino]phenyl]methanol (5.96 g, 80%) as a white foam. Mass spectrum: MH⁺ 512 [4-Methyl-3-[methyl-[2-[(3-morpholin-4-yl-5-phenylmethoxy-phenyl)amino]pyrimidin-4-yl]amino]phenyl]methanol (2.15 g, 4.20 mmol) and pyridine (0.75 ml, 9.25 mmol) in acetic anhydride (9.32 ml) was heated at 70° C. for 2 hrs. After evaporation of the solvents, saturated aqueous sodium bicarbonate was added. The mixture was extracted with EtOAc. After evaporation of the organic layer, the residue was purified by chromatography on silica gel (eluant: EtOAc) to give [3-[[2-[acetyl-(3-morpholin-4-yl-5-phenylmethoxy-phenyl)amino]pyrimidin-4-yl]-methyl-amino]-4-methyl-phenyl]methyl acetate (2.23 g, 89%). Mass spectrum: MH⁺ 596.

A mixture of [3-[[2-[acetyl-(3-morpholin-4-yl-5-phenylmethoxy-phenyl)amino]pyrimidin-4-yl]-methyl-amino]-4-methyl-phenyl]methyl acetate (1.1 g, 1.85 mmol) in ethanol (15 ml)-EtOAc (15 ml)-DMF (8 drops) was hydrogenated at room temperature under 50 PSI for 3 hrs in the presence of 10% palladium on charcoal (400 mg). After filtration of the solids, the mixture was concentrated to dryness and purified by chromatography on silica gel (eluant: 0 to 3% methanol in EtOAc) to give [3-[[2-[acetyl-(3-hydroxy-5-morpholin-4-yl-phenyl)amino]pyrimidin-4-yl]-methyl-amino]-4-methyl-phenyl]methyl acetate (636 mg, 68%) as a white foam. NMR Spectrum: (DMSOd₆) 2.06 (s, 3H), 2.07 (s, 3H), 2.24 (s, 3H), 2.97-3.08 (m, 4H), 3.29 (s, 3H), 3.64-3.75 (m, 4H), 5.06 (s, 2H), 5.67 (s, 1H), 6.12 (s, 1H), 6.25 (s, 1H), 6.29 (s, 1H), 7.30 (s, 1H), 7.34 (d, 1H), 7.42 (d, 1H), 7.99 (s, 1H), 9.33 (s, 1H); Mass spectrum: MH⁺ 506.

### EXAMPLE 17

According to the procedure described in Example 16, [3-[[2-[acetyl-(3-hydroxy-5-morpholin-4-yl-phenyl)amino]pyrimidin-4-yl]-methyl-amino]-4-methyl-phenyl]methyl acetate and the corresponding alcohol were reacted to made the following compounds, except that triphenylphosphine supported on polymer (3 mmol/g) was used, the first step was not purified and after the deprotection step, the compound was injected on an HPLC column (C18, 5 microns, 19 mm diameter, 100 mm length) of a preparative HPLC-MS system eluting with a mixture of water and acetonitrile containing 2 g/l of ammonium carbonate (gradient).

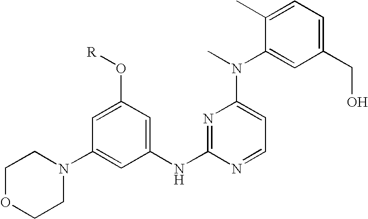

| | | | Molecular | |
|---|---|---|---|---|
| Example | Name | R | ion (MH⁺) | NMR Spectrum |
| 17.1 | [4-methyl-3- | Pr | 464 | |
| | [methyl-[2-[(3 | | | 0.96 (t, 3 H), 1.75-1.76 (m, |
| | morpholin-4-yl-5- | | | 2 H), 2.08 (s, 3 H), 3.06 (bs, |
| | propoxy- | | | 4 H), 3.37 (s, 3 H), 3.68-3.75 |
| | phenyl)amino]py- | | | (m, 4 H), 3.87 (bs, 2 H), 4.49 |
| | rimidin-4- | | | (d, 2 H), 5.22 (t, 1 H), 5.27 |
| | yl]amino] phenyl] | | | (bs, 1 H), 6.07 (s, 1 H), 7.06 |
| | methanol | | | (s, 1 H), 7.10 (s, 1 H), 7.18 (s, |
| | | | | 1 H), 7.26 (d, 1 H), 7.34 (d, |
| | | | | 1 H), 7.79 (s, 1 H), 8.96 (s, |
| | | | | 1 H) |
| 17.2 | [4-methyl-3- | iPr | 464 | 1.25 (d, 6 H), 2.08 (s, 3 H), |
| | [methyl-[2-[(3 | | | 3.05 (bs, 4 H), 3.37 (s, 3 H), |
| | morpholin-4-yl-5- | | | 3.67-3.75 (m, 4 H), 4.49 (d, |
| | propan-2-yloxy- | | | 2 H), 4.52 (bs, 1 H), 5.22 (t, |
| | phenyl)amino]py- | | | 1 H), 5.27 (bs, 1 H), 6.04 (s, |
| | rimidin-4- | | | 1 H), 7.02 (bs, 1 H), 7.09 (bs, |
| | yl]amino] phenyl] | | | 1 H), 7.18 (s, 1 H), 7.26 (d, |
| | methanol | | | 1 H), 7.34 (d, 1 H), 7.79 (bs, |
| | | | | 1 H), 8.94 (bs, 1 H) |
| 17.3 | [4-methyl-3- | iBu | 478 | 0.97 (s, 6 H), 1.95-2.05 (m, |
| | [methyl-[2-[[3-(2- | | | 1 H), 2.09 (s, 3 H), 3.07 (bs, |
| | methylpropoxy)- | | | 4 H), 3.38 (s partially hidden |
| | 5-morpholin-4-yl- | | | by H2O, 3 H), 3.66-3.77 (m, |
| | phenyl]amino]py- | | | 6 H), 4.50 (d, 2 H), 5.23 (t, |
| | rimidin-4- | | | 1 H), 5.28 (bs, 1 H), 6.08 (s, |
| | yl]amino] phenyl] | | | 1 H), 7.03 (bs, 1 H), 7.15 (bs, |
| | methanol | | | 1 H), 7.19 (s, 1 H), 7.27 (d, |
| | | | | 1 H), 7.35 (d, 1 H), 7.80 (bs, |
| | | | | 1 H), 8.96 (bs, 1 H) |
| 17.4 | [3-[[2-[[3- | CH₂cPr | 476 | 0.23-0.36 (m, 2 H), 0.50-0.62 |
| | (cyclopropylmeth- | | | (m, 2 H), 1.15-1.27 (m, 1 H), |
| | oxy)-5- | | | 2.02 (s, 3 H), 3.07 (bs, 4 H), |
| | morpholin-4-yl- | | | 3.38 (s partially hidden by |
| | phenyl[amino[py- | | | H2O, 3 H), 3.72 (bs, 4 H), |
| | rimidin-4-yl[- | | | 3.77 (d, 2 H), 4.50 (d, 2 H), |
| | methyl-amino[-4 | | | 5.23 (t, 1 H), 5.28 (bs, 1 H), |
| | methyl-phenyl[ | | | 6.08 (s, 1 H), 7.06 (bs, 1 H), |
| | methanol | | | 7.09 (bs, 1 H), 7.19 (s, 1 H), |
| | | | | 7.27 (d, 1 H), 7.35 (d, 1 H), |
| | | | | 7.80 (bs, 1 H), 8.96 (bs, 1 H) |
| 17.5 | [3-[[2-[(3- | cBu | 476 | (DMSOd6 at 323° K.) 1.59- |
| | cyclobutyloxy-5- | | | 1.70 (m, 1 H), 1.75-1.84 (m, |
| | morpholin-4-yl- | | | 1 H), 1.98-2.12 (m, 2 H), 2.10 |
| | phenyl)amino]py- | | | (s, 3 H), 2.39-2.48 (m, 2 H), |
| | rimidin-4-yl]- | | | 3.03-3.12 (m, 4 H), 3.38 (s, |
| | methyl-amino]-4 | | | 3 H), 3.70-3.77 (m, 4 H), 4.51 |
| | methyl-phenyl] | | | (d, 2 H), 4.57-4.67 (m, 1 H), |
| | methanol | | | 5.09 (bs, 1 H), 5.36 (bs, 1 H), |
| | | | | 5.78 (s, 1 H), 6.89 (s, 1 H), |
| | | | | 7.10 (bs, 1 H), 7.19 (s, 1 H), |
| | | | | 7.27 (d, 1 H), 7.34 (d, 1 H), |
| | | | | 7.82 (d, 1 H), 8.76 (bs, 1 H) |
| 17.6 | [3-[[2-[[3-(3- | —CH₂CH₂CH— | 508 | (DMSOd6 at 323° K.) 1.15 |
| | methoxybutoxy)- | (Me)OMe | | (d, 3 H), 1.77-1.92 (m, 2 H), |
| | 5-morpholin-4-yl- | | | 2.10 (s, 3 H), 3.05-3.11 (m, |
| | phenyl]amino]py- | | | 4 H), 3.24 (s, 3 H), 3.38 (s, |
| | rimidin-4-yl]- | | | 3H), 3.47-3.54 (m, 1 H), 3.71- |
| | methyl-amino]-4 | | | 3.76 (m, 4 H), 3.95-4.04 (m, |
| | methyl-phenyl] | | | 2 H), 4.51 (s, 2 H), 5.09 (bs, |
| | methanol | | | 1 H), 5.36 (bs, 1 H), 6.08 (dd, |
| | | | | 1 H), 7.02 (bs, 1 H), 7.09 (bs, |
| | | | | 1 H), 7.19 (s, 1 H), 7.27 (d, |
| | | | | 1 H), 7.34 (d, 1 H), 7.81 (d, |
| | | | | 1 H), 8.77 (bs, 1 H) |
| 17.7 | [3-[[2-[[3-(2- | —CH₂CH₂OEt | 494 | (DMSOd6 at 323° K.) 1.15 (t, |
| | ethoxyethoxy)-5- | | | 3 H), 2.10 (s, 3 H), 3.04-3.13 |
| | morpholin-4-yl- | | | (m, 4 H), 3.38 (s, 3 H), 3.52 |
| | phenyl]amino]py- | | | (q, 2 H), 3.69 (t, 2 H), 3.71- |
| | rimidin-4-yl]- | | | 3.77 (m, 4 H), 3.99-4.09 (m, |
| | methyl-amino]-4 | | | 2 H), 4.51 (s, 2 H), 5.10 (bs, |
| | methyl-phenyl] | | | 1 H), 5.36 (bs, 1 H), 6.10 (s, |
| | methanol | | | 1 H), 7.05 (s, 2 H), 7.19 (s, |
| | | | | 1 H), 7.27 (d, 1 H), 7.34 (d, |
| | | | | 1 H), 7.81 (d, 1 H), 8.78 (bs, |
| | | | | 1 H) |
| 17.8 | [4-methyl-3-[methyl-[2-[[3-morpholin-4-yl-5-(oxolan-3-yloxy)phenyl]ami-no]pyrimidin-4-yl]amino]phenyl]methanol | 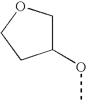 | 492 | (DMSOd6): 1.94-2.04 (m,1 H), 2.10 (s, 3 H), 2.15-2.25(m, 1 H), 3.05-3.12 (m, 4 H),3.37 (s, 3 H), 3.68-3.80 (m,6 H), 3.81-3.93 (m, 2 H),4.51 (s, 2 H), 4.94 (s, 1 H),5.10 (s, 1 H), 5.37 (s, 1 H),6.05 (s, 1 H), 6.96 (s, 1 H),7.12 (bs, 1 H), 7.19 (s, 1 H),7.26 (d, 1 H), 7.34 (d, 1 H),7.82 (d, 1 H), 8.78 (bs, 1 H) |
| 17.9 | [4-methyl-3-[methyl-[2-[[3-morpholin-4-yl-5-[(3S)-oxolan-3-yl]oxyphenyl]ami-no]pyrimidin-4-yl]amino]phenyl]methanol | 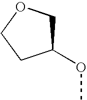 | 492 | (DMSOd6): 1.93-2.03 (m,1 H), 2.10 (s, 3 H), 2.14-2.24(m, 1 H), 3.04-3.12 (m, 4 H),3.38 (s, 3 H), 3.69-3.80 (m,6 H), 3.81-3.93 (m, 2 H), 4.51(d, 2 H), 4.96 (s, 1 H), 5.24 (t,1 H), 5.30 (s, 1 H), 6.07 (s,1 H), 6.97 (s, 1 H), 7.19 (bs,1 H), 7.20 (s, 1 H), 7.28 (d,1 H), 7.35 (d, 1 H), 7.81 (d,1 H), 8.99 (bs, 1 H) |
| 17.10 | [4-methyl-3-[methyl-[2-[[3-morpholin-4-yl-5-(oxan-4-yloxy)phenyl]ami-no]pyrimidin-4-yl]amino]phenyl]methanol | 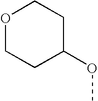 | 506 | DMSOd6): 1.53-1.64 (m,2 H), 1.91-2.02 (m, 2 H), 2.09(s, 3 H), 3.09 (bs, 4 H), 3.38(s, 3 H), 3.43-3.51 (m, 2 H),3.68-3.76 (m, 4 H), 3.81-3.90(m, 2 H), 4.47 (bs, 1 H), 4.50(d, 2 H), 5.23 (t, 1 H), 5.28(bs, 1 H), 6.12 (s, 1 H), 7.07(s, 1 H), 7.12 (s, 1 H), 7.19 (s,1 H), 7.27 (d, 1 H), 7.35 (d,H), 7.80 (bs, 1 H), 8.95 (bs,1 H), |

### EXAMPLE 18

A mixture of 3-[[4-[[5-(hydroxymethyl)-2-methyl-phenyl]-methyl-amino]pyrimidin-2-yl]amino]-5-morpholin-4-yl-benzoic acid (120 mg, 0.27 mmol), the corresponding amine (1.07 mmol), diisopropylethylamine (61 μl, 0.35 mmol) and TBTU (112 mg, 0.35 mmol) in DMF (1.5 ml) was stirred at 30° C. for 18 hrs. After evaporation of the solvents, the residue was directly injected on an HPLC column (C18, 5 microns, 21 mm diameter, 100 mm length) of a preparative HPLC-MS system eluting with a mixture of water and acetonitrile containing 2 g/l of ammonium carbonate (gradient) to give the corresponding amide after evaporation of the solvents.

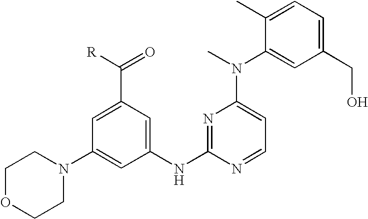

| | | | Molecular | |
|---|---|---|---|---|
| Example | Name | R | ion (MH⁺) | NMR Spectrum |
| 18.1 | [3-[[4-[[5-(hydroxymethyl)-2-methyl-phenyl]-methyl-amino]pyrimidin-2-yl]amino]-5-morpholin-4-yl-phenyl]-morpholin-4-yl-methanone | 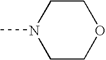 | 519 | (DMSO-d6 at 323° K.) 2.10(s, 3 H), 3.09-3.16 (m, 4 H),3.37 (s, 3 H), 3.50 (bs, 4 H),3.61 (bs, 4 H), 3.75 (t, 4 H),4.61 (d, 2 H), 5.09 (t, 1 H),5.39 (bs, 1 H), 6.49 (s, 1 H),7.19 (s, 1 H), 7.27 (d, 1 H),7.31-7.37 (m, 2 H), 7.72 (bs,1 H), 7.84 (d, 1 H), 8.97 (s,1 H) |
| 18.2 | [3-[[4-[[5-(hydroxymethyl)-2-methyl-amino]pyrimidin-2-yl]amino]-5-morpholin-4-yl-phenyl]-pyrrolidin-1-yl-methanone | 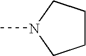 | 503 | (DMSO-d6 at 323° K.) 1.85(bs, 4 H), 2.10 (s, 3 H), 3.12(bs, 4 H), 3.36 (s, 3 H), 3.40(bs, 2 H), 3.45 (bs, 2 H),3.75 (t, 4 H), 4.51 (d, 2 H),5.09 (t, 1 H), 5.39 (bs, 1 H),6.57 (s, 1 H), 7.19 (s, 1 H),7.26 (d, 1 H), 7.34 (d, 1 H),7.40 (s, 1 H), 7.55 (bs, 1 H),7.83 (d, 1 H), 8.95 (s, 1 H) |
| 18.3 | 3-[[4-[[5- | NHiBu | 505 | (DMSO-d6 at 323° K.) 0.91 |
| | (hydroxymethyl)-2- | | | (d, 6 H), 1.81-1.91 (m, 1 H), |
| | methyl-phenyl]- | | | 2.10 (s, 3 H), 3.08 (t, 2 H), |
| | methyl- | | | 3.13-3.17 (m, 4 H), 3.38 (s, |
| | amino]pyrimidin-2- | | | 3 H), 3.77 (t, 4 H), 4.51 (d, |
| | yl]amino]-N-(2- | | | 2 H), 5.09 (t, 1 H), 5.36 (bs, |
| | methylpropyl)-5- | | | 1 H), 6.94 (s, 1 H), 7.18 (s, |
| | morpholin-4-yl- | | | 1 H), 7.27 (d, 1 H), 7.34 (d, |
| | benzamide | | | 1 H), 7.55 (s, 1 H), 7.82 (d, |
| | | | | 1 H), 8.11 (t, 1 H), 8.93 (s, |
| | | | | 1 H) |
| 18.4 | N-ethyl-3-[[4-[[5- | N(Me)Et | 491 | (DMSO-d6 at 323° K.) 1.11 |
| | (hydroxymethyl)-2- | | | (t, 3 H), 2.10 (s, 3 H), 2.92 |
| | methyl-phenyl]- | | | (s, 3 H), 3.11 (bs, 4 H), 3.29 |
| | methyl- | | | (bs, 2 H), 3.36 (s, 3 H), 3.71- |
| | amino]pyrimidin-2- | | | 3.80 (m, 4 H), 4.51 (d, 2 H), |
| | yl]amino]-N- | | | 5.09 (t, 1 H), 5.38 (bs, 1 H), |
| | methyl-5- | | | 6.44 (s, 1 H), 7.18 (s, 1 H), |
| | morpholin-4-yl- | | | 7.26 (d, 1 H), 7.31 (bs, 1 H), |
| | benzamide | | | 7.33 (d, 1 H), 7.49 (s, 1 H), |
| | | | | 7.84 (d, 1 H), 8.97 (s, 1 H) |
| 18.5 | N- | NHCH2cPr | 503 | (DMSO-d6 at 323° K.) |
| | (cyclopropylmethyl)- | | | 0.20-0.25 (m, 2 H), 0.39- |
| | 3-[[4-[[5- | | | 0.46 (m, 2 H), 0.96-1.07 (m, |
| | (hydroxymethyl)-2- | | | 1 H), 2.10 (s, 3 H), 3.11-3.16 |
| | methyl-phenyl]- | | | (m, 4 H), 3.38 (s, 3 H), 3.73- |
| | methyl- | | | 3.80 (m, 4 H), 4.50 (d, 2 H), |
| | amino]pyrimidin-2- | | | 5.08 (t, 1 H), 5.35 (bs, 1 H), |
| | yl]amino]-5- | | | 6.94 (s, 1 H), 7.17 (s, 1 H), |
| | morpholin-4-yl- | | | 7.25 (d, 1 H), 7.33 (d, 1 H), |
| | benzamide | | | 7.52 (s, 1 H), 7.80 (d, 1 H), |
| | | | | 7.84 (s, 1 H), 8.20 (bs, 1 H), |
| | | | | 8.93 (s, 1 H) |
| 18.6 | 3-[[4-[[5- | N(Me)iPr | 505 | (DMSO-d6 at 323° K.) 1.13 |
| | (hydroxymethyl)-2- | | | (d, 6 H), 2.10 (s, 3 H), 2.79 |
| | methyl-phenyl]- | | | (s, 3 H), 3.08-3.16 (m, 4 H), |
| | methyl- | | | 3.36 (s, 3 H), 3.72-3.79 (m, |
| | amino]pyrimidin-2- | | | 4 H), 3.99 (bs, 1 H), 4.51 (d, |
| | yl]amino]-N- | | | 2 H), 5.09 (t, 1 H), 5.37 (bs, |
| | methyl-5- | | | 1 H), 6.42 (s, 1 H), 7.19 (s, |
| | morpholin-4-yl-N- | | | 1 H), 7.26 (d, 1 H), 7.30 (bs, |
| | propan-2-yl- | | | 1 H), 7.34 (d, 1 H), 7.50 (bs, |
| | benzamide | | | 1 H), 7.83 (d, 1 H), 8.97 (s, |
| | | | | 1 H) |
| 18.7 | 3-[[4-[[5-(hydroxymethyl)-2-methyl-phenyl]-methyl-amino]pyrimidin-2-yl]amino]-N-(2-methoxyethyl)-N-methyl-5-morpholin-4-yl-benzamide | 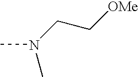 | 521 | (DMSO-d6 at 323° K) 2.10(s, 3 H), 2.97 (s, 3 H), 3.08-3.16 (m, 4 H), 3.26 (bs, 3 H),3.36 (s, 3 H), 3.51 (bs, 4 H),3.72-3.79 (m, 4 H), 4.51 (d,2 H), 5.09 (t, 1 H), 5.38 (bs,1 H), 6.46 (s, 1 H), 7.18 (s,1 H), 7.27 (d, 1 H), 7.29-7.38 (m, 2 H), 7.49 (bs, 1 H),7.83 (d, 1 H), 8.96 (s, 1 H) |

3-[[4-[[5-(Hydroxymethyl)-2-methyl-phenyl]-methyl-amino]pyrimidin-2-yl]amino]-5-morpholin-4-yl-benzoic acid used as starting material was made as follows:

A mixture of ethyl 3-fluoro-5-nitrobenzoate (7 g, 32.8 mmol) and morpholine (5.7 ml, 66 mmol) in DMSO (5 ml) was heated at 120° C. for 6 hrs. After cooling, the mixture was diluted with water. The formed precipitate was filtered, washed with water and dried. Further purification by chromatography on silica gel (eluant: DCM) gave ethyl 3-morpholin-4-yl-5-nitro-benzoate (6.73 g, 73%) as a yellow solid: NMR Spectrum: (DMSOd₆) 1.35 (t, 3H), 3.32 (m, 4H), 3.76 (m, 4H), 4.36 (q, 2H), 7.81 (s, 1H), 7.90 (s, 1H), 8.01 (s, 1H); Mass spectrum: MH⁺ 281.

Hydrogenation of ethyl 3-morpholin-4-yl-5-nitro-benzoate using Method 9, except that platinum(IV) oxide was used gave ethyl 3-amino-5-morpholin-4-yl-benzoate (6 g, 100%) as a white solid. NMR Spectrum: (DMSOd₆) 1.28 (t, 3H), 3.03 (m, 4H), 3.71 (m, 4H), 4.23 (q, 2H), 5.21 (s, 2H), 6.38 (s, 1H), 6.70 (s, 2H); Mass spectrum: MH⁺ 251. Ethyl 3-amino-5-morpholin-4-yl-benzoate (3 g, 12 mmol) and [3-[(2-chloropyrimidin-4-yl)-methyl-amino]-4-methyl-phenyl]methanol (2.64 g, 10 mmol) were reacted according to Example 1 to give ethyl 3-[[4-[[5-(hydroxymethyl)-2-methyl-phenyl]-methyl-amino]pyrimidin-2-yl]amino]-5-morpholin-4-yl-benzoate (4.43 g, 93%) as a white foam, except that the reaction was heated at 85° C. for 18 hrs. Mass spectrum: MH⁺ 478.

A mixture of sodium hydroxide (3.18 g, 79.6 mmol) and ethyl 3-[[4-[[5-(hydroxymethyl)-2-methyl-phenyl]-methyl-amino]pyrimidin-2-yl]amino]-5-morpholin-4-yl-benzoate (3.8 g, 7.96 mmol) in methanol (40 ml)—water (20 ml) was stirred at room temperature for 2 hrs. After evaporation of the solvents, 2N hydrochloric acid was added to adjust the pH to 3. The formed precipitate was filtered and dried to give 3-[[4-[[5-(hydroxymethyl)-2-methyl-phenyl]-methyl-amino]pyrimidin-2-yl]amino]-5-morpholin-4-yl-benzoic acid (3.21 g, 90%) as a solid. NMR Spectrum: (DMSOd₆) 2.09 (s, 3H), 3.06-3.19 (m, 4H), 3.39 (s, 1H), 3.70-3.81 (m, 4H), 4.50 (s, 2H), 5.23 (bs, 1H), 5.32 (bs, 1H), 7.08 (s, 1H), 7.19 (s, 1H), 7.27 (d, 1H), 7.35 (d, 1H), 7.66 (s, 1H), 7.79 (bs, 1H), 8.06 (s, 1H), 8.31 (bs, 1H); Mass spectrum: MH⁺ 450.

### EXAMPLE 19

3-[[4-[(3-Chlorophenyl)-methyl-amino]pyrimidin-2-yl]amino]-5-morpholin-4-yl-benzoic (100 mg, 0.23 mmol) was reacted according to the procedure in Example 18 to give the corresponding amides:

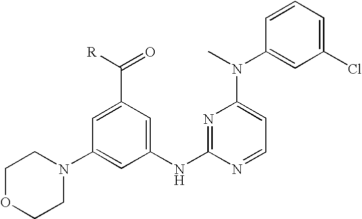

| | | | Molecular | |
|---|---|---|---|---|
| Example | Name | R | ion (MH⁺) | NMR Spectrum |
| 19.1 | 3-[[4-[(3- | NMe₂ | 467 | 2.89 (bs, 3 H), 2.95 (bs, |
| | chlorophenyl)-methyl- | | | 3 H), 3.03-3.09 (m, 4 H), |
| | amino]pyrimidin-2- | | | 3.42 (s, 3 H), 3.69-3.75 (m, |
| | yl]amino]-N,N- | | | 4 H), 5.90 (d, 1 H), 6.45 (s, |
| | dimethyl-5-morpholin- | | | 1 H), 7.25 (s, 1 H), 7.35 (d, |
| | 4-yl-benzamide | | | 1 H), 7.38 (d, 1 H), 7.43 (s, |
| | | | | 1 H), 7.45-7.51 (m, 2 H), |
| | | | | 7.95 (d, 1 H), 9.17 (s, 1 H) |
| 19.2 | 3-[[4-[(3-chlorophenyl)-methyl-amino]pyrimidin-2-yl]amino]-N-(2-hydroxyethyl)-N-methyl-5-morphohn-4-yl-benzamide | 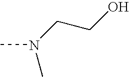 | 497 | (DMSOd6 at 323° K.) 2.97(s, 3 H), 3.06-3.11 (m, 4 H),3.38 (bs, 1 H), 3.44 (s, 3 H),3.56 (bs, 3 H), 3.72-3.77 (m,4 H), 4.64 (bs, 1 H), 5.90 (d,1 H), 6.48 (s, 1 H), 7.27 (bs,1 H), 7.34 (ddd, 1 H), 7.38(ddd, 1 H), 7.41 (s, 1 H),7.46 (dd, 1 H), 7.50 (dd,1 H), 7.95 (d, 1 H), 8.00 (s,1 H) |
| 19.3 | 3-[[4-[(3-chlorophenyl)-methyl-amino]pyrimidin-2-yl]amino]-N-(2-methoxyethyl)-N-methyl-5-morpholin-4-yl-benzamide | 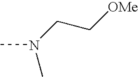 | 511 | (DMSOd6 at 323° K.) 2.96(s, 3 H), 3.06-3.11 (m, 4 H),3.25 (bs partially hidden byH2O, 3 H), 3.44 (s, 3 H),3.50 (bs, 4 H), 3.72-3.77 (m,4 H), 5.90 (d, 1 H), 6.46 (s,1 H), 7.28 (s, 1 H), 7.35(ddd, 1 H), 7.38 (ddd, 1 H),7.42 (s, 1 H), 7.46 (dd, 1 H),7.49 (dd, 1 H), 7.94 (d, 1 H),9.02 (s, 1 H) |
| 19.4 | 3-[[4-[(3-chlorophenyl)-methyl-amino]pyrimidin-2-yl]amino]-N-(3-hydroxypropyl)-N-methyl-5-morpholin-4-yl-benzamide | 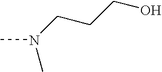 | 511 | (DMSOd6 at 323° K.) 1.65-1.79 (m, 2 H), 2.91 (s, 3 H),3.05-3.13 (m, 4 H), 3.40 (bs,4 H), 3.44 (s, 3 H), 3.71-3.78(m, 4 H), 4.34 (bs, 1 H), 5.89(d, 1 H), 6.44 (s, 1 H), 7.27(bs, 1 H), 7.35 (ddd, 1 H),7.39 (ddd, 1 H), 7.42 (s,1 H), 7.47 (dd, 1 H), 7.50(dd, 1 H), 7.94 (d, 1 H), 9.01(s, 1 H) |
| 19.5 | 3-[[4-[(3-chlorophenyl)-methyl-amino]pyrimidin-2-yl]amino]-N-[(2S)-2-hydroxypropyl]-N-methyl-5-morpholin-4-yl-benzamide | 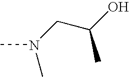 | 511 | 0.91 (d, 1.5 H), 1.10 (d,1.5 H), 2.95 (s, 1.5 H), 2.97(s, 1.5 H), 3.03-3.11 (m,4 H), 3.12-3.50 (m, 2 H),3.43 (s, 3 H), 3.70-3.77 (m,4 H), 3.84 (bs, 0.5 H), 3.95(bs, 0.5 H), 4.76 (bs, 0.5 H),4.80 (bs, 0.5 H), 5.88 (d,1 H), 6.47 (s, 0.5 H), 6.52 (s,0.5 H), 7.24 (bs, 0.5 H), 7.32(bs, 0.5 H), 7.36 (dd, 1 H),7.39 (dd, 1 H), 7.42 (bs,1 H), 7.47-7.43(m, 2 H),7.94 (d, 1 H), 9.17 (bs, 1 H) |
| 19.6 | 3-[[4-[(3-chlorophenyl)-methyl-amino]pyrimidin-2-yl]amino[-N-[(2S)1-hydroxypropan-2-yl]-N-methyl-5-morpholin-4-yl-benzamide | 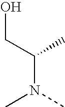 | 511 | (DMSOd6 at 323° K.) 1.06(bs, 3 H), 2.80 (s, 3 H), 3.05-3.11 (m, 4 H), 3.34 (bs, 1 H),3.44 (s, 3 H), 3.46 (bs, 1 H),3.70-3.78 (m, 4 H), 3.85 (ns,0.5 H), 4.57 (bs, 0.5 H), 4.64(t, 1 H), 5.88 (d, 1 H), 6.46(s, 1 H), 7.23 (bs, 1 H), 7.35(ddd, 1 H), 7.38 (dd, 1 H),7.44 (bs, 1 H), 7.47 (dd,1 H), 7.50 (dd, 1 H), 7.94 (d,1 H), 9.00 (bs, 1 H) |
| 19.7 | 3-[[4-[(3- | N(Me)Et | 481 | (DMSOd6 at 323° K.) 1.04- |
| | chlorophenyl)-methyl- | | | 1.18 (m, 3 H), 2.91 (s, 3 H), |
| | amino]pyrimidin-2- | | | 3.05-3.13 (m, 4 H), 3.32 (bs, |
| | yl]amino]-N-ethyl-N- | | | 2 H), 3.44 (s, 3 H), 3.69-3.80 |
| | methyl-5-morpholin-4- | | | (m, 4 H), 5.90 (d, 1 H), 6.43 |
| | yl-benzamide | | | (s, 1 H), 7.27 (bs, 1 H), 7.34 |
| | | | | (dd, 1 H), 7.38 (dd, 1 H), |
| | | | | 7.42 (s, 1 H), 7.47 (dd, 1 H), |
| | | | | 7.49 (dd, 1 H), 7.95 (d, 1 H), |
| | | | | 9.02 (bs, 1 H) |
| 19.8 | [3-[[4-[(3- | N(Me)iPr | 495 | (DMSOd6 at 323° K.) 1.12 |
| | chlorophenyl)-methyl- | | | (d, 6 H), 2.78 (s, 3 H), 3.04- |
| | amino]pyrimidin-2- | | | 3.13 (m, 4 H), 3.44 (s, 3 H), |
| | yl]amino]-N-methyl-5- | | | 3.70-3.78 (m, 4 H), 3.97 (bs, |
| | morpholin-4-yl-N- | | | 1 H), 5.89 (d, 1 H), 6.42 (s, |
| | propan-2-yl- | | | 1 H), 7.26 (bs, 1 H), 7.35 |
| | benzamide | | | (ddd, 1 H), 7.38 (ddd, 1 H), |
| | | | | 7.43 (s, 1 H), 7.46 (dd, 1 H), |
| | | | | 7.49 (dd, 1 H), 7.94 (d, 1 H), |
| | | | | 9.02 (s, 1 H) |
| 19.9 | 3-[[4-[(3-chlorophenyl)-methyl-amino]pyrimidin-2-yl]amino]-5-morpholin-4-yl-phenyl]-pyrrolidin-1-yl-methanone | 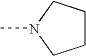 | 493 | (DMSOd6 at 323° K.) 1.73-1.93 (m, 4 H), 3.03-3.13 (m,4 H), 3.37 (bs, 2 H), 3.44 (s,3 H), 3.46 (bs, 2 H), 3.71-3.78 (m, 4 H), 5.91 (d, 1 H),6.57 (s, 1 H), 7.32-7.41 (m,3 H), 7.44-7.48 (m, 2 H),7.49 (dd, 1 H), 7.95 (d, 1 H),9.01 (bs, 1 H) |
| 19.10 | [3-[[4-[(3-chlorophenyl)-methyl-amino]pyrimidin-2-yl]amino]-5-morpholin-4-yl-phenyl]-morpholin-4-yl-methanone | 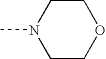 | 509 | (DMSOd6 at 323° K.) 3.05-3.03 (m, 4 H), 3.35 (s, 3 H),3.49 (bs, 4 H), 3.60 (bs,4 H), 3.70-3.80 (m, 4 H),5.90 (d, 1 H), 6.49 (s, 1 H),7.29 (s, 1 H), 7.35 (dd, 1 H),7.39 (dd, 1 H), 7.44-7.49(m, 2 H), 7.50 (dd, 1 H),7.95 (d, 1 H), 9.03 (bs, 1 H) |
| 19.11 | [3-[[4-[(3-chlorophenyl)-methyl-amino]pyrimidin-2-yl]amino]-5-morpholin-4-yl-phenyl]-(4-methylpiperazin-1-yl)methanone | 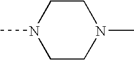 | 522 | DMSOd6 at 323° K.) 2.22(s, 3 H), 2.32 (bs, 4 H), 3.06-3.13 (m, 4 H), 3.45 (s, 3 H),3.48 (bs, 4 H), 3.7 1-3.78 (m,4 H), 5.90 (d, 1 H), 6.45 (s,1 H), 7.28 (s, 1 H), 7.35(ddd, 1 H), 7.38 (ddd, 1 H),7.45 (dd, 1 H), 7.47 (dd,1 H), 7.50 (dd, 1 H), 7.95 (d,1 H), 9.03 (bs, 1 H) |
| 19.12 | [3-[[4-[(3-chlorophenyl)-methyl-amino]pyrimidin-2-yl]amino]-5-morpholin-4-yl-phenyl]-(4-hydroxy-1-piperidyl) methanone | 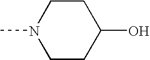 | 523 | 1.34 (bs, 2 H), 1.69 (bs,1 H), 1.78 (bs, 1 H), 3.01-3.11 (m, 4 H), 3.12 (bs, 2 H),3.44 (s, 3 H), 3.53 (bs, 1 H),3.69-3.78 (m, 5 H), 4.03 (bs,1 H), 4.79 (d, 1 H), 5.89 (d,1 H), 6.44 (s, 1 H), 7.26 (s,1 H), 7.36 (ddd, 1 H), 7.39(ddd, 1 H), 7.45 (dd, 1 H),7.49 (d, 1 H), 7.50 (dd, 1 H),7.94 (d, 1 H), 9.19 (s, 1 H) |
| 19.13 | 3-[[4-[(3- | NHcBu | 493 | (DMSOd6 at 323° K.) 1.63- |
| | chlorophenyl)-methyl- | | | 1.76 (m, 2 H), 2.02-2.14 (m, |
| | amino]pyrimidin-2- | | | 2 H), 2.19-2.28 (m, 2 H), |
| | yl]amino]-N- | | | 3.09-3.16 (m, 4 H), 3.47 (s, |
| | cyclobutyl-5- | | | 3 H), 3.72-3.8 1 (m, 4 H), |
| | morpholin-4-yl- | | | 4.36-4.47 (m, 1 H), 5.87 (d, |
| | benzamide | | | 1 H), 6.93 (s, 1 H), 7.35 |
| | | | | (ddd, 1 H), 7.39 (ddd, 1 H), |
| | | | | 7.46 (dd, 1 H), 7.48 (s, 1 H), |
| | | | | 7.50 (dd, 1 H), 7.77 (s, 1 H), |
| | | | | 7.73 (d, 1 H), 8.28 (d, 1 H), |
| | | | | 8.99 (bs, 1 H) |
| 19.14 | 3-[[4-[(3- | NHiPr | 481 | (DMSOd6 at 323° K.) 1.17 |
| | chlorophenyl)-methyl- | | | (d, 6 H), 3.09-3.15 (m, 4 H), |
| | amino]pyrimidin-2- | | | 3.47 (s, 3 H), 3.73-3.81 (m, |
| | yl]amino]-5- | | | 4 H), 4.04-4.15 (m, 1 H), |
| | morpholin-4-yl-N- | | | 5.86 (d, 1 H), 6.93 (s, 1 H), |
| | propan-2-yl- | | | 7.35 (ddd, 1 H), 7.39 (ddd, |
| | benzamide | | | 1 H), 7.47 (s, 1 H), 7.48 (s, |
| | | | | 1 H), 7.50 (dd, 1 H), 7.78 (s, |
| | | | | 1 H), 7.87 (d, 1 H), 7.93 (d, |
| | | | | 1 H), 8.98 (bs, 1 H) |
| 19.15 | 3-[[4-[(3-chlorophenyl)-methyl-amino]pyrimidin-2-yl]amino]-N-(1-methoxypropan-2-yl)-5-morpholin-4-yl-benzamide | 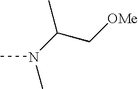 | 511 | (DMSOd6 at 323 0K) 1.15(d, 3 H), 3.09-3.15 (m, 4 H),3.29 (s, 3 H), 3.30 (dd, 1 H),3.42 (dd, 1 H), 3.47 (s, 3 H),3.74-3.80 (m, 4 H), 4.14-4.25 (m, 1 H), 5.86 (d, 1 H),6.94 (s, 1 H), 7.35 (ddd,1 H), 7.39 (ddd, 1 H), 7.46(dd, 1 H), 7.49 (dd, 1 H),7.50 (dd, 1 H), 7.78 (s, 1 H),7.86 (d, 1 H), 7.93 (d, 1 H),9.00 (bs, 1 H) |
| 19.16 | 3-[[4-[(3- | NHCH2cPr | 493 | (DMSOd6 at 323° K.) 0.22- |
| | chlorophenyl)-methyl- | | | 0.28 (m, 2 H), 0.42-0.48 (m, |
| | amino]pyrimidin-2- | | | 2 H), 1.01-1.10 (m, 1 H), |
| | yl]amino]-N- | | | 3.10-3.18 (m, 6 H), 3.48 (s, |
| | (cyclopropylmethyl)- | | | 3 H), 3.47-3.81 (m, 4 H), |
| | 5-morpholin-4-yl- | | | 5.88 (d, 1 H), 6.96 (s, 1 H), |
| | benzamide | | | 7.36 (ddd, 1 H), 7.40 (ddd, |
| | | | | 1 H), 7.47 (dd, 1 H), 7.49- |
| | | | | 7.54 (m, 2 H), 7.79 (s, 1 H), |
| | | | | 7.94 (d, 1 H), 8.21 (t, 1 H), |
| | | | | 9.01 (bs, 1 H) |
| 19.17 | 3-[[4-[(3- | NHiBu | 495 | (DMSOd6 at 323° K.) 0.91 |
| | chlorophenyl)-methyl- | | | (d, 6 H), 1.81-1.92 (m, 1 H), |
| | amino]pyrimidin-2- | | | 3.08 (dd, 2 H), 3.10-3.15 |
| | yl]amino]-N-(2- | | | (m, 4 H), 3.46 (s, 3 H), 3.72- |
| | methylpropyl)-5- | | | 3.80 (m, 4 H), 5.87 (d, 1 H), |
| | morphohn-4-yl- | | | 6.94 (s, 1 H), 7.35 (ddd, |
| | benzamide | | | 1 H), 7.39 (ddd, 1 H), 7.46 |
| | | | | (dd, 1 H), 7.50 (dd, 1 H), |
| | | | | 7.51 (dd, 1 H), 7.74 (s, 1 H), |
| | | | | 7.93 (d, 1 H), 8.11 (t, 1 H), |
| | | | | 9.00 (bs, 1 H) |
| 19.18 | 3-[[4-[(3- | NHCH₂CH₂OMe | 497 | (DMSOd6 at 323° K.) 3.09- |
| | chlorophenyl)-methyl- | | | 3.14 (m, 4 H), 3.29 (s, 3 H), |
| | amino]pyrimidin-2- | | | 3.39-3.45 (m, 2 H), 3.45- |
| | yl]amino]-N-(2- | | | 3.50 (m, 5 H), 3.74-3.80 (m, |
| | methoxyethyl)-5- | | | 4 H), 5.87 (d, 1 H), 6.96 (s, |
| | morpholin-4-yl- | | | 1 H), 7.35 (ddd, 1 H), 7.39 |
| | benzamide | | | (ddd, 1 H), 7.46 (dd, 1 H), |
| | | | | 7.48-7.53 (m, 2 H), 7.77 (bs, |
| | | | | 1 H), 7.93 (d, 1 H), 8.15 (t, |
| | | | | 1 H), 9.00 (s, 1 H) |
| 19.19 | 3-[[4-[(3-chlorophenyl)-methyl-amino]pyrimidin-2-yl]amino]-N-(1-hydroxy-2-methyl-propan-2-yl)-5-morpholin-4-yl-benzamide | 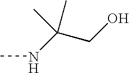 | 511 | (DMSOd6 at 323° K.) 1.33(s, 6 H), 3.08-3.14 (m, 4 H),3.47 (s, 3 H), 3.49 (d, 2 H),3.73-3.79 (m, 4 H), 4.88 (t,1 H), 5.87 (d, 1 H), 6.86 (s,1 H), 7.20 (bs, 1 H), 7.35(ddd, 1 H), 7.39 (ddd, 1 H),7.47 (dd, 1 H), 7.48-7.53(m, 2 H), 7.71 (s, 1 H), 7.93(d, 1 H), 8.89 (bs, 1H) |
| 19.20 | 3-[[4-[(3- | NHtBu | 495 | (DMSOd6 at 323° K.) 1.39 |
| | chlorophenyl)-methyl- | | | (s, 9 H), 3.08-3.14 (m, 4 H), |
| | amino]pyrimidin-2- | | | 4 H), 5.86 (d, 1 H), 6.87 (s, |
| | yl]amino]-5- | | | 1 H), 7.35 (ddd, 1 H), 7.38 |
| | morpholin-4-yl-N-tert- | | | (s, 1 H), 7.39 (ddd, 1 H), |
| | butyl-benzamide | | | 7.47 (dd, 1 H), 7.48 (dd, |
| | | | | 1 H), 7.50 (dd, 1 H), 7.90 (s, |
| | | | | 1 H), 7.93 (d, 1 H), 8.97 (bs, |
| | | | | 1 H) |
| 19.21 | 3-[[4-[(3-chlorophenyl)-methyl-amino]pyrimidin-2-yl]amino]-N-[(2S)-1-hydroxypropan-2-yl]-5-morpholin-4-yl-benzamide | 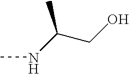 | 497 | (DMSOd6 at 323° K.) 1.15(d, 3 H), 3.10-3.15 (m, 4 H),3.33-3.40 (m, 1 H), 3.44-3.51 (m, 1 H), 3.47 (s, 3 H),3.74-3.80 (m, 4 H), 3.97-4.06 (m, 1 H), 4.59 (t, 1 H),5.86 (d, 1 H), 6.94 (s, 1 H),7.35 (ddd, 1 H), 7.39 (ddd,1 H), 7.47 (dd, 1 H), 7.49(dd, 1 H), 7.50 (dd, 1 H),7.73 (d, 1 H), 7.79 (s, 1 H),7.93 (d, 1 H), 8.99 (bs, 1 H) |
| 19.22 | [3-[[4-[(3-chlorophenyl)-methyl-amino]pyrimidin-2-yl]amino]-5-morpholin-4-yl-phenyl]-[(3R)-3-hydroxypyrrolidin-1-yl]methanone | 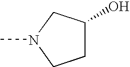 | 509 | 1.74-2.00 (m, 2 H), 3.05-3.11 (m, 4 H), 3.21 (d,0.5 H), 3.39-3.42 (mpartially hidden by H2O,0.5 H), 3.44 (s, 3 H), 3.46-3.60 (m, 3 H), 3.7 1-3.79 (m,4 H), 4.23 (bs, 0.5 H), 4.34(bs, 0.5 H), 4.94 (d, 0.5 H),5.00 (d, 0.5 H), 5.90 (d,0.5 H) 5.91 (d, 0.5 H), 6.58(s, 1 H), 7.34-7.42 (m, 3 H),7.47-7.54 (m, 3 H), 7.95 (d,0.5 H), 7.96 (d, 0.5 H), 9.19(s, 1 H) |
| 19.23 | 3-[[4-[(3-chlorophenyl)-methyl-amino]pyrimidin-2-yl]amino]-N-[(2R)-1-hydroxypropan-2-yl]-5-morpholin-4-yl-benzamide | 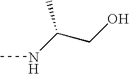 | 497 | (DMSOd6 at 323° K.) 1.14(d, 3 H), 3.09-3.16 (m, 4 H),3.33-3.41 (m, 1 H), 3.44-3.51 (m, 1 H), 3.47 (s, 3 H),3.74-3.80 (m, 4 H), 3.97-4.07 (m, 1 H), 4.59 (bs, 1 H),5.86 (d, 1 H), 6.94 (s, 1 H),7.35 (ddd, 1 H), 7.39 (ddd,1 H), 7.46 (dd, 1 H), 7.49(dd, 1 H), 7.50 (dd, 1 H),7.73 (d, 1 H), 7.79 (s, 1 H),7.93 (d, 1 H), 8.99 (bs, 1 H) |
| 19.24 | 3-[[4-[(3-chlorophenyl)-methylamino]pyrimi-din-2-yl]amino]-5-morpholin-4-yl-N-(oxan-4-yl)benzamide | 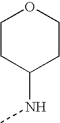 | 523 | (DMSOd6): 1.51-1.63 (m,2 H), 1.70-1.78 (m, 2 H),3.07-3.14 (m, 4 H), 3.34-3.42 (in partially hidden byH2O, 2 H), 3.47 (s, 3 H),3.73-3.79(m, 4 H), 3.85-3.92 (m, 2 H), 3.94-4.04 (m,1 H), 5.85 (d, 1 H), 6.93 (s,1 H), 7.36 (ddd, 1 H), 7.40(ddd, 1 H), 7.47 (s, 1 H),7.48-7.53 (m, 2 H), 7.84 (s,1 H), 7.92 (d, 1 H), 8.14 (d,1 H), 9.18 (s, 1 H) |
| 19.25 | 3-[[4-[(3- | Et- | 467 | (DMSOd6): 1.10 (t, 3 H), |
| | chlorophenyl)- | | | 3.04-3.15 (m, 4 H), 3.21- |
| | methylamino]pyrimi- | | | 3.30 (m, 2 H), 3.46 (s, 3 H), |
| | din-2-yl]amino]-N- | | | 3.71-3.79 (m, 4 H), 5.85 (d, |
| | ethyl-5-morpholin-4- | | | 1 H), 6.93 (s, 1 H), 7.35 (d, |
| | ylbenzamide | | | 1 H), 7.39 (d, 1 H), 7.44- |
| | | | | 7.54 (m, 3 H), 7.79 (s, 1 H), |
| | | | | 7.92 (d, 1 H), 8.26 (t, 1 H), |
| | | | | 8.16 (s, 1 H) |

3-[[4-[(3-Chlorophenyl)-methyl-amino]pyrimidin-2-yl]amino]-5-morpholin-4-yl-benzoic used as starting material was made as follows:

Sodium hydride (4.89 g, 73.4 mmol, 60% in oil) was added portionwise to an ice-cooled solution of N-methyl-3-chloroaniline (8 g, 56.5 mmol) in DMF (40 ml). This mixture was then added to an ice-cooled solution of 2,4-dichloropyrimidine (16.8 g, 113 mmol) in DMF (40 ml). The resulting mixture was heated at 70° C. for 18 hrs. Additional sodium hydride (large excess) was added and the mixture was heated at 100° C. for 2 hrs. After cooling, the mixture was poured in saturated aqueous sodium bicarbonate and extracted with DCM. The organic layer was dried over magnesium sulfate and filtered. After evaporation of the solvents, the residue was purified by chromatography on silica gel (eluant: 0 to 20% EtOAc in DCM, then 10% methanol in EtOAc) to give 4-[(3-chlorophenyl)-methyl-amino]-1H-pyrimidin-2-one as a light brown solid (4 g, 30%). NMR Spectrum: (DMSOd₆ and CF₃CO₂D) 3.53 (s, 3H), 7.46 (m, 1H), 7.67-7.60 (m, 3H), 7.78 (m, 1H); Mass spectrum: MH⁺ 236.

DMF (5 drops) was added to a mixture of 4-[(3-chlorophenyl)-methyl-amino]-1H-pyrimidin-2-one (3.08 g, 13.1 mmol) in phosphorus oxychloride (30 ml). The mixture was stirred at 100° C. for 1.5 hr. After evaporation of the solvent, the residue was diluted with DCM and slowly added to a cooled solution of saturated aqueous sodium bicarbonate. The mixture was extracted with DCM. The organic layer was concentrated. The residue was purified by chromatography on silica gel (eluant: DCM) to give 2-chloro-N-(3-chlorophenyl)-N-methyl-pyrimidin-4-amine (2.56 g, 77%) as a white solid. NMR Spectrum: (DMSOd₆) 3.38 (s, 3H), 6.40 (d, 1H), 7.38 (m, 1H), 7.45 (m, 1H), 7.54 (m, 2H), 8.05 (d, 1H); Mass spectrum: MH⁺ 254

Ethyl 3-amino-5-morpholin-4-yl-benzoate (Example 18, starting material) and 2-chloro-N-(3-chlorophenyl)-N-methyl-pyrimidin-4-amine were reacted according to procedure described in Example 18, starting material to give:

Ethyl 3-[[4-[(3-chlorophenyl)-methyl-amino]pyrimidin-2-yl]amino]-5-morpholin-4-yl-benzoate (3.15 g, 89%), white solid. Mass spectrum: MH⁺ 468

3-[[4-[(3-chlorophenyl)-methyl-amino]pyrimidin-2-yl]amino]-5-morpholin-4-yl-benzoic acid (3.18 g, quantitative), white solid. NMR Spectrum: (DMSOd₆) 3.09 (m, 4H), 3.46 (s, 3H), 3.74 (m, 4H), 5.87 (d, 1H), 7.08 (s, 1H), 7.42-7.35 (m, 2H), 7.50 (m, 2H), 7.59 (s, 1H), 7.93 (d, 1H), 7.99 (s, 1H), 9.38 (m, 1H); Mass spectrum: MH⁺ 440.

### EXAMPLE 20

### N2-(3,5-dimorpholinophenyl)-N4-isopropyl-N4-(3-methoxyphenyl)pyrimidine-2,4-diamine

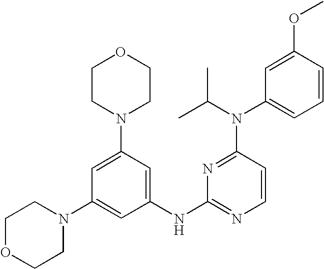

N2-(3,5-dimorpholinophenyl)-N4-isopropyl-N2-(4-methoxybenzyl)-N4-(3-methoxyphenyl)pyrimidine-2,4-diamine (170 mg, 0.23 mmol) and anisole (126 μl, 1.16 mmol) in TFA (5 ml) were stirred at 130° C. for 7 hrs. The reaction mixture was concentrated to dryness, diluted with DCM (30 ml), washed with a saturated aqueous solution of sodium hydrogencarbonate (1×70 ml), dried over magnesium sulfate and concentrated to afford the crude product as a off-white gum. The crude product was purified by flash chromatography on silica gel eluting with ethyl acetate. The solvent was evaporated to dryness to afford N2-(3,5-dimorpholinophenyl)-N4-isopropyl-N4-(3-methoxyphenyl)pyrimidine-2,4-diamine (76 mg, 65.1%) as a white foam. NMR Spectrum: (DMSOd₆) 1.09 (s, 6H), 3.03-3.11 (m, 8H), 3.70-3.77 (m, 8H), 3.78 (s, 3H), 5.19 (d, 1H), 5.31-5.41 (m, 1H), 6.11 (t, 1H), 6.77 (dd, 1H), 6.79 (d, 1H), 6.98 (d, 2H), 7.04 (dd, 1H), 7.45 (dd, 1H), 7.73 (dd, 1H), 8.82 (s, 1H); Mass spectrum: MH⁺ 505.

N2-(3,5-dimorpholinophenyl)-N4-isopropyl-N2-(4-methoxybenzyl)-N4-(3-methoxyphenyl)pyrimidine-2,4-diamine used as starting material was made as follows: 4N Hydrogen chloride in dioxane (0.050 mL, 0.20 mmol) was added to a stirred suspension of 4-chloro-N-(3,5-dimorpholinophenyl)-N-(4-methoxybenzyl)pyrimidin-2-amine (2 g, 4.03 mmol), and 3-methoxyaniline (0.473 mL, 4.23 mmol) in 2-propanol (20 mL) under nitrogen. The resulting suspension was stirred at 80° C. for 1 hr.

The reaction mixture was allowed to cool to room temperature and the solvent was evaporated. The residue was dissolved in DCM, quenched with a saturated aqueous solution of sodium hydrogenocarbonate and extracted with DCM (1×30 ml). The organic phase was dried over magnesium sulfate and concentrated to afford the crude product as a pale yellow gum. The crude product was purified by flash chromatography on silica gel eluting with 0 to 50% ethyl acetate in DCM. The solvent was evaporated to dryness to afford N2-(3,5-dimorpholinophenyl)-N2-(4-methoxybenzyl)-N4-(3-methoxyphenyl)pyrimidine-2,4-diamine (2.28 g, 97%) as a clear white foam. Mass spectrum: MH⁺ 583.

Sodium hydride (13.8 mg, 0.34 mmol, 60% in oil) was added in one portion to a stirred mixture of N2-(3,5-dimorpholinophenyl)-N2-(4-methoxybenzyl)-N4-(3-methoxyphenyl)pyrimidine-2,4-diamine (134 mg, 0.23 mmol) and 2-chloropropane (0.069 mL, 0.69 mmol) dissolved in DMF (3 mL) at 25° C. under argon. The resulting suspension was stirred at 90° C. for 3 hrs. Another portion of sodium hydride (28 mg, 0.68 mmol) was added following by 2-chloropropane (0.14 mL, 1.4 mmol). The resulting mixture was stirred at 90° C. for 18 hrs. The reaction mixture was allowed to cool to room temperature under stirring and quenched with a saturated aqueous solution of ammonium chloride. The resulting precipitate was collected by filtration, washed with water (100 ml) and dried to a constant weight to afford N2-(3,5-dimorpholinophenyl)-N4-isopropyl-N2-(4-methoxybenzyl)-N4-(3-methoxyphenyl)pyrimidine-2,4-diamine (145 mg, quantitative) as a beige solid. Mass spectrum: MH⁺ 625.

### EXAMPLE 21

### (3-((2-(3,5-dimorpholinophenylamino)pyrimidin-4-yl)(2-methoxyethyl)amino)-4-methylphenyl)methanol

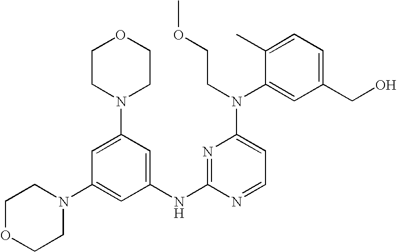

2-Bromoethyl methyl ether (0.103 mL, 1.10 mmol) was added dropwise to a stirred suspension of (3-(2-chloropyrimidin-4-ylamino)-4-methylphenyl)methanol (250 mg, 1.00 mmol, Method 2) and cesium carbonate (652 mg, 2.00 mmol) in DMF (4 mL) at 25° C. The resulting suspension was stirred at 25° C. overnight. The reaction mixture was filtered and the filtrate was concentrated to dryness. 3,5-Dimorpholin-4-ylaniline (264 mg, 1.00 mmol, Method 9) and hydrochloric acid 4N in dioxan (2 drops) were suspended in 2-propanol (3 mL) and sealed into a microwave tube. The reaction was heated to 140° C. over a period of 10 minutes in a Personal Chemistry EMRYS™ Optimizer EXP microwave synthesizer. The solvent was removed and the residue was dissolved in DMF (1.5 mL). Aqueous ammonia (100 μL) was added and the mixture was purified by preparative HPLC using a Waters X-Terra reverse-phase column (5 microns silica, 30 mm diameter, 150 mm length) and decreasingly polar mixtures of water (containing 0.2% ammonium carbonate) and acetonitrile as eluent. The fractions were evaporated to dryness to afford (3-((2-(3,5-dimorpholinophenylamino)pyrimidin-4-yl)(2-methoxyethyl)amino)-4-methylphenyl)methanol (192 mg, 36%) as a pink solid. NMR Spectrum: (DMSOd6 at 297° K) 2.07 (s, 3H), 3.01-3.13 (m, 8H), 3.20 (s, 3H), 3.40-3.51 (m, 2H), 3.68-3.78 (m, 8H), 3.94-4.04 (m, 1H), 4.18-4.28 (m, 1H), 4.50 (d, 2H), 5.18 (d, 1H), 5.22 (t, 1H), 6.11 (s, 1H), 6.97 (s, 2H), 7.18 (s, 1H), 7.26 (d, 1H), 7.34 (d, 1H), 7.78 (d, 1H), 8.83 (s, 1H); Mass spectrum: MH⁺ 535.

Using the procedure above with the corresponding halide, the following compounds were prepared

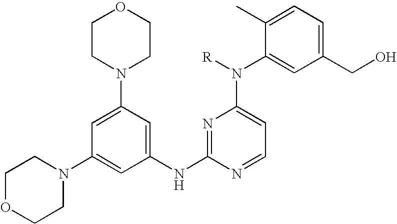

| | | R | | |
|---|---|---|---|---|
| | | (starting | Molecular | |
| Example | Name | halide) | ion (MH⁺) | NMR Spectrum (DMSOd6) |
| 21.1 | [3-[[2-[(3,5- | iPr | 519 | (DMSOd6 at 297° K) 0.94(d, |
| | dimorpholin-4- | (2-bromo | | 3 H), 1.28(d, 3 H), 2.07(s, |
| | ylphenyl)amino]py- | propane) | | 3 H), 3.01-3.15(m, 8 H), 3.69- |
| | rimidin-4-yl]- | | | 3.80(m, 8 H), 4.52(d, 2 H), |
| | propan-2-yl- | | | 5.04(bs, 1 H), 5.25(t, 1 H), |
| | amino]-4-methyl- | | | 5.28(bs, 1 H), 6.11(s, 1 H), |
| | phenyl]methanol | | | 7.00(s, 2 H), 7.11(s, 1 H), 7.29 |
| | | | | (d, 1 H), 7.36(d, 1 H), 7.74(d, |
| | | | | 1 H), 8.82(s, 1 H) |
| 21.2 | [3-[[2-[(3,5- | Et | 505 | (DMSOd6 at 297° K) 1.14(t, |
| | dimorpholin-4- | (2-bromo | | 3 H), 2.08(s, 3 H), 2.98-3.14 |
| | ylphenyl)amino]py- | ethane) | | (m, 8 H), 3.66-3.82(m, 9 H), |
| | rimidin-4-yl]- | | | 4.07-4.21(m, 1 H), 4.51(d, |
| | ethyl-amino]-4- | | | 2 H), 5.18(bs, 1 H), 5.23(t, |
| | methyl-phenyl] | | | 1 H), 6.11(s, 1 H), 7.00(s, 2 H), |
| | methanol | | | 7.16(s, 1 H), 7.27(d, 1 H), |
| | | | | 7.35(d, 1 H), 7.77(d, 1 H), |
| | | | | 8.83(s, 1 H) |
| 21.3^{a} | 2-[[2-[(3,5- | CH2CH2OH | 521 | (DMSOd6 at 297° K) 2.07(s, |
| | dimorpholin-4- | (2-bromo | | 3 H), 2.99-3.17(m, 8 H), 3.53- |
| | ylphenyl)amino]py- | ethylacetate) | | 3.64(m, 2 H), 3.65-3.81(m, |
| | rimidin-4-yl]-[5- | | | 9 H), 4.14-4.27(m, 1 H), 4.51 |
| | (hydroxymethyl)- | | | (d, 2 H), 4.68(t, 1 H), 5.17(s, |
| | 2-methyl- | | | 1 H), 5.23(t, 1 H), 6.11(s, 1 H), |
| | phenyl]amino] | | | 6.98(s, 2 H), 7.22(s, 1 H), 7.26 |
| | ethanol | | | (d, 1 H), 7.33(d, 1 H), 7.77(d, |
| | | | | 1 H), 8.81(s, 1 H) |

^{a}After reaction with 3,5-dimorpholin-4-ylaniline and cooling of the mixture, aqueous 2N sodium hydroxide (0.661 mL, 1.32 mmol) was added and the reaction mixture was stirred at room temperature overnight. The compound was then isolated as previously

### EXAMPLE 22

Using the procedure described in Example 21 with 2-chloro-N-(5-methoxy-2-methyl-phenyl)-N-methyl-pyrimidin-4-amine (200 mg, 0.8 mmol) and the corresponding halide, the following compounds were prepared:

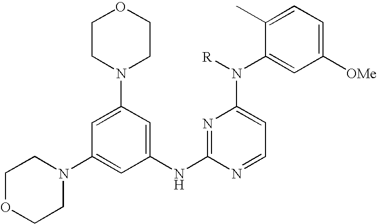

| | | R | | |
|---|---|---|---|---|
| | | (starting | Molecular | |
| Example | Name | halide) | ion (MH⁺) | NMR Spectrum (DMSOd6) |
| 22.1 | N-(3,5- | CH2CH2OMe | 535 | (DMSOd6 at 297° K) 2.01 |
| | dimorpholin-4- | (2-bromoethyl | | (s, 3 H), 3.00-3.14(m, 8 H), |
| | ylphenyl)-N′-(2- | methyl ether) | | 3.20(s, 3 H), 3.47(t, 2 H), |
| | methoxyethyl)- | | | 3.67-3.79(m, 1 H), 3.97-4.07 |
| | N′-(5-methoxy- | | | (m, 1 H), 4.17-4.28(m, 1 H), |
| | 2-methyl- | | | 5.27(bs, 1 H), 6.12(s, 1 H), |
| | phenyl) | | | 6.84(d, 1 H), 6.92(dd, 1 H), |
| | pyrimidine-2,4- | | | 6.98(s, 2 H), 7.28(d, 1 H), |
| | diamine | | | 7.80(d, 1 H), 8.84(s, 1 H) |
| 22.2 | N-(3,5- | iPr | 519 | (DMSOd6 at 297° K) 2.01 |
| | dimorpholin-4- | (2-bromo | | (s, 3 H), 2.99-3.17(m, 8 H), |
| | ylphenyl)-N′-(5- | propane) | | 3.52-3.66(m, 2 H), 3.69-3.79 |
| | methoxy-2- | | | (m, 9 H), 3.74(s, 3 H), 4.23 |
| | methyl-phenyl)- | | | (bs, 1 H), 4.70(t, 1 H), 5.22 |
| | N′-propan-2-yl- | | | (bs, 1 H), 6.11(s, 1 H), 6.89- |
| | pyrimidine-2,4- | | | 6.94(m, 2 H), 6.99(s, 2 H), |
| | diamine | | | 7.28(d, 1 H), 7.78(d, 1 H), |
| | | | | 8.81(bs, 1 H) |
| 22.3 | N-(3,5- | Et | 505 | DMSOd6 at 297° K) 1.13 |
| | dimorpholin-4- | (2-bromoethane) | | (t, 3 H), 2.01(s, 3 H), 3.00- |
| | ylphenyl)-N′- | | | 3.14(m, 8 H), 3.70-3.78(m, |
| | ethyl-N′-(5- | | | 8 H), 3.75(s, 3 H), 3.78-3.88 |
| | methoxy-2- | | | (m, 1 H), 4.04-4.15(m, 1 H), |
| | methyl-phenyl) | | | 5.22(bs, 1 H), 6.11(s, 1 H), |
| | pyrimidine-2,4- | | | 6.80(d, 1 H), 6.92(dd, 1 H), |
| | diamine | | | 7.00(s, 2 H), 7.30(d, 1 H), |
| | | | | 7.78(d, 1 H), 8.83(bs, 1 H) |
| 22.4^{a} | 2-[[2-[(3,5- | CH2CH2OH | 521 | (DMSOd6 at 297° K) 0.96 |
| | dimorpholin-4- | (2-bromo | | (d, 3 H), 1.29(d, 3 H), 2.00 |
| | ylphenyl)amino] | ethylacetate) | | (s, 3 H), 3.00-3,15(m, 8 H), |
| | pyrimidin-4- | | | 3.69-3.78(m, 8 H), 3.75(s, |
| | yl]-(5-methoxy- | | | 3 H), 5.09(bs, 1 H), 5.22-5.33 |
| | 2-methyl- | | | (m, 1 H), 6.11(s, 1 H), 6.71 |
| | phenyl)amino] | | | (d, 1 H), 6.96(dd, 1 H), 7.00 |
| | ethanol | | | (s, 2 H), 7.32(d, 1 H), 7.75 |
| | | | | (d, 1 H), 8.82(s, 1 H) |

^{a}After reaction with 3,5-dimorpholin-4-ylaniline and cooling of the mixture, aqueous 2N sodium hydroxide (0.481 mL, 0.96 mmol) was added and the reaction mixture was stirred at room temperature overnight. The compound was then isolated as previously.

### EXAMPLE 23

### 1-[3-({4-[(3-chlorophenyl)(methyl)amino]pyrimidin-2-yl}amino)-5-morpholin-4-ylphenyl]piperidin-4-ol

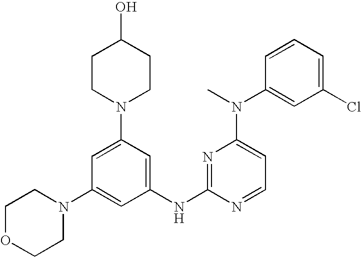

A suspension of 2-chloro-N-(3-chlorophenyl)-N-methylpyrimidin-4-amine (180 mg, 0.71 mmol), 1-(3-amino-5-morpholinophenyl)piperidin-4-ol (196 mg, 0.71 mmol) and HCl/dioxane 4N (0.195 ml, 0.78 mmol) in 2-pentanol (4 ml) was stirred at 120° C. over a period of 1.5 hr in a sealed tube. A precipitate was formed after cooling at room temperature. The precipitate was filtered, washed with 2-pentanol and dried. The solid was dissolved with DCM (10 ml) and a 5N solution of NH3 in methanol (2 ml). The insoluble was removed by filtration. The filtrate was concentrated to dryness, dissolved in DCM, filtered and concentrated to dryness. The resulting foam was triturated in ether and pentane to afford 1-[3-({4-[(3-chlorophenyl)(methyl)amino]pyrimidin-2-yl}amino)-5-morpholin-4-ylphenyl]piperidin-4-ol (190 mg, 54.2%) as a pink solid. Mass Spectrum: M+H⁺ 495. NMR Spectrum (DMSOd6): 1.39-1.50 (m, 2H), 1.74-1.84 (m, 2H), 2.71-2.81 (m, 2H), 2.96-3.05 (m, 4H), 3.41-3.50 (m, 5H), 3.54-3.63 (m, 1H), 3.66-3.74 (m, 4H), 4.65 (d, 1H), 5.82 (d, 1H), 6.08 (s, 1H), 6.90 (s, 1H), 6.95 (s, 1H), 7.35 (dd, 1H), 7.38 (dd, 1H), 7.49 (s, 1H), 7.50 (dd, 1H), 7.90 (d, 1H), 8.85 (s, 1H)

The 2-chloro-N-(3-chlorophenyl)-N-methylpyrimidin-4-amine used as starting material was made as follows:—

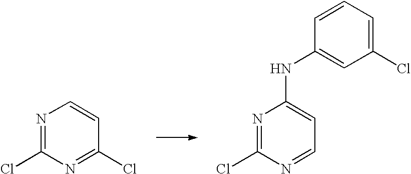

A solution of 2,4-dichloropyrimidine (4 g, 26.85 mmol), 3-chloroaniline (2.84 ml, 26.85 mmol) and triethylamine (4.49 ml, 32.22 mmol) in EtOH (40 ml) was heated at 80° C. overnight. The solvents were evaporated. The residue was diluted with AcOEt and a 1M aqueous solution of hydrochloric acid. The organic phase was washed with water, with a saturated aqueous solution of sodium hydrogencarbonate and a saturated aqueous solution of brine, dried over magnesium sulfate and concentrated to afford the crude product as a beige solid. The crude was triturated in AcOEt (40 ml), filtered, washed with CH₂Cl₂ and dried to afford 00150-62-01 (3 g) as a white solid. Mass Spectrum: M+H⁺ 240 and 242. NMR Spectrum (DMSOd6): 6.80 (d, 1H), 7.14 (dd, 1H), 7.40 (dd, 1H), 7.51 (dd, 1H), 7.82 (s, 1H), 8.22 (d, 1H), 10.20 (s, 1H)

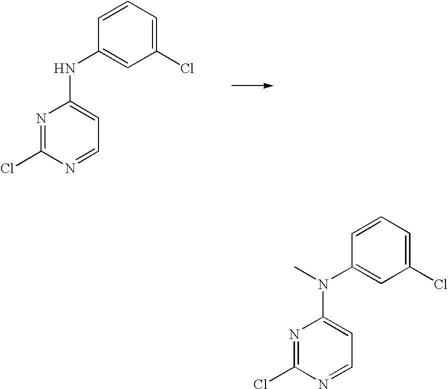

A suspension of 2-chloro-N-(3-chlorophenyl)pyrimidin-4-amine (2.5 g, 10.41 mmol) and cesium carbonate (6.79 g, 20.83 mmol) in DMF (15 ml) was stirred at 25° C. for 10 min then methyl iodide (0.648 ml, 10.41 mmol) was added. After 3 hrs at 25° C., the insoluble was removed by filtration. The filtrate was concentrated to dryness and diluted with AcOEt and water. The organic phase was washed with brine, dried over magnesium sulfate and concentrated to dryness. The solid was triturated in ether (20 ml), filtered, dried to afford 2-chloro-N-(3-chlorophenyl)-N-methylpyrimidin-4-amine (1.5 g, 56.7%) as a white solid. Mass Spectrum: M+H⁺ 254 and 256. NMR Spectrum (CDCl3): 3.48 (s, 3H), 6.20 (d, 1H), 7.15 (ddd, 1H), 7.26 (dd, 1H), 7.36 (ddd, 1H), 7.42 (dd, 1H), 7.94 (d, 1H)

### EXAMPLE 24

### N4-(3-chlorophenyl)-N4-methyl-N2-[3-(4-methylpiperazin-1-yl)-5-morpholin-4-ylphenyl]pyrimidine-2,4-diamine

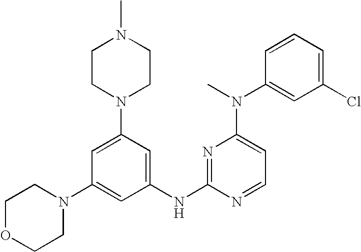

Using a procedure similar to the one described in example 23, 2-chloro-N-(3-chlorophenyl)-N-methylpyrimidin-4-amine (180 mg, 0.71 mmol) was reacted with 3-(4-methylpiperazin-1-yl)-5-morpholinoaniline (196 mg, 0.71 mmol) to give N4-(3-chlorophenyl)-N4-methyl-N2-[3-(4-methylpiperazin-1-yl)-5-morpholin-4-ylphenyl]pyrimidine-2,4-diamine (213 mg, 60.9%) as a white solid. Mass Spectrum: M+H⁺ 494. NMR Spectrum (DMSOd6): 2.21 (s, 3H), 2.38-2.45 (m, 4H), 2.98-3.04 m (m, 4H), 3.04-3.09 (m, 4H), 3.44 (s, 3H), 3.67-3.74 (m, 4H), 5.83 (d, 1H), 6.08 (s, 1H), 6.94 (d, 1H), 7.35 (dd, 1H), 7.38 (dd, 1H), 7.48 (dd, 1H), 7.49 (s, 1H), 7.90 (d, 1H), 8.85 (s, 1H)

### EXAMPLE 25

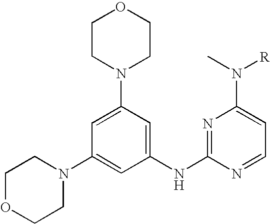

The following compounds were prepared as described for the synthesis of example 6.

| | | | Molecular | NHR Spectrum |
|---|---|---|---|---|
| Example | Name | R | ion (MH⁺) | (DMSOd6) |
| 25.1^{a} | (2-((2-(3,5-dimorpholinophenylamino)pyrimidin-4-yl)(methyl)amino)-3-methylphenyl)methanol | 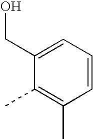 | 491 | DMSOd6: 2.08(s, 3 H),3.03-3.12(m, 8 H), 3.31(s, 3 H), 3.69(3.76(m,8 H), 4.25(dd, 1 H), 4.41(dd, 1 H), 5.15(d, 1 H),5.18(t, 1 H), 6.12(t, 1 H),7.04(d, 2 H), 7.29(d,1 H), 7.34(dd, 1 H), 7.45(d, 1 H), 7.76(d, 1 H),8.86(s, 1 H) |
| 25.2^{b} | N2-(3,5-dimorpholinophenyl)-N4-(2-methoxy-6-methylphenyl)-N4-methylpyrimidine-2,4-diamine | 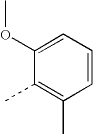 | 491 | DMSOd6: 2.09(s, 3 H),3.05-3.11(m, 8 H), 3.28(s, 3 H), 3.68-3.75(m,8 H), 3.73(s, 3 H), 5.23(d, 1 H), 6.10(s, 1 H),6.94(d, 1 H), 6.97-7.05(m, 3 H), 7.28(dd, 1 H),7.73(d, 1 H), 8.61(s, 1 H) |
| 25.3^{c} | (3-((2-(3,5-dimorpholinophenylamino)pyrimidin-4-yl)(methyl)amino)-4-fluorophenyl)methanol | 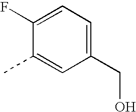 | 495 | DMSOd6: 3.00-3.09(m,8 H), 3.40(s, 3 H), 3.67-3.75(m, 8 H), 4.51(d,2 H), 5.32(t, 1 H), 5.63(bs, 1 H), 6.11(s, 1 H),6.97(s, 2 H), 7.33(dd,1 H), 7.35(s, 1 H), 7.40(d, 1 H), 7.89(d, 1 H),8.87(s, 1 H) |
| 25.4^{d} | (3-((2-(3,5-dimorpholinophenylamino)pyrimidin-4-yl)(methyl)amino)-2-fluorophenyl)methanol | 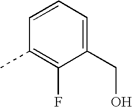 | 495 | DMSOd6 at 323° K:3.01-3.09(m, 8 H), 3.32(s, 3 H), 8.68-8.74(m,8 H), 4.28(bs, 1 H), 4.39(bs, 1 H), 5.22(bs, 1 H),5.38(bs, 1 H), 6.09(s,1 H), 6.96(s, 2 H), 7.18(ddd, 1 H), 7.29(dd, 1 H),7.36(dd, 1 H), 7.81(d,1 H), 8.64(bs, 1 H) |
| 25.5^{e} | 1-(3-((2-(3,5-dimorpholinophenylamino)pyrimidin-4-yl)(methyl)amino)-4-methylphenyl)ethanone | 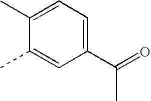 | 503 | DMSOd6: 2.18(s, 3 H),2.57(s, 3 H), 3.06(bs,8 H), 3.40(s, 3 H), 3.71(bs, 8 H), 5.25(bs, 1 H),6.11(s, 1 H), 7.03(s,2 H), 7.55(d, 1 H), 7.81(bs, 1 H), 7.84(s, 1 H),7.90(d, 1 H), 8.89(s, 1 H) |
| 25.6^{f} | N2-(3,5-dimorpholinophenyl)-N4-(2-fluoro-5-methoxyphenyl)-N4-methylpyrimidine-2,4-diamine | 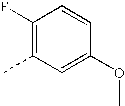 | 495 | CDCl3: 3.13-3.20(m,8 H), 3.45(s, 3 H), 3.79(s, 3 H), 3.81-3.88(m,8 H), 5.74(d, 1 H), 6.16(t, 1 H), 6.80(dd, 1 H),6.84(dd, 1 H), 6.86(d,2 H), 7.06(bs, 1 H), 7.12(dd, 1 H), 7.88(d, 1 H) |
| 25.7^{g} | (2-((2-(3,5-dimorpholinophenylamino)pyrimidin-4-yl)(methyl)amino)-4-methoxyphenyl)methanol | 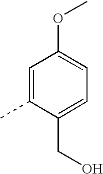 | 507 | DMSOd6 at 323° K:3.08(bs, 8 H), 3.38(s,3 H), 3.73(bs, 8 H), 3.78(s, 3 H), 4.28(bs, 2 H),4.93(s, 1 H), 5.40(bs,1 H), 6.11(s, 1 H), 6.83(s, 1 H), 7.96-7.06(m,3 H), 7.51(d, 1 H), 7.80(d, 1 H), 8.65(s, 1 H) |
| 25.8^{h} | (4-chloro-2-((2-(3,5-dimorpholinophenylamino)pyrimidin-4-yl)(methyl)amino)phenyl)methanol | 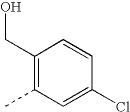 | 510 | DMSOd6 at 323° K:3.00-3.09(m, 8 H), 3.34(s, 3 H), 3.68-3.76(m,8 H), 4.32(bs, 2 H), 5.14(t, 2 H), 5.43(bs, 1 H),6.09(t, 1 H), 6.95(s, 1 H),7.37(d, 1 H), 7.47(dd,1 H), 7.63(d, 1 H), 7.84(d, 1 H), 8.66(s, 1 H) |

^{a}The (2-((2-chloropyrimidin-4-yl)(methyl)amino)-3-methylphenyl)methanol used as starting material was made as follows:-

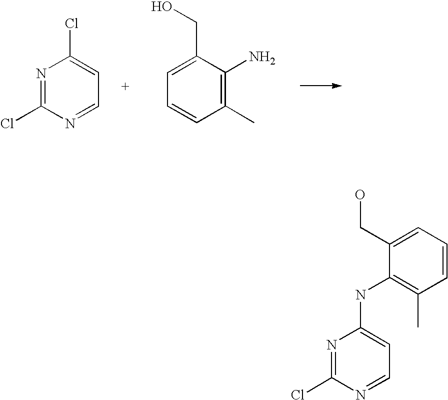

A mixture of 2,4-dichloropyrimidine (1.5 g, 10.07 mmol), 2-amino-3-methylbenzyl alcohol (commercial) (1.285 g, 9.36 mmol) and triethylamine (1.544 ml, 11.08 mmol) in ethanol (13 ml) was stirred at reflux for 20 hrs as described in Method 1. Work-up and purification gave (2-(2-chloropyrimidin-4-ylamino)-3-methylphenyl)methanol (0.477 g, 18.97%) as a beige foam. Mass Spectrum: M+H⁺ 250. NMR Spectrum (DMSOd6 at 323° K): 2.12 (s, 3H), 4.40 (bs, 2H), 4.97 (bs, 1H), 7.22 (s, 1H), 7.26 (s, 1H), 7.39 (d, 1H), 8.02 (s, 1H), 9.19 (bs, 1H)

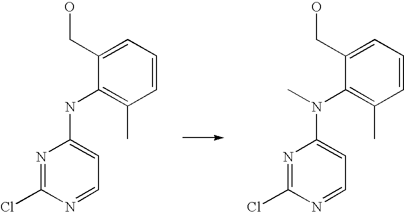

A suspension of (2-(2-chloropyrimidin-4-ylamino)-3-methylphenyl)methanol (470 mg, 1.88 mmol), cesium carbonate (1227 mg, 3.76 mmol) and methyl iodide (129 μl, 2.07 mmol) in DMF (5 ml) was a stirred over a period of 20 hrs at 22° C. under argon as described in Method 1. Work-up and purification gave (2-((2-chloropyrimidin-4-yl)(methyl)amino)-3-methylphenyl)methanol (386 mg, 78%) as a pale yellow solid. Mass Spectrum: M+H⁺ 264. NMR Spectrum (DMSOd6 at 323° K): 2.05 (s, 3H), 3.27 (s, 3H), 4.22 (dd, 1H), 4.36 (dd, 1H), 5.25 (t, 1H), 5.70 (d, 1H), 7.31 (d, 1H), 7.37 (dd, 1H), 7.45 (d, 1H), 7.91 (d, 1H)

^{b}: The 2-chloro-N-(2-methoxy-6-methylphenyl)-N-methylpyrimidin-4-amine used as starting material was made as follows:—

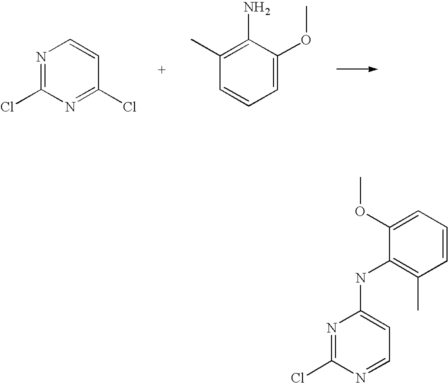

A solution of 2,4-dichloropyrimidine (1.5 g, 10.07 mmol), 2-methoxy-6-methylaniline (1.285 g, 9.36 mmol) and n,n-diisopropylethylamine (1.929 ml, 11.08 mmol) in n-butanol (13 ml), was stirred 15 hrs at reflux as described in Method 1. Work-up and purification gave 2-chloro-N-(2-methoxy-6-methylphenyl)pyrimidin-4-amine (1.450 g, 57.7%) as a beige solid. Mass Spectrum: M+H⁺ 250. NMR Spectrum (DMSOd6 at 323° K): 2.14 (s, 3H), 3.73 (s, 3H), 6.16 (bs, 1H), 6.89 d, 1H), 6.95 (d, 1H), 7.21 (dd, 1H), 7.99 (d, 1H), 9.10 (s, 1H)

The above product was methylated using a procedure similar to the one described in note (a). The crude product was purified by flash chromatography on silica gel eluting with 10 to 30% ethyl acetate in petroleum ether. The solvent was evaporated to dryness to afford 2-chloro-N-(2-methoxy-6-methylphenyl)-N-methylpyrimidin-4-amine (816 mg, 97%) as a colorless gum. Mass Spectrum: M+H⁺ 264. NMR Spectrum (DMSOd6): DMSOd6: 2.08 (s, 3H), 3.23 (s, 3H), 3.74 (s, 3H), 5.77 (d, 1H), 6.98 (d, 1H), 7.04 (d, 1H), 7.34 (dd, 1H), 7.91 (d, 1H)

^{c}: The (3-((2-chloropyrimidin-4-yl)(methyl)amino)-4-fluorophenyl)methanol used as starting material was made as follows:—

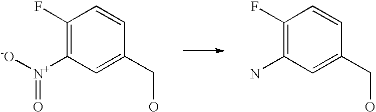

A suspension of (4-fluoro-3-nitrophenyl)methanol (2.5 g, 14.61 mmol) and palladium 10% on carbon (0.25 g, 2.35 mmol) in ethanol (70 ml), was hydrogenated under 1 atm of hydrogen at 21° C. for 1 hour. The resulting suspension was filtered and the filtrate was concentrated to dryness to afford the crude (3-amino-4-fluorophenyl)methanol (1.900 g, 92%) as a pale beige solid. Mass Spectrum: M+H⁺ 142. NMR Spectrum (DMSOd6): 4.32 (d, 2H), 5.00-5.07 (m, 3H), 6.43 (ddd, 1H), 6.73 (dd, 1H), 6.88 (dd, 1H)

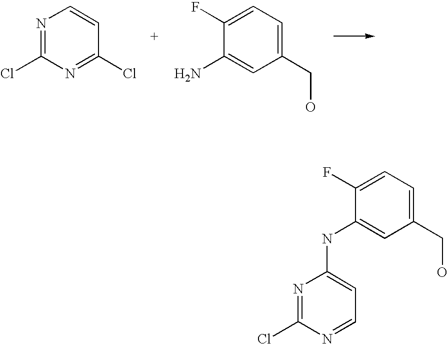

A solution of 2,4-dichloropyrimidine (1.5 g, 10.07 mmol), (3-amino-4-fluorophenyl)methanol (1.421 g, 10.07 mmol) and n,n-diisopropylethylamine (1.929 ml, 11.08 mmol) in n-butanol (13 ml), was stirred 15 hrs at reflux as described in Method 1. Work-up and purification gave (3-(2-chloropyrimidin-4-ylamino)-4-fluorophenyl)methanol (1.170 g, 45.8%) as a beige solid. Mass Spectrum: M+H⁺ 254. NMR Spectrum (DMSOd6): 4.48 (d, 2H), 5.29 (t, 1H), 6.72 (d, 1H), 7.18 (ddd, 1H), 7.27 (dd, 1H), 7.60 (d, 1H), 8.17 (d, 1H), 9.81 (bs, 1H)

The above product was methylated using a procedure similar to the one described in note (a) to give (3-((2-chloropyrimidin-4-yl)(methyl)amino)-4-fluorophenyl)methanol (60%) as a white solid. Mass Spectrum: M+H⁺ 268. NMR Spectrum (DMSOd6 at 323° K): 3.37 (s, 3H), 4.17 (d, 2H), 5.20 (t, 1H), 6.32 (bs, 1H), 7.34 (dd, 1H), 7.37-7.42 (m, 2H), 8.07 (d, 1H)

^{d}: The (3-((2-chloropyrimidin-4-yl)(methyl)amino)-2-fluorophenyl)methanol used as starting material was made as follows:—

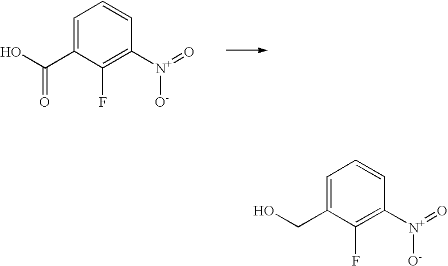

NMR Spectrum (DMSOd6): 4.85 (d, 2H), 5.72 (t, 1H), 7.38 (ddd, 1H), 7.60 (dd, 1H), 8.21 (dd, 1H)

A commercial solution of BH₃ (22.7 mL; 22.7 mmol; 1 N solution in THF) at 0° C. was added dropwise to 2-fluoro-3-nitrobenzoic acid (2.8 g; 15.14 mmol) in solution in THF (30 mL). Upon completion of the addition, the ice bath was removed and stirring was continued for 3 days. The reaction mixture was cooled to 0° C. and methanol was slowly added. Once gas evolution had ceased, the solvent were removed under vacuum and the residue diluted with ethyl acetate, washed with brine and dried over magnesium sulphate. The filtrate was adsorbed onto silica, poured on the top of a flash chromatography column and eluted with dichloromethane. Evaporation of the solvent gave (3-nitro-2-fluorophenyl)methanol (2.4 g; 93%)

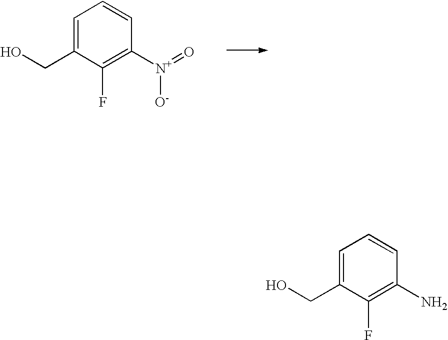

NMR Spectrum (DMSOd6): 4.34 (d, 2H), 4.74 (bs, 2H), 5.12 (t, 1H), 6.59 (dd, 1H), 6.77 (ddd, 1H), 6.91 (dd, 1H)

(3-nitro-2-fluorophenyl)methanol (2.1 g, 12.28 mmol) in solution in AcOEt was reduced with hydrogen (30 Psi) in presence of Pt/C over 1 h. The catalyst was removed by filtration and the solvent evaporated to give (3-amino-2-fluorophenyl)methanol as a yellow solid. (1.64 g, 95%)

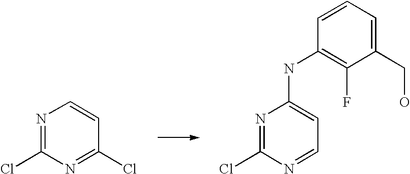

A solution of 2,4-dichloropyrimidine (300 mg, 2.01 mmol), (3-amino-2-fluorophenyl)methanol (256 mg, 1.81 mmol) and triethylamine (309 μl, 2.22 mmol) in 2-propanol (5 ml) was stirred over a period of 15 hrs at 90° C. as described in Method 1. Work-up and purification gave (3-(2-chloropyrimidin-4-ylamino)-2-fluorophenyl)methanol (206 mg, 40.3%) as a white solid. Mass Spectrum: M+H⁺ 254.

NMR Spectrum (DMSOd6): 4.44 (s, 2H), 5.39 (bs, 1H), 6.51 (bs, 1H), 7.13 (ddd, 1H), 7.30 (dd, 1H), 7.36 (dd, 1H), 8.09 (d, 1H), 9.41 (bs, 1H)

The above product was methylated using a procedure similar to the one described in note (a) to give (3-((2-chloropyrimidin-4-yl)(methyl)amino)-2-fluorophenyl)methanol (73%) as a white solid. Mass Spectrum: M+H⁺ 268. NMR Spectrum (DMSOd6 at 323° K): 3.28 (s, 3H), 4.23 (dd, 1H), 4.37 (dd, 1H), 5.25 (t, 1H), 5.86 (bs, 1H), 7.22 (dd, 1H), 7.33 (dd, 1H), 7.38 (d, 1H), 7.96 (bs, 1H)

^{e}: A mixture of 3,5-dimorpholinoaniline (87 mg, 0.31 mmol), 1-(3-((2-chloropyrimidin-4-yl)(methyl)amino)-4-methylphenyl)ethanone (81 mg, 0.29 mmol) and hydrochloric acid (4M in dioxane) (3.67 μL, 0.01 mmol) in iPrOH (1 mL) was stirred at 80° C. for 6 hrs. The reaction mixture was concentrated to dryness and diluted with DCM:methanolic ammonia 7N (95:5, 5 mL). The resulting precipitate was removed by filtration and washed with DCM. The filtrate was concentrated down and purified by flash chromatography on silica gel eluting with 0 to 100% ethyl acetate in DCM to afford 1-(3-((2-(3,5-dimorpholinophenylamino)pyrimidin-4-yl)(methyl)amino)-4-methylphenyl)ethanone (106 mg, 71.8%) as a reddish solid.

The 1-(3-((2-chloropyrimidin-4-yl)(methyl)amino)-4-methylphenyl)ethanone used as starting material was made as follows:—

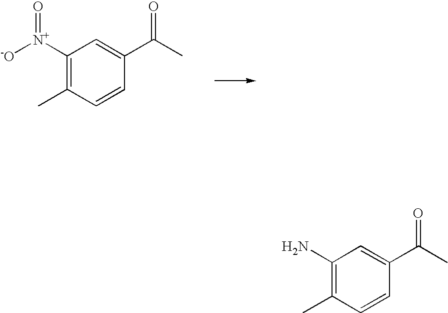

A solution of 4-methyl-3-nitroacetophenone (500 mg, 2.79 mmol) and ADAMS' platinum oxide (44 mg, 0.2 mmol) in ethyl acetate (5 mL) and ethanol (5.00 mL) was hydrogenated under 1.3 atm at room temperature for 30 minutes. The resulting solution was filtered through a pad of celite and the filtrate was concentrated to dryness to afford the yellow oil, which was used without further purification. Mass Spectrum: M+H⁺ 150. NMR Spectrum (CDCl3): 2.22 (s, 3H), 2.55 (s, 3H), 3.72 (bs, 2H), 7.12 (d, 1H), 7.24 (d, 1H), 7.29 (dd, 1H)

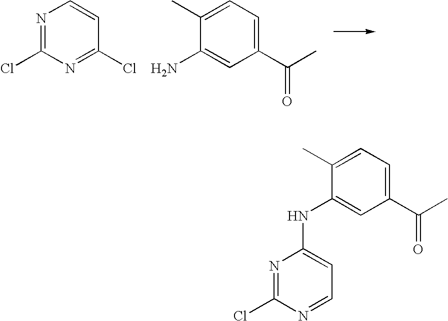

A solution of 2,4-dichloropyrimidine (200 mg, 1.34 mmol), 1-(3-amino-4-methylphenyl)ethanone (200 mg, 1.34 mmol) and n,n-diisopropylethylamine (0.257 mL, 1.48 mmol) in 2-propanol (2 mL) was stirred at 80° C. for 3 days. The reaction mixture was concentrated to dryness, diluted with DCM, washed with an aqueous solution of 10% citric acid, dried over MgSO4 and concentrated in presence of silica gel. The crude product was purified by flash chromatography on silica gel eluting with 20 to 50% ethyl acetate in petroleum ether. The solvent was evaporated to dryness to afford 1-(3-(2-chloropyrimidin-4-ylamino)-4-methylphenyl)ethanone (181 mg, 51.5%) as a white solid. Mass Spectrum: M+H⁺ 262. NMR Spectrum (CDCl3): 2.34 (s, 3H), 2.60 (s, 3H), 6.33 (d, 1H), 6.87 (bs, 1H), 7.42 (d, 1H), 7.81 (dd, 1H), 7.74 (d, 1H), 8.13 (d, 1H)

The above product was methylated using a procedure similar to the one described in note (a) to give 1-(3-((2-chloropyrimidin-4-yl)(methyl)amino)-4-methylphenyl)ethanone (90%) as a white solid. Mass Spectrum: M+H⁺ 276. NMR Spectrum (CDCl3): 2.21 (s, 3H), 2.60 (s, 3H), 3.44 (s, 3H), 5.76 (d, 1H), 7.47 (d, 1H), 7.76 (d, 1H), 7.88 (d, 1H), 7.91 (d, 1H)

^{f}: Coupling between 3,5-dimorpholinoaniline (144 mg, 0.51 mmol), 2-chloro-N-(2-fluoro-5-methoxyphenyl)-N-methylpyrimidin-4-amine (130 mg, 0.49 mmol) was done as described in note (e) and gave N2-(3,5-dimorpholinophenyl)-N4-(2-fluoro-5-methoxyphenyl)-N4-methylpyrimidine-2,4-diamine (197 mg, 82%) as a clear yellow foam.

The 2-chloro-N-(2-fluoro-5-methoxyphenyl)-N-methylpyrimidin-4-amine used as starting material was made as follows:—

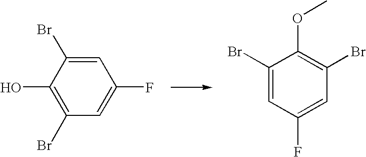

A suspension of 2,6-dibromo-4-fluorophenol (5 g, 18.53 mmol), potassium carbonate (3.84 g, 27.79 mmol) and dimethyl sulfate (1.928 ml, 20.38 mmol) in acetone (110 ml) was a stirred over a period of 6 hours at reflux. The reaction mixture was allowed to cool to room temperature, the salt was filtered, the filtrate was concentrated to dryness, diluted with diethyl ether, washed with water and brine, dried over magnesium sulfate and concentrated to afford the product 1,3-dibromo-5-fluoro-2-methoxybenzene (5.20 g, 99%) as a white solid. NMR Spectrum (CDCl3): 3.86 (s, 3H), 7.28 (d, 2H)

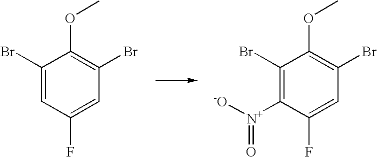

A solution of fuming HNO3 90% (0.35 ml) in H2SO4 conc (6.5 ml) was added dropwise to a stirred suspension of 1,3-dibromo-5-fluoro-2-methoxybenzene (2 g, 7.04 mmol) in H2SO4 conc (8.5 ml) over a period of 10 minutes while maintaining a temperature between 0 and +5° C. The resulting suspension was stirred in an ice bath for 2 hours, quenched into ice (30 g) and extracted with dichloromethane (2×20 ml). The organic phase was washed with a saturated aqueous solution of sodium hydrogencarbonate (×2), dried over magnesium sulfate and concentrated to afford the desired 1,3-dibromo-5-fluoro-2-methoxy-4-nitrobenzene (2.100 g, 91%) as a pale yellow oil. The crude product was used without further purification in the next step. NMR Spectrum (CDCl3): 3.92 (s, 3H), 7.53 (d, 1H)

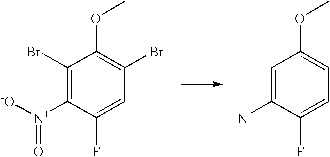

A suspension of 1,3-dibromo-5-fluoro-2-methoxy-4-nitrobenzene (2.1 g, 6.38 mmol) and palladium 10% on carbon 50% wet paste (0.5 g, 4.70 mmol) in ethanol (60 ml) was hydrogenated in a Parr reactor (500 ml) under 60 psi at 21° C. for 25 hours. The resulting suspension was filtered and the filtrate was concentrated to dryness. The solid obtained was diluted with methanol and a solution 7N of NH3 in methanol was added. The mixture was concentrated to dryness, diluted with dichloromethane, the solid was removed by filtration and the solvent evaporated. The product was purified by flash chromatography on silica gel (15-40 μm) eluting with dichloromethane. The solvent was evaporated to dryness to afford 2-fluoro-5-methoxyaniline (0.720 g, 80%) as a white solid. Mass Spectrum: M+H⁺ 142. NMR Spectrum (CDCl3): 3.71 (bs, 2H), 3.74 (s, 3H), 6.20 (ddd, 1H), 6.32 (dd, 1H), 6.88 (dd, 1H)

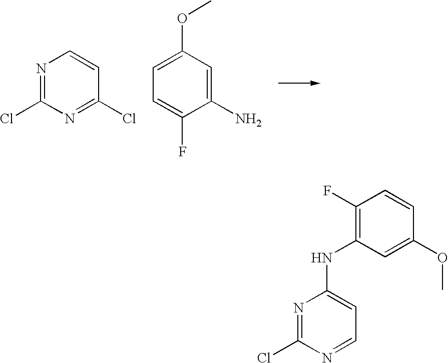

A suspension of 2,4-dichloropyrimidine (200 mg, 1.34 mmol), 2-fluoro-5-methoxyaniline (189 mg, 1.34 mmol) and n,n-diisopropylethylamine (0.257 mL, 1.48 mmol) in 2-propanol (1.5 mL) was stirred at reflux for 3 days as described in Method 1. Work-up and purification gave 2-chloro-N-(2-fluoro-5-methoxyphenyl)pyrimidin-4-amine (220 mg, 64.6%) as an oil, which crystallised on standing. Mass Spectrum: M+H⁺ 254.

NMR Spectrum (CDCl3): 3.82 (s, 3H), 3.58 (d, 1H), 3.67 (ddd, 1H), 6.90 (bs, 1H), 7.08 (dd, 1H), 7.49 (bs, 1H), 8.20 (d, 1H)

The above product was methylated using a procedure similar to the one described in note (a) to give 2-chloro-N-(2-fluoro-5-methoxyphenyl)-N-methylpyrimidin-4-amine (92%) as a colorless oil. Mass Spectrum: M+H⁺ 268. NMR Spectrum (CDCl3): 3.45 (s, 3H), 3.80 (s, 3H), 6.11 (d, 1H), 6.77 (ddd, 1H), 6.89 (ddd, 1H), 7.15 (dd, 1H), 7.95 (d, 1H)

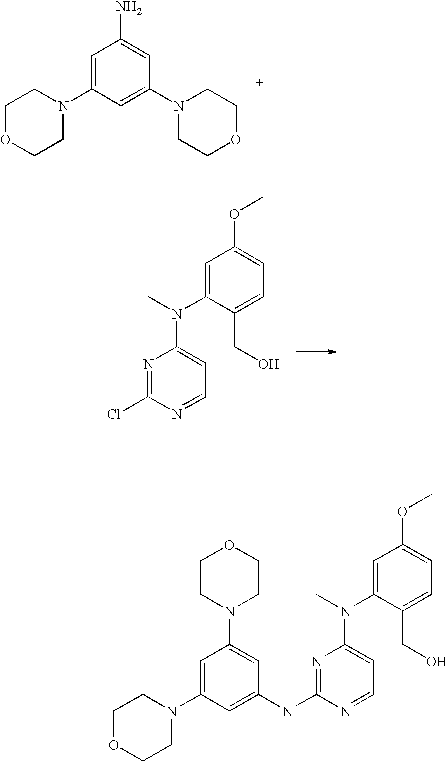

^{g}: A mixture of 3,5-dimorpholinoaniline (254 mg, 0.97 mmol), (2-((2-chloropyrimidin-4-yl)(methyl)amino)-4-methoxyphenyl)methanol (180 mg, 0.64 mmol) and hydrogen chloride 5N in iPrOH (5 drops) in 2-pentanol (5 mL) was stirred at 100° C. for 3 hours. The reaction was concentrated to dryness, the residue was taken up in DMF (1.5 ml) and NH3 7N in MeOH (100 μl) was added. The solution was filtered and purified by preparative HPLC using a Waters X-Bridge reverse-phase column (C-18, 5 microns silica, 19 mm diameter, 100 mm length, flow rate of 40 ml/minute) and decreasingly polar mixtures of water (containing 0.2% ammonium carbonate) and acetonitrile as eluent. The fractions containing the desired compound were evaporated to dryness to afford a dark brown solid which was further purified by flash chromatography on silica gel eluting with 0 to 5% methanol in dichloromethane. The solvent was evaporated to dryness to afford (2-((2-(3,5-dimorpholinophenylamino)pyrimidin-4-yl)(methyl)amino)-4-methoxyphenyl)methanol as a pale brown foam. This foam was triturated in diethyl ether, diluted with petroleum ether, sonicated for 10 minutes and filtered to afford (2-((2-(3,5-dimorpholinophenylamino) pyrimidin-4-yl)(methyl)amino)-4-methoxyphenyl)methanol (125 mg, 38.3%) as a beige solid.

The (2-((2-chloropyrimidin-4-yl)(methyl)amino)-4-methoxyphenyl)methanol used as starting material was made as follows:—

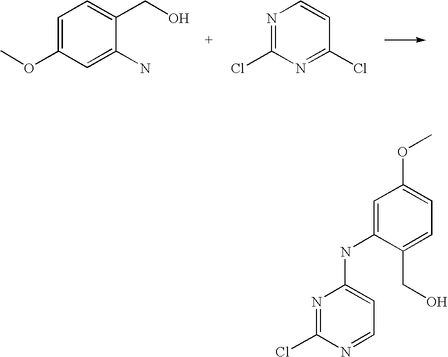

(2-amino-4-methoxyphenyl)methanol (555 mg, 3.62 mmol), 2,4-dichloropyrimidine (1080 mg, 7.25 mmol) and N-ethyl-N-isopropylpropan-2-amine (DIPEA) (1.893 mL, 10.87 mmol) in EtOH (10 mL) were stirred at 80° C. over the weekend. The reaction mixture was concentrated to dryness and partitioned between ethyl acetate (30 ml) and water. The aqueous phase was extracted with ethyl acetate (2×30 ml) and the combined organic phases were washed with brine, dried over magnesium sulfate and concentrated to afford the crude product as a yellow oil. The crude product was purified by flash chromatography on silica gel eluting with 0 to 50% ethyl acetate in dichloromethane. The solvent was evaporated to dryness to afford (2-(2-chloropyrimidin-4-ylamino)-4-methoxyphenyl)methanol (340 mg, 35.3%) as a pale yellow solid.

Mass Spectrum: [M−H]⁻ 264. NMR Spectrum (DMSOd6): 3.75 (s, 3H), 4.41 (s, 2H), 5.14 (bs, 1H), 6.60 (d, 1H), 6.84 (dd, 1H), 7.02 (s, 1H), 7.39 (d, 1H), 8.10 (d, 1H), 9.40 (bs, 1H)

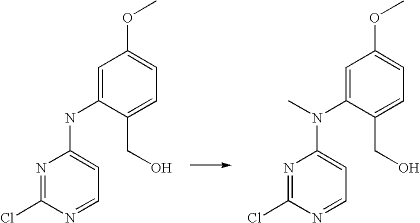

Iodomethane (0.082 mL, 1.32 mmol) was added to a stirred suspension of (2-(2-chloropyrimidin-4-ylamino)-4-methoxyphenyl)methanol (320 mg, 1.20 mmol) and potassium carbonate (250 mg, 1.81 mmol) in DMF (10 mL) at 0° C. under nitrogen. The resulting suspension was stirred at room temperature overnight. The reaction mixture was filtrated, concentrated to dryness and partitioned between ethyl acetate (20 ml) and water (20 ml). The aqueous phase was extracted with ethyl acetate (2×10 ml) and the combined organic phases were washed with brine, dried over magnesium sulfate and concentrated to afford the crude product as a yellow gum. The crude product was purified by flash chromatography on silica gel eluting with 0 to 40% ethyl acetate in dichloromethane. The solvent was evaporated to dryness to afford (2-((2-chloropyrimidin-4-yl)(methyl)amino)-4-methoxyphenyl)methanol (200 mg, 59.4%) as a pale yellow gum.

Mass Spectrum: M+H⁺ 280.

NMR Spectrum (DMSOd6): 3.32 (s partially hidden by H2O, 3H), 3.76 (s, 3H), 4.18 (d, 1H), 4.28 (d, 1H), 5.09 (bs, 1H), 5.85 (d, 1H), 6.90 (s, 1H), 7.04 (d, 1H), 7.51 (d, 1H), 7.91 (d, 1H)

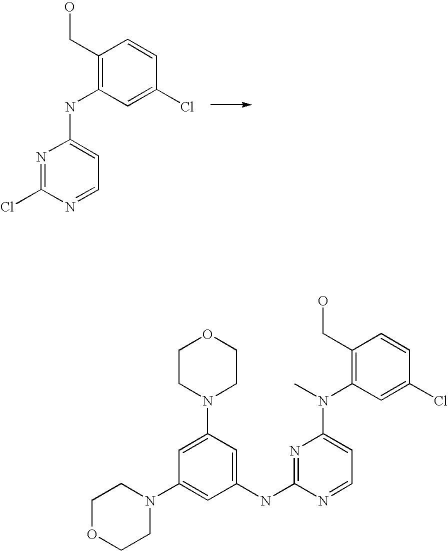

^{h}: A mixture of (4-chloro-2-(2-chloropyrimidin-4-ylamino)phenyl)methanol (35 mg, 0.13 mmol), iodomethane (8.87 μL, 0.14 mmol) and potassium carbonate (26.9 mg, 0.19 mmol) in DMF (1 mL) was stirred at room temperature overnight under nitrogen. The reaction was concentrated to dryness and partitioned between ethyl acetate and water. The aqueous phase was extracted with ethyl acetate and the combined organic phases were washed with a saturated aqueous solution of brine, dried over magnesium sulfate and concentrated to dryness to afford the crude intermediate as a yellow gum.

The above intermediate, 3,5-dimorpholinoaniline (51.2 mg, 0.19 mmol) and hydrogen chloride 5N in iPrOH (3 drops) were suspended in 2-pentanol (1 mL) and sealed into a microwave tube. The reaction was heated to 140° C. for 15 minutes in the microwave reactor. The reaction mixture was concentrated to dryness and partitioned between ethyl acetate and a saturated aqueous solution of sodium hydrogencarbonate. The aqueous phase was extracted with ethyl acetate and the combined organic phases were washed with a saturated aqueous solution of brine, dried over magnesium sulfate and concentrated to dryness to afford the crude product which was purified by flash chromatography on silica gel eluting with 0 to 5% methanol in dichloromethane. The solvent was evaporated to dryness to afford (4-chloro-2-((2-(3,5-dimorpholinophenylamino)pyrimidin-4-yl)(methyl)amino)phenyl)methanol (20.00 mg, 30.2%) as an orange solid.

The (4-chloro-2-(2-chloropyrimidin-4-ylamino)phenyl)methanol used as starting material was made as follows:—

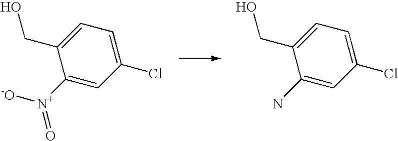

A suspension of (4-chloro-2-nitrophenyl)methanol (1 g, 5.33 mmol, ALDRICH) and platinum(IV) oxide (100 mg, 0.44 mmol) in ethanol (100 mL) was hydrogenated under 1300 mbar at room temperature for 1 hour. The resulting suspension was filtered, washed with ethanol and the filtrate concentrated to dryness to afford the crude (2-amino-4-chlorophenyl)methanol as a pale orange solid. The crude product was purified by flash chromatography on silica gel eluting with 0 to 3% methanol in dichloromethane. The solvent was evaporated to dryness to afford (2-amino-4-chlorophenyl)methanol (0.650 g, 77%) as a pale orange solid.

NMR Spectrum (DMSOd6): 4.34 (d, 2H), 5.06 (t, 1H), 5.20 (s, 2H), 6.51 (dd, 1H), 6.64 (d, 1H), 7.05 (d, 1H)

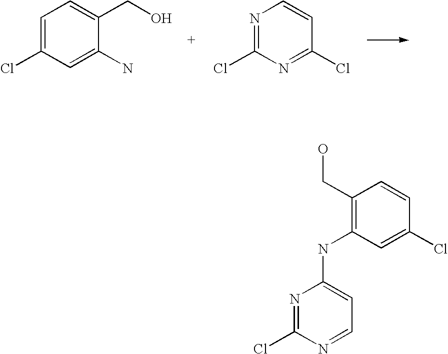

(2-amino-4-chlorophenyl)methanol (100 mg, 0.63 mmol), 2,4-dichloropyrimidine (99 mg, 0.67 mmol) and N-ethyl-N-isopropylpropan-2-amine (DIPEA) (0.221 mL, 1.27 mmol) in 2-pentanol (2 mL) were stirred at 100° C. for 3.5 days. The reaction mixture was concentrated to dryness and partitioned between ethyl acetate and water. The aqueous phase was extracted with ethyl acetate, the combined organic phases were washed with brine, dried over magnesium sulfate and concentrated to afford the crude product as an orange oil. The crude product was purified by flash chromatography on silica gel eluting with 0 to 50% ethyl acetate in dichloromethane. The solvent was evaporated to dryness to afford (4-chloro-2-(2-chloropyrimidin-4-ylamino)phenyl)methanol (38.0 mg, 22%) as a colorless oil which crystallised on standing. Mass Spectrum: M+H⁺ 270.

NMR Spectrum (DMSOd6): 4.46 (s, 2H), 5.36 (bs, 1H), 6.67 (d, 1H), 7.32 (dd, 1H), 7.51 (d, 1H), 7.55 (d, 1H), 8.15 (d, 1H), 9.47 (bs, 1H)

### Method Section

### Method 1

### 2-chloro-N-(3-chloro-2,4-difluoro-phenyl)pyrimidin-4-amine

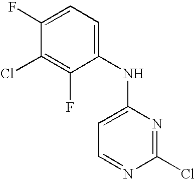

A mixture of 2,4-dichloropyrimidine (6.0 g, 40.5 mmol), 3-chloro-2,4-difluoroaniline (6.28 g, 38.5 mmol), diisopropylethylamine (9.16 ml, 52.7 mmol) and pentanol (20 ml) was refluxed for 24 hrs. After concentration under reduced pressure, the residue was dissolved in ethyl acetate and the solution washed with water, dried and concentrated. The crude material was triturated in methylene chloride and the white solid collected by filtration to give 2.1 g of the desired product. The filtrate was concentrated and purified on silica gel (10 to 50% EtOAc in petroleum ether) to give another 1.9 g of material (total yield 4.0 g, 36%). NMR Spectrum (500 MHz, DMSOd6) 6.80 (d, 1H), 7.38 (ddd, 1H), 7.74 (ddd, 1H), 8.21 (d, 1H), 9.95 (bs, 1H); Mass Spectrum MH⁺ 276.

### 2-chloro-N-(3-chloro-2,4-difluoro-phenyl)-N-methyl-pyrimidin-4-amine

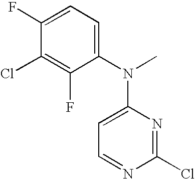

To a suspension of 2-chloro-N-(3-chloro-2,4-difluoro-phenyl)pyrimidin-4-amine (2.45 g, 8.91 mmol) and Cs2CO3 (5.79 g, 17.8 mmol) in 15 mL acetonitrile, iodomethane (0.55 mL, 8.91 mmol) was added dropwise and the mixture was stirred overnight. After evaporation under reduced pressure, the residue was dissolved in methylene chloride and the solution was filtered and evaporated. Purification of the residue on silica gel (10 to 30% EtOAc in petroleum ether) gave 2-chloro-N-(3-chloro-2,4-difluoro-phenyl)-N-methyl-pyrimidin-4-amine (2.20 g, 86%) as an oil which solidified on standing.

NMR Spectrum (500 MHz, DMSOd6) 3.36 (s, 3H), 6.53 (bs, 1H), 7.49 (ddd, 1H), 7.62 (ddd, 1H), 8.15 (bs, 1H); Mass Spectrum MH⁺ 290.

### Method 2

### [3-[(2-chloropyrimidin-4-yl)amino]-4-methyl-phenyl]methanol

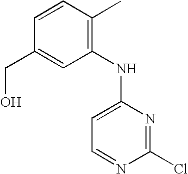

A mixture of 2,4-dichloropyrimidine (4 g, 27 mmol), (3-amino-4-methylphenyl)methanol (3.7 g, 27 mmol), triethylamine (4.13 ml, 29.7 mmol) in ethanol (20 ml) was refluxed for 18 hrs. After concentration under reduced pressure, the residue was dissolved in ethyl acetate and the solution was washed with water, dried and concentrated. The crude material was purified by chromatography on silica gel (eluant: 5% methanol in DCM). After collection of the fractions and evaporation of the solvent, the residue was stirred in DCM (60 ml), filtered and dried under vacuum to give the title compound (2.6 g, 39%).

NMR Spectrum (500 MHz, DMSOd6) 2.15 (s, 3H), 4.46 (d, 2H), 5.18 (t, 1H), 6.44 (m, 1H), 7.14 (d, 1H), 7.22 (s, 1H), 7.25 (d, 1H), 8.07 (d, 1H), 9.55 (bs, 1H); Mass Spectrum MH⁺ 250.

### [3-[(2-chloropyrimidin-4-yl)-methyl-amino]-4-methyl-phenyl]methanol

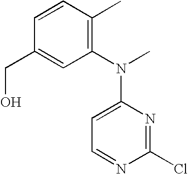

Iodomethane (1.37 ml, 22.1 mmol) was added dropwise to a suspension of [3-[(2-chloropyrimidin-4-yl)amino]-4-methyl-phenyl]methanol (5.0 g, 20.1 mmol) and Cs2CO3 (13.1 g, 40.2 mmol) in DMF (30 ml) at room temperature. The mixture was stirred at room temperature overnight. After evaporation under reduced pressure, the residue was dissolved in DCM and the solution was filtered and evaporated. Purification of the residue by chromatography on silica gel (20 to 100% EtOAc in petroleum ether) gave [3-[(2-chloropyrimidin-4-yl)-methyl-amino]-4-methyl-phenyl]methanol (3.2 g, 61%) as a solid. NMR Spectrum (500 MHz, DMSOd6) 2.06 (s, 3H), 3.32 (s, 3H), 4.50 (d, 2H), 5.25 (t, 1H), 5.80 (d, 1H), 7.20 (s, 1H), 7.31 (m, 1H), 7.38 (m, 1H), 7.94 (d, 1H); Mass Spectrum MH⁺ 264.

### Method 3

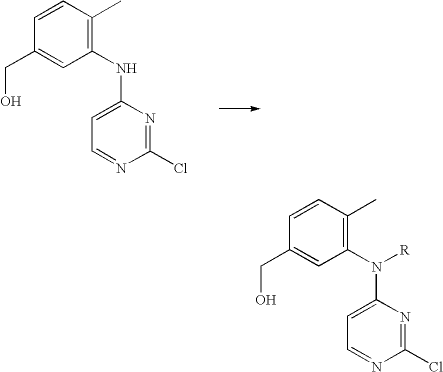

The corresponding bromo derivative (6.02 mmol) was added dropwise to a suspension of [3-[(2-chloropyrimidin-4-yl)amino]-4-methyl-phenyl]methanol (Method 2, 1.5 g, 6.02 mmol), Cs2CO3 (2.94 g, 9.04 mmol) and tetrabutylammonium iodide (100 mg) at room temperature for 48 hrs. After filtration of the solids and evaporation of the filtrate under reduced pressure, the residue was dissolved in DCM and the resulting solution was filtered and evaporated. Purification of the residue by chromatography on silica gel (10 to 50% EtOAc in petroleum ether) gave the desired compound:

| | Starting | | | |
|---|---|---|---|---|
| Name | bromoalkane | yield | MH+ | NMR (500 MHz, DMSOd6) |
| [3-[(2-chloropyrimidin-4- | 2- | 26% | 292 | 0.96 (d, 3H), 1.28 (d, 3H), |
| yl)-propan-2-yl-amino]-4- | bromopropane | | | 2.04 (s, 3H), 4.51 (d, 2H), |
| methyl-phenyl]methanol | | | | 4.97 (m, 1H), 5.26 (t, 1H), |
| | | | | 5.59 (m, 1H), 7.10 (s, 1H), |
| | | | | 7.33 (d, 1H), 7.39 (d, 1H), |
| | | | | 7.91 (m, 1H) |
| [3-[(2-chloropyrimidin-4- | 1-bromo-2- | 37% | 306 | 0.92 (d, 3H), 0.93 (d, 3H), |
| yl)-(2- | methylpropane | | | 1.90 (m, 1H), 2.05 (s, 3H), |
| methylpropyl)amino]-4- | | | | 3.30 (m, 1H), 4.02 (m, 1H), |
| methyl-phenyl]methanol | | | | 4.50 (br d, 2H), 5.25 (br s, |
| | | | | 1H), 5.73 (m, 1H), 7.17 (s, |
| | | | | 1H), 7.30 (d, 1H), 7.38 (d, |
| | | | | 1H), 7.93 (m, 1H) |
| [3-[(2-chloropyrimidin-4- | Bromomethyl- | 71% | 304 | 0.15 (m, 2H), 0.42 (m, 2H), |
| yl)- | cyclopropane | | | 1.05 (m, 1H), 2.09 (s, 3H), |
| (cyclopropylmethyl)amino]- | | | | 3.65 (m, 1H), 3.75 (m, 1H), |
| 4-methyl- | | | | 4.50 (d, 1H), 5.24 (br t, 2H), |
| phenyl]methanol | | | | 5.73 (d, 1H), 7.22 (s, 1H), |
| | | | | 7.30 (d, 1H), 7.37 (d, 1H), |
| | | | | 7.93 (d, 1H) |
| [3-[(2-chloropyrimidin-4- | bromoethane | 57% | 278 | 1.14 (t, 3H), 2.06 (s, 3H), |
| yl)-ethyl-amino]-4-methyl- | | | | 3.65 (m, 1H), 4.04 (m, 1H), |
| phenyl]methanol | | | | 4.50 (d, 2H), 5.24 (br t, 1H), |
| | | | | 5.73 (d, 1H), 7.16 (s, 1H), |
| | | | | 7.32 (d, 1H), 7.39 (d, 1H), |
| | | | | 7.92 (d, 1H) |

### Method 4

### 4-(3-morpholin-4-yl-5-nitro-phenyl)morpholine

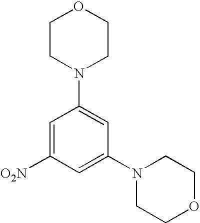

A mixture of 3,5-difluoro-1-nitrobenzene (50 g, 314 mmol) and anhydrous DMSO (25 ml) in morpholine (164 ml, 1.89 mol) was heated at 160° C. for 24 hrs. Additional anhydrous DMSO (12.5 ml) was added and the mixture was heated at 160° C. for 66 hrs more. After cooling, the mixture was diluted in DCM, washed with water and brine and dried over MgSO4. After evaporation of the solvents, the residue was purified by chromatography on silica gel (eluant: 5% EtOAc in DCM) to give 4-(3-morpholin-4-yl-5-nitro-phenyl)morpholine (53.3 g, 58%) as an orange solid.

NMR Spectrum: (DMSOd6) 3.21 (m, 8H), 3.73 (m, 8H), 6.84 (s, 1H), 7.15 (s, 2H); Mass spectrum: MH⁺ 294

### Method 5

### 4-(3-iodo-5-nitro-phenyl)morpholine

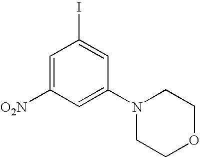

A mixture of 1-fluoro-3-iodo-5-nitrobenzene (0.97 g, 3.63 mmol), morpholine (3.16 ml, 36.3 mmol) and DMSO (3 ml) was heated at 90° C. for 4 hrs. After cooling, water was added and the resulting yellow precipitate was collected by filtration (1.12 g, 93%). NMR Spectrum (500 MHz, DMSOd6) 3.24-3.26 (m, 4H), 3.71-3.73 (m, 4H), 6.64 (s, 1H), 7.67 (s, 1H), 7.83 (s, 1H). Mass spectrum: MH⁺ 335.

### Method 6

### 4-(3-nitro-5-thiomorpholin-4-yl-phenyl)morpholine

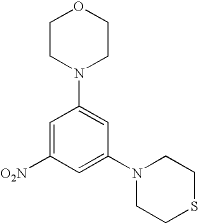

A mixture of 4-(3-iodo-5-nitro-phenyl)morpholine (Method 5, 300 mg, 1.0 mmol), caesium carbonate (1.66 g, 5.1 mmol), Pd(OAc)₂ (11 mg, 0.051 mmol), BINAP (12 mg, 0.02 mmol) and thiomorpholine (205 μL, 2.0 mmol) in toluene degassed with argon (10 mL) was heated at reflux for 3 hrs. After cooling, the solvent was removed under vacuum, the residue was dissolved in ethyl acetate, filtered on celite and the filtrate was washed with water, dried and evaporated. The crude product was purified on silica gel (100% methylene chloride) to yield 140 mg (44%) of a yellow solid. NMR Spectrum (500 MHz, DMSOd6) 2.65-2.66 (m, 4H), 3.19-3.21 (m, 4H), 3.63-3.65 (m, 4H), 3.72-3.74 (m, 4H), 6.80 (s, 1H), 7.10 (s, 1H), 7.12 (s, 1H).

The following compounds were obtained using the same procedure with the appropriate amine instead of morpholine:

| Name | Starting amine | yield | MH+ | NMR(500 MHz, DMSO-d6) |
|---|---|---|---|---|
| 4-[3-(4-methylpiperazin-1-yl)-5-nitro-phenyl]morpholine | 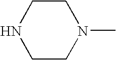 | 75% | 307 | 2.21(s, 3 H), 2.42-2.44(m, 4 H),3.19-3.24(m, 8 H), 3.71-3.74(m,4 H), 6.83(s, 1 H), 7.12(s, 1 H), 7.14(s, 1 H). |
| 4-(3-morpholin-4-yl-5-nitro-phenyl)-1,4-oxazepane | 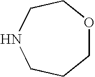 | 52% | 308 | 1.86-1.91(m, 2 H), 3.18-3.20(m,4 H), 3.56-3.66(m, 6 H), 3.71-3.74(m, 6 H), 6.57(s, 1 H), 6.97(s, 2 H). |
| 4-[3-nitro-5-(1-piperidyl)phenyl]morpholine | 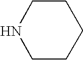 | 64% | 292 | 1.55-1.60(m, 6 H), 3.18-3.20(m,4 H), 3.22-3.24(m, 4 H), 3.71-3.73(m, 4 H), 6.80(s, 1 H), 7.08(s, 1 H),7.12(s, 1 H). |
| 4-(3-nitro-5-pyrrolidin-1-yl-phenyl)morpholine | 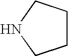 | 73% | 278 | 1.94-1.97(m, 4 H), 3.17-3.19(m,4 H), 3.27-3.29(m, 4 H), 3.72-3.74(m, 4 H), 6.36(s, 1 H), 6.75(s, 1 H),6.97(s, 1 H). |
| (3R)-1-(3-morpholin-4-yl-5-nitro-phenyl)pyrrolidin-3-ol | 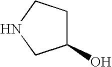 | 10% | 294 | 1.89-1.92(m, 1 H), 1.99-2.05(m,1 H), 3.13(d, 1 H), 3.17-3.19(m, 4 H),3.33-3.39(m, 2 H), 3.46(dd, 1 H),3.72-3.74(m, 4 H), 4.40(bs, 1 H),4.99(d, 1 H), 6.34(s, 1 H), 6.73(s,1 H), 6.97(s, 1 H). |
| (3S)-1-(3-morpholin-4-yl-5-nitro-phenyl)pyrrolidin-3-ol | 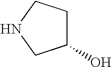 | 20% | 294 | 1.89-1.92(m, 1 H), 1.99-2.05(m,1 H), 3.13(d, 1 H), 3.17-3.19(m, 4 H),3.33-3.39(m, 2 H), 3.46(dd, 1 H),3.72-3.74(m, 4 H), 4.40(bs, 1 H),4.99(d, 1 H), 6.34(s, 1 H), 6.73(s,1 H), 6.97(s, 1 H). |

### Method 7

### 1-iodo-3-(2-methoxyethoxy)-5-nitro-benzene

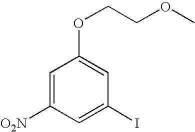

Sodium hydride (60%, 408 mg, 10.2 mmol) was added to a solution of 2-methoxyethanol (2.68 ml, 34 mmol) in DMA (8 ml). After 5 minutes at room temperature, 1-iodo-3,5-dinitrobenzene (2.0 g, 6.8 mmol) was added and the mixture was heated at 100®C for 3 hrs. After cooling, water was added and the resulting mixture was extracted with diethyl ether. The organic phase was dried and evaporated and the residue was purified on silica gel (100% DCM) to yield an orange oil (1.3 g, 59%). NMR Spectrum (500 MHz, DMSOd6) 3.30 (s, 3H), 3.66 (t, 2H), 4.24 (t, 2H), 7.72 (s, 1H), 7.78 (s, 1H), 8.06 (s, 1H).

### Method 8

### 4-(3-methoxy-5-nitro-phenyl)morpholine

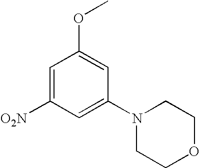

A mixture of 1.0 g (3.4 mmol) 1-iodo-3-methoxy-5-nitrobenzene (J. Med. Chem. 2000, vol. 43, p. 1670-1683), caesium carbonate (5.54 g, 17 mmol), Pd(OAc)₂ (38 mg, 0.17 mmol), BINAP (42 mg, 0.068 mmol) and morpholine (355 μL, 4.1 mmol) in toluene degassed with argon (40 mL) was heated at reflux for 3 hrs. After cooling, the solvent was removed under vacuum, the residue was dissolved in ethyl acetate, filtered on celite and the filtrate was washed with water, dried and evaporated. The crude product was purified on silica gel (100% methylene chloride) to yield 775 mg (96%) of a yellow solid. NMR Spectrum (500 MHz, DMSOd6) 3.21-3.23 (m, 4H), 3.72-3.74 (m, 4H), 3.84 (s, 3H), 6.88 (t, 1H), 7.14 (t, 1H), 7.32 (t, 1H). Mass spectrum: MH⁺ 239.

### 4-[3-(2-methoxyethoxy)-5-nitro-phenyl]morpholine

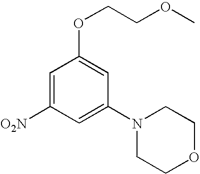

Following the same procedure as above the title compound was prepared from 1-iodo-3-(2-methoxyethoxy)-5-nitro-benzene (Method 7, 71% yield). NMR Spectrum (500 MHz, DMSOd6) 3.21-3.23 (m, 4H), 3.30 (s, 3H), 3.66 (t, 2H), 3.72-3.74 (m, 4H), 4.19 (t, 2H), 6.90 (t, 1H), 7.15 (t, 1H), 7.32 (t, 1H). Mass spectrum: MH⁺ 283.

### Method 9

### 3,5-dimorpholin-4-ylaniline

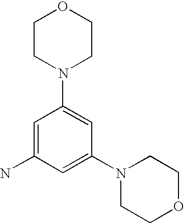

4-(3-Morpholin-4-yl-5-nitro-phenyl)morpholine (Method 4, 53.3 g, 182 mmol) in ethanol (700 ml) was hydrogenated at atmospheric pressure and room temperature in the presence of 10% palladium on charcoal (5 g) for 17 hrs. After filtration of the solids and washing with DMF, the resulting filtrate was concentrated under vacuum. The residue was triturated in ether and dried under vacuum. This solid was solubilised in DCM. The resulting solution was filtered and ether was added. The resulting solid was filtered and dried under high vacuum to give 3,5-dimorpholin-4-ylaniline (46.5 g, 97%) as a beige solid.

NMR Spectrum: (DMSOd6) 2.97 (m, 8H), 3.68 (m, 8H), 4.74 (s, 2H), 5.69 (s, 2H), 5.73 (s, 1H); Mass spectrum: MH⁺ 264

Following the same procedure, the following compounds were obtained:

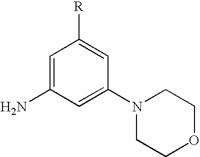

| Name | R | Yield | MH+ | NMR(500 MHz, DMSOd6) |
|---|---|---|---|---|
| 3-morpholin-4-yl-5-thiomorpholin-4-yl-aniline | 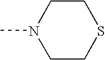 | 34% | 280 | |
| 3-(4-methylpiperazin-1-yl)-5-morpholin-4-yl-aniline | 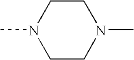 | 100%^{a} | 277 | 2.19(s, 3 H), 2.38-2.40(m, 4 H),2.96-3.01(m, 8 H), 3.67-3.69(m,4 H), 4.71(s, 2 H), 5.66(s, 1 H), 5.69(s, 1 H), 5.72(s, 1 H). |
| 3-morpholin-4-yl-5-(1,4-oxazepan-4-yl)aniline | 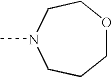 | 100%^{a} | 278 | 1.83-1.88(m, 2 H), 2.95-2.97(m,4 H), 3.43-3.46(m, 4 H), 3.52-3.55(t, 2 H), 3.65-3.69(m, 6 H), 4.63(s,2 H), 5.52(s, 1 H), 5.53(s, 1 H), 5.55(s, 1 H). |
| 3-morpholin-4-yl-5-(1-piperidyl)aniline | 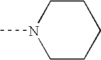 | 100%^{a} | 262 | 1.48-1.51(m, 2 H), 1.54-1.58(m,4 H), 2.94-3.01(m, 8 H), 3.66-3.68(m, 4 H), 4.68(bs, 2 H), 5.64(s, 1 H),5.69(s, 1 H), 5.71(s, 1 H). |
| 3-morpholin-4-yl-5-pyrrolidin-1-yl-aniline | 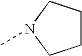 | 94%^{a} | 248 | 1.87-1.91(m, 4 H), 2.96-2.99(m,4 H), 3.12-3.15(m, 4 H), 3.68-3.70(m, 4 H), 4.63(s, 2 H), 5.38(s, 2 H),5.52(s, 1 H). |
| (3R)-1-(3-amino-5-morpholin-4-yl-phenyl)pyrrolidin-3-ol | 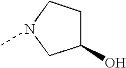 | 74%^{a} | 264 | 1.80-1.84(m, 1 H), 1.95-1.99(m,1 H), 2.95-2.97(m, 5 H), 3.13-3.17(m, 1 H), 3.20-3.24(m, 1 H), 3.29-3.34(m, 1 H partially hidden underH2O), 3.67-3.69(m, 4 H), 4.32-4.34(m, 1 H), 4.63(s, 2 H), 4.85(d, 1 H),5.33(s, 1 H), 5.34(s, 1 H), 5.51(s,1 H). |
| (3S)-1-(3-amino-5-morpholin-4-yl-phenyl)pyrrolidin-3-ol | 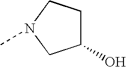 | 59%^{a} | 264 | 1.80-1.84(m, 1 H), 1.95-1.99(m,1 H), 2.95-2.97(m, 5 H), 3.13-3.17(m, 1 H), 3.20-3.24(m, 1 H), 3.29-3.34(m, 1 H partially hidden underH2O), 3.67-3.69(m, 4 H), 4.32-4.34(m, 1 H), 4.63(s, 2 H), 4.85(d, 1 H),5.33(s, 1 H), 5.34(s, 1 H), 5.51(s,1 H). |
| 3-methoxy-5-morpholin-4-yl-aniline | 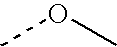 | 100% | 209 | 2.96-2.98(m, 4 H), 3.61(s, 3 H),3.67-3.69(m, 4 H), 4.90(bs, 2 H),5.68(s, 1 H), 5.69(s, 1 H), 5.75(s,1 H). |
| 3-(2-methoxyethoxy)-5-morpholin-4-yl-aniline | 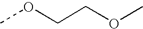 | 100% | 253 | 2.96-2.98(m, 4 H), 3.28(s, 3 H),3.59(dd, 2 H), 3.67-3.69(m, 4 H),3.93(dd, 2 H), 4.88(bs, 2 H), 5.67(s, 1 H), 5.69(s, 1 H), 5.75(s, 1 H). |

^{a}Platinum (IV) oxide was used as a catalyst instead of palladium on charcoal.

### Method 10

### 4-(3-methylsulfonyl-5-nitro-phenyl)morpholine

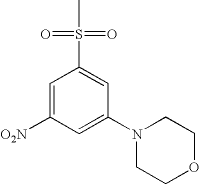

A mixture of 1-fluoro-3-iodo-5-nitrobenzene (8.36 g, 31.3 mmol), copperI iodide (11.9 g, 62.6 mmol) and sodium methanesulfinate (5.65 g, 85% purity, 47 mmol) in DMF (50 ml) was heated at 110° C. overnight. After cooling, the mixture was poured into a mixture of ethyl acetate and water and filtered. The organic layer was separated, dried and concentrated under vacuum. The residue was purified by chromatography on silica gel (eluant: DCM) to give 1-fluoro-3-methylsulfonyl-5-nitrobenzene (4.49 g, 65%) as a pale solid; NMR Spectrum (500 MHz, DMSOd6) 3.42 (s, 3H), 8.33 (m, 1H), 8.52 (m, 2H).

A mixture of 1-fluoro-3-methylsulfonyl-5-nitrobenzene (2.42 g, 15.2 mmol) and morpholine (5.3 ml, 60.9 mmol) in DMSO (5 ml) was heated at 110° C. for 30 minutes. After cooling, water was added. The resulting precipitate was filtered, washed with water and dried under high vacuum to give 4-(3-methylsulfonyl-5-nitro-phenyl)morpholine (2.91 g, 67%) as an orange solid. NMR Spectrum (500 MHz, DMSOd6) 3.30-3.42 (m+s, 7H), 3.77 (m, 4H), 7.77 (m, 1H), 7.94 (m, 2H).

The following compounds were obtained using the same procedure with the appropriate amine instead of morpholine in the second step:

| Name | Starting amine | Yield | MH+ | NMR(500 MHz) |
|---|---|---|---|---|
| 1-methyl-4-(3-methylsulfonyl-5-nitro-phenyl)piperazine | 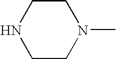 | 67%^{a} | 300 | (DMSO) 2.23(s, 3 H), 2.45-2.48(m,4 H), 3.34(s hidden by H2O, 3 H),3.38-3.40(m, 4 H), 7.75(s, 1 H), 7.90(s, 1 H), 7.92(s, 1 H). |
| 2-[4-(3-methylsulfonyl-5-nitro-phenyl)piperazin-1-yl]ethanol | 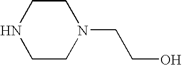 | 83% | 330 | (DMSO-d6 + TFA-d) 3.21-3.35(m,6 H), 3.35(s, 3 H), 3.64(d, 2 H), 3.79(t, 2 H), 4.18(d, 2 H), 7.86(s, 1 H),8.03(s, 1 H), 8.06(s, 1 H). |
| 1-[4-(3-methylsulfonyl-5-nitro-phenyl)piperazin-1-yl]ethanone | 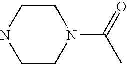 | 73%^{b} | 328 | (DMSO-d6) 2.06(s, 3 H), 3.34(s,hidden by H2O, 3 H), 3.40-3.42(m,2 H), 3.46-3.48(m, 2 H), 3.60-3.62(m, 4 H), 7.76(t, 1 H), 7.93-7.95(m,2 H). |
| 1-(3-methylsulfonyl-5-nitro-phenyl)piperidin-4-ol | 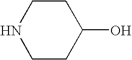 | 75%^{c} | 301 | (CDCl3) 1.70-1.74(m, 2 H), 2.02-2.06(m, 2 H), 3.10(s, 3 H), 3.21-3.26(m, 2 H), 3.71-3.76(m, 2 H), 4.00-4.03(m, 1 H), 7.67(t, 1 H), 7.90(t,1 H), 8.06(t, 1 H). |

^{a}The product was purified on silica gel (0 to 5% MeOH in EtOAc)
^{b}The product was purified on silica gel (0 to 2% MeOH in DCM)
^{c}The product was purified on a preparative HPLC system

### Method 11

### 3-methylsulfonyl-5-morpholin-4-yl-aniline

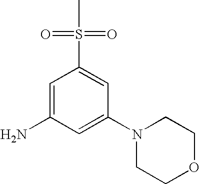

4-(3-Methylsulfonyl-5-nitro-phenyl)morpholine (Method 10, 4.85 g, 16.9 mmol) in ethanol (70 ml) was hydrogenated at a 1.4 bar pressure and room temperature in the presence of 10% palladium on charcoal (1 g) for 3 hrs. After filtration of the solids and washing with DMF, the resulting filtrate was concentrated under vacuum. The residue was purified by chromatography on silica gel (eluant: 50% to 70% EtOAc in DCM) to give 3-methylsulfonyl-5-morpholin-4-yl-aniline (4.12 g, 95%) as a yellow solid.

NMR Spectrum: (DMSOd6) 3.08 (m, 7H), 3.72 (m, 4H), 5.49 (s, 2H), 6.37 (s, 1H), 6.56 (m, 2H); Mass spectrum: MH⁺ 257.

Following the same procedure, the following compounds were obtained:

| Name | Yield | MH+ | NMR (500 MHz) |
|---|---|---|---|
| 3-(4-methylpiperazin-1-yl)-5- | 100%^{a} | 270 | (DMSO) 2.21 (s, 3H), 2.42-2.44 (m, 4H), |
| methylsulfonyl-aniline | | | 3.08 (s, 3H), 3.10-3.12 (m, 4H), 5.45 (s, 2H), |
| | | | 6.37 (s, 1H), 6.52 (s, 1H), 6.55 (s, 1H). |
| 2-[4-(3-amino-5- | 87%^{b} | 300 | (CDCl3) 2.60-2.63 (m, 2H), 2.65-6.7 (m, 4H), |
| methylsulfonyl- | | | 3.02 (s, 3H), 3.24-3.26 (m, 4H), 3.66-3.68 (m, |
| phenyl)piperazin-1-yl]ethanol | | | 2H), 6.37 (s, 1H), 6.68 (s, 1H), 6.85 (s, 1H). |
| 1-[4-(3-amino-5- | 51%^{b} | 298 | (DMSO-d6) 2.03 (s, 3H), 3.08-3.10 (m + s, |
| methylsulfonyl- | | | 5H), 3.14-3.16 (m, 2H), 3.55-3.57 (m, 4H), |
| phenyl)piperazin-1- | | | 5.50 (s, 2H), 6.38 (s, 1H), 6.55 (s, 1H), |
| yl]ethanone | | | 6.57 (s, 1H). |
| 1-(3-amino-5-methylsulfonyl- | 72% | 271 | (DMSO-d6) 1.41-1.44 (m, 2H), 1.77-1.81 (m, |
| phenyl)piperidin-4-ol | | | 2H), 2.82-2.87 (m, 2H), 3.07 (s, 3H), |
| | | | 3.47-3.50 (m, 2H), 3.61-3.64 (m, 1H), 4.69 (d, 1H), |
| | | | 5.42 (s, 2H), 6.37 (t, 1H), 6.47 (t, 1H), 6.54 (t, |
| | | | 1H). |

^{a}Crude yield
^{b}PtO2 was used as the catalyst

### Method 12

### 4-[3-(bromomethyl)-5-nitro-phenyl]morpholine

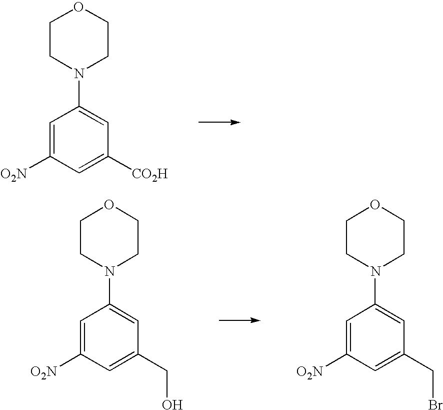

A solution of borane-THF complex in THF (7.14 ml, 1M in THF, 7.14 mmol) was added portionwise to an ice-cooled solution of 3-morpholin-4-yl-5-nitro-benzoic acid (1.2 g, 4.76 mmol; Glaxo Int. Pat. Appl. WO2003101959 Ex 327 part a, p 228) under argon. The mixture was warmed to room temperature, stirred for 18 hrs and, then slowly quenched by addition of methanol. After evaporation of the solvents, the residue was diluted with brine and extracted twice with ethyl acetate. The organic layer was washed with water and brine, and dried over MgSO₄. Evaporation of the solvents afforded (3-morpholin-4-yl-5-nitro-phenyl)methanol (1.1 g, 97%) as a yellow solid. NMR Spectrum (500 MHz, DMSOd6) 3.23 (m, 4H), 3.75 (m, 4H), 4.56 (d, 2H), 5.44 (t, 1H), 7.33 (s, 1H), 7.54 (s, 1H), 7.60 (s, 1H).

Triphenyphosphine (3.96 g, 15.1 mmol) and carbon tetrabromide (5 g, 15.1 mmol) were added to a solution of (3-morpholin-4-yl-5-nitro-phenyl)methanol (1.8 g, 7.56 mmol) in DCM (130 ml) at room temperature. The mixture was stirred at room temperature for 18 hrs. After evaporation of the solvents, the residue was purified by chromatography on silica gel (eluant: DCM) to give 4-[3-(bromomethyl)-5-nitro-phenyl]morpholine (1.6 g, 70%) as a yellow solid. NMR Spectrum (500 MHz, DMSOd6) 3.25 (m, 4H), 3.75 (m, 4H), 4.76 (s, 2H), 7.51 (s, 1H), 7.60 (s, 1H), 7.71 (s, 1H).

### Method 13

### 4-[3-(methoxymethyl)-5-nitro-phenyl]morpholine

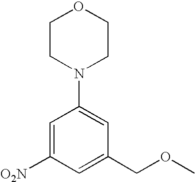

4-[3-(Bromomethyl)-5-nitro-phenyl]morpholine (Method 12, 800 mg, 2.66 mmol) was added to a solution of sodium methoxide (7.44 mmol) in THF [prepared by addition of methanol (0.301 ml, 7.44 mmol) into an ice-cooled suspension of sodium hydride (298 mg, 60% in oil, 7.44 mmol) in THF (15 ml)] at room temperature. The mixture was stirred at room temperature for 18 hrs. After evaporation of the solvents, the residue was diluted with water and extracted with DCM. The organic layer was dried over magnesium sulfate and evaporated under vacuum. The residue was purified by chromatography on silica gel (eluant: 0% to 7% EtOAc in DCM) to give 4-[3-(methoxymethyl)-5-nitro-phenyl]morpholine (582 mg, 87%) as an oil which crystallised on standing. NMR Spectrum (500 MHz, DMSOd6) 3.24 (m, 4H), 3.33 (s, 3H), 3.75 (m, 4H), 4.48 (s, 2H), 7.33 (s, 1H), 7.56 (s, 1H), 7.59 (s, 1H); Mass spectrum: MH⁺ 253.

### 4-[3-nitro-5-(propan-2-yloxymethyl)phenyl]morpholine

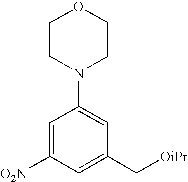

Using the same procedure as above, 4-[3-(bromomethyl)-5-nitro-phenyl]morpholine (700 mg, 2.33 mmol) was reacted with sodium isopropoxide to give 4-[3-nitro-5-(propan-2-yloxymethyl)phenyl]morpholine (375 mg, 58%) as an oil. NMR Spectrum (500 MHz, DMSOd6) 1.16 (d, 6H), 3.23 (m, 4H), 3.65 (m, 1H), 3.75 (m, 4H), 4.52 (s, 2H), 7.33 (s, 1H), 7.57 (s, 2H); Mass spectrum: MH⁺ 281.

### Method 14

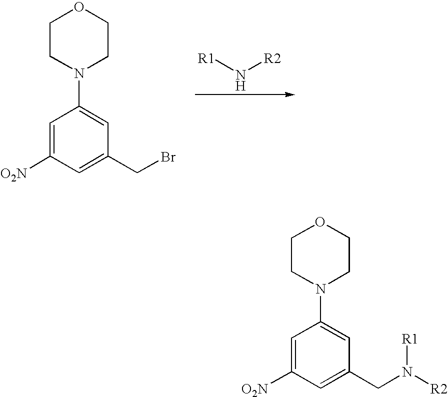

A mixture of 4-[3-(bromomethyl)-5-nitro-phenyl]morpholine (Method 12, 325 mg, 1.08 mmol), the corresponding amine (1.3 mmol), potassium carbonate (298 mg, 2.2 mmol) and tetrabutylammonium iodide (30 mg) in acetonitrile (4 ml) was heated at 45° C. for 3 hrs. After cooling, the mixture was diluted with DCM. The resulting solids were filtered off.

Concentration of the filtrate gave the corresponding amine as solids:

| | | | | NMR(500 MHz, |
|---|---|---|---|---|
| Name | Starting amine | Yield | MH+ | DMSOd6) |
| 4-[3-Nitro-5-(pyrrolidin-1-ylmethyl)phenyl]morpholine | 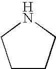 | 99% | 292 | 1.70(m, 4 H), 2.45(m,4 H), 3.22(m, 4 H), 3.63(s, 2 H), 3.75(m, 4 H),7.32(s, 1 H), 7.54(s, 1 H),7.56(s, 1 H) |
| 4-[3-(Morpholin-4-ylmethyl)-5-nitro-phenyl]morpholine | 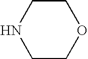 | 100% | 308 | 2.37-2.38(m, 4 H), 3.22-3.24(m, 4 H), 3.53(s,2 H), 3.57-3.59(m, 4 H),3.74-3.76(m, 4 H), 7.33(s, 1 H), 7.55(s, 1 H), 7.58(s, 1 H). |
| 4-[3-[(4-Methylpiperazin-1-yl)methyl]-5-nitro-phenyl]morpholine | 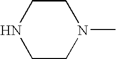 | 100% | 321 | |
| 1-[(3-Morpholin-4-yl-5-nitro-phenyl)methyl]piperidin-4-ol | 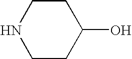 | 99% | 322 | |

### Method 15

### N,N-dimethyl-3-morpholin-4-yl-5-nitro-benzamide

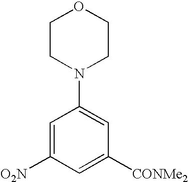

Oxalyl chloride (4.03 ml, 47.6 mmol) followed by DMF (one drop) was added to a suspension of 3-morpholin-4-yl-5-nitro-benzoic acid (800 mg, 3.17 mmol) in DCM (4 ml). The mixture was refluxed for 30 minutes. After cooling, the mixture was concentrated to dryness and diluted in DCM (2 ml) to give a solution of 3-morpholin-4-yl-5-nitro-benzoyl chloride, which was added dropwise to an ice-cooled solution of dimethylamine hydrochloride (517 mg, 6.34 mmol) and diisopropylethylamine (1.66 ml, 9.52 mmol) in DCM (4 ml). After stirring at 0° C. for 20 minutes, the mixture was diluted with water and extracted with DCM. The organic layer was dried over magnesium sulfate and evaporated under vacuum. The residue was purified by chromatography on silica gel (eluant: 0% to 20% EtOAc in DCM) to give N,N-dimethyl-3-morpholin-4-yl-5-nitro-benzamide (847 mg, 96%).

NMR Spectrum (500 MHz, DMSOd6) 2.90 (s, 3H), 2.99 (s, 3H), 3.30 (m, 4H), 3.74 (m, 4H), 7.37 (s, 1H), 7.54 (s, 1H), 7.69 (s, 1H). Mass spectrum: MH⁺ 280.

### Method 16

### 3-morpholin-4-yl-5-nitro-benzonitrile

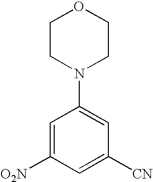

The procedure described in Method 15 was repeated with 3-morpholin-4-yl-5-nitro-benzoic acid (1.5 g, 5.95 mmol) and 7N methanolic ammonia (1.7 ml) instead of dimethylamine hydrochloride to give 3-morpholin-4-yl-5-nitro-benzamide (961 mg, 64%) as a yellow solid, except that the compound was isolated by trituration in a mixture of water and DCM, filtered and dried. NMR Spectrum (500 MHz, DMSOd6) 3.30 (m, 4H), 3.77 (m, 4H), 7.64 (s, 1H), 7.78 (s, 1H), 7.82 (s, 1H), 8.08 (s, 1H), 8.26 (s, 1H); Mass spectrum: MH⁺ 252.

Trichloromethylchloroformate (2.77 ml, 23 mmol) was added dropwise to an ice-cooled mixture of 3-morpholin-4-yl-5-nitro-benzamide (961 mg, 3.83 mmol) in trimethylphosphate (5 ml). The mixture was heated at 60° C. for 24 hrs. After cooling, water was added to the mixture. The resulting precipitate was filtered, washed with water and dried. This solid was purified by chromatography on silica gel (eluant: DCM) to give 3-morpholin-4-yl-5-nitro-benzonitrile (693 mg, 78%). NMR Spectrum (500 MHz, DMSOd6) 3.34 (m, 4H), 3.74 (m, 4H), 7.83 (s, 1H), 7.91 (s, 1H), 7.99 (s, 1H).

### Method 17

### 3-(methoxymethyl)-5-morpholin-4-yl-aniline

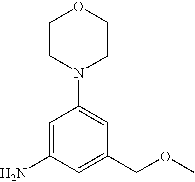

A solution of the 4-[3-(methoxymethyl)-5-nitro-phenyl]morpholine (Method 13, 580 mg, 2.30 mmol) in ethanol (15 ml) was stirred under an atmosphere of hydrogen (atmospheric pressure) in the presence of platinum(IV) oxide (60 mg) at room temperature until absorption of hydrogen stopped. After filtration of the catalyst, evaporation of the solvents gave 3-(methoxymethyl)-5-morpholin-4-yl-aniline (510 mg, 100%) as an oil which crystallised on standing; NMR Spectrum (500 MHz, DMSOd6) 2.99 (m, 4H), 3.23 (s, 3H), 3.70 (m, 4H), 4.19 (s, 2H), 4.89 (m, 2H), 6.05 (m, 3H); Mass spectrum: MH⁺ 223.

The following anilines were obtained using the same procedure:

| | | | NMR(500 MHz, |
|---|---|---|---|
| Name | Structure | MH+ | DMSOd6) |
| (3-amino-5-morpholin-4-yl-phenyl)methanol^{a} | 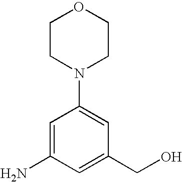 | 209 | 2.97-2.99(m, 4 H), 3.68-3.70(m, 4 H), 4.28(d, 2 H),4.83(s, 2 H), 4.90(t, 1 H),6.01(s, 1 H), 6.05(s, 1 H),6.09(s, 1 H). |
| 3-morpholin-4-yl-5-(propan-2-yloxymethyl)aniline | 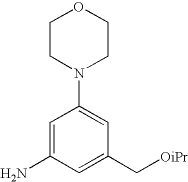 | 251 | 1.11(d, 6 H), 2.98(m, 4 H),3.57(m, 1 H), 3.70(m, 4 H),4.24(s, 2 H), 4.87(m, 2 H),6.05(m, 3 H) |
| 3-morpholin-4-yl-5-(morpholin-4-ylmethyl)aniline | 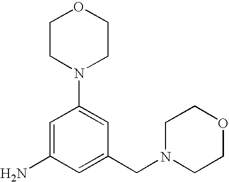 | 278 | 2.36(m, 4 H), 2.98(m, 4 H),3.22(s, 2 H), 3.55(m, 4 H),3.69(m, 4 H), 4.86(m, 2 H),6.02(s, 1 H), 6.07(s, 2 H) |
| 3-morpholin-4-yl-5-(pyrrolidin-1-ylmethyl)aniline | 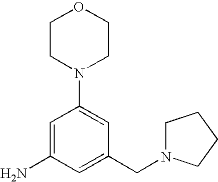 | 262 | 1.67(m, 4 H), 2.41(m, 4 H),2.98(m, 4 H), 3.35(s, 2 H),3.69(m, 4 H), 4.69(m, 2 H),6.02(s, 1 H), 6.07(s, 2 H) |
| 3-[(4-methylpiperazin-1-yl)methyl]-5-morpholin-4-yl-aniline)^{a} | 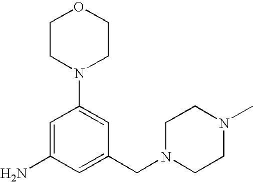 | 291 | |
| 1-[(3-amino-5-morpholin-4-yl-phenyl)methyl]piperidin-4-ol | 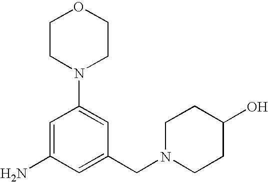 | 292 | |

^{a}Palladium on charcoal was used as a catalyst instead of platinum oxide

### Method 18

### 3-amino-N,N-dimethyl-5-morpholin-4-yl-benzamide

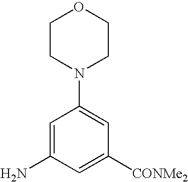

A solution of the N,N-dimethyl-3-morpholin-4-yl-5-nitro-benzamide (Method 15, 833 mg, 2.98 mmol) in ethyl acetate (20 ml)—ethanol (20 ml) was stirred under an atmosphere of hydrogen (40 psi) in the presence of 10% palladium on charcoal (200 mg) at room temperature until absorption of hydrogen has stopped. After filtration of the catalyst, evaporation of the solvents gave 3-amino-N,N-dimethyl-5-morpholin-4-yl-benzamide (510 mg, 100%) as white solid. NMR Spectrum (500 MHz, DMSOd6) 2.50 (s, 3H), 3.01 (s, 3H), 3.34 (m, 4H), 3.70 (m, 4H), 5.07 (m, 2H), 6.01 (s, 1H), 6.06 (s, 1H), 6.17 (s, 1H). Mass spectrum: MH⁺ 250.

### Method 19

### 3-amino-5-morpholin-4-yl-benzonitrile

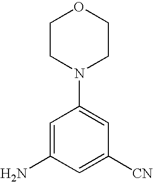

A mixture of 3-morpholin-4-yl-5-nitro-benzonitrile (Method 16, 693 mg, 2.97 mmol), ammonium formate (1.87 g, 29.7 mmol) and 10% palladium on charcoal (150 mg) in THF (15 ml) was heated at reflux for 3 hrs. After cooling and evaporation of the solvents, the mixture was partitioned in an mixture of water and ethyl acetate. After filtration of the catalyst, the organic layer was dried over MgSO₄ and evaporated under vacuum to give 3-amino-5-morpholin-4-yl-benzonitrile (527 mg, 87%) as a white solid.

NMR Spectrum (500 MHz, DMSOd6) 3.05 (m, 4H), 3.70 (m, 4H), 5.41 (m, 2H), 6.33 (s, 1H), 6.39 (s, 1H), 6.49 (s, 1H); Mass spectrum: MH⁺ 204.

### Method 20

### 1-(3-amino-5-morpholin-4-yl-phenyl)piperidin-4-ol

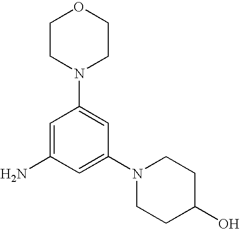

A solution of 3,5-difluoro-nitrobenzene (500 mg, 3.1 mmol) and 4-hydroxypiperidine (317 mg, 3.1 mmol) in DMSO (3 ml) is heated for 2 hrs at 100° C. Morpholine is then added (1.3 ml) and the mixture is heated at 155° C. for 16 hrs. DMSO (1 ml) and morpholine (1 ml) are added and the mixture is further heated at 177° C. for 3 hrs. The reaction mixture was partitioned between diethyl ether and water and the organic layer was washed with a saturated solution of sodium bicarbonate, dried and evaporated. The crude material was purified on silica gel (0 to 100% methylene chloride/ethyl acetate) to give 116 mg (12% yield) of 1-(3-morpholin-4-yl-5-nitro-phenyl)piperidin-4-ol as an orange solid. NMR Spectrum (500 MHz, DMSOd6) 1.42-1.46 (m, 2H), 1.79-1.82 (m, 2H), 2.92-2.98 (m, 2H), 3.18-3.20 (m, 4H), 3.60-3.67 (m, 3H), 3.72-3.74 (m, 4H), 4.69 (d, 1H), 6.82 (s, 1H), 7.08 (s, 1H), 7.13 (s, 1H).; Mass spectrum: MH⁺ 308.

A solution of 1-(3-morpholin-4-yl-5-nitro-phenyl)piperidin-4-ol (106 mg, 0.34 mmol) in ethyl acetate (3 ml) and ethanol (3 ml) was stirred under an atmosphere of hydrogen (50 psi) in the presence of platinum (IV) oxide (80 mg) at room temperature until absorption of hydrogen has stopped. After filtration of the catalyst, evaporation of the solvents gave the title compound (96 mg, 100%). Mass spectrum: MH⁺ 278.

### Method 21

### 4-chloro-N-(3,5-dimorpholin-4-ylphenyl)pyrimidin-2-amine

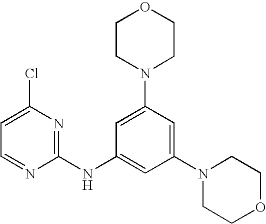

Sodium hydride (60%, 0.99 g, 24.7 mmol) was added portionwise to a ice-cooled solution of N-(3,5-dimorpholin-4-ylphenyl)formamide (4.5 g, 15 mmol) [prepared by heating 3,5-dimorpholin-4-ylaniline (Method 9, 10 g) in formic acid (100 ml) for 3 h at reflux, evaporation of the solvent, partitioning with ethyl acetate/aq. sodium bicarbonate and chromatography on silica gel (1 to 4% MeOH in DCM)] in THF (130 ml). The mixture was stirred at room temperature for 15 minutes, then cooled at 0° C. 4-Chloro-2-methylsulfonylpyrimidine (3.26 g, 17 mmol, L. Xu et al, J. Org. Chem. 2003, 68, 5388) was added portionwise to the mixture. The reaction was warmed to room temperature and stirred overnight. An aqueous solution of sodium hydroxide (2N, 14 ml) and methanol (20 ml) were added and the mixture stirred for 1 hr. After concentration under vacuum, the residue was dissolved in methylene chloride, washed with water, dried and evaporated to provide a beige solid after trituration in diethyl ether (4.2 g, 73%). NMR Spectrum (500 MHz, DMSO) 3.05-3.07 (m, 8H), 3.72-3.73 (m, 8H), 6.19 (s, 1H), 6.88 (s, 2H), 6.91 (d, 1H), 8.41 (d, 1H), 9.73 (s, 1H); Mass Spectrum MH⁺ 376.

### Method 22

### 3-methylaminophenyl)methanol

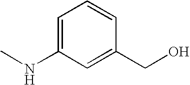

3-aminobenzyl alcohol (1.0 g, 8.1 mmol) was refluxed in formic acid (15 ml) for 2 hrs. Formic acid was removed under vacuum and the residue was dissolved in ethyl acetate and washed with a saturated solution of sodium bicarbonate, then brine, dried and concentrated to give (3-formamidophenyl)methyl formate (1.28 g, 88%) as an oil. Mass Spectrum MH⁺ 180.

Cesium carbonate (1.7 g, 5.3 mmol) was added to a solution of (3-formamidophenyl)methyl formate (0.64 g, 3.5 mmol) in DMF (15 ml) then methyl iodide (0.24 ml, 3.9 mmol) was added and the mixture was stirred at room temperature for 3 hrs. The reaction mixture was concentrated under vacuum and the residue taken in methylene chloride. Filtration of the precipitated solids and concentration of the filtrate provided [3-(formyl-methyl-amino)phenyl]methyl formate as an oil. This product was dissolved in methanol (3 ml) and a 4N aqueous solution of sodium hydroxide was added (3 ml). The reaction mixture was heated at 70° C. for 2 hrs after which the solution was concentrated and neutralized with a saturated solution of ammonium chloride. The aqueous solution was extracted with ether and the organic layer was washed with brine, dried and evaporated to provide (3-methylaminophenyl)methanol as an oil (400 mg, 82% yield over 2 steps). NMR Spectrum (500 MHz, DMSO) 2.65 (d, 3H), 4.37 (d, 2H), 4.99 (t, 1H), 5.54 (d, 1H), 6.38 (d, 1H), 6.47 (d, 1H), 6.50 (s, 1H), 7.00 (t, 1H); Mass Spectrum MH⁺ 138. Similarly were made the following anilines:

| | | | NMR(500 MHz, |
|---|---|---|---|
| Name | Structure | MH+ | DMSOd6) |
| (2-chloro-5-methylamino-phenyl)methanol^{a} | 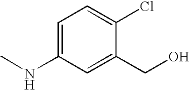 | 172 | 2.65(d, 3 H), 4.45(d,2 H), 5.23(t, 1 H), 5.79(bq, 1 H), 6.39(dd, 1 H),6.74(d, 1 H), 7.04(d, 1 H) |
| (4-chloro-3-methylamino-phenyl)methanol^{a} | 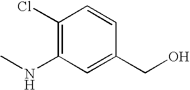 | 172 | 2.75(d, 3 H), 4.41(d,2 H), 5.13(t, 1 H), 5.41(bq, 1 H), 6.52(d, 1 H),6.60(s, 1 H), 7.15(d, 1 H) |
| (3-chloro-5-methylamino-phenyl)methanol^{a} | 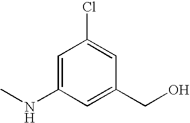 | 172 | 2.65(d, 3 H), 4.36(d,2 H), 5.15(t, 1 H), 5.92(m, 1 H), 6.37(s, 1 H),6.44(s, 1 H), 6.48(s, 1 H) |
| (3-methoxy-5-methylamino-phenyl)methanol^{a} | 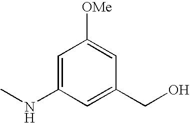 | | 2.63(d, 3 H), 3.66(s, 3 H),5.00(t, 1 H), 5.56(m,1 H), 5.93(s, 1 H), 6.09(s,1 H), 6.12(s, 1 H) |

^{a}the deprotection step was carried out with 5N hydrogen chloride in isopropanol (4 ml) using methanol (20 ml) as the solvent at room temperature.

### Method 23

### 2-chloro-N-(5-methoxy-2-methyl-phenyl)pyrimidin-4-amine

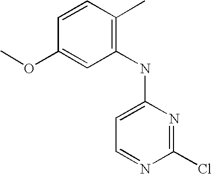

Prepared following the same procedure as in Method 2 using 2-methyl-5-methoxyaniline. The crude product was purified on silica gel (10 to 40% ethyl acetate in petroleum ether) to provide the title compound in 40% yield. NMR Spectrum (500 MHz, DMSOd6) 2.11 (s, 3H), 3.72 (s, 3H), 6.52 (bs, 1H), 6.78 (dd, 1H), 6.95 (s, 1H), 7.19 (d, 1H), 8.08 (d, 1H), 9.49 (bs, 1H).

### 2-chloro-N-(5-methoxy-2-methyl-phenyl)-N-methyl-pyrimidin-4-amine

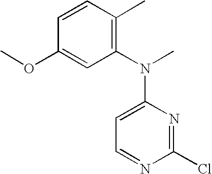

Prepared following the same procedure as in Method 2 using 2-chloro-N-(5-methoxy-2-methyl-phenyl)pyrimidin-4-amine. NMR Spectrum (500 MHz, DMSOd6) 2.00 (s, 3H), 3.30 (s, 3H), 3.74 (s, 3H), 5.84 (d, 1H), 6.91 (s, 1H), 6.95 (d, 1H), 7.33 (d, 1H), 7.93 (d, 1H); Mass Spectrum MH⁺ 264.

### Method 24

### N-[2-[(2-chloropyrimidin-4-yl)amino]-4-methoxy-phenyl]acetamide

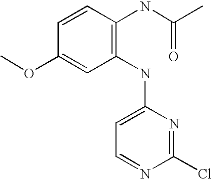

Prepared following the same procedure as in Method 2 using N-(2-amino-4-methoxy-phenyl)acetamide but the reaction was carried out in DMF at 110° C. for 15 hrs. NMR Spectrum (500 MHz, DMSOd6) 1.99 (s, 3H), 3.74 (s, 3H), 6.64 (d, 1H), 6.80 (dd, 1H), 7.13 (s, 1H), 7.40 (d, 1H), 8.12 (d, 1H), 9.24 (s, 1H), 9.34 (s, 1H).

### N-[2-[(2-chloropyrimidin-4-yl)-methyl-amino]-4-methoxy-phenyl]acetamide

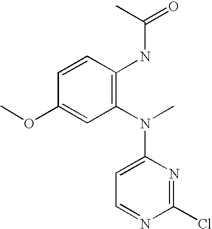

Prepared following the same procedure as in Method 2 using 2-chloro-N-(5-methoxy-2-methyl-phenyl)pyrimidin-4-amine. NMR Spectrum (500 MHz, DMSOd6) 1.91 (s, 3H), 3.27 (s, 3H), 3.74 (s, 3H), 5.96 (bs, 1H), 6.94-6.98 (m, 2H), 7.66 (d, 1H), 7.94 (bs, 1H), 9.25 (bs, 1H); Mass Spectrum MH⁺ 307.

### Method 25

### 4-chloro-N-(3,5-dimorpholin-4-ylphenyl)-N-[(4-methoxyphenyl)methyl]pyrimidin-2-amine

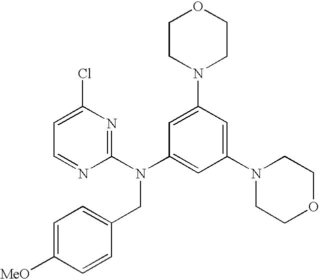

Sodium hydride (1.66 g, 41.6 mmol, 60% in oil) was added portionwise to an ice-cooled solution of 4-chloro-N-(3,5-dimorpholin-4-ylphenyl)pyrimidin-2-amine (13 g, 34.6 mmol) in THF (100 ml). After stirring 2 hrs at room temperature, 4-methoxybenzyl bromide (6.57 ml, 45.1 mmol) and potassium iodide (100 mg) were added to the mixture followed by DMF (10 ml). The resulting mixture was stirred at room temperature for 15 hrs. The mixture was partitioned with saturated aqueous ammonium chloride and ethyl acetate, and further extracted with ethyl acetate. The organic layers were combined and dried over magnesium sulfate. After evaporation of the solvents, the residue was purified by chromatography on silica gel (eluant: 40% to 100% ethyl acetate in petroleum ether) to give the title compound (12.37 g, 72%) as a white solid after trituration in ether/petroleum ether. NMR Spectrum: (DMSOd₆) 3.01 (m, 8H), 3.70 (m, 11H), 5.05 (s, 2H), 6.23 (s, 2H), 6.33 (s, 1H), 6.82 (m, 3H), 7.17 (d, 2H), 8.29 (d, 1H); Mass spectrum: MH⁺ 496.

1. A compound of formula I 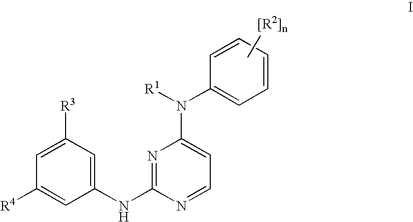
wherein:
R¹ is a (1-4C)alkyl, (3-4C)cycloalkyl or cyclopropylmethyl group which is optionally substituted by one or more substituent groups selected from —OR⁵ (wherein R⁵ is selected from hydrogen or (1-2C)alkyl), cyano, halo, or —NR⁶R⁷ (where R⁶ and R⁷ are independently selected from hydrogen, (1-2C)alkyl or (1-2C)alkanoyl);
n is 0, 1, 2 or 3;
each R² group present is independently selected from (1-2C)alkyl, (1-2C)alkoxy, fluoro, chloro, cyano, hydroxy(1-2C)alkyl, or a group of sub-formula:
-Q-R⁸
where Q is selected from —CO—, —NR^{a}—, —NR^{a}CO—, —NR^{a}—COO—, NR^{a}CONR^{b}, —CONR^{a}—, —S(O)_{z}— (where z is 0, 1 or 2); —SO₂NR^{a}—, and —NR^{a}SO₂—, R^{a} and R^{b} are each independently selected from hydrogen or methyl, and R⁸ is hydrogen or (1-2C)alkyl;
R³ is selected from:
(i) hydrogen, halo, nitro, cyano, or hydroxy;
(ii) an optionally substituted (1-6C)alkyl, (2-6C)alkenyl, or (2-6C)alkynyl group wherein the optional substituents are selected from:
cyano;
halo;
a group of sub-formula:
—W—R⁹
wherein W is selected from —O—, —S(O)ₚ— (where p is 0, 1 or 2), —CO—, —NR^{b}CO—, —CONR^{b}—, —NR^{b}CONR^{b}—, —SO₂NR^{b}—, —NR^{b}SO₂—, or —NR^{b}COO—;
R^{b} is selected from hydrogen or (1-2C)alkyl;
and R⁹ is selected from hydrogen or (1-4C)alkyl;
or —NR¹⁰R¹¹, where R¹⁰ and R¹¹ are independently selected from hydrogen, or a (1-4C)alkyl, (3-6C)cycloalkyl or (3-6C)cycloalkyl(1-2C)alkyl group which is optionally substituted by halo, hydroxy, cyano, or (1-4C)alkoxy, or R¹⁰ and R¹¹ are linked to form a 4, 5, 6 or 7 membered heterocyclic ring which optionally comprises, in addition to the nitrogen atom to which R¹⁰ and R¹¹ are attached, one or two further heteroatoms selected from O, N or S, and wherein any S atoms that are present may be optionally oxidised to form an SO and SO₂ group, and wherein any carbon atom present in the ring is optionally substituted by oxo, halo, hydroxy, cyano, (1-4C)alkyl, hydroxy(1-4C)alkyl, (1-4C)alkoxy, (1-2C)alkoxy-(1-4C)alkyl, (1-4C)alkanoyl, (1-4C)alkanesulfonyl, (1-4C)alkoxycarbonyl, (1-6C)alkylaminocarbonyl or di-(1-6C)alkylaminocarbonyl and any available nitrogen atom present in the ring is optionally substituted by (1-4C)alkyl, hydroxy(1-4C)alkyl, (1-2C)alkoxy-(1-4C)alkyl, or (1-4C)alkanoyl;
(iii) a group —NR¹²R¹³, wherein R¹² and R¹³ are each independently selected from hydrogen or a (1-6C)alkyl (3-6C)cycloalkyl or (3-6C)cycloalkyl(1-2C)alkyl group which is optionally substituted by halo, hydroxy, cyano, or (1-4C)alkoxy, or R¹² and R¹³ are linked to form a 4, 5, 6 or 7-membered heterocyclic ring which optionally comprises, in addition to the nitrogen atom to which R¹² and R¹³ are attached, one or two further heteroatoms selected from O, N or S, and wherein any S atoms that are present may be optionally oxidised to form an SO and SO₂ group, and wherein any carbon atom present in the ring is optionally substituted by oxo, halo, hydroxy, cyano, (1-4C)alkyl, hydroxy(1-4C)alkyl, (1-4C)alkoxy, (1-2C)alkoxy-(1-4C)alkyl, (1-4C)alkanoyl, (1-4C)alkanesulfonyl, (1-4C)alkoxycarbonyl, (1-6C)alkylaminocarbonyl or di-(1-6C)alkylaminocarbonyl and any available nitrogen atom present in the ring is optionally substituted by (1-4C)alkyl, hydroxy(1-4C)alkyl, (1-2C)alkoxy-(1-4C)alkyl, or (1-4C)alkanoyl; or
(iv) a group of formula (II):
—X—R¹⁴
wherein X is selected from —O—, —S(O)ₚ— (where p is 0, 1 or 2), —CO—, —NR^{c}CO—, —CONR^{c}—, —NR^{c}COO—, and —NR^{c}SO₂—,
where R^{c} is selected hydrogen or (1-2C)alkyl;
R¹⁴ is a (1-4C)alkyl, (3-6C)cycloalkyl, (3-6C)cycloalkyl(1-2C)alkyl, oxanyl or oxolanyl group, which is optionally substituted by halo, hydroxy, cyano, or (1-4C)alkoxy, or R¹⁴ is
—NR¹⁵R¹⁶
where R¹⁵ and R¹⁶ are independently selected from hydrogen, (1-2C)alkanoyl or (1-2C)alkyl, or R¹⁵ and R¹⁶ are linked to form a 4, 5, 6 or 7-membered heterocyclic ring which optionally comprises, in addition to the nitrogen atom to which R¹⁵ and R¹⁶ are attached, one or two further heteroatoms selected from O, N or S, and wherein any S atoms that are present may be optionally oxidised to form an SO and SO₂ group, and wherein any carbon atom present in the ring is optionally substituted by oxo, halo, hydroxy, cyano, (1-4C)alkyl, hydroxy(1-4C)alkyl, (1-4C)alkoxy, (1-2C)alkoxy-(1-4C)alkyl, (1-4C)alkanoyl, (1-4C)alkanesulfonyl, (1-4C)alkoxycarbonyl, (1-6C)alkylaminocarbonyl or di-(1-6C)alkylaminocarbonyl and any available nitrogen atom is optionally substituted by (1-4C)alkyl, hydroxy(1-4C)alkyl, (1-2C)alkoxy-(1-4C)alkyl, or (1-4C)alkanoyl;
R⁴ is a group —NR¹⁷R¹⁸, wherein R¹⁷ and R¹⁸ are linked to form a 4, 5, 6 or 7 membered heterocyclic ring which optionally comprises, in addition to the nitrogen atom to which R¹⁷ and R¹⁸ are attached, one or two further heteroatoms selected from O, N or S, and wherein any S atoms that are present may be optionally oxidised to form an SO or SO₂ group, and wherein any carbon atom present in the ring is optionally substituted by oxo, halo, hydroxy, cyano, (1-4C)alkyl, hydroxy(1-4C)alkyl, (1-4C)alkoxy, (1-2C)alkoxy-(1-4C)alkyl, (1-4C)alkanoyl, (1-4C)alkanesulfonyl, (1-4C)alkoxycarbonyl, (1-6C)alkylaminocarbonyl or di-(1-6C)alkylaminocarbonyl and any available nitrogen atom present in the ring is optionally substituted by (1-4C)alkyl, hydroxy(1-4C)alkyl, (1-2C)alkoxy-(1-4C)alkyl, or (1-4C)alkanoyl;
subject to the following provisos:
when n is 1 and R² is (1-2C)alkoxy, the alkoxy group is not located in the para or 4-position relative to the —NR¹— group:
when n is 1 and R² is ethoxy, the ethoxy group is not located in the meta or 3-position relative to the —NR¹— group;
R⁴ is not a 4-methylpiperazin-1-yl group when R² is a group of sub-formula -Q-R⁸, in which Q is —NR^{a}—CO—, R^{a} is hydrogen, and R⁸ is (1-2C)alkyl;
or a pharmaceutically acceptable salt thereof.

2. A compound of formula I 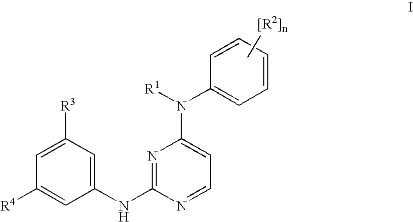
wherein:
R¹ is a (1-4C)alkyl group which is optionally substituted by one or more substituent groups selected from —OR⁵ (wherein R⁵ is selected from hydrogen or (1-2C)alkyl), cyano, halo, or —NR⁶R⁷ (where R⁶ and R⁷ are independently selected from hydrogen, (1-2C)alkyl or (1-2C)alkanoyl);
n is 0, 1, 2 or 3;
each R² group present is independently selected from (1-2C)alkyl, (1-2C)alkoxy, fluoro, chloro, cyano, hydroxy(1-2C)alkyl, or a group of sub-formula:
-Q-R⁸
where Q is selected from —CO—, —NR^{a}—, —NR^{a}—CO—, —NR^{a}—COO—, NR^{a}CONR^{b}, —CONR^{a}—, —S(O)_{z}— (where z is 0, 1 or 2); —SO₂NR^{a}—, and —NR^{a}SO₂—, R^{a} and R^{b} are each independently selected from hydrogen or methyl, and R⁸ is hydrogen or (1-2C)alkyl;
R³ is selected from:
(i) hydrogen, halo, nitro, cyano, or hydroxy;
(ii) an optionally substituted (1-6C)alkyl, (2-6C)alkenyl, or (2-6C)alkynyl group wherein the optional substituents are selected from: cyano; halo; a group of sub-formula:
—W—R⁹
wherein W is selected from —O—, —S(O)ₚ— (where p is 0, 1 or 2), —CO—, —NR^{b}CO—, —CONR^{b}—, —NR^{b}CONR^{b}—, —SO₂NR^{b}—, —NR^{b}SO₂—, or —NR^{b}COO—;
R^{b} is selected from hydrogen or (1-2C)alkyl;
and R⁹ is selected from hydrogen or (1-4C)alkyl;
or —NR¹⁰R¹¹, where R¹⁰ and R¹¹ are independently selected from hydrogen,
or (1-2C)alkyl, or R¹⁰ and R¹¹ are linked to form a 4, 5, 6 or 7 membered heterocyclic ring which optionally comprises, in addition to the nitrogen atom to which R¹⁰ and R¹¹ are attached, one or two further heteroatoms selected from O, N or S, and wherein any S atoms that are present may be optionally oxidised to form an SO and SO₂ group, and wherein any carbon atom present in the ring is optionally substituted by oxo, halo, hydroxy, cyano, (1-4C)alkyl, hydroxy(1-4C)alkyl, (1-4C)alkoxy, (1-2C)alkoxy-(1-4C)alkyl, (1-4C)alkanoyl, (1-4C)alkanesulfonyl, (1-4C)alkoxycarbonyl, (1-6C)alkylaminocarbonyl or di-(1-6C)alkylaminocarbonyl and any available nitrogen atom present in the ring is optionally substituted by (1-4C)alkyl, hydroxy(1-4C)alkyl, (1-2C)alkoxy-(1-4C)alkyl, or (1-4C)alkanoyl;
(iii) a group —NR¹²R¹³, wherein R¹² and R¹³ are each independently selected from hydrogen or (1-6C)alkyl, or R¹² and R¹³ are linked to form a 4, 5, 6 or 7-membered heterocyclic ring which optionally comprises, in addition to the nitrogen atom to which R¹² and R¹³ are attached, one or two further heteroatoms selected from O, N or S, and wherein any S atoms that are present may be optionally oxidised to form an SO and SO₂ group, and wherein any carbon atom present in the ring is optionally substituted by oxo, halo, hydroxy, cyano, (1-4C)alkyl, hydroxy(1-4C)alkyl, (1-4C)alkoxy, (1-2C)alkoxy-(1-4C)alkyl, (1-4C)alkanoyl, (1-4C)alkanesulfonyl, (1-4C)alkoxycarbonyl, (1-6C)alkylaminocarbonyl or di-(1-6C)alkylaminocarbonyl and any available nitrogen atom present in the ring is optionally substituted by (1-4C)alkyl, hydroxy(1-4C)alkyl, (1-2C)alkoxy-(1-4C)alkyl, or (1-4C)alkanoyl; or
(iv) a group of formula (II):
—X—R¹⁴
wherein X is selected from —O—, —S(O)ₚ— (where p is 0, 1 or 2), —CO—, —NR^{c}CO—, —CONR^{c}—, —NR^{c}COO—, and —NR^{c}SO₂—,
where R^{c} is selected hydrogen or (1-2C)alkyl;
R¹⁴ is a (1-4C)alkyl group which is optionally substituted by halo, hydroxy, cyano, (1-4C)alkoxy, or R¹⁴ is
—NR¹⁵R¹⁶
where R¹⁵ and R¹⁶ are independently selected from hydrogen, (1-2C)alkanoyl or (1-2C)alkyl, or R¹⁵ and R¹⁶ are linked to form a 4, 5, 6 or 7-membered heterocyclic ring which optionally comprises, in addition to the nitrogen atom to which R¹⁵ and R¹⁶ are attached, one or two further heteroatoms selected from O, N or S, and wherein any S atoms that are present may be optionally oxidised to form an SO and SO₂ group, and wherein any carbon atom present in the ring is optionally substituted by oxo, halo, hydroxy, cyano, (1-4C)alkyl, hydroxy(1-4C)alkyl, (1-4C)alkoxy, (1-2C)alkoxy-(1-4C)alkyl, (1-4C)alkanoyl, (1-4C)alkanesulfonyl, (1-4C)alkoxycarbonyl, (1-6C)alkylaminocarbonyl or di-(1-6C)alkylaminocarbonyl and any available nitrogen atom is optionally substituted by (1-4C)alkyl, hydroxy(1-4C)alkyl, (1-2C)alkoxy-(1-4C)alkyl, or (1-4C)alkanoyl;
R⁴ is a group —NR¹⁷R¹⁸, wherein R¹⁷ and R¹⁸ are linked to form a 4, 5, 6 or 7 membered heterocyclic ring which optionally comprises, in addition to the nitrogen atom to which R¹⁷ and R¹⁸ are attached, one or two further heteroatoms selected from O, N or S, and wherein any S atoms that are present may be optionally oxidised to form an SO or SO₂ group, and wherein any carbon atom present in the ring is optionally substituted by oxo, halo, hydroxy, cyano, (1-4C)alkyl, hydroxy(1-4C)alkyl, (1-4C)alkoxy, (1-2C)alkoxy-(1-4C)alkyl, (1-4C)alkanoyl, (1-4C)alkanesulfonyl, (1-4C)alkoxycarbonyl, (1-6C)alkylaminocarbonyl or di-(1-6C)alkylaminocarbonyl and any available nitrogen atom present in the ring is optionally substituted by (1-4C)alkyl, hydroxy(1-4C)alkyl, (1-2C)alkoxy-(1-4C)alkyl, or (1-4C)alkanoyl;
subject to the following provisos:
when R² is (1-2C)alkoxy, the alkoxy group is not located in the para or 4-position relative to the —NR¹— group;
R⁴ is not a 4-methylpiperazin-1-yl group when R² is a group of sub-formula -Q-R⁸, in which Q is —NR^{a}—CO—, R^{a} is hydrogen, and R⁸ is (1-2C)alkyl.

3. A compound according to claim 1, or a pharmaceutically acceptable salt thereof, wherein R¹ is a (1-4C)alkyl group which is optionally substituted by one or more substituent groups selected from —OR⁵ (wherein R⁵ is selected from hydrogen or (1-2C)alkyl).

4. A compound according to claim 1, or a pharmaceutically acceptable salt thereof, wherein each R² group present is independently selected from (1-2C)alkyl, (1-2C)alkoxy, fluoro, chloro, cyano, hydroxy(1-2C)alkyl, or a group of sub-formula:
-Q-R⁸
where Q is selected from —CO—, —NR^{a}—CO—, —S(O)_{z}— (where z is 0, 1 or 2); R^{a} is selected from hydrogen or methyl, and R⁸ is hydrogen or (1-2C)alkyl.

5. A compound according to claim 1, or a pharmaceutically acceptable salt thereof, wherein R³ is a group —NR¹²R¹³, wherein R¹² and R¹³ are each independently selected from hydrogen or (1-6C)alkyl, or R¹² and R¹³ are linked to form a 5, 6 or 7-membered heterocyclic ring, and wherein, in addition to the nitrogen atom to which R¹² and R¹³ are attached, the ring optionally comprises one or two further heteroatoms selected from O, N or S, and wherein the ring is optionally substituted on any available carbon atom by one or two substituent groups selected from oxo, halo, hydroxy, cyano, (1-4C)alkyl, or (1-4C)alkanesulfonyl, and any available nitrogen atom present in the ring is optionally substituted by (1-4C)alkyl or (1-4C)alkanoyl.

6. A compound according to claim 1, or a pharmaceutically acceptable salt thereof, wherein R⁴ is a group —NR¹⁷R¹⁸, wherein R¹⁷ and R¹⁸ are linked to form a 6 membered heterocyclic ring which optionally comprises, in addition to the nitrogen atom to which R¹⁷ and R¹⁸ are attached, one or two further heteroatoms selected from O, N or S, and wherein the ring is optionally substituted on any available carbon atom by one or two substituent groups selected from oxo, halo, hydroxy, cyano, or (1-4C)alkyl, and any available nitrogen atom is optionally substituted by (1-4C)alkyl, hydroxy(1-4C)alkyl or (1-4C)alkanoyl.

7. A compound according to claim 1, or a pharmaceutically acceptable salt thereof, which is any one of Examples 1 to 25.

8. A compound according to claim 1 which is [3-[[2-[(3,5-dimorpholin-4-ylphenyl)amino]pyrimidin-4-yl]-methyl-amino]-4-methyl-phenyl]methanol, or a pharmaceutically acceptable salt thereof.

9. A compound according to claim 8, which is:
the freebase form of [3-[[2-[(3,5-dimorpholin-4-ylphenyl)amino]pyrimidin-4-yl]-methyl-amino]-4-methyl-phenyl]methanol;
the besylate salt of [3-[[2-[(3,5-dimorpholin-4-ylphenyl)amino]pyrimidin-4-yl]-methyl-amino]-4-methyl-phenyl]methanol; or
the tosylate salt of [3-[[2-[(3,5-dimorpholin-4-ylphenyl)amino]pyrimidin-4-yl]-methyl-amino]-4-methyl-phenyl]methanol.

10. A compound according to claim 8 in crystalline form.

11. A compound according to claim 10 which is [3-[[2-[(3,5-dimorpholin-4-ylphenyl)amino]pyrimidin-4-yl]-methyl-amino]-4-methyl-phenyl]methanol freebase Form 1 and has an X-ray powder diffraction pattern with specific peaks at about 2θ=7.5, 22.2, 22.7 and 24.7° when measured using CuKa radiation.

12. A compound according to claim 10 which is:
[3-[[2-[(3,5-dimorpholin-4-ylphenyl)amino]pyrimidin-4-yl]-methyl-amino]-4-methyl-phenyl]methanol freebase, Form 1, which has an X-ray powder diffraction pattern with specific peaks at 2θ=7.5, 10.1, 11.9, 12.3, 13.0, 13.6, 15.2, 16.3, 16.6, 17.4, 18.0, 18.6, 19.0, 19.8, 20.2, 20.6, 21.1, 22.2, 22.7, 23.8, 24.2, 24.7, 25.2, 26.2, 26.7, 27.6, 28.1, 28.8, 29.4, 31.5, 32.3, 34.0, 35.6, 36.2, 37.5 and 38.1° when measured using CuKa radiation;
[3-[[2-[(3,5-dimorpholin-4-ylphenyl)amino]pyrimidin-4-yl]-methyl-amino]-4-methyl-phenyl]methanol besylate, Form 1, which has an X-ray powder diffraction pattern with specific peaks at 2θ=5.6, 8.7, 9.5, 10.3, 11.0, 11.3, 12.9, 14.8, 15.1, 15.4, 15.9, 16.3, 17.3, 18.2, 19.0, 19.6, 20.4, 20.8, 21.1, 21.4, 22.1, 23.0, 23.7, 24.3, 24.6, 25.2, 26.1, 26.7, 27.7, 28.5, 30.1, 31.0, 31.9 and 33.8° when measured using CuKa radiation; or
[3-[[2-[(3,5-dimorpholin-4-ylphenyl)amino]pyrimidin-4-yl]-methyl-amino]-4-methyl-phenyl]methanol besylate, Form 2, which has an X-ray powder diffraction pattern with specific peaks at 2θ=5.6, 8.6, 9.8, 11.1, 16.0, 16.7, 17.3, 17.7, 18.6, 20.9, 23.3, 23.9, 26.0 and 27.7° when measured using CuKa radiation.

13. A compound according to claim 10 which is:
[3-[[2-[(3,5-dimorpholin-4-ylphenyl)amino]pyrimidin-4-yl]-methyl-amino]-4-methyl-phenyl]methanol freebase, Form 1, having an X-ray powder diffraction pattern substantially the same as the X-ray powder diffraction pattern shown in Figure A;
[3-[[2-[(3,5-dimorpholin-4-ylphenyl)amino]pyrimidin-4-yl]-methyl-amino]-4-methyl-phenyl]methanol besylate, Form 1, having an X-ray powder diffraction pattern substantially the same as the X-ray powder diffraction pattern shown in Figure B; or
[3-[[2-[(3,5-dimorpholin-4-ylphenyl)amino]pyrimidin-4-yl]-methyl-amino]-4-methyl-phenyl]methanol besylate, Form 2, having an X-ray powder diffraction pattern substantially the same as the X-ray powder diffraction pattern shown in Figure C.

14. A pharmaceutical product which comprises a compound of formula I as defined in claim 1, or a pharmaceutically acceptable salt thereof, and a VEGF receptor tyrosine kinase inhibitor.

15. A pharmaceutical product according to claim 14 which comprises [3-[[2-[(3,5-dimorpholin-4-ylphenyl)amino]pyrimidin-4-yl]-methyl-amino]-4-methyl-phenyl]methanol, or a pharmaceutically acceptable salt thereof, and 4-(4-fluoro-2-methylindol-5-yloxy)-6-methoxy-7-(3-pyrrolidin-1-ylpropoxy)quinazoline, or a pharmaceutically acceptable salt thereof.

16. A pharmaceutical composition which comprises a compound according to claim 1, or a pharmaceutically acceptable salt thereof, in association with a pharmaceutically acceptable diluent or carrier.

17. A compound according to claim 1, or a pharmaceutically acceptable salt thereof, for use as a medicament.

18. A compound according to claim 1, or a pharmaceutically acceptable salt thereof, for use in the treatment of cancer.

19. Use of a compound according to claim 1, or a pharmaceutically acceptable salt thereof, for the manufacture of a medicament for the treatment of cancer.

20. A process for the manufacture of a compound of formula I as defined in claim 1 provided that any functional groups can be optionally protected and L is a suitable leaving group, which comprises the reaction of a compound of formula (VII) 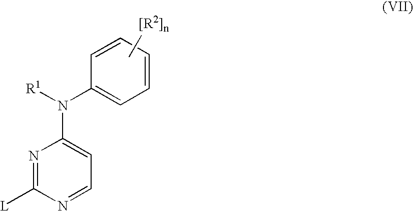
with a compound of formula (VI) 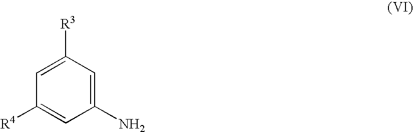
and thereafter, if necessary:
(i) converting a compound of Formula (I) into another compound of Formula (I);
(ii) removing any protecting groups; and/or
(iii) forming a salt thereof.
